(19) United States
(12) Patent Application Publication
(10) Pub. No.: US 2023/0271953 A1
(43) Pub. Date: Aug. 31, 2023

(54) Novel compounds for treatment of diseases related to DUX4 expression

(71) Applicant: Facio Intellectual Property B.V., Leiden (NL)

(72) Inventors: Pui Leng Loke, Abingdon, Oxfordshire (GB); Joris Herman De Maeyer, Mechelen (BE); Robert David Matthew Pace, Abingdon, Oxfordshire (GB); Simon Fletcher Elwood, Abingdon, Oxfordshire (GB); Gregory Foulkes, Abingdon, Oxfordshire (GB); Andrew Anighoro, Reading, Berkshire (GB); Ainoa Rueda-Zubiaurre, Madrid (ES); Jonathan Philip Richards, Abingdon, Oxfordshire (GB); Adam James Davenport, Abingdon, Oxfordshire (GB); Cristina Lecci, Abingdon, Oxfordshire (GB); Anthony Paul Dickie, Abingdon, Oxfordshire (GB); Gerd Schnorrenberg, Biberach (DE)

(73) Assignee: Facio Intellectual Property B.V., Leiden (NL)

(21) Appl. No.: 17/779,137

(22) PCT Filed: Nov. 27, 2020
(86) PCT No.: PCT/EP2020/083803

§ 371 Date: May 24, 2022

(30) Foreign Application Priority Data
Nov. 29, 2019 (EP) 19212729.8

(51) Int. Cl. C07D 471/04; C07D 519/00

(52) U.S. Cl. CPC C07D 471/04; C07D 519/00

(57) ABSTRACT

The present invention relates to compounds that act as DUX4 repressors, suitable for the treatment of diseases related to DUX4 expression, such as muscular dystrophies. It also relates to use of such compounds, or to methods of use of such compounds.

### FIELD OF THE INVENTION

The present invention relates to compounds that act as DUX4 repressors, suitable for the treatment of diseases related to DUX4 expression, such as muscular dystrophies and cancer. It also relates to use of such compounds, or to methods of use of such compounds.

### BACKGROUND ART

Facioscapulohumeral muscular dystrophy (FSHD) is the most prevalent hereditary muscular dystrophy. Symptoms begin before the age of 20, with weakness and atrophy of the muscles around the eyes and mouth, shoulders, upper arms and lower legs. Later, weakness can spread to abdominal muscles and sometimes hip muscles with approximately 20% of patients eventually becoming wheelchair-bound. Patients currently rely on treatment of symptoms like pain and fatigue, involving the use of pain medication, cognitive therapy and physical exercise, sometimes supplemented with medical devices used to maintain the patient’s mobility. Furthermore, increased scapular function may be obtained by surgical treatment of the scapula. At best, these interventions remain symptomatic in nature and do not affect disease progression, illustrating the need for a therapy that is able to modify disease progression.

Significant progress has been made in recent years in the understanding of the molecular basis of FSHD. This resulted in the identification and characterization of the fundamental genetic lesions causing FSHD, giving rise to the pathogenesis model in which gain-of-function of the Double Homeobox 4 (DUX4) retrogene in muscle cells underlies FSHD etiology (Lemmers et al., 2010, DOI: 10.1126/science.1189044; Sharma et al., 2016, DOI:10.4172/2157-7412.1000303, Snider et al., 2010, DOI: 10.1371/journal.pgen.1001181; Tawil et al., 2014, DOl: 10.1186/2044-5040-4-12). DUX4 is a transcription factor that targets several genes and triggers pathology by initiating a transcription deregulation cascade that inhibits myogenesis and causes muscle atrophy, inflammation, and oxidative stress, ultimately resulting in progressive muscle cell dysfunction and death (Kowaljow et al., 2007, DOI: 10.1016/j.nmd.2007.04.002 ; Vanderplanck et al., 2011, doi: 10.1371/journal.pone.0026820 ; Geng et al., 2012, DOI: 10.1016/j.devcel.2011.11.013 ; Yao et al., 2014, DOI: 10.1093/hmg/ddu251 ; Wallace et al., 2011, DOI: 10.1002/ana.22275 ). DUX4 is normally abundantly expressed in germ cells of human testes, while being epigenetically repressed in somatic tissues. The DUX4 gene is located within a DNA tandem array (D4Z4) that is located in the subtelomeric region of chromosome 4q35.

FSHD is sometimes divided in two subtypes, namely FSHD1 and FSHD2. In the majority of patients (FSHD1), the disease is associated with large deletions within the D4Z4 array. Healthy, genetically unaffected individuals are defined as having between 10 and 100 D4Z4 repeat units on both 4q chromosome arms, whereas individuals with FSHD1 have between 1 and 10 D4Z4 repeat units on one 4q chromosome arm. The deletions of D4Z4 repeats that characterize FSHD remove a substantial portion of regulatory chromatin from this region, including several hundreds of histones and a significant amount of CpG-rich DNA. These elements are essential in the establishment of DNA methylation and heterochromatin and their loss significantly alters the epigenetic status of the D4Z4 array leading to derepression of the region. Patients carrying a smaller number of repeats (1-3 units) are on average more severely affected than those with a higher number of repeats (8-9) (Tawil et al., 1996, DOI: 10.1002/ana.410390610). The contraction of D4Z4 is by itself not pathogenic. Only when the contraction of D4Z4 occurs on a disease-permissive 4qA allele, containing a polymorphism that could affect the polyadenylation of the distal DUX4 transcript, the altered epigenetic context is associated with alternative splicing and increased expression of DUX4 in skeletal muscles of FSHD1 patients. In the much rarer form FSHD2, patients manifest similar symptoms, but genetically differ from FSHD1. These patients have longer D4Z4 repeats but exhibit similar derepression of the D4Z4 locus leading to DUX4 expression (Calandra et al., 2016; Jones et al., 2014; 2015). This loss of chromatin repression is caused by mutated forms of an epigenetic factor such as SMCHD1 or DNMT3B. Both forms of FSHD converge on undue DUX4 expression (Van den Boogaard et al., 2016, DOI: 10.1016/j.ajhg.2016.03.013).

In healthy individuals, DUX4 is expressed in the germline, but is epigenetically silenced in somatic tissues. In FSHD patients, burst-like DUX4 expression in only a small fraction of myofibers causes myocyte death ultimately leading to muscle weakness and wasting (Lemmers et al., 2010). In the simplest terms, DUX4-overexpression is a primary pathogenic insult underlying FSHD, and its repression is a promising therapeutic approach for FSHD. In support of this, short repeat sizes are generally associated with a severe FSHD phenotype. Moderate repeat contractions have a milder and more variable clinical severity. Patients with less than 10 D4Z4 repeat units (FSHD1) that also have a mutation in SMCHD1 (FSHD2) have a very severe clinical phenotype, illustrating that a combination of repeat size and activity of epigenetic modifiers, both contributing to derepression of DUX4, determines the eventual disease severity in FSHD.

Because of its causative role in FSHD, suppressing DUX4 is a primary therapeutic approach for halting disease progression. This approach could also be useful for treating other diseases, such as cancers including acute lymphoblastic leukemia (Yasuda et al., 2016, doi: 10.1038/ng.3535) and sarcomas (Oyama et al., 2017 DOI: 10.1038/s41598-017-04967-0 ; Bergerat et al., 2017, DOI: 10.1016/j.prp.2016.11.015), etc. It has recently been shown that DUX4 is also reexpressed in diverse solid cancers. Both cis-acting inherited genetic variation and somatically acquired mutations in trans-acting repressors contribute to DUX4 re-expression in cancer. DUX4-expressing cancers were characterized by reduced markers of anti-tumor cytolytic activity and lower major histocompatibility complex (MHC) class I gene expression. DUX4 expression blocks interferon-y-mediated induction of MHC class I, implicating suppression of antigen presentation and a potential trole of DUX4 in immune evasion of the tumor. Clinical data in metastatic melanoma showed that DUX4 expression was associated with significantly reduced progression-free and overall survival in response to anti-CTLA-4. These data suggest that cancers can escape immune surveillance by reactivating DUX4 expresison and that DUX4-mediated suppression of MHC class I-dependent antigen presentation is a clinically relevant biomarker for response to immune checkpoint blockade. This implies that repression of DUX4 is also a therapeutically relevant approach for several oncology indications and can be an adjuvant treatment to increase responsiveness to immune therapy in oncology (Chew et al., 2019, DOI: 10.1016/j.devcel.2019.06.011).

The mechanisms behind DUX4 expression are poorly understood and corresponding drug targets are poorly defined. As a result, there is no treatment for FSHD at present, and there is a need for compounds and compositions that can be used to suppress DUX4 expression.

### SUMMARY OF THE INVENTION

The invention provides a compound of general formula (I-cyc) or (I):

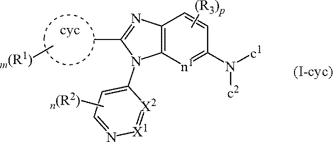

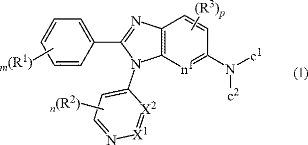

wherein cyc is a phenyl ring, a 5-membered heteroaryl ring, or a 6-membered heteroaryl ring; R¹ is H, halogen, nitrile, —C₁₋₄alkyl, —C₁₋₃alkyl-nitrile, —C₁₋₄haloalkyl, —C₁₋₃haloalkyl-nitrile, —O—C₁₋₄alkyl, —O—C₁₋₃alkyl-nitrile, —O—C₁₋₄haloalkyl, —O—C₁₋₃haloalkyl-nitrile, —S—C₁₋₄alkyl, —S—C₁₋₃alkyl-nitrile, —S—C₁₋ ₄haloalkyl, or —S—C₁₋₃haloalkyl-nitrile; m is 0, 1, 2, or 3; n¹ is N, CH, or C(CH₃); R² is H, halogen, nitrile, —C₁₋₄alkyl, —C₁₋₃alkyl-nitrile, —C₁₋₄haloalkyl, —C₁₋₃haloalkyl-nitrile, —O—C₁₋₄alkyl, —O—C₁₋₃alkyl-nitrile, —O—C₁₋₄haloalkyl, —O—C₁₋₃haloalkyl-nitrile, —S—C₁₋₄alkyl, —S—C₁₋₃alkyl-nitrile, —S—C₁₋₄haloalkyl, —S—C₁₋₃haloalkyl-nitrile, or R² together with Q forms a bridging moiety; n is 0, 1, or 2; R³ is halogen or C₁₋₄alkyl; p is 0, 1, or 2; X¹ is CH, C(R²), N, or C(Q); X² is CH, C(R²), or N; Q is H, halogen, C₁₋ ₆alkyl, —OH, —O—C₁₋₆alkyl, —O—C₁₋₆acyl, —NH₂, —NH—(C₁₋₆alkyl), —N(C₁₋₆alkyl)₂, —NH(C₁₋₈acyl), —N(C₁₋ ₈acyl)₂, —C₁₋₄alkyl—OH, —C₁₋₄alkyl—O—C₁₋₆alkyl, —C₁₋₄alkyl—O—C₁₋₆acyl, —C₁₋₄alkyl—NH₂, —C₁₋₄alkyl—NH—(C₁₋₆alkyl), —C₁₋₄alkyl—N(C₁₋₆alkyl)₂, —C₁₋₄alkyl—NH(C₁₋₈acyl),—C₁₋₄alkyl—N(C₁₋₈acyl)₂, —C₁₋₄alkyl—N—C(O)—NH—C₁₋₆alkyl, —C₁₋₄alkyl—N—C(O)—N(C₁₋₆alkyl)₂, —C₁₋₄alkyl—O—C(O)—NH—C₁₋₆alkyl, —C₁₋₄alkyl—O—C(O)—N(C₁₋₆alkyl)₂, —C₁₋₄alkyl—N—C(O)—O—C₁₋₆alkyl, or Q together with R² forms a bridging moiety selected from —NH—CH═CH—, —NH—(C₂₋₄alkyl)—, and —(C₁₋₃alkyl)—NH—(C₁₋₃alkyl)—; c¹ is H and c² is C₄₋ ₈cycloalkyl, C₄₋₈heterocycloalkyl, C₄₋₈cycloalkyl-C₁₋₃alkyl, C₄₋₈heterocycloalkyl-C₁₋₃alkyl, C₁₋₃alkyl-C₄₋ ₈cycloalkyl, or C₁₋₃alkyl-C₄₋₈heterocycloalkyl, or c¹ and c² together form cyclic structure A; A is a C₅₋ ₁₂cycloalkylthat can be cyclic, bicyclic, and tricyclic, and which is optionally unsaturated, and which is optionally substituted with halogen, C₁₋₄alkyl, C₃₋₆cycloalkyl, C₃₋₆heterocycloalkyl, —O—C₁₋₄alkyl, -SO₂-C₁₋₄alkyl, hydroxyl, —C(═O)—NH₂, —C(═O)—NH(CH₃), —C(═O)—N(CH₃)₂, —NH₂, —NH(C₁₋₄alkyl), or —N(C₁₋₄alkyl)₂; wherein each instance of acyl, alkyl, cycloalkyl, or heterocycloalkyl individually is optionally unsaturated, and optionally substituted with halogen, oxy, hydroxyl, methyl, ethyl, propyl, methoxy, ethoxy, or trifluoromethyl, or optionally interrupted by one or more heteroatoms; or a salt thereof.

Preferably, R¹ is H, fluorine, chlorine, —CH₃, —CF₃, —O—CH₃, or nitrile; m is 0 or 1; n¹ is N or CH; R² is H, fluorine, chlorine, or forms a bridging moiety; n is 0; R³ is —CH₃; p is 0 or 1; X¹ is C(Q); X² is H; Q is H, F, —CH₃, —CH₂F, —CHF₂, —CF₃, —OCH₃, —OCH₂F, —OCHF₂, —OCF₃, —NH—C(O)—CH₃, —NH—C(O)—cyclopropyl, —NH—C(O)—phenyl, —NH—C(O)—halophenyl, —NH—C(O)—piperidinyl, —NH—C(O)—pyridinyl, —NH—C(O)—morpholinyl, —NH—C(O)—oxanyl, —NH₂, —NH(CH₃), -NH(cyclopentyl), —CH₂—NH—C(O)—CH₃, —CH₂—N(CH₃)₂, —CH₂—NH₂, —CH₂—NH—(CH₃), —CH₂—NH—(cyclopentyl), or together with R² forms —NH—CH═CH—; and/or c¹ is H and c² is pyridyl, —CH₂—pyridyl, piperidinyl, N-methylpiperidinyl, —CH₂—piperidinyl, —CH₂—(N—methylpiperidinyl), cyclopentyl, hydroxycyclopentyl, —CH₂—cyclopentyl, —CH₂—hydroxycyclopentyl, pyrrolidinyl, N-methylpyrrolidinyl, —CH₂—pyrrolidinyl, —CH₂—(N—methylpyrrolidinyl), or c¹ and c² together form cyclic structure A. More preferably R¹ is H, fluorine, or chlorine; R² is H or forms a bridging moiety; p is 0; and/or wherein Q is H, —CH₃, —CHF₂, —OCH₃, —NH—C(O)—CH₃, —NH—C(O)—cyclopropyl, —NH—C(O)—phenyl, —NH—C(O)—halophenyl, —NH—C(O)—piperidinyl, —NH—C(O)—pyridinyl, —NH—C(O)—morpholinyl, —NH—C(O)—oxanyl, —NH₂, —CH₂—NH—(CH₃), or together with R² forms —NH—CH═CH—. In preferred embodiments A is optionally substituted and optionally unsaturated azetidinyl, pyrrolidinyl, imidazolidinyl, oxazolidinyl, piperidinyl, piperazinyl, morpholinyl, azacycloheptyl, diazacycloheptyl, or oxoazacycloheptyl; wherein each optional substitution can be a substitution with halogen, C₁₋₆alkyl, C₃₋₆cycloalkyl, C₃₋₆heterocycloalkyl, —O—C₁₋ ₄alkyl, hydroxyl, —NH₂, —NH(C₁₋₄alkyl), or —N(C₁₋₄alkyl)₂; preferably each optional substitution is independently selected from methyl, dimethylamine, methoxyl, propyl, hydroxyl, a bridging C₁₋₃alkyl moiety, spiro azetidinyl, spiro N-methylazetidinyl, spiro oxetanyl, oxetanyl, spiro piperidinyl, difluoropiperidinyl, spiro N-methylpiperidinyl, spiro cyclopropyl, fused pyrrolidinyl, or fused N-methylpyrrolidinyl.

The compounds can be of general formula (I-A-cyc) or (I-A):

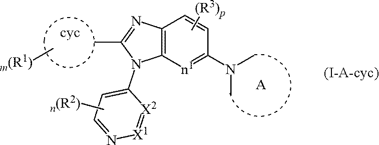

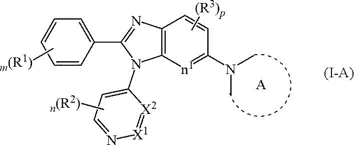

In preferred embodiments the compound is of general formula (II-cyc) or (II), more preferably of general formula (II-A-cyc) or (II-A):

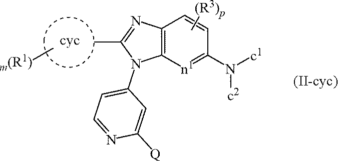

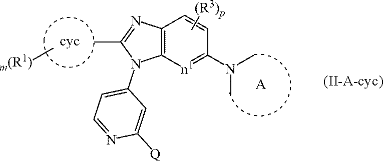

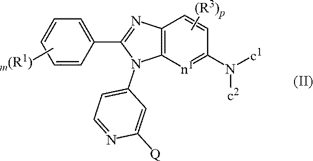

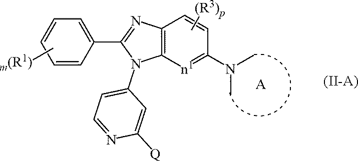

In preferred embodiments the compound is of general formula (III-cyc) or (III), more preferably of general formula (III-A-cyc) or (III-A):

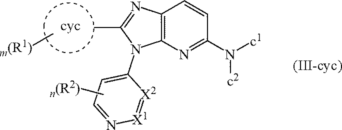

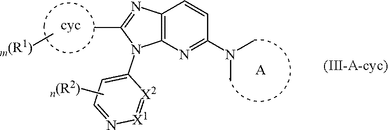

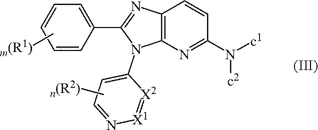

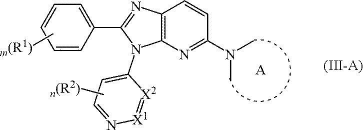

Preferably, A is bicyclic, spiro-cyclic, or bridged, preferably selected from A3-A9, A12, A13, A15-A19, and A22; more preferably it is bicyclic or bridged, even more preferably selected from A3-A6 and A9. Preferably, m is 1 and wherein R¹ is ortho, meta, or para to the bicyclic core of the compound, preferably wherein R¹ is halogen, more preferably fluorine or chlorine, more preferably fluorine. The compound is preferably selected from compounds 1-203 as listed in table 1. More preferably it it is selected from compounds 5, 22, 25, 26, 28, 45, 47, 1, 3, 4, 12, 13, 16, 17, 18, 19, 27, 29, 32, 42, 44, 2, 6, 7, 8, 9, 10, 11, 15, 20, 21, 23, 24, 30, 33, 37, 38, 39, 40, 41, 43, and 46 as listed in table 1; more preferably from compounds 1, 3, 4, 12, 13, 16, 17, 18, 19, 27, 29, 32, 42, 44, 2, 6, 7, 8, 9, 10, 11, 15, 20, 21, 23, 24, 30, 33, 37, 38, 39, 40, 41, 43, and 46; most preferably from compounds 2, 6, 7, 8, 9, 10, 11, 15, 20, 21, 23, 24, 30, 33, 37, 38, 39, 40, 41, 43, and 46.

The invention also provides a composition comprising at least one compound of general formula (I) as defined above, and a pharmaceutically acceptable excipient. The invention also provides the compound or composition as defined above for use as a medicament, wherein the medicament is preferably for use in the treatment of a disease or condition associated with DUX4 expression, and wherein the compound of general formula (I) reduces DUX4 expression, wherein more preferably said disease or condition associated with DUX4 expression is a muscular dystrophy or cancer, even more preferably wherein said disease or condition associated with DUX4 expression is a muscular dystrophy, most preferably facioscapulohumeral muscular dystrophy (FSHD).

The invention also provides an in vivo, in vitro, or ex vivo method for reducing DUX4 expression, the method comprising the step of contacting a cell with a compound of general formula (I) as defined above, or with a composition as defined above. The invention also provides a method for reducing DUX4 expression in a subject in need thereof, the method comprising the step of administering an effective amount of a compound of general formula (I) as defined above, or a composition as defined above.

### DESCRIPTION OF EMBODIMENTS

### Compound

The inventors have identified new compounds that function as DUX4 repressors. The invention provides a compound of general formula (I-cyc) or (I):

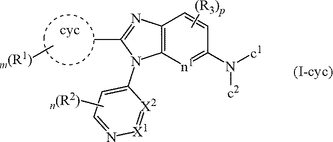

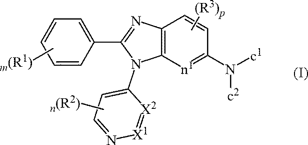

wherein
cyc is a phenyl ring, a 5-membered heteroaryl ring, or a 6-membered heteroaryl ring;
R¹ is H, halogen, nitrile, —C₁₋₄alkyl, —C₁₋₃alkyl-nitrile, —C₁₋₄haloalkyl, —C₁₋₃haloalkyl-nitrile, —O—C₁₋ ₄alkyl, —O—C₁₋₃alkyl-nitrile, —O—C₁₋₄haloalkyl, —O—C₁₋₃haloalkyl-nitrile, —S—C₁₋₄alkyl, —S—C₁₋₃alkyl-nitrile, —S—C₁₋₄haloalkyl, or —S—C₁₋₃haloalkyl-nitrile;
m is 0, 1, 2, or 3;
n¹ is N, CH, or C(CH₃);
R² is H, halogen, nitrile, —C₁₋₄alkyl, —C₁₋₃alkyl-nitrile, —C₁₋₄haloalkyl, —C₁₋₃haloalkyl-nitrile, —O—C₁₋ ₄alkyl, —O—C₁₋₃alkyl-nitrile, —O—C₁₋₄haloalkyl, —O—C₁₋₃haloalkyl-nitrile, —S—C₁₋₄alkyl, —S—C₁₋₃alkyl-nitrile, —S—C₁₋₄haloalkyl, —S—C₁₋₃haloalkyl-nitrile, or R² together with Q forms a bridging moiety;
n is 0, 1, or 2;
R³ is halogen or C₁₋₄alkyl;
p is 0, 1, or 2;
X¹ is CH, C(R²), N, or C(Q);
X² is CH, C(R²), or N;
Q is H, halogen, C₁₋₆alkyl, —OH, —O—C₁₋₈alkyl, —O—C₁₋₈acyl, —NH₂, —NH—(C₁₋₆alkyl), —N(C₁₋₆alkyl)₂, —NH(C₁₋₈acyl), —N(C₁₋₈acyl)₂, —C₁₋₄alkyl—OH, —C₁₋₄alkyl—O—C₁₋₆alkyl, —C₁₋₄alkyl—O—C₁₋₆acyl, —C₁₋ ₄alkyl—NH₂, —C₁₋₄alkyl—NH—(C₁₋₆alkyl), —C₁₋₄alkyl—N(C₁₋₆alkyl)₂, —C₁₋₄alkyl—NH(C₁₋₈acyl), —C₁₋₄alkyl—N(C₁₋₈acyl)₂, —C₁₋₄alkyl—N—C(O)—NH—C₁₋₆alkyl, —C₁₋₄alkyl—N—C(O)—N(C₁₋₆alkyl)₂, —C₁₋₄alkyl—O—C(O)—NH—C₁₋₆alkyl, —C₁—₄alkyl—O—C(O)—N(C₁₋₈alkyl)₂, —C₁₋₄alkyl—N—C(O)—O—C₁₋₆alkyl, or Q together with R² forms a bridging moiety selected from —NH—CH═CH—, —NH—(C₂₋₄alkyl)—, and —(C₁₋₃alkyl)—NH—(C₁₋₃alkyl)—;
c¹ is H and c² is C₄₋₈cycloalkyl, C₄₋₈heterocycloalkyl, C₄₋₈cycloalkyl-C₁₋₃alkyl, C₄₋ ₈heterocycloalkyl-C₁₋₃alkyl, C₁₋₃alkyl-C₄₋₈cycloalkyl, or C₁₋₃alkyl-C₄₋₈heterocycloalkyl, or c¹ and c² together form cyclic structure A;
A is a C₅₋₁₂cycloalkyl that can be cyclic, bicyclic, and tricyclic, and which is optionally unsaturated, and which is optionally substituted with halogen, C₁₋₄alkyl, —O—C₁₋₄alkyl, —SO₂—C₁₋ ₄alkyl, hydroxyl, —C(═O)—NH₂, —C(═O)—NH(CH₃), —C(═O)—N(CH₃)₂, —NH₂, —NH(C₁₋₄alkyl), or —N(C₁₋ ₄alkyl)₂;
wherein each instance of acyl, alkyl, cycloalkyl, or heterocycloalkyl individually is optionally unsaturated, and optionally substituted with halogen, oxy, hydroxyl, methyl, ethyl, propyl, methoxy, ethoxy, or trifluoromethyl, or optionally interrupted by one or more heteroatoms;
or a salt thereof. Such a compound is referred to herein as a compound according to the invention. In preferred embodiments, the compound is a salt, more preferably an acid addition salt, most preferably a pharmaceutically acceptable acid addition salt.

Preferably c¹ and c² together form cyclic structure A. In preferred embodiments a compound of general formula (I-cyc) or (I) is of general formula (I-A-cyc) or (I-A), more preferably (I-A):

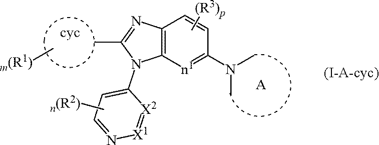

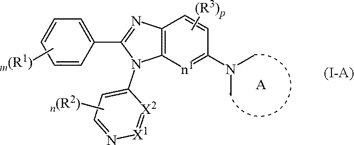

### Bicyclic Core of the Compound

Compounds according to the invention have a central five-membered ring that is fused to a six-membered ring, forming a bicyclic aromatic system that comprises at least two nitrogen atoms. This moiety is referred to hereinafter as the bicyclic core. This core has a variable in n¹, and it is optionally substituted with 0, 1, or 2 instances of R³. The amount of substitution by R³ is denoted by p, which can be 0, 1, or 2. In preferred embodiments, p is 0 or 1. In preferred embodiments, p is 1 or 2. In preferred embodiments, p is 0 or 2. In preferred embodiments, p is 1. In preferred embodiments, p is 2. Most preferably p is 0.

R³ is a substituent that is halogen or C₁₋₄alkyl. This C₁₋₄alkyl is preferably methyl, isopropyl, ethyl, or tert-butyl. More preferably it is C₁₋₃alkyl, even more preferably C₁₋₂alkyl, most preferably it is methyl. As a halogen it is preferably fluoride or chloride, most preferably fluoride. In particular embodiments, R³ is methyl or F.

In preferred embodiments, instances of alkyl within R³ are not unsaturated. In preferred embodiments, instances of alkyl within R³ are optionally unsaturated. In preferred embodiments, instances of alkyl within R³ are unsaturated. In preferred embodiments, instances of alkyl within R³ are not substituted with halogen, oxy, hydroxyl, methyl, ethyl, propyl, methoxy, ethoxy, trifluoromethyl, and not optionally interrupted by one or more heteroatoms. In preferred embodiments, instances of alkyl within R³ are optionally substituted with halogen, oxy, hydroxyl, methyl, ethyl, propyl, methoxy, ethoxy, trifluoromethyl, and not interrupted by one or more heteroatoms. In preferred embodiments, instances of alkyl within R³ are optionally substituted with halogen, oxy, hydroxyl, methyl, ethyl, propyl, methoxy, ethoxy, trifluoromethyl, and/or optionally interrupted by one or more heteroatoms, and/or optionally unsaturated.

n¹ is N, CH, or C(CH₃). In some embodiments, n¹ is N or C(CH₃). In some embodiments, n¹ is CH or C(CH3). In preferred embodiments, n¹ is N or CH. In other preferred embodiments, n¹ is C(CH₃). In other preferred embodiments, n¹ is CH. Most preferably n¹ is N. Preferably, when R³ is present, n¹ is CH or C(CH₃), preferably CH. Preferably, when no R³ is present, n¹ is N.

In preferred embodiments the bicyclic core of the compound is as shown below (reference name shown below the structures). BC-1BC7 are preferred, BC1-BC4 are particularly preferred, BC1, BC2, and BC4 are even more preferred, BC1 is most preferred.

| | | | |
|---|---|---|---|
| 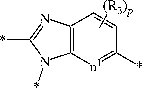 | 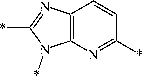 [SMILES: *c1ccc2nc(*)n(*)c2n1] | 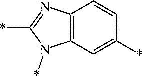 [SMILES: *c1ccc2nc(*)n(*)c2c1] | 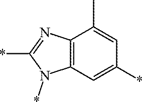 [SMILES: *c1cc(C)c2nc(*)n(*)c2c1] |
| Orientation of cores | BC1 | BC2 | BC3 |
| 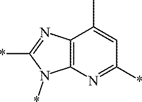 [SMILES: *c1cc(C)c2nc(*)n(*)c2n1] | 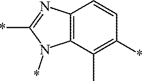 [SMILES: *c1ccc2nc(*)n(*)c2c1C] | 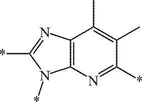 [SMILES: *c1nc2c(nc(*)n2*)c(C)c1C] | 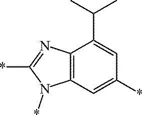 [SMILES: *c1cc(C(C)C)c2nc(*)n(*)c2c1] |
| BC4 | BC5 | BC6 | BC7 |
| 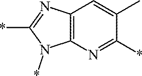 [SMILES: *c1nc2c(cc1C)nc(*)n2*] | 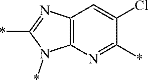 [SMILES: *c1nc2c(cc1Cl)nc(*)n2*] | 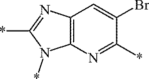 [SMILES: *c1nc2c(cc1Br)nc(*)n2*] | 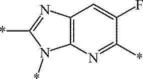 [SMILES: *c1nc2c(cc1F)nc(*)n2*] |
| BC8 | BC9 | BC10 | BC11 |

Compounds of general formula (I-cyc) or (I) are preferably of generally formula (III-cyc) or (III), more preferably of general formula (III-A-cyc) or (III-A), most preferably (III-A):

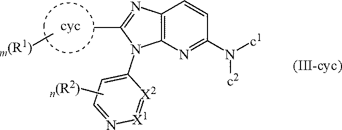

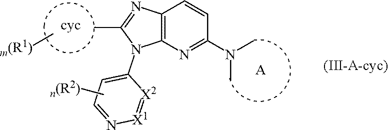

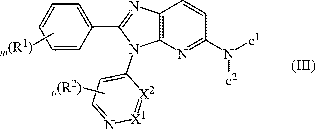

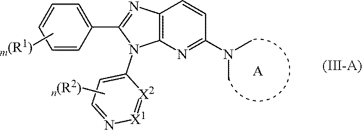

### C-Bonded Ring Moiety of the Compound

The compounds have a phenylic, 5-membered heteroarylic or 6-membered heteroarylic moiety that is attached to the carbon that separates the two nitrogen atoms in the five-membered part of the bicyclic core of compounds according to the invention. It is substituted with 0, 1, 2, or 3 instances of R¹. This moiety is herein referred to as the C-bonded ring moiety. If the C-bonded ring moiety is a (substituted) phenyl group, the C-bonded ring moiety may also be referred to as the phenylic moiety of the compound. The amount of substitution by R¹ is denoted by m, which can be 0, 1, 2, or 3. In preferred embodiments, m is 0, 1, or 2. In preferred embodiments, m is 1, 2, or 3. In preferred embodiments, m is 1 or 2. In preferred embodiments, m is 0. In preferred embodiments, m is 1. In preferred embodiments, m is 2. In preferred embodiments, m is 3. Most preferably m is 0 or 1.

cyc is a phenyl ring, a 5-membered heteroaryl ring, or a 6-membered heteroaryl ring. A 5-membered heteroaryl ring may be any aromatic 5-membered organic ring comprising an endocyclic heteroatom, wherein said heteroatom is preferably selected from the group consisting of nitrogen, oxygen and sulfur. In a preferred embodiment, a 5-membered heteroaryl ring is a pyrrole, imidazole, pyrazole, furan, oxazole, isoxazole, thiophene, thiazole or isothiazole. In a more preferred embodiment, a 5-membered ring is thiophene or thiazole. A 6-membered heteroaryl ring may be any aromatic 6-membered organic ring comprising an endocyclic heteroatom, wherein said heteroatom is preferably selected from the group consisting of nitrogen, oxygen and sulfur. In a preferred embodiment, a 6-membered heteroaryl ring is a pyridine, pyridazine, pyrimidine, pyrazine or pyrylium. In a more preferred embodiment, a 6-membered heteroaryl ring is a pyridine. A 5-membered heteroaryl ring is preferably 2-linked to the core of compounds of the invention. A 6-membered heteroaryl ring is preferably 2- or 3-linked to the core of compounds of the invention.

In preferred embodiments, cyc is 2-pyridinyl, 3-pyridinyl, 4-pyridinyl, 2-thiophenyl or 2-thiazolyl. In more preferred embodiments, cyc is 2-pyridinyl, 3-pyridinyl or 4-pyridinyl. In more preferred embodiments, cyc is 2-thiophenyl or 2-thiazolyl.

R¹ is a substituent that is H, halogen, nitrile, —C₁₋₄alkyl, —C₁₋₃alkyl-nitrile, —C₁₋₄haloalkyl, —C₁₋ shaloalkyl-nitrile, —O—C₁₋₄alkyl, —O—C₁₋₃alkyl-nitrile, —O—C₁₋₄haloalkyl, —O—C₁₋₃haloalkyl-nitrile, —S—C₁₋ ₄alkyl, —S—C₁₋₃alkyl-nitrile, —S—C₁₋₄haloalkyl, or —S—C₁₋₃haloalkyl-nitrile; preferably it is H, halogen, -C₁₋ ₄alkyl, —C₁₋₄haloalkyl, —O—C₁₋₄alkyl, —O—C₁₋₄haloalkyl, —S—C₁₋₄alkyl, or —S—C₁₋₄haloalkyl; in preferred embodiments R¹ is H, fluorine, chlorine, —CH₃, —CF₃, —O—CH₃, or nitrile; more preferably it is H, fluorine, chlorine, —CH₃, —CF₃, or —O—CH₃. Here, —C₁₋₄alkyl and —C₁₋₄haloalkyl are preferably —C₁₋₃alkyl or C₁₋₃haloalkyl, more preferably C₁ variants or isopropyl, most preferably C₁ variants.

In preferred embodiments, R¹ is halogen, —C₁₋₄alkyl, —C₁₋₄haloalkyl, —O—C₁₋₄alkyl, —O—C₁₋ ₄haloalkyl, —S—C₁₋₄alkyl, or —S—C₁₋₄haloalkyl. In preferred embodiments, R¹ is H, -C₁₋₄alkyl, —C₁₋ ₄haloalkyl, —O—C₁₋₄alkyl, —O—C₁₋₄haloalkyl, —S—Cₗ₋₄alkyl, or —S—C₁₋₄haloalkyl. In preferred embodiments, R¹ is H, halogen, —O—C₁₋₄alkyl, —O—C₁₋₄haloalkyl, —S—C₁₋₄alkyl, or —S—C₁₋₄haloalkyl. In preferred embodiments, R¹ is H, halogen, —Cₗ₋₄alkyl, —C₁₋₄haloalkyl, —S—C₁₋₄alkyl, or —S—C₁₋₄haloalkyl. In preferred embodiments, R¹ is H, halogen, —C₁₋₄alkyl, -C₁₋₄haloalkyl, —O—C₁₋₄alkyl, or —O—C₁₋₄haloalkyl.

When m is not 0, the C-bonded ring moiety has at least one R¹. When R¹ is present, it is preferably meta or para to the bicyclic core. In preferred embodiments it is ortho to the bicyclic core. In preferred embodiments it is meta to the bicyclic core. In preferred embodiments it is para to the bicyclic core. In preferred embodiments it is ortho or meta to the bicyclic core. In preferred embodiments it is ortho or para to the bicyclic core. Most preferably a single R¹ is para to the bicyclic core when present. In preferred embodiments m is 1 and R¹ is ortho, meta, or para to the bicyclic core of the compound, preferably herein R¹ is halogen, more preferably fluorine or chlorine, preferably fluorine.In preferred embodiments is provided the compound according to the invention, wherein m is 1, and wherein R¹ is para to the bicyclic core, preferably wherein R¹ is halogen, more preferably fluorine.

In preferred embodiments the C-bonded ring moiety is a phenylic moiety. A compound according to these embodiments may be represented by general structure (I). In more preferred embodiments the phenylic moiety of the compound represented by general structure (I) is as shown below, with a reference name shown below each structure. Ph1-Ph9 and Ph10-Ph19 are particularly preferred, Ph1-Ph9 and Ph17 are more preferred, Ph1-Ph8 and Ph17 are even more preferred, Ph4, Ph6, Ph8, and Ph17 are greatly preferred, Ph6, Ph8, and Ph17 are even more preferred. In some highly preferred embodiments Ph is Ph6. In some highly preferred embodiments Ph is Ph8. In some highly preferred embodiments Ph is Ph17.

| | | | | |
|---|---|---|---|---|
| 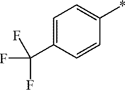 [SMILES: *c1ccc(C(F)(F)F)cc1] | 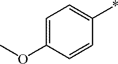 [SMILES: *c1ccc(OC)cc1] | 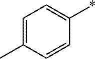 [SMILES: *c1ccc(C)cc1] | 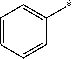 [SMILES: *c1ccccc1] | 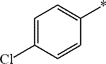 [SMILES: *c1ccc(Cl)cc1] |
| Ph1 | Ph2 | Ph3 | Ph4 | Ph5 |
| 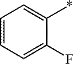 [SMILES: *c1ccccc1F] | 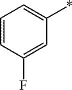 [SMILES: *c1cccc(F)c1] | 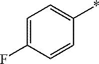 [SMILES: *c1ccc(F)cc1] | 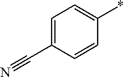 [SMILES: *c1ccc(C#N)cc1] | 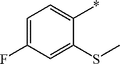 [SMILES: *c1ccc(F)cc1SC] |
| Ph6 | Ph7 | Ph8 | Ph9 | Ph10 |
| 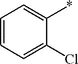 [SMILES: *c1ccccc1Cl] | 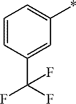 [SMILES: *c1cccc(C(F)(F)F)c1] | 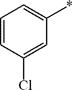 [SMILES: *c1cccc(Cl)c1] | 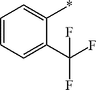 [SMILES: *c1ccccc1C(F)(F)F] | 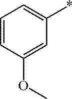 [SMILES: *c1cccc(OC)c1] |
| Ph11 | Ph12 | Ph13 | Ph14 | Ph15 |
| 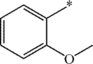 [SMILES: *c1ccccc1OC] | 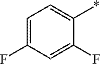 [SMILES: *c1ccc(F)cc1F] | 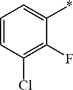 [SMILES: *c1cccc(Cl)c1F] | 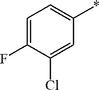 [SMILES: *c1ccc(F)c(Cl)c1] | 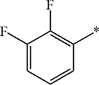 [SMILES: *c1cccc(F)c1F] |
| Ph16 | Ph17 | Ph18 | Ph19 | Ph20 |
| 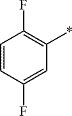 [SMILES: *c1cc(F)ccc1F] | 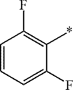 [SMILES: *c1c(F)cccc1F] | 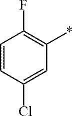 [SMILES: *c1cc(Cl)ccc1F] | 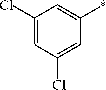 [SMILES: *c1cc(Cl)cc(Cl)c1] | 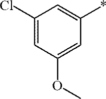 [SMILES: *c1cc(Cl)cc(OC)c1] |
| Ph21 | Ph22 | Ph23 | Ph24 | Ph25 |
| 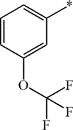 [SMILES: *c1cccc(OC(F)(F)F)c1] | 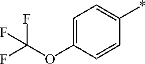 [SMILES: *c1ccc(OC(F)(F)F)cc1] | 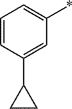 [SMILES: *c1cccc(C2CC2)c1] | 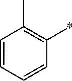 [SMILES: *c1ccccc1C] | 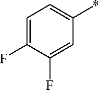 [SMILES: *c1ccc(F)c(F)c1] |
| Ph26 | Ph27 | Ph28 | Ph29 | Ph30 |
| 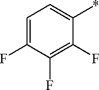 [SMILES: *c1ccc(F)c(F)c1F] | 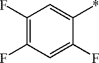 [SMILES: *c1cc(F)c(F)cc1F] | 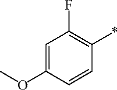 [SMILES: *c1ccc(OC)cc1F] | 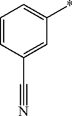 [SMILES: *c1cccc(C#N)c1] | |
| Ph31 | Ph32 | Ph33 | Ph34 | |

In preferred embodiments, the C-bonded ring moiety is a 5-membered heteroaryl ring or a 6-membered heteroaryl ring. In more preferred embodiments the C-bonded ring moiety is as shown below, with a reference name shown below each structure. In more preferred embodiments, the C-bonded ring moiety is Ph35, Ph36, Ph41, Ph42 or Ph43. In more preferred embodiments, the C-bonded ring moiety is Ph37, Ph38, Ph39 or Ph40.

| | | | | |
|---|---|---|---|---|
| 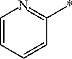 [SMILES: *c1ccccn1] | 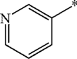 [SMILES: *c1cccnc1] | 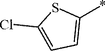 [SMILES: *c1ccc(Cl)s1] | 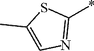 [SMILES: *c1ncc(C)s1] | 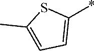 [SMILES: *c1ccc(C)s1] |
| Ph35 | Ph36 | Ph37 | Ph38 | Ph39 |
| 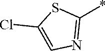 [SMILES: *c1ncc(Cl)s1] | 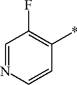 [SMILES: *c1ccncc1F] | 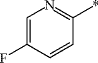 [SMILES: *c1ccc(F)cn1] | 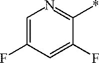 [SMILES: *c1ncc(F)cc1F] | |
| Ph40 | Ph41 | Ph42 | Ph43 | |

In preferred embodiments, the C-bonded ring moiety is selected from the group Ph1-Ph43.

In preferred embodiments, instances of alkyl or haloalkyl within R¹ are not unsaturated. In preferred embodiments, instances of alkyl or haloalkyl within R¹ are optionally unsaturated. In preferred embodiments, instances of alkyl or haloalkyl within R¹ are unsaturated. In preferred embodiments, instances of alkyl or haloalkyl within R¹ are not substituted with halogen, oxy, hydroxyl, methyl, ethyl, propyl, methoxy, ethoxy, trifluoromethyl, and not optionally interrupted by one or more heteroatoms. In preferred embodiments, instances of alkyl or haloalkyl within R¹ are optionally substituted with halogen, oxy, hydroxyl, methyl, ethyl, propyl, methoxy, ethoxy, trifluoromethyl, and not interrupted by one or more heteroatoms. In preferred embodiments, instances of alkyl or haloalkyl within R¹ are optionally substituted with halogen, oxy, hydroxyl, methyl, ethyl, propyl, methoxy, ethoxy, trifluoromethyl, and/or optionally interrupted by one or more heteroatoms, and/or optionally unsaturated.

### Pyridinic Moiety of the Compound

Compounds according to the invention have a pyridinyl-like moiety that is attached to a nitrogen atom of the bicyclic core of the compound according to the invention. It is substituted with 0, 1, or 2 instances of R². It is to be understood that this does not encompass R² when it is comprised in X¹ or X². This aromatic heterocycle is herein referred to as the pyridinic moiety. An amount of substitution by R² is denoted by n, which can be 0, 1, or 2. In preferred embodiments, n is 0 or 1. In preferred embodiments, n is 1 or 2. In preferred embodiments, n is 1. In preferred embodiments, n is 2. Most preferably n is 0. When n is 0, R² can still be present in X¹ or X².

When n is not 0, the pyridinic moiety has at least one R². When such an R² is present, it is ortho or meta to the bicyclic core. In preferred embodiments it is ortho to the bicyclic core. In preferred embodiments it is meta to the bicyclic core.

R² is a substituent that is H, halogen, nitrile, —C₁₋₄alkyl, —C₁₋₃alkyl-nitrile, —C₁₋₄haloalkyl, —C₁₋ shaloalkyl-nitrile, —O—C₁₋₄alkyl, —O—C₁₋₃alkyl-nitrile, —O—C₁₋₄haloalkyl, —O—C₁₋₃haloalkyl-nitrile, —S—C₁₋ ₄alkyl, —S—C₁₋₃alkyl-nitrile, —S—C₁₋₄haloalkyl, —S—C₁₋₃haloalkyl-nitrile, or R² together with Q forms a bridging moiety; preferably it is H, halogen, —C₁₋₄alkyl, —C₁₋₄haloalkyl, —O—C₁₋₄alkyl, —O—C₁₋₄haloalkyl, —S—C₁₋₄alkyl, —S—C₁₋₄haloalkyl, or R² together with Q forms a bridging moiety; in preferred embodiments R² is H, fluorine, chlorine, or together with Q forms a bridging moiety; more preferably it is H, fluorine, or chlorine. Here, —C₁₋₄alkyl and —C₁₋₄haloalkyl are preferably —C₁₋₃alkyl or C₁₋ shaloalkyl, more preferably C₁ variants or isopropyl, most preferably C₁ variants.

In preferred embodiments, instances of alkyl or haloalkyl within R² are not unsaturated. In preferred embodiments, instances of alkyl or haloalkyl within R² are optionally unsaturated. In preferred embodiments, instances of alkyl or haloalkyl within R² are unsaturated. In preferred embodiments, instances of alkyl or haloalkyl within R² are not substituted with halogen, oxy, hydroxyl, methyl, ethyl, propyl, methoxy, ethoxy, trifluoromethyl, and not optionally interrupted by one or more heteroatoms. In preferred embodiments, instances of alkyl or haloalkyl within R² are optionally substituted with halogen, oxy, hydroxyl, methyl, ethyl, propyl, methoxy, ethoxy, trifluoromethyl, and not interrupted by one or more heteroatoms. In preferred embodiments, instances of alkyl or haloalkyl within R² are optionally substituted with halogen, oxy, hydroxyl, methyl, ethyl, propyl, methoxy, ethoxy, trifluoromethyl, and/or optionally interrupted by one or more heteroatoms, and/or optionally unsaturated.

X¹ is CH, C(R²), N, or C(Q); in preferred embodiment X¹ is CH, C(R²), or N; in preferred embodiment X¹ is CH, C(R²), or N; in preferred embodiment X¹ is CH, C(R²), or C(Q); in preferred embodiment X¹ is CH, N, or C(Q); in preferred embodiment X¹ is C(R²), N, or C(Q); in preferred embodiment X¹ is CH or C(R²); in preferred embodiment X¹ is CH or C(Q); in preferred embodiment X¹ is CH or N; in preferred embodiment X¹ is N or C(R²); in preferred embodiment X¹ is C(Q) or C(R²); in preferred embodiment X¹ is N or C(Q); in preferred embodiment X¹ is CH; in preferred embodiment X¹ is C(R²); in preferred embodiment X¹ is N; in the most highly preferred embodiment X¹ is C(Q).

X² is CH, C(R²), or N; in preferred embodiment X¹ is C(R²) or N; in preferred embodiment X¹ is CH or N; in preferred embodiment X¹ is CH or C(R²); in preferred embodiment X¹ is C(R²); in preferred embodiment X¹ is N; most preferably X² is CH. When X² is C(R²), the R² preferably forms a bridging moiety with Q.

Preferably, at most one of X¹ and X² is N. More preferably, when one of X¹ and X² is not CH, the other of X¹ and X² is CH.

Q is H, halogen, C₁₋₆alkyl, —OH, —O—C₁₋₆alkyl, —O—C₁₋₆acyl, —NH₂, —NH—(C₁₋₆alkyl), —N(C₁₋ ₆alkyl)₂, —NH(C₁₋₈acyl), —N(C₁₋₈acyl)₂, —C₁₋₄alkyl-OH, —C₁₋₄alkyl—O—C₁₋₆alkyl, —C₁₋₄alkyl—O—C₁₋₆acyl, —C₁₋₄alkyl—NH₂, —C₁₋₄alkyl—NH—(C₁₋₆alkyl), —C₁₋₄alkyl—N(C₁₋₆alkyl)₂, —C₁₋₄alkyl—NH(C₁₋₈acyl), —C₁₋₄alkyl—N(C₁₋₈acyl)₂, —C₁₋₄alkyl—N—C(O)—NH—C₁₋₆alkyl, —C₁₋₄alkyl—N—C(O)—N(C₁₋₆alkyl)₂, —C₁₋₄alkyl—O—C(O)—NH—C₁₋₆alkyl, —C₁₋₄alkyl—O—C(O)—N(C₁₋₆alkyl)₂, —C₁₋₄alkyl—N—C(O)—O—C₁₋₆alkyl, or Q together with R² forms a bridging moiety selected from —NH—CH═CH—, —NH—(C₂₋₄alkyl)—, and —(C₁₋₃alkyl)—NH—(C₁₋₃alkyl)—; preferably, Q is H, F, —CH₃, —CH₂F, —CHF₂, —CF₃, —OCH₃, —OCH₂F, —OCHF₂, —OCF₃, —NH—C(O)—CH₃, —NH—C(O)—cyclopropyl, —NH—C(O)—phenyl, —NH—C(O)—halophenyl, —NH—C(O)—piperidinyl, —NH—C(O)—pyridinyl, —NH—C(O)—morpholinyl, —NH—C(O)—oxanyl, —NH₂, —NH(CH₃), -NH(cyclopentyl), —CH₂—NH—C(O)—CH₃, —CH₂—N(CH₃)₂, —CH₂—NH₂, —CH₂—NH—(CH₃), —CH₂—NH—(cyclopentyl), or together with R² forms a bridging moiety that is preferably —NH—CH═CH—; more preferably, Q is H, —CH₃, —CHF₂, —OCH₃, —NH—C(O)—CH₃, —NH—C(O)—cyclopropyl, —NH—C(O)—phenyl, —NH—C(O)—halophenyl, —NH—C(O)—piperidinyl, —NH—C(O)—pyridinyl, —NH—C(O)—morpholinyl, —NH—C(O)—oxanyl, —NH₂, —CH₂—NH—(CH₃), or together with R² forms a bridging moiety that is preferably —NH—CH═CH—; eve more preferably, Q is H, F, —NH—C(O)—CH₃, —NH—C(O)—cyclopropyl, —NH—C(O)—phenyl, —NH—C(O)—halophenyl, —NH₂, —NH(CH₃), -NH(cyclopentyl), —CH₂—NH—C(O)—CH₃, —CH₂—NH—(cyclopentyl), or together with R² forms a bridging moiety that is preferably —NH—CH═CH—. Here, -alkyl and -acyl when terminal to a moiety are preferably —C₁₋₄alkyl or C₂₋₄acyl or C₃₋₆cycloalkyl or C₅₋₆aryl, more preferably C₃₋₆cycloalkyl or C₅₋₆aryl. Here, —C₁₋₄alkyl- when preceding a heteroatom is preferably C₁₋₂alkyl, more preferably —CH₂— or —CH₂CH₂—, most preferably —CH₂—. It is to be understood that for —N(C₁₋₆alkyl)₂, —N(C₁₋₈acyl)₂, —C₁₋₄alkyl—N(C₁₋₆alkyl)₂, —C₁₋₄alkyl—N(C₁₋₈acyl)₂, —C₁₋₄alkyl—N—C(O)—N(C₁₋₆alkyl)₂, and —C₁₋₄alkyl—O—C(O)—N(C₁₋₆alkyl)₂, the latter two alkyl or acyl moieties can, together with the N to which they are attached, form a heterocycle, preferably a C₄₋₆heterocycle or a C₅₋₆heteroaryl, most preferably a C₅₋ ₆heterocycle or a C₅₋₆heteroaryl, most preferably a C₅₋₆heterocycle.

A bridging moiety as formed by Q and R² is selected from —NH—CH═CH—, —NH—(C₂₋₄alkyl)—, and —(C₁₋₃alkyl)—NH—(C₁₋₃alkyl)—. Preferred examples are —NH—CH═CH—, —NH—CH₂—CH₂—, —NH—CH₂—, —N═CH—CH₂—CH₂—, —CH₂—CH₂—NH—CH₂—CH₂, and —CH₂—NH—CH₂.

In preferred embodiments, instances of alkyl or acyl within Q are not unsaturated. In preferred embodiments, instances of alkyl or acyl within Q are optionally unsaturated. In preferred embodiments, instances of alkyl or acyl within Q are unsaturated. In preferred embodiments, instances of alkyl or acyl within Q are not substituted with halogen, oxy, hydroxyl, methyl, ethyl, propyl, methoxy, ethoxy, trifluoromethyl, and not optionally interrupted by one or more heteroatoms. In preferred embodiments, instances of alkyl or acyl within Q are optionally substituted with halogen, oxy, hydroxyl, methyl, ethyl, propyl, methoxy, ethoxy, trifluoromethyl, and not interrupted by one or more heteroatoms. In preferred embodiments, instances of alkyl or acyl within Q are optionally substituted with halogen, oxy, hydroxyl, methyl, ethyl, propyl, methoxy, ethoxy, trifluoromethyl, and/or optionally interrupted by one or more heteroatoms, and/or optionally unsaturated.

In preferred embodiments the pyridinic moiety of the compound is as shown below, with a reference name shown below each structure. Py1-Py27 are particularly preferred, Py1-Py18 are even more preferred, Py1-Py12 are still more preferred, Py1-Py4 are greatly preferred, and Py1 is most preferred.

| | | | |
|---|---|---|---|
| 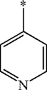 [SMILES: *c1ccncc1] | 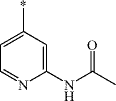 [SMILES: *c1ccnc(NC(C)=O)c1] | 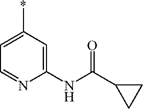 [SMILES: *c1ccnc(NC(=O)C2CC2)c1] | 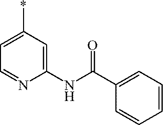 [SMILES: *c1ccnc(NC(=O)c2ccccc2)c1] |
| Py1 | Py2 | Py3 | Py4 |
| 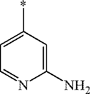 [SMILES: *c1ccnc(N)c1] | 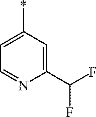 [SMILES: *c1ccnc(C(F)F)c1] | 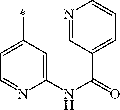 [SMILES: *c1ccnc(NC(=O)c2cccnc2)c1] | 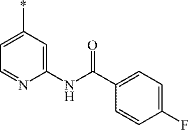 [SMILES: *c1ccnc(NC(=O)c2ccc(F)cc2)c1] |
| Py5 | Py6 | Py7 | Py8 |
| 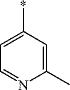 [SMILES: *c1ccnc(C)c1] | 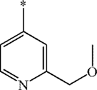 [SMILES: *c1ccnc(COC)c1] | 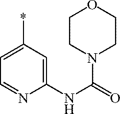 [SMILES: *c1ccnc(NC(=O)N2CCOCC2)c1] | 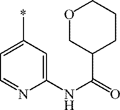 [SMILES: *c1ccnc(NC(=O)C2CCCOC2)c1] |
| Py9 | Py10 | Py11 | Py12 |
| 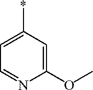 [SMILES: *c1ccnc(OC)c1] | 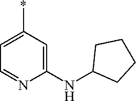 [SMILES: *c1ccnc(NC2CCCC2)c1] | 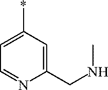 [SMILES: *c1ccnc(CNC)c1] | 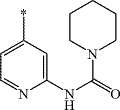 [SMILES: *c1ccnc(NC(=O)N2CCCCC2)c1] |
| Py13 | Py14 | Py15 | Py16 |
| 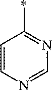 [SMILES: *c1ccncn1] | 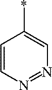 [SMILES: *c1ccnnc1] | 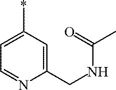 [SMILES: *c1ccnc(CNC(C)=O)c1] | 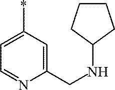 [SMILES: *c1ccnc(CNC2CCCC2)c1] |
| Py17 | Py18 | Py19 | Py20 |
| 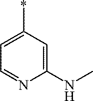 [SMILES: *c1ccnc(NC)c1] | 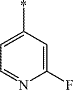 [SMILES: *c1ccnc(F)c1] | 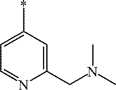 [SMILES: *c1ccnc(CN(C)C)c1] | 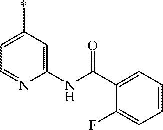 [SMILES: *c1ccnc(NC(=O)c2ccccc2F)c1] |
| Py21 | Py22 | Py23 | Py24 |
| 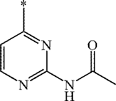 [SMILES: *c1ccnc(NC(C)=O)n1] | 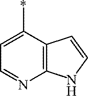 [SMILES: *c1ccnc2[nH]ccc12] | 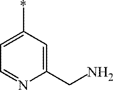 [SMILES: *c1ccnc(CN)c1] | 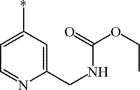 [SMILES: *c1ccnc(CNC(=O)OCC)c1] |
| Py25 | Py26 | Py27 | Py28 |
| 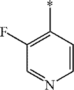 [SMILES: *c1ccncc1F] | 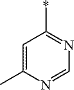 [SMILES: *c1cc(C)ncn1] | 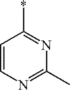 [SMILES: *c1ccnc(C)n1] | |
| Py29 | Py30 | Py31 | |

Compounds of general formula (I-cyc) or (I) are preferably of general formula (II-cyc) or (II), more preferably of general formula (II-A-cyc) or (II-A), most preferably (II-A):

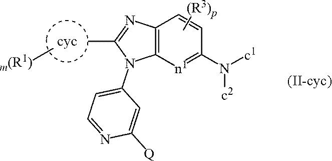

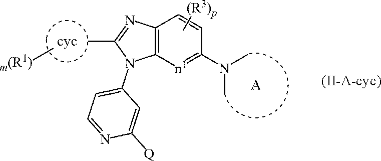

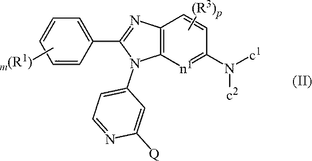

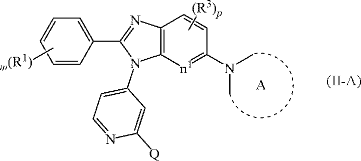

### Arylamine Moiety of the Compound

Compounds according to the invention have an arylamine moiety that is attached adjacent to n¹ of the bicyclic core of the compound according to the invention. It is N,N′-disusbstituted with c¹ and c².

c¹ is H and c² is C₄₋₈cycloalkyl, C₄₋₈heterocycloalkyl, C₄₋₈cycloalkyl-C₁₋₃alkyl, C₄₋ ₈heterocycloalkyl-C₁₋₃alkyl, C₁₋₃alkyl-C₄₋₈cycloalkyl, or C₁₋₃alkyl-C₄₋₈heterocycloalkyl, or c¹ and c² together form cyclic structure A; when c¹ is H, it is preferred that c² is pyridyl, —CH₂-pyridyl, piperidinyl, N-methylpiperidinyl, —CH₂-piperidinyl, —CH₂-(N-methylpiperidinyl), cyclopentyl, hydroxycyclopentyl, —CH₂-cyclopentyl, —CH₂-hydroxycyclopentyl, pyrrolidinyl, N-methylpyrrolidinyl, substituted piperidinyl such as hydroxylpiperidinyl (such as piperidin-3-ol-5-yl) or alkylated piperidinyl (such as 1-methylpiperidin-3-yl), alkylated pyrrolidinyl such as 1-(2,2-difluoroethyl)pyrrolidin-3-yl or 1-methylpyrrolidin-3-yl or 4,4-difluoro-1-methylpyrrolidin-3-yl, oxolanyl such as oxolan-3-yl, —CH₂-pyrrolidinyl, —CH₂—(N-methylpyrrolidinyl). Most preferably c¹ and c² together form cyclic structure A.

In c², C₁₋₃alkyl is preferably —CH₂CH₂— or —CH₂—, most preferably —CH₂—. In c², alkyl is preferably not unsaturated or substituted. In preferred embodiments C₄₋₈cycloalkyl and C₄₋ ₈heterocycloalkyl are unsaturated when comprised in c². In preferred embodiments C₄₋₈cycloalkyl and C₄₋₈heterocycloalkyl are not unsaturated when comprised in c². In preferred embodiments C₄₋ ₈cycloalkyl and C₄₋₈heterocycloalkyl are not substituted when comprised in c². In preferred embodiments C₄₋₈cycloalkyl and C₄₋₈heterocycloalkyl are substituted as described elsewhere herein when comprised in c².

When c¹ is H, preferred embodiments for c² are shown below, with a reference name shown below each structure. In preferred embodiments c² is C2_1-C2_4. In preferred embodiments c² is C2_5-C2_8. In preferred embodiments c² is C2_3-C2_7. In preferred embodiments c² is C2_1-C2_3 or C2_8. In preferred embodiments c² is C2_1-C2_3.

| | | | |
|---|---|---|---|
| 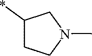 [SMILES: *C1CCN(C)C1] | 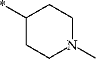 [SMILES: *C1CCN(C)CC1] | 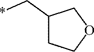 [SMILES: *CC1CCOC1] | 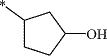 [SMILES: *C1CCC(O)C1] |
| C2_1 | C2_2 | C2_3 | C2_4 |
| 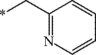 [SMILES: *Cc1ccccn1] | 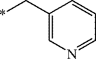 [SMILES: *Cc1cccnc1] | 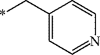 [SMILES: *Cc1ccncc1] | 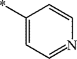 [SMILES: *c1ccncc1] |
| C2_5 | C2_6 | C2_7 | C2_8 |
| 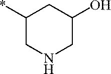 [SMILES: *C1CNCC(O)C1] | 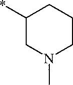 [SMILES: *C1CCCN(C)C1] | 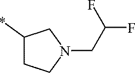 [SMILES: *C1CCN(CC(F)F)C1] | 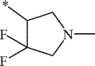 [SMILES: *C1CN(C)CC1(F)F] |
| C2_9 | C2_10 | C2_11 | C2_12 |
| 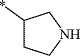 [SMILES: *C1CCNC1] | | | |
| C2_13 | | | |

In preferred embodiments, C2_1 has an absolute configuration (3R) or (3S). In preferred embodiments, C2_13 has an absolute configuration (3R) or (3S).A is a C₄₋₁₂heterocycloalkyl that can be cyclic, bicyclic, and tricyclic, and which is optionally unsaturated, and which is optionally substituted with halogen, C₁₋₆alkyl, C₂₋₄acyl, —O—C₁₋₄alkyl, —SO₂—C₁₋₄alkyl, hydroxyl, —C(═O)—NH₂, —C(═O)—NH(CH₃), —C(═O)—N(CH₃)₂, —NH₂, —NH(C₁₋₄alkyl), or —N(C₁₋₄alkyl)₂. In preferred embodiments there are no such optional substitutions. In these optional substitutions, alkyl is preferably C₁₋₃alkyl, more preferably C₁₋₂alkyl, most preferably —CH₃. Multicyclic structures can be fused, bridged, or spiro. In preferred embodiments, A is not multicyclic. In preferred embodiments, A is cyclic or multicyclic wherein it is fused or bridged. In preferred embodiments, A is cyclic or multicyclic wherein it is fused or spiro. In preferred embodiments, A is cyclic or multicyclic wherein it is spiro or bridged. In preferred embodiments, A is cyclic or multicyclic wherein it is fused. A moiety attached as a spiro-cycle is preferably 3- or 4-membered. A cycle that is fused to A is preferably 4-6-membered, more preferably 5-6-membered. A bridging moiety is preferably 1 or 2 atoms long, most preferably 1. It should be understood that when A is unsaturated it can be a C₅₋₁₂heteroaryl. In preferred embodiments, A is a C₄₋₁₂heterocycloalkyl or a C₅₋₁₂heteroaryl that can be cyclic, bicyclic, and tricyclic, and which is optionally substituted with halogen, C₁₋₆alkyl, —O—C₁₋₄alkyl, hydroxyl, —NH₂, —NH(C₁₋₄alkyl), or —N(C₁₋₄alkyl)₂. Here, C₄₋₁₂ is preferably C₅₋₁₂, more preferably C₅₋₁₀, even more preferably C₅₋₈, most preferably C₅₋₆. In preferred embodiments, for determining the amount of C in an A moiety, only the carbon atoms in the single ring comprising the N of the amide of general structure (I) are counted. In other preferred embodiments all carbon atoms in all cycles of moiety A are counted. In other preferred embodiments all carbon atoms in the entire moiety A are counted.

Preferably, A is selected from optionally substituted and optionally unsaturated azetidinyl, pyrrolidinyl, imidazolidinyl, oxazolidinyl, piperidinyl, piperazinyl, morpholinyl, azacycloheptyl, diazacycloheptyl, or oxoazacycloheptyl (preferably pyrrolidinyl, imidazolidinyl, oxazolidinyl, piperidinyl, piperazinyl, morpholinyl, azacycloheptyl, diazacycloheptyl, or oxoazacycloheptyl); wherein each optional substitution can be a substitution with halogen, C₁₋₆alkyl, C₃₋₆cycloalkyl, C₃₋ ₆heterocycloalkyl, —O—C₁₋₄alkyl, hydroxyl, —NH₂, —NH(C₁₋₄alkyl), or —N(C₁₋₄alkyl)₂; preferably each optional substitution is independently selected from methyl, dimethylamine, methoxyl, propyl, hydroxyl, a bridging C₁₋₃alkyl moiety, spiro azetidinyl, spiro N-methylazetidinyl, spiro oxetanyl, oxetanyl, spiro piperidinyl, difluoropiperidinyl, spiro N-methylpiperidinyl, spiro cyclopropyl, fused pyrrolidinyl, or fused N-methylpyrrolidinyl. In more preferred embodiments, A is not substituted and not unsaturated. In other more preferred embodiments, A is substituted and not unsaturated. In other more preferred embodiments, A is not substituted and is unsaturated. In other more preferred embodiments, A is substituted and unsaturated. Preferably A is not aromatic.

In preferred embodiments the cyclic structure A is as shown below, with a reference name shown below each structure. A1-A9 are particularly preferred, A1-A7 are even more preferred, A1-A3, A6, and A9 are still more preferred, A1, A6, and A9 are even more preferred, and A1 is most preferred. In other preferred embodiments, cyclic structure A comprises an amine or basic nitrogen, more preferably cyclic structure A is selected from A1-A9, A11-A13, A16-A20, A22, A23, A25-A38, A41, and A43. More preferred such cyclic structures A are A1, A2, A3, A5, A6, and A25-A31. Other preferred such embodiments A is A1 or A2; in other preferred such embodiments A is A3, A5, A6, or A25-A32. In other preferred embodiments, cyclic structure A comprises a second heteroatom, more preferably cyclic structure A is selected from A1-A9 and A11-A43. In other preferred embodiments, cyclic structure A is bicyclic, spiro-cyclic, or bridged, preferably selected from A3-A9, A12, A13, A15-A19, A22, A25-A35, and A37-A42; even more preferably it is bicyclic or bridged, preferably selected from A3-A6, A9, A25-A31, A33, and A41. A1-A43 as defined below can be optionally methylated, preferably N-methylated, wherein N-methylation is preferably at a nitrogen that is not attached to the bicyclic core.

| | | | |
|---|---|---|---|
| 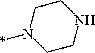 [SMILES: *N1CCNCC1] | 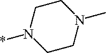 [SMILES: *N1CCN(C)CC1] | 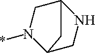 [SMILES: *N1CC2CC1CN2] | 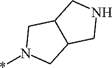 [SMILES: *N1CC2CNCC2C1] |
| A1 | A2 | A3 | A4 |
| 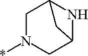 [SMILES: *N1CC2CC(C1)N2] | 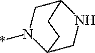 [SMILES: *N1CC2CCC1CN2] | 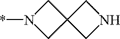 [SMILES: *N1CC2(CNC2)C1] | 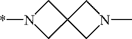 [SMILES: *N1CC2(C1)CN(C)C2] |
| A5 | A6 | A7 | A8 |
| 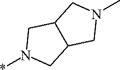 [SMILES: *N1CC2CN(C)CC2C1] | 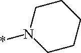 [SMILES: *N1CCCCC1] | 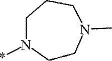 [SMILES: *N1CCCN(C)CC1] | 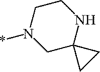 [SMILES: *N1CCNC2(CC2)C1] |
| A9 | A10 | A11 | A12 |
| 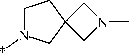 [SMILES: *N1CCC2(C1)CN(C)C2] | 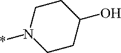 [SMILES: *N1CCC(O)CC1] | 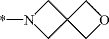 [SMILES: *N1CC2(COC2)C1] | 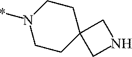 [SMILES: *N1CCC2(CC1)CNC2] |
| A13 | A14 | A15 | A16 |
| 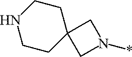 [SMILES: *N1CC2(CCNCC2)C1] | 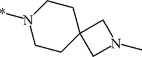 [SMILES: *N1CCC2(CC1)CN(C)C2] | 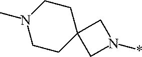 [SMILES: *N1CC2(CCN(C)CC2)C1] | 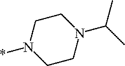 [SMILES: *N1CCN(C(C)C)CC1] |
| A17 | A18 | A19 | A20 |
| 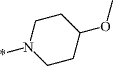 [SMILES: *N1CCC(OC)CC1] | 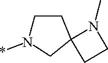 [SMILES: *N1CCC2(CCN2C)C1] | 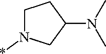 [SMILES: *N1CCC(N(C)C)C1] | 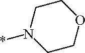 [SMILES: *N1CCOCC1] |
| A21 | A22 | A23 | A24 |
| 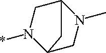 [SMILES: *N1CC2CC1CN2C] | 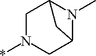 [SMILES: *N1CC2CC(C1)N2C] | 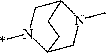 [SMILES: *N1CC2CCC1CN2C] | 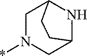 [SMILES: *N1CC2CCC(C1)N2] |
| A25 | A26 | A27 | A28 |
| 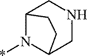 [SMILES: *N1C2CCC1CNC2] | 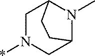 [SMILES: *N1CC2CCC(C1)N2C] | 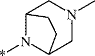 [SMILES: *N1C2CCC1CN(C)C2] | 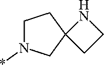 [SMILES: *N1CCC2(CCN2)C1] |
| A29 | A30 | A31 | A32 |
| 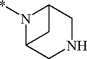 [SMILES: *N1C2CNCC1C2] | 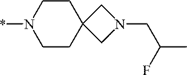 [SMILES: *N1CCC2(CC1)CN(CC(C)F)C2] | 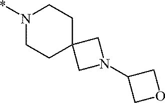 [SMILES: *N1CCC2(CC1)CN(C1COC1)C2] | 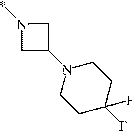 [SMILES: *N1CC(N2CCC(F)(F)CC2)C1] |
| A33 | A34 | A35 | A36 |
| 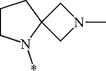 [SMILES: *N1CCCC12CN(C)C2] | 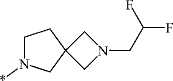 [SMILES: *N1CCC2(C1)CN(CC(F)F)C2] | 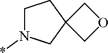 [SMILES: *N1CCC2(COC2)C1] | 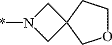 [SMILES: *N1CC2(CCOC2)C1] |
| A37 | A38 | A39 | A40 |
| 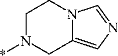 [SMILES: *N1CCn2cncc2C1] | 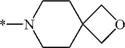 [SMILES: *N1CCC2(CC1)COC2] | 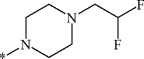 [SMILES: *N1CCN(CC(F)F)CC1] | 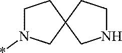 [SMILES: *N1CCC2(CCNC2)C1] |
| A41 | A42 | A43 | A44 |
| 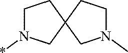 [SMILES: *N1CCC2(CCN(C)C2)C1] | 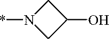 [SMILES: *N1CC(O)C1] | 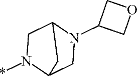 [SMILES: *N1CC2CC1CN2C1COC1] | 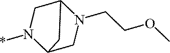 [SMILES: *N1CC2CC1CN2CCOC] |
| A45 | A46 | A47 | A48 |
| 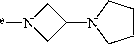 [SMILES: *N1CC(N2CCCC2)C1] | 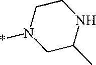 [SMILES: *N1CCNC(C)C1] | 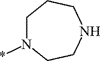 [SMILES: *N1CCCNCC1] | 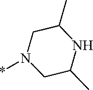 [SMILES: *N1CC(C)NC(C)C1] |
| A49 | A50 | A51 | A52 |
| 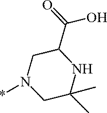 [SMILES: *N1CC(C(=O)O)NC(C)(C)C1] | 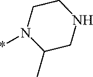 [SMILES: *N1CCNCC1C] | 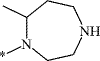 [SMILES: *N1CCNCCC1C] | 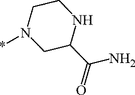 [SMILES: *N1CCNC(C(N)=O)C1] |
| A53 | A54 | A55 | A56 |
| 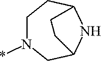 [SMILES: *N1CCC2CCC(C1)N2] | 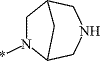 [SMILES: *N1CC2CNCC1C2] | 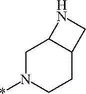 [SMILES: *N1CCC2CNC2C1] | 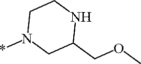 [SMILES: *N1CCNC(COC)C1] |
| A57 | A58 | A59 | A60 |
| 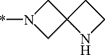 [SMILES: *N1CC2(CCN2)C1] | 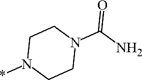 [SMILES: *N1CCN(C(N)=O)CC1] | 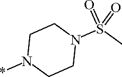 [SMILES: *N1CCN(S(C)(=O)=O)CC1] | 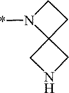 [SMILES: *N1CCC12CNC2] |
| A61 | A62 | A63 | A64 |
| 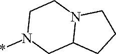 [SMILES: *N1CCN2CCCC2C1] | 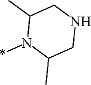 [SMILES: *N1C(C)CNCC1C] | 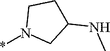 [SMILES: *N1CCC(NC)C1] | 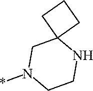 [SMILES: *N1CCNC2(CCC2)C1] |
| A65 | A66 | A67 | A68 |
| 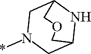 [SMILES: *N1CC2COCC(C1)N2] | 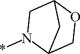 [SMILES: *N1CC2CC1CO2] | 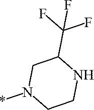 [SMILES: *N1CCNC(C(F)(F)F)C1] | 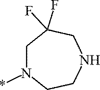 [SMILES: *N1CCNCC(F)(F)C1] |
| A69 | A70 | A71 | A72 |
| 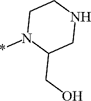 [SMILES: *N1CCNCC1CO] | | | |
| A73 | | | |

In preferred embodiments, A3 has an absolute configuration (1S,4S) or (1R,4R). In preferred embodiments, A6 has an absolute configuration (1S,4S) or (1R,4R). In preferred embodiments, A28 has an absolute configuration (1R,5S). In preferred embodiments, A29 has an absolute configuration (1R,5S). In preferred embodiments, A33 has an absolute configuration (1R,5S). In preferred embodiments, A47 has an absolute configuration (1R,4R). In preferred embodiments, A48 has an absolute configuration (1R,4R). In preferred embodiments, A50 has an absolute configuration (3R) or (3S). In preferred embodiments, A52 has an absolute configuration (3R,5S) or (3S,5S). A54 has an absolute configuration (2R) or (2S). In preferred embodiments, A57 has an absolute configuration (1R,6S). In preferred embodiments, A59 has an absolute configuration (1S,6R). In preferred embodiments, A60 has an absolute configuration (3R) or (3S). In preferred embodiments, A65 has an absolute configuration (8aR) or (8aS). In preferred embodiments, A66 has an absolute configuration (2R,6R). In preferred embodiments, A69 has an absolute configuration (1R,5S). In preferred embodiments, A70 has an absolute configuration (1R,4R). In preferred embodiments, A74 has an absolute configuration (3S). In preferred embodiments, A76 has an absolute configuration (3R) or (3S).

### Further Definitions of the Compound

In preferred embodiments is provided the compound according to the invention, wherein
R¹ is H, fluorine, chlorine, —CH₃, —CF₃, —O—CH₃, or nitrile;
m is 0 or 1;
n¹ is N or CH;
R² is H, fluorine, chlorine, or forms a bridging moiety;
n is 0;
R³ is —CH₃;
p is 0 or 1;
X¹ is C(Q);
X² is CH;
Q is H, F, —CH₃, —CH₂F, —CHF₂, —CF₃, —OCH₃, —OCH₂F, —OCHF₂, —OCF₃, —NH—C(O)—CH₃, —NH—C(O)—cyclopropyl, —NH—C(O)—phenyl, —NH—C(O)—halophenyl, —NH—C(O)—piperidinyl, —NH—C(O)—pyridinyl, —NH—C(O)—morpholinyl, —NH—C(O)—oxanyl, —NH₂, —NH(CH₃), -NH(cyclopentyl), —CH₂—NH—C(O)—CH₃, —CH₂—N(CH₃)₂, —CH₂—NH₂, —CH₂—NH—(CH₃), —CH₂—NH—(cyclopentyl), or together with R² forms —NH—CH═CH—; and/or wherein
c¹ is H and c² is pyridyl, —CH₂—pyridyl, piperidinyl, N-methylpiperidinyl, —CH₂—piperidinyl, —CH₂—(N—methylpiperidinyl), cyclopentyl, hydroxycyclopentyl, —CH₂—cyclopentyl, —CH₂—hydroxycyclopentyl, pyrrolidinyl, N-methylpyrrolidinyl, —CH₂—pyrrolidinyl, —CH₂—(N—methylpyrrolidinyl), or c¹ and c² together form cyclic structure A.

In preferred embodiments is provided the compound according to the invention, wherein R¹ is H, fluorine, or chlorine; R² is H or forms a bridging moiety; p is 0; and/or wherein Q is H, —CH₃, —CHF₂, —OCH₃, —NH—C(O)—CH₃, —NH—C(O)—cyclopropyl, —NH—C(O)—phenyl, —NH—C(O)—halophenyl, —NH—C(O)—piperidinyl, —NH—C(O)—pyridinyl, —NH—C(O)—morpholinyl, —NH—C(O)—oxanyl, —NH₂, —CH₂—NH—(CH₃), or together with R² forms —NH—CH═CH—.

In preferred embodiments, the compound according to the invention comprises:
i) a cyclic ring A selected from A1-A73 or c¹ is H and c² is selected from C2_1-C2_13; preferably the compound comprises a cyclic ring A selected from A1-A73;
ii) a pyridinic moiety selected from Py1-Py31;
iii) a C-bonded ring moiety selected from Ph1-Ph43; and/or
iv) a bicyclic core selected from BC1-BC11.
In more preferred embodiments, both i) and ii) apply. In other more preferred embodiments, both i) and iii) apply. In other more preferred embodiments, both i) and iv) apply. In other more preferred embodiments, both ii) and iii) apply. In other more preferred embodiments, both ii) and iv) apply. In other more preferred embodiments, both iii) and iv) apply. In even more preferred embodiments, each of i), ii) and iii) apply. In other even more preferred embodiments, each of i), ii), and iv) apply. In other even more preferred embodiments, each of i), iii), and iv) apply. In other even more preferred embodiments, each of ii), iii), and iv) apply. In the most preferred embodiments each of i), ii), iii), and iv) apply.

In other preferred embodiments, the compound according to the invention is of general formula (IV) or (IV-A), most preferably (IV-A):

| | |
|---|---|
| 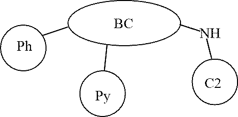 | 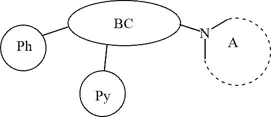 |
| (IV) | (IV-A) |

wherein the cyclic structure A is as defined above, preferably it is selected from A1-A73, more preferably from A1-A24, even more preferably from A1-A9, still more preferably from A1-A7, even more preferably from A1-A3, most preferably it is A1;
wherein c² is as defined above, preferably it is selected from C2_1-C2_8, more preferably it is C2_1-C2_4 or C2_5-C2_8 or C2_3-C2_7, most preferably it is C2_1-C2_3;
wherein the pyridinic moiety Py is as defined above, preferably it is selected from Py1-Py27, more preferably from Py1-Py18, even more preferably from Py1-Py12, still more preferably from Py1-Py4, most preferably it is Py1;
wherein the C-bonded ring moiety Ph is as defined above, preferably it is selected from Ph1-Ph10, more preferably from Ph1-Ph9 and Ph11-Ph19, even more preferably from Ph1-Ph8, still more preferably from Ph4 and Ph8, most preferably it is Ph8;
wherein the bicyclic core BC is as defined above, preferably it is selected from BC1-BC11, more preferably from BC1-BC3, most preferably it is BC1.

In preferred embodiments the compounds according to the invention are compounds 1-203, more preferably compounds 1-47, even more preferably compounds 1-36 listed in table 1 shown below, or salts thereof. More preferred compounds are compounds 1-34 or more preferably 1-31, even more preferred are compounds 1-30, still more preferred are compounds 1-26, even more preferred are compounds 1-20, still more preferred are compounds 1-12, most preferred are compounds 1-4, particularly compound 1. In other preferred embodiments the compound according to the invention is selected from compounds 5, 22, 25, 26, 28, 45, 47, 1, 3, 4, 12, 13, 16, 17, 18, 19, 27, 29, 32, 42, 44, 2, 6, 7, 8, 9, 10, 11, 15, 20, 21, 23, 24, 30, 33, 37, 38, 39, 40, 41, 43, and 46 as listed in table 1; more preferably from compounds 1, 3, 4, 12, 13, 16, 17, 18, 19, 27, 29, 32, 42, 44, 2, 6, 7, 8, 9, 10, 11, 15, 20, 21, 23, 24, 30, 33, 37, 38, 39, 40, 41, 43, and 46; most preferably from compounds 2, 6, 7, 8, 9, 10, 11, 15, 20, 21, 23, 24, 30, 33, 37, 38, 39, 40, 41, 43, and 46.

TABLE 1

preferred compounds according to the invention

| | |
|---|---|
| 1 | N-{4-[2-(4-fluorophenyl)-5-(piperazin-1-yl)-3H-imidazo[4,5-b]pyridin-3-yl]pyridin-2-yl}oxane-3-carboxamide |
| 2 | N-{4-[2-(4-fluorophenyl)-5-(piperazin-1-yl)-3H-imidazo[4,5-b]pyridin-3-yl]pyridin-2-yl}pyridine-3-carboxamide |
| 3 | N-{4-[2-(4-fluorophenyl)-5-(piperazin-1-yl)-3H-imidazo[4,5-b]pyridin-3-yl]pyridin-2-yl}cyclopropanecarboxamide |
| 4 | N-{4-[2-(4-fluorophenyl)-5-(piperazin-1-yl)-3H-imidazo[4,5-b]pyridin-3-yl]pyridin-2-yl}acetamide |
| 5 | N-{4-[2-(4-fluorophenyl)-5-(piperazin-1-yl)-3H-imidazo[4,5-b]pyridin-3-yl]pyridin-2-yl}benzamide |
| 6 | 4-fluoro-N-{4-[2-(4-fluorophenyl)-5-(piperazin-1-yl)-3H-imidazo[4,5-b]pyridin-3-yl]pyridin-2-yl}benzamide |
| 7 | 4-fluoro-N-{4-[2-(4-fluorophenyl)-5-(4-methylpiperazin-1-yl)-3H-imidazo[4,5-b]pyridin-3-yl]pyridin-2-yl}benzamide |
| 8 | N-(4-{5-[2,5-diazabicyclo[2.2.1]heptan-2-yl]-2-(4-fluorophenyl)-3H-imidazo[4,5-b]pyridin-3-yl}pyridin-2-yl)benzamide |
| 8-SS | N-(4-{5-[(1S,4S)-2,5-diazabicyclo[2.2.1]heptan-2-yl]-2-(4-fluorophenyl)-3H-imidazo[4,5-b]pyridin-3-yl}pyridin-2-yl)benzamide |
| 9 | N-{4-[2-(4-fluorophenyl)-5-(piperazin-1-yl)-3H-imidazo[4,5-b]pyridin-3-yl]pyridin-2-yl}morpholine-4-carboxamide |
| 10 | N-(4-{5-[2,5-diazabicyclo[2.2.1]heptan-2-yl]-2-(4-fluorophenyl)-3H-imidazo[4,5-b]pyridin-3-yl}pyridin-2-yl)-4-fluorobenzamide |
| 10-SS | N-(4-{5-[(1S,4S)-2,5-diazabicyclo[2.2.1]heptan-2-yl]-2-(4-fluorophenyl)-3H-imidazo[4,5-b]pyridin-3-yl}pyridin-2-yl)-4-fluorobenzamide |
| 11 | N-(4-{5-[2,5-diazabicyclo[2.2.1]heptan-2-yl]-2-(4-fluorophenyl)-3H-imidazo[4,5-b]pyridin-3-yl}pyridin-2-yl)cyclopropanecarboxamide |
| 11-SS | N-(4-{5-[(1S,4S)-2,5-diazabicyclo[2.2.1]heptan-2-yl]-2-(4-fluorophenyl)-3H-imidazo[4,5-b]pyridin-3-yl}pyridin-2-yl)cyclopropanecarboxamide |
| 12 | 1-[2-(4-fluorophenyl)-3-[2-(methoxymethyl)pyridin-4-yl]-3H-imidazo[4,5-b]pyridin-5-yl]piperazine |
| 13 | 1-[2-(4-fluorophenyl)-3-(2-methylpyridin-4-yl)-3H-imidazo[4,5-b]pyridin-5-yl]piperazine |
| 14 | 1-[2-(4-fluorophenyl)-3-(pyridin-4-yl)-3H-imidazo[4,5-b]pyridin-5-yl]-4-methylpiperazine |
| 15 | 4-[2-(4-fluorophenyl)-5-(piperazin-1-yl)-3H-imidazo[4,5-b]pyridin-3-yl]pyridin-2-amine |
| 16 | 1-{3-[2-(difluoromethyl)pyridin-4-yl]-2-(4-fluorophenyl)-3H-imidazo[4,5-b]pyridin-5-yl}piperazine |
| 17 | 1-[2-(4-fluorophenyl)-3-(pyridin-4-yl)-3H-imidazo[4,5-b]pyridin-5-yl]piperazine |
| 18 | 1-[2-(4-chlorophenyl)-3-(pyridin-4-yl)-3H-imidazo[4,5-b]pyridin-5-yl]piperazine |
| 19 | 1-[2-(3-fluorophenyl)-3-(pyridin-4-yl)-3H-imidazo[4,5-b]pyridin-5-yl]piperazine |
| 20 | 1-[2-(2-fluorophenyl)-3-(pyridin-4-yl)-3H-imidazo[4,5-b]pyridin-5-yl]piperazine |
| 21 | 2-[2-(4-fluorophenyl)-3-(pyridin-4-yl)-3H-imidazo[4,5-b]pyridin-5-yl]-2,5-diazabicyclo[2.2.2]octane |
| 21-RR | (1R,4R)-2-[2-(4-fluorophenyl)-3-(pyridin-4-yl)-3H-imidazo[4,5-b]pyridin-5-yl]-2,5-diazabicyclo[2.2.2]octane |
| 21-SS | (1S,4S)-2-[2-(4-fluorophenyl)-3-(pyridin-4-yl)-3H-imidazo[4,5-b]pyridin-5-yl]-2,5-diazabicyclo[2.2.2]octane |
| 22 | 4-[2-(4-fluorophenyl)-5-(piperazin-1-yl)-3H-imidazo[4,5-b]pyridin-3-yl]pyridazine |
| 23 | 4-{5-[octahydropyrrolo[3,4-c]pyrrol-2-yl]-2-(4-fluorophenyl)-3H-imidazo[4,5-b]pyridin-3-yl}pyridine |
| 23-RS | 4-{5-[(3R,6S)-octahydropyrrolo[3,4-c]pyrrol-2-yl]-2-(4-fluorophenyl)-3H-imidazo [4,5-b]pyridin-3-yl}pyridine |
| 24 | 4-{5-[octahydropyrrolo[3,4-c]pyrrol-2-yl]-2-(4-fluorophenyl)-3H-imidazo[4,5-b]pyridin-3-yl}-2-methylpyridine |
| 24-RS | 4-{5-[(3R,6S)-octahydropyrrolo[3,4-c]pyrrol-2-yl]-2-(4-fluorophenyl)-3H-imidazo [4,5-b]pyridin-3-yl}-2-methylpyridine |
| 25 | ({4-[2-(4-fluorophenyl)-5-(piperazin-1-yl)-3H-imidazo[4,5-b]pyridin-3-yl]pyridin-2-yl}methyl)(methyl)amine |
| 26 | 2-(4-fluorophenyl)-6-(piperazin-1-yl)-1-(pyrimidin-4-yl)-1H-1,3-benzodiazole |
| 27 | 4-[2-(4-fluorophenyl)-5-(piperazin-1-yl)-3H-imidazo[4,5-b]pyridin-3-yl]pyrimidine |
| 28 | 2-(4-fluorophenyl)-6-(piperazin-1-yl)-1-(pyridin-4-yl)-1H-1,3-benzodiazole |
| 29 | 2-(4-fluorophenyl)-1-(2-methylpyridin-4-yl)-6-(piperazin-1-yl)-1H-1,3-benzodiazole |
| 30 | 3-[2-(4-fluorophenyl)-3-(pyridin-4-yl)-3H-imidazo[4,5-b]pyridin-5-yl]-3,6-diazabicyclo[3.1.1]heptane |
| 31 | 2-[2-(4-fluorophenyl)-3-(2-methylpyridin-4-yl)-3H-imidazo[4,5-b]pyridin-5-yl]-2,6-diazaspiro[3.3]heptane |
| 32 | 1-[2-(4-fluorophenyl)-3-(2-methoxypyridin-4-yl)-3H-imidazo[4,5-b]pyridin-5-yl]piperazine |
| 33 | N-{4-[2-(4-fluorophenyl)-5-(piperazin-1-yl)-3H-imidazo[4,5-b]pyridin-3-yl]pyridin-2-yl}piperidine-1-carboxamide |
| 34 | 1-[2-(2,4-difluorophenyl)-3-(pyridin-4-yl)-3H-imidazo[4,5-b]pyridin-5-yl]piperazine |
| 35 | 2-[2-(4-fluorophenyl)-3-(pyridin-4-yl)-3H-imidazo[4,5-b]pyridin-5-yl]-2,5-diazabicyclo[2.2.1]heptane |
| 35-RR | (1R,4R)-2-[2-(4-fluorophenyl)-3-(pyridin-4-yl)-3H-imidazo[4,5-b]pyridin-5-yl]-2,5-diazabicyclo[2.2.1]heptane |
| 35-SS | (1S,4S)-2-[2-(4-fluorophenyl)-3-(pyridin-4-yl)-3H-imidazo[4,5-b]pyridin-5-yl]-2,5-diazabicyclo[2.2.1]heptane |
| 36 | 2-[2-(2-fluorophenyl)-3-(pyridin-4-yl)-3H-imidazo[4,5-b]pyridin-5-yl]-2,5-diazabicyclo[2.2.2]octane |
| 36-SS | (1S,4S)-2-[2-(2-fluorophenyl)-3-(pyridin-4-yl)-3H-imidazo[4,5-b]pyridin-5-yl]-2,5-diazabicyclo[2.2.2]octane |
| 36-RR | (1R,4R)-2-[2-(2-fluorophenyl)-3-(pyridin-4-yl)-3H-imidazo[4,5-b]pyridin-5-yl]-2,5-diazabicyclo[2.2.2]octane |
| 37 | 1-[2-(2-chlorophenyl)-3-(pyridin-4-yl)-3H-imidazo[4,5-b]pyridin-5-yl]piperazine |
| 38 | 3-[2-(4-fluorophenyl)-3-(pyridin-4-yl)-3H-imidazo[4,5-b]pyridin-5-yl]-3,8-diazabicyclo[3.2.1]octane |
| 38-RS | (1R,5S)-3-[2-(4-fluorophenyl)-3-(pyridin-4-yl)-3H-imidazo[4,5-b]pyridin-5-yl]-3,8-diazabicyclo[3.2.1]octane |
| 39 | 1-[3-(pyridin-4-yl)-2-[3-(trifluoromethyl)phenyl]-3H-imidazo[4,5-b]pyridin-5-yl]piperazine |
| 40 | 1-[2-(3-chlorophenyl)-3-(pyridin-4-yl)-3H-imidazo[4,5-b]pyridin-5-yl]piperazine |
| 41 | 8-[2-(4-fluorophenyl)-3-(pyridin-4-yl)-3H-imidazo[4,5-b]pyridin-5-yl]-3,8-diazabicyclo[3.2.1]octane |
| 41-RS | (1R,5S)-8-[2-(4-fluorophenyl)-3-(pyridin-4-yl)-3H-imidazo[4,5-b]pyridin-5-yl]-3,8-diazabicyclo[3.2.1]octane |
| 42 | 1-[2-(4-methoxyphenyl)-3-(pyridin-4-yl)-3H-imidazo[4,5-b]pyridin-5-yl]piperazine |
| 43 | 5-{[2-(4-fluorophenyl)-3-(pyridin-4-yl)-3H-imidazo[4,5-b]pyridin-5-yl]amino}piperidin-3-ol |
| 44 | 1-[3-(pyridin-4-yl)-2-[2-(trifluoromethyl)phenyl]-3H-imidazo[4,5-b]pyridin-5-yl]piperazine |
| 45 | 1-[3-(pyridin-4-yl)-2-[4-(trifluoromethyl)phenyl]-3H-imidazo[4,5-b]pyridin-5-yl]piperazine |
| 46 | 1-[2-(3-methoxyphenyl)-3-(pyridin-4-yl)-3H-imidazo[4,5-b]pyridin-5-yl]piperazine |
| 47 | 1-[2-(2-methoxyphenyl)-3-(pyridin-4-yl)-3H-imidazo[4,5-b]pyridin-5-yl]piperazine |
| 48 | 1-[2-(4-fluorophenyl)-7-methyl-3-(pyridin-4-yl)-3H-imidazo[4,5-b]pyridin-5-yl]piperazine |
| 49 | 2-[2-(4-fluorophenyl)-3-(pyridin-4-yl)-3H-imidazo[4,5-b]pyridin-5-yl]-2,7-diazaspiro[3.5]nonane |
| 50 | 1-[2-(2-fluorophenyl)-7-methyl-3-(pyridin-4-yl)-3H-imidazo[4,5-b]pyridin-5-yl]piperazine |
| 51 | 1-[2-(2,4-difluorophenyl)-7-methyl-3-(pyridin-4-yl)-3H-imidazo[4,5-b]pyridin-5-yl]piperazine |
| 52 | 1-[2-(3-chloro-2-fluorophenyl)-7-methyl-3-(pyridin-4-yl)-3H-imidazo[4,5-b]pyridin-5-yl]piperazine |
| 53 | 6-[2-(2,4-difluorophenyl)-3-(pyridin-4-yl)-3H-imidazo[4,5-b]pyridin-5-yl]-3,6-diazabicyclo[3.1.1]heptane |
| 53-RS | (1R,5S)-6-[2-(2,4-difluorophenyl)-3-(pyridin-4-yl)-3H-imidazo[4,5-b]pyridin-5-yl]-3,6-diazabicyclo[3.1.1]heptane |
| 54 | 1-[2-(3-chlorophenyl)-7-methyl-3-(pyridin-4-yl)-3H-imidazo[4,5-b]pyridin-5-yl]piperazine |
| 55 | 1-[2-(3-chloro-4-fluorophenyl)-7-methyl-3-(pyridin-4-yl)-3H-imidazo[4,5-b]pyridin-5-yl]piperazine |
| 56 | 2-[2-(2,4-difluorophenyl)-3-(pyridin-4-yl)-3H-imidazo[4,5-b]pyridin-5-yl]-2,5-diazabicyclo[2.2.2]octane |
| 56-RR | (1R,4R)-2-[2-(2,4-difluorophenyl)-3-(pyridin-4-yl)-3H-imidazo[4,5-b]pyridin-5-yl]-2,5-diazabicyclo[2.2.2]octane |
| 56-SS | (1S,4S)-2-[2-(2,4-difluorophenyl)-3-(pyridin-4-yl)-3H-imidazo[4,5-b]pyridin-5-yl]-2,5-diazabicyclo[2.2.2]octane |
| 57 | 2-[2-(2,3-difluorophenyl)-3-(pyridin-4-yl)-3H-imidazo[4,5-b]pyridin-5-yl]-2,5-diazabicyclo[2.2.2]octane |
| 58 | 6-[2-(2-fluorophenyl)-3-(pyridin-4-yl)-3H-imidazo[4,5-b]pyridin-5-yl]-3,6-diazabicyclo[3.1.1]heptane |
| 58-RS | (1R,5S)-6-[2-(2-fluorophenyl)-3-(pyridin-4-yl)-3H-imidazo[4,5-b]pyridin-5-yl]-3,6-diazabicyclo[3.1.1]heptane |
| 59 | 2-[2-(2,5-difluorophenyl)-3-(pyridin-4-yl)-3H-imidazo[4,5-b]pyridin-5-yl]-2,5-diazabicyclo[2.2.2]octane |
| 60 | N-[2-(4-fluorophenyl)-3-(pyridin-4-yl)-3H-imidazo[4,5-b]pyridin-5-yl]pyrrolidin-3-amin e |
| 60-R | (3R)-N-[2-(4-fluorophenyl)-3-(pyridin-4-yl)-3H-imidazo[4,5-b]pyridin-5-yl]pyrrolid in-3-amine |
| 60-S | (3S)-N-[2-(4-fluorophenyl)-3-(pyridin-4-yl)-3H-imidazo[4,5-b]pyridin-5-yl]pyrrolidin-3-amine |
| 61 | 2-[2-(pyridin-2-yl)-3-(pyridin-4-yl)-3H-imidazo[4,5-b]pyridin-5-yl]-2,5-diazabicyclo[2.2.2]octane |
| 62 | 2-[2-(2,6-difluorophenyl)-3-(pyridin-4-yl)-3H-imidazo[4,5-b]pyridin-5-yl]-2,5-diazabicyclo[2.2.2]octane |
| 63 | 2-[2-(5-chloro-2-fluorophenyl)-3-(pyridin-4-yl)-3H-imidazo[4,5-b]pyridin-5-yl]-2,5-diazabicyclo[2.2.2]octane |
| 64 | 2-[2-(3-methoxyphenyl)-3-(pyridin-4-yl)-3H-imidazo[4,5-b]pyridin-5-yl]-2,5-diazabicyclo[2.2.2]octane |
| 65 | 2-[2-(3,5-dichlorophenyl)-3-(pyridin-4-yl)-3H-imidazo[4,5-b]pyridin-5-yl]-2,5-diazabicyclo[2.2.2]octane |
| 66 | 2-[2-(3-chloro-5-methoxyphenyl)-3-(pyridin-4-yl)-3H-imidazo[4,5-b]pyridin-5-yl]-2,5-diazabicyclo[2.2.2]octane |
| 67 | 2-[2-(3-chlorophenyl)-3-(pyridin-4-yl)-3H-imidazo[4,5-b]pyridin-5-yl]-2,5-diazabicyclo[2.2.2]octane |
| 68 | 2-[2-(pyridin-3-yl)-3-(pyridin-4-yl)-3H-imidazo[4,5-b]pyridin-5-yl]-2,5-diazabicyclo[2.2.2]octane |
| 69 | 2-[3-(pyridin-4-yl)-2-[3-(trifluoromethoxy)phenyl]-3H-imidazo[4,5-b]pyridin-5-yl]-2,5-diazabicyclo[2.2.2]octane |
| 70 | N-[2-(4-fluorophenyl)-3-(pyridin-4-yl)-3H-imidazo[4,5-b]pyridin-5-yl]-1-methylpyrrolidin-3-amine |
| 70-R | (3R)-N-[2-(4-fluorophenyl)-3-(pyridin-4-yl)-3H-imidazo[4,5-b]pyridin-5-yl]-1-methylpyrrolidin-3-amine |
| 70-S | (3S)-N-[2-(4-fluorophenyl)-3-(pyridin-4-yl)-3H-imidazo[4,5-b]pyridin-5-yl]-1-methylpyrrolidin-3-amine |
| 71 | 2-[2-(3-chlorophenyl)-3-(pyridin-4-yl)-3H-imidazo[4,5-b]pyridin-5-yl]-2,5-diazabicyclo[2.2.1]heptane |
| 71-RR | (1R,4R)-2-[2-(3-chlorophenyl)-3-(pyridin-4-yl)-3H-imidazo[4,5-b]pyridin-5-yl]-2,5-diazabicyclo[2.2.1]heptane |
| 72 | 2-[2-(2,4-difluorophenyl)-3-(pyridin-4-yl)-3H-imidazo[4,5-b]pyridin-5-yl]-2,5-diazabicyclo[2.2.1]heptane |
| 72-RR | (1R,4R)-2-[2-(2,4-difluorophenyl)-3-(pyridin-4-yl)-3H-imidazo[4,5-b]pyridin-5-yl]-2,5-diazabicyclo[2.2.1]heptane |
| 73 | 2-[2-(5-chloro-2-fluorophenyl)-3-(pyridin-4-yl)-3H-imidazo[4,5-b]pyridin-5-yl]-2,5-diazabicyclo[2.2.1]heptane |
| 73-RR | (1R,4R)-2-[2-(5-chloro-2-fluorophenyl)-3-(pyridin-4-yl)-3H-imidazo[4,5-b]pyridin-5-yl]-2,5-diazabicyclo[2.2.1]heptane |
| 74 | 1-[2-(4-fluorophenyl)-6-methyl-3-(pyridin-4-yl)-3H-imidazo[4,5-b]pyridin-5-yl]piperazine |
| 75 | 1-[2-(5-chloro-2-fluorophenyl)-3-(pyridin-4-yl)-3H-imidazo[4,5-b]pyridin-5-yl]piperazine |
| 76 | 2-[3-(pyridin-4-yl)-2-[4-(trifluoromethoxy)phenyl]-3H-imidazo[4,5-b]pyridin-5-yl]-2,5-diazabicyclo[2.2.2]octane |
| 77 | 3-[2-(2,5-difluorophenyl)-3-(pyridin-4-yl)-3H-imidazo[4,5-b]pyridin-5-yl]-3,8-diazabicyclo[3.2.1]octane |
| 77-RS | (1R,5S)-3-[2-(2,5-difluorophenyl)-3-(pyridin-4-yl)-3H-imidazo[4,5-b]pyridin-5-yl]-3,8-diazabicyclo[3.2.1]octane |
| 78 | 3-[2-(5-chloro-2-fluorophenyl)-3-(pyridin-4-yl)-3H-imidazo[4,5-b]pyridin-5-yl]-3,8-diazabicyclo[3.2.1]octane |
| 78-RS | (1R,5S)-3-[2-(5-chloro-2-fluorophenyl)-3-(pyridin-4-yl)-3H-imidazo[4,5-b]pyridin-5-yl]-3,8-diazabicyclo[3.2.1]octane |
| 79 | 3-[2-(2,4-difluorophenyl)-7-methyl-3-(pyridin-4-yl)-3H-imidazo[4,5-b]pyridin-5-yl]-3,8-diazabicyclo[3.2.1]octane |
| 79-RS | (1R,5S)-3-[2-(2,4-difluorophenyl)-7-methyl-3-(pyridin-4-yl)-3H-imidazo[4,5-b]pyridin-5-yl]-3,8-diazabicyclo[3.2.1]octane |
| 80 | 3-[2-(5-chloro-2-fluorophenyl)-7-methyl-3-(pyridin-4-yl)-3H-imidazo[4,5-b]pyridin-5-yl]-3,8-diazabicyclo[3.2.1]octane |
| 80-RS | (1R,5S)-3-[2-(5-chloro-2-fluorophenyl)-7-methyl-3-(pyridin-4-yl)-3H-imidazo[4,5-b]pyridin-5-yl]-3,8-diazabicyclo[3.2.1]octane |
| 81 | 3-[2-(4-fluorophenyl)-7-methyl-3-(pyridin-4-yl)-3H-imidazo[4,5-b]pyridin-5-yl]-3,8-diazabicyclo[3.2.1]octane |
| 81-RS | (1R,5S)-3-[2-(4-fluorophenyl)-7-methyl-3-(pyridin-4-yl)-3H-imidazo[4,5-b]pyridin-5-yl]-3,8-diazabicyclo[3.2.1]octane |
| 82 | 2-[2-(4-fluorophenyl)-7-methyl-3-(pyridin-4-yl)-3H-imidazo[4,5-b]pyridin-5-yl]-2,5-diazabicyclo[2.2.1]heptane |
| 82-SS | (1S,4S)-2-[2-(4-fluorophenyl)-7-methyl-3-(pyridin-4-yl)-3H-imidazo[4,5-b]pyridin-5-yl]-2,5-diazabicyclo[2.2.1]heptane |
| 82-RR | (1R,4R)-2-[2-(4-fluorophenyl)-7-methyl-3-(pyridin-4-yl)-3H-imidazo[4,5-b]pyridin-5-yl]-2,5-diazabicyclo[2.2.1]heptane |
| 83 | 2-[2-(2,4-difluorophenyl)-7-methyl-3-(pyridin-4-yl)-3H-imidazo[4,5-b]pyridin-5-yl]-2,5-diazabicyclo[2.2.1]heptane |
| 83-SS | (1S,4S)-2-[2-(2,4-difluorophenyl)-7-methyl-3-(pyridin-4-yl)-3H-imidazo[4,5-b]pyridin-5-yl]-2,5-diazabicyclo[2.2.1]heptane |
| 83-RR | (1R,4R)-2-[2-(2,4-difluorophenyl)-7-methyl-3-(pyridin-4-yl)-3H-imidazo[4,5-b]pyridin-5-yl]-2,5-diazabicyclo[2.2.1]heptane |
| 84 | 2-[2-(5-chloro-2-fluorophenyl)-7-methyl-3-(pyridin-4-yl)-3H-imidazo[4,5-b]pyridin-5-yl]-2,5-diazabicyclo[2.2.1]heptane |
| 84-SS | (1S,4S)-2-[2-(5-chloro-2-fluorophenyl)-7-methyl-3-(pyridin-4-yl)-3H-imidazo[4,5-b]pyridin-5-yl]-2,5-diazabicyclo[2.2.1]heptane |
| 85 | 1-[2-(2,5-difluorophenyl)-3-(pyridin-4-yl)-3H-imidazo[4,5-b]pyridin-5-yl]piperazine |
| 86 | 2-[2-(4-fluorophenyl)-3-(pyridin-4-yl)-3H-imidazo[4,5-b]pyridin-5-yl]-5-methyl-2,5-diazabicyclo[2.2.1]heptane |
| 86-RR | (1R,4R)-2-[2-(4-fluorophenyl)-3-(pyridin-4-yl)-3H-imidazo[4,5-b]pyridin-5-yl]-5-methyl-2,5-diazabicyclo[2.2.1]heptane |
| 87 | 2-[2-(3-chloro-4-fluorophenyl)-3-(pyridin-4-yl)-3H-imidazo[4,5-b]pyridin-5-yl]-2,5-diazabicyclo[2.2.1]heptane |
| 87-RR | (1R,4R)-2-[2-(3-chloro-4-fluorophenyl)-3-(pyridin-4-yl)-3H-imidazo[4,5-b]pyridin-5-yl]-2,5-diazabicyclo[2.2.1]heptane |
| 88 | 1-[6-chloro-2-(4-fluorophenyl)-3-(pyridin-4-yl)-3H-imidazo[4,5-b]pyridin-5-yl]piperazine |
| 89 | 2-[2-(4-fluorophenyl)-7-methyl-3-(pyridin-4-yl)-3H-imidazo[4,5-b]pyridin-5-yl]-5-methyl-2,5-diazabicyclo[2.2.1]heptane |
| 89-RR | (1R,4R)-2-[2-(4-fluorophenyl)-7-methyl-3-(pyridin-4-yl)-3H-imidazo[4,5-b]pyridin-5-yl]-5-methyl-2,5-diazabicyclo[2.2.1]heptane |
| 90 | 2-[2-(2,4-difluorophenyl)-7-methyl-3-(pyridin-4-yl)-3H-imidazo[4,5-b]pyridin-5-yl]-5-methyl-2,5-diazabicyclo[2.2.1]heptane |
| 90-RR | (1R,4R)-2-[2-(2,4-difluorophenyl)-7-methyl-3-(pyridin-4-yl)-3H-imidazo[4,5-b]pyridin-5-yl]-5-methyl-2,5-diazabicyclo[2.2.1]heptane |
| 91 | 2-[2-(4-fluorophenyl)-3-(pyridin-4-yl)-3H-imidazo[4,5-b]pyridin-5-yl]-2,7-diazaspiro[4.4]nonane |
| 92 | 2-[2-(5-chloro-2-fluorophenyl)-3-(pyridin-4-yl)-3H-imidazo[4,5-b]pyridin-5-yl]-2,7-diazaspiro[4.4]nonane |
| 93 | 2-[2-(3-cyclopropylphenyl)-3-(pyridin-4-yl)-3H-imidazo[4,5-b]pyridin-5-yl]-2,5-diazabicyclo[2.2.1]heptane |
| 93-RR | (1R,4R)-2-[2-(3-cyclopropylphenyl)-3-(pyridin-4-yl)-3H-imidazo[4,5-b]pyridin-5-yl]-2,5-diazabicyclo[2.2.1]heptane |
| 94 | 2-[2-(4-methylphenyl)-3-(pyridin-4-yl)-3H-imidazo[4,5-b]pyridin-5-yl]-2,5-diazabicyclo[2.2.1]heptane |
| 94-RR | (1R,4R)-2-[2-(4-methylphenyl)-3-(pyridin-4-yl)-3H-imidazo[4,5-b]pyridin-5-yl]-2,5-diazabicyclo[2.2.1]heptane |
| 95 | 2-[2-(4-fluorophenyl)-3-(pyridin-4-yl)-3H-imidazo[4,5-b]pyridin-5-yl]-7-methyl-2,7-diazaspiro[4.4]nonane |
| 96 | 2-[2-(2-methylphenyl)-3-(pyridin-4-yl)-3H-imidazo[4,5-b]pyridin-5-yl]-2,5-diazabicyclo[2.2.1]heptane |
| 96-RR | (1R,4R)-2-[2-(2-methylphenyl)-3-(pyridin-4-yl)-3H-imidazo[4,5-b]pyridin-5-yl]-2,5-diazabicyclo[2.2.1]heptane |
| 97 | 1-[6-bromo-2-(4-fluorophenyl)-3-(pyridin-4-yl)-3H-imidazo[4,5-b]pyridin-5-yl]piperazine |
| 98 | 1-[2-(4-fluorophenyl)-3-(pyridin-4-yl)-3H-imidazo[4,5-b]pyridin-5-yl]azetidin-3-oI |
| 99 | 2-[2-(4-fluorophenyl)-3-(pyridin-4-yl)-3H-imidazo[4,5-b]pyridin-5-yl]-5-(oxetan-3-yl)-2,5-diazabicyclo[2.2.1]heptane |
| 99-RR | (1R,4R)-2-[2-(4-fluorophenyl)-3-(pyridin-4-yl)-3H-imidazo[4,5-b]pyridin-5-yl]-5-(oxetan-3-yl)-2,5-diazabicyclo[2.2.1]heptane |
| 100 | 1-[2-(5-chlorothiophen-2-yl)-3-(pyridin-4-yl)-3H-imidazo[4,5-b]pyridin-5-yl]piperazine |
| 101 | 2-[2-(5-chloro-2-fluorophenyl)-3-(pyridin-4-yl)-3H-imidazo[4,5-b]pyridin-5-yl]-7-methyl-2,7-diazaspiro[4.4]nonane |
| 102 | 1-[2-(5-methyl-1,3-thiazol-2-yl)-3-(pyridin-4-yl)-3H-imidazo[4,5-b]pyridin-5-yl]piperazine |
| 103 | 1-[2-(5-methylthiophen-2-yl)-3-(pyridin-4-yl)-3H-imidazo[4,5-b]pyridin-5-yl]piperazine |
| 104 | 2-[2-(3-fluorophenyl)-3-(pyridin-4-yl)-3H-imidazo[4,5-b]pyridin-5-yl]-2,5-diazabicyclo[2.2.2]octane |
| 104-RR | (1R,4R)-2-[2-(3-fluorophenyl)-3-(pyridin-4-yl)-3H-imidazo[4,5-b]pyridin-5-yl]-2,5-diazabicyclo[2.2.2]octane |
| 104-SS | (1S,4S)-2-[2-(3-fluorophenyl)-3-(pyridin-4-yl)-3H-imidazo[4,5-b]pyridin-5-yl]-2,5-diazabicyclo[2.2.2]octane |
| 105 | 2-[2-(4-fluorophenyl)-3-(pyridin-4-yl)-3H-imidazo[4,5-b]pyridin-5-yl]-5-(2-methoxyethyl)-2,5-diazabicyclo[2.2.1]heptane |
| 105-RR | (1R,4R)-2-[2-(4-fluorophenyl)-3-(pyridin-4-yl)-3H-imidazo[4,5-b]pyridin-5-yl]-5-(2-methoxyethyl)-2,5-diazabicyclo[2.2.1]heptane |
| 106 | 4-[2-(4-fluorophenyl)-5-[3-(pyrrolidin-1-yl)azetidin-1-yl]-3H-imidazo[4,5-b]pyridin-3-yl]pyridine |
| 107 | 1-[2-(5-chloro-1,3-thiazol-2-yl)-3-(pyridin-4-yl)-3H-imidazo[4,5-b]pyridin-5-yl]piperazine |
| 108 | 1-[2-(4-fluorophenyl)-3-(pyridin-4-yl)-3H-imidazo[4,5-b]pyridin-5-yl]-3-methylpiperazine |
| 108-S | (3S)-1-[2-(4-fluorophenyl)-3-(pyridin-4-yl)-3H-imidazo[4,5-b]pyridin-5-yl]-3-methylpiperazine |
| 108-R | (3R)-1-[2-(4-fluorophenyl)-3-(pyridin-4-yl)-3H-imidazo[4,5-b]pyridin-5-yl]-3-methylpiperazine |
| 109 | 4-[2-(2,4-difluorophenyl)-5-(piperazin-1-yl)-3H-imidazo[4,5-b]pyridin-3-yl]pyrimidine |
| 110 | 1-[6-fluoro-2-(4-fluorophenyl)-3-(pyridin-4-yl)-3H-imidazo[4,5-b]pyridin-5-yl]piperazine |
| 111 | 1-[2-(4-fluorophenyl)-3-(pyridin-4-yl)-3H-imidazo[4,5-b]pyridin-5-yl]-1,4-diazepane |
| 112 | 4-[2-(5-chloro-2-fluorophenyl)-5-(piperazin-1-yl)-3H-imidazo[4,5-b]pyridin-3-yl]pyrimidine |
| 113 | 7-[2-(4-fluorophenyl)-3-(pyridin-4-yl)-3H-imidazo[4,5-b]pyridin-5-yl]-4,7-diazaspiro[2.5]octane |
| 114 | 2-[2-(5-chloro-2-fluorophenyl)-3-(pyrimidin-4-yl)-3H-imidazo[4,5-b]pyridin-5-yl]-2,5-diazabicyclo[2.2.1]heptane |
| 114-RR | (1R,4R)-2-[2-(5-chloro-2-fluorophenyl)-3-(pyrimidin-4-yl)-3H-imidazo[4,5-b]pyridin-5-yl]-2,5-diazabicyclo[2.2.1]heptane |
| 115 | 2-[2-(2,4-difluorophenyl)-3-(pyrimidin-4-yl)-3H-imidazo[4,5-b]pyridin-5-yl]-2,5-diazabicyclo[2.2.2]octane |
| 115-RR | (1R,4R)-2-[2-(2,4-difluorophenyl)-3-(pyrimidin-4-yl)-3H-imidazo[4,5-b]pyridin-5-yl]-2,5-diazabicyclo[2.2.2]octane |
| 116 | 2-[2-(5-chloro-2-fluorophenyl)-3-(pyrimidin-4-yl)-3H-imidazo[4,5-b]pyridin-5-yl]-2,5-diazabicyclo[2.2.2]octane |
| 116-RR | (1R,4R)-2-[2-(5-chloro-2-fluorophenyl)-3-(pyrimidin-4-yi)-3H-imidazo[4,5-b]pyridin-5-yi]-2,5-diazabicyclo[2.2.2]octane |
| 117 | 2-[2-(4-fluorophenyl)-3-(pyrimidin-4-yl)-3H-imidazo[4,5-b]pyridin-5-yl]-2,5-diazabicyclo[2.2.2]octane |
| 117-RR | (1R,4R)-2-[2-(4-fluorophenyl)-3-(pyrimidin-4-yl)-3H-imidazo[4,5-b]pyridin-5-yl]-2,5-diazabicyclo[2.2.2]octane |
| 118 | 2-[2-(2,4-difluorophenyl)-3-(pyrimidin-4-yl)-3H-imidazo[4,5-b]pyridin-5-yl]-2,5-diazabicyclo[2.2.1]heptane |
| 118-RR | (1R,4R)-2-[2-(2,4-difluorophenyl)-3-(pyrimidin-4-yl)-3H-imidazo[4,5-b]pyridin-5-yl]-2,5-diazabicyclo[2.2.1]heptane |
| 119 | 2-[2-(4-fluorophenyl)-3-(pyrimidin-4-yl)-3H-imidazo[4,5-b]pyridin-5-yl]-2,5-diazabicyclo[2.2.1]heptane |
| 119-RR | (1R,4R)-2-[2-(4-fluorophenyl)-3-(pyrimidin-4-yl)-3H-imidazo[4,5-b]pyridin-5-yl]-2,5-diazabicyclo[2.2.1]heptane |
| 120 | 1-[2-(3,4-difluorophenyl)-3-(pyridin-4-yl)-3H-imidazo[4,5-b]pyridin-5-yl]piperazine |
| 121 | 2-[2-(3,4-difluorophenyl)-3-(2-methylpyridin-4-yl)-3H-imidazo[4,5-b]pyridin-5-yl]-2,5-diazabicyclo[2.2.2]octane |
| 121-RR | (1R,4R)-2-[2-(3,4-difluorophenyl)-3-(2-methylpyridin-4-yl)-3H-imidazo[4,5-b]pyridin-5-yl]-2,5-diazabicyclo[2.2.2]octane |
| 122 | 2-[2-(2-fluorophenyl)-3-(2-methylpyridin-4-yl)-3H-imidazo[4,5-b]pyridin-5-yl]-2,5-diazabicyclo[2.2.2]octane |
| 122-RR | (1R,4R)-2-[2-(2-fluorophenyl)-3-(2-methylpyridin-4-yl)-3H-imidazo[4,5-b]pyridin-5-yl]-2,5-diazabicyclo[2.2.2]octane |
| 123 | 2-[2-(2,4-difluorophenyl)-3-(2-methylpyridin-4-yl)-3H-imidazo[4,5-b]pyridin-5-yl]-2,5-diazabicyclo[2.2.2]octane |
| 123-RR | (1R,4R)-2-[2-(2,4-difluorophenyl)-3-(2-methylpyridin-4-yl)-3H-imidazo[4,5-b]pyridin-5-yl]-2,5-diazabicyclo[2.2.2]octane |
| 124 | 2-[2-(4-fluorophenyl)-3-(2-methylpyridin-4-yl)-3H-imidazo[4,5-b]pyridin-5-yl]-2,5-diazabicyclo[2.2.2]octane |
| 124-RR | (1R,4R)-2-[2-(4-fluorophenyl)-3-(2-methylpyridin-4-yl)-3H-imidazo[4,5-b]pyridin-5-yl]-2,5-diazabicyclo[2.2.2]octane |
| 125 | 1-[2-(5-chloro-2-fluorophenyl)-3-(2-methylpyridin-4-yl)-3H-imidazo[4,5-b]pyridin-5-yl]piperazine |
| 126 | 1-[2-(4-fluorophenyl)-3-(3-methylpyridin-4-yl)-3H-imidazo[4,5-b]pyridin-5-yl]piperazine |
| 127 | 2-[2-(3-fluoropyridin-4-yl)-3-(pyridin-4-yl)-3H-imidazo[4,5-b]pyridin-5-yl]-2,5-diazabicyclo[2.2.1]heptane |
| 127-RR | (1R,4R)-2-[2-(3-fluoropyridin-4-yl)-3-(pyridin-4-yl)-3H-imidazo[4,5-b]pyridin-5-yl]-2,5-diazabicyclo[2.2.1]heptane |
| 128 | 2-[2-(2-fluorophenyl)-3-(pyridin-4-yl)-3H-imidazo[4,5-b]pyridin-5-yl]-2,5-diazabicyclo[2.2.1]heptane |
| 128-RR | (1R,4R)-2-[2-(2-fluorophenyl)-3-(pyridin-4-yl)-3H-imidazo[4,5-b]pyridin-5-yl]-2,5-diazabicyclo[2.2.1]heptane |
| 129 | 2-[2-(5-fluoropyridin-2-yl)-3-(pyridin-4-yl)-3H-imidazo[4,5-b]pyridin-5-yl]-2,5-diazabicyclo[2.2.1]heptane |
| 129-RR | (1R,4R)-2-[2-(5-fluoropyridin-2-yl)-3-(pyridin-4-yl)-3H-imidazo[4,5-b]pyridin-5-yl]-2,5-diazabicyclo[2.2.1]heptane |
| 130 | 2-[2-phenyl-3-(pyridin-4-yl)-3H-imidazo[4,5-b]pyridin-5-yl]-2,5-diazabicyclo[2.2.1]heptane |
| 130-RR | (1R,4R)-2-[2-phenyl-3-(pyridin-4-yl)-3H-imidazo[4,5-b]pyridin-5-yl]-2,5-diazabicyclo[2.2.1]heptane |
| 131 | 3-[2-(4-fluorophenyl)-3-(pyridin-4-yl)-3H-imidazo[4,5-b]pyridin-5-yl]-3,9-diazabicyclo[4.2.1]nonane |
| 131-RS | (1R,6S)-3-[2-(4-fluorophenyl)-3-(pyridin-4-yl)-3H-imidazo[4,5-b]pyridin-5-yl]-3,9-diazabicyclo[4.2.1]nonane |
| 132 | 2-[2-(4-fluorophenyl)-3-(2-methylpyridin-4-yl)-3H-imidazo[4,5-b]pyridin-5-yl]-2,5-diazabicyclo[2.2.1]heptane |
| 132-RR | (1R,4R)-2-[2-(4-fluorophenyl)-3-(2-methylpyridin-4-yl)-3H-imidazo[4,5-b]pyridin-5-yl]-2,5-diazabicyclo[2.2.1]heptane |
| 133 | 1-[2-(4-fluorophenyl)-3-(2-methylpyridin-4-yl)-3H-imidazo[4,5-b]pyridin-5-yl]-3-methylpiperazine |
| 133-S | (3S)-1-[2-(4-fluorophenyl)-3-(2-methylpyridin-4-yl)-3H-imidazo[4,5-b]pyridin-5-yl]-3-methylpiperazine |
| 133-R | (3R)-1-[2-(4-fluorophenyl)-3-(2-methylpyridin-4-yl)-3H-imidazo[4,5-b]pyridin-5-yl]-3-methylpiperazine |
| 134 | 7-[2-(4-fluorophenyl)-3-(2-methylpyridin-4-yl)-3H-imidazo[4,5-b]pyridin-5-yl]-4,7-diazaspiro[2.5]octane |
| 135 | 1-[2-(4-fluorophenyl)-3-(pyridin-4-yl)-3H-imidazo[4,5-b]pyridin-5-yl]-3,5-dimethylpiperazine |
| 135-RS | (3R,5S)-1-[2-(4-fluorophenyl)-3-(pyridin-4-yl)-3H-imidazo[4,5-b]pyridin-5-yl]-3,5-dimethylpiperazine |
| 135-SS | (3S,5S)-1-[2-(4-fluorophenyl)-3-(pyridin-4-yl)-3H-imidazo[4,5-b]pyridin-5-yl]-3,5-dimethylpiperazine |
| 136 | 1-[2-(4-fluorophenyl)-3-(pyridin-4-yl)-3H-imidazo[4,5-b]pyridin-5-yl]-3,3-dimethylpiperazine |
| 137 | 1-[2-(2,4-difluorophenyl)-3-(pyridin-4-yl)-3H-imidazo[4,5-b]pyridin-5-yl]-1,4-diazepane |
| 138 | 7-[2-(2,4-difluorophenyl)-3-(2-methylpyridin-4-yl)-3H-imidazo[4,5-b]pyridin-5-yl]-4,7-diazaspiro[2.5]octane |
| 139 | 1-[2-(2,4-difluorophenyl)-3-(2-methylpyridin-4-yl)-3H-imidazo[4,5-b]pyridin-5-yl]piperazine |
| 140 | 1-[2-(2-fluorophenyl)-3-(2-methylpyridin-4-yl)-3H-imidazo[4,5-b]pyridin-5-yl]piperazine |
| 141 | 1-[2-phenyl-3-(pyridin-4-yl)-3H-imidazo[4,5-b]pyridin-5-yl]piperazine |
| 142 | 2-{3-[2-(difluoromethyl)pyridin-4-yl]-2-(2,4-difluorophenyl)-3H-imidazo[4,5-b]pyridin-5-yl}-2,5-diazabicyclo[2.2.1]heptane |
| 142-RR | (1R,4R)-2-{3-[2-(difluoromethyl)pyridin-4-yl]-2-(2,4-difluorophenyl)-3H-imidazo[4,5-b]pyridin-5-yl}-2,5-diazabicyclo[2.2.1]heptane |
| 143 | 2-{3-[2-(difluoromethyl)pyridin-4-yl]-2-(4-fluorophenyl)-3H-imidazo[4,5-b]pyridin-5-yl}-2,5-diazabicyclo[2.2.1]heptane |
| 143-RR | (1R,4R)-2-{3-[2-(difluoromethyl)pyridin-4-yl]-2-(4-fluorophenyl)-3H-imidazo[4,5-b]pyridin-5-yl}-2,5-diazabicyclo[2.2.1]heptane |
| 144 | (2R)-1-{6-bromo-3-[2-(difluoromethyl)pyridin-4-yl]-2-(4-fluorophenyl)-3H-imidazo[4,5-b]pyridin-5-yl}-2-methylpiperazine |
| 145 | 1-[2-(4-fluorophenyl)-3-(pyridin-4-yl)-3H-imidazo[4,5-b]pyridin-5-yl]-2-methylpiperazine |
| 145-S | (2S)-1-[2-(4-fluorophenyl)-3-(pyridin-4-yl)-3H-imidazo[4,5-b]pyridin-5-yl]-2-methylpiperazine |
| 145-R | (2R)-1-[2-(4-fluorophenyl)-3-(pyridin-4-yl)-3H-imidazo[4,5-b]pyridin-5-yl]-2-methylpiperazine |
| 146 | 7-[2-(3-fluorophenyl)-3-(pyridin-4-yl)-3H-imidazo[4,5-b]pyridin-5-yl]-4,7-diazaspiro[2.5]octane |
| 147 | 7-[2-(3-chlorophenyl)-3-(pyridin-4-yl)-3H-imidazo[4,5-b]pyridin-5-yl]-4,7-diazaspiro[2.5]octane |
| 148 | 7-[2-(2,4-difluorophenyl)-3-(pyridin-4-yl)-3H-imidazo[4,5-b]pyridin-5-yl]-4,7-diazaspiro[2.5]octane |
| 149 | 7-[2-(2-fluorophenyl)-3-(pyridin-4-yl)-3H-imidazo[4,5-b]pyridin-5-yl]-4,7-diazaspiro[2.5]octane |
| 150 | 4-[2-(2,4-difluorophenyl)-5-(piperazin-1-yl)-3H-imidazo[4,5-b]pyridin-3-yl]-6-methylpyrimidine |
| 151 | 6-[2-(4-fluorophenyl)-3-(pyridin-4-yl)-3H-imidazo[4,5-b]pyridin-5-yl]-3,6-diazabicyclo[3.2.1]octane |
| 152 | 2-{3-[2-(difluoromethyl)pyridin-4-yl]-2-(5-fluoropyridin-2-yl)-3H-imidazo[4,5-b]pyridin-5-yl}-2,5-diazabicyclo[2.2.1]heptane |
| 152-RR | (1R,4R)-2-{3-[2-(difluoromethyl)pyridin-4-yl]-2-(5-fluoropyridin-2-yl)-3H-imidazo[4,5-b]pyridin-5-yl}-2,5-diazabicyclo[2.2.1]heptane |
| 153 | 4-[2-(2,4-difluorophenyl)-5-(piperazin-1-yl)-3H-imidazo[4,5-b]pyridin-3-yl]-2-methylpyrimidine |
| 154 | 3-[2-(4-fluorophenyl)-3-(pyridin-4-yl)-3H-imidazo[4,5-b]pyridin-5-yl]-3,8-diazabicyclo[4.2.0]octane |
| 154-SR | (1S,6R)-3-[2-(4-fluorophenyl)-3-(pyridin-4-yl)-3H-imidazo[4,5-b]pyridin-5-yl]-3,8-diazabicyclo[4.2.0]octane |
| 155 | 7-[2-(2-fluorophenyl)-3-(pyridin-4-yl)-3H-imidazo[4,5-b]pyridin-5-yl]-4-methyl-4,7-diazaspiro[2.5]octane |
| 156 | 1-[2-(2,4-difluorophenyl)-6-methyl-3-(pyridin-4-yl)-3H-imidazo[4,5-b]pyridin-5-yl]piperazine |
| 157 | 1-[2-(4-fluorophenyl)-3-(pyridin-4-yl)-3H-imidazo[4,5-b]pyridin-5-yl]-3-(methoxymethyl)piperazine |
| 157-R | (3R)-1-[2-(4-fluorophenyl)-3-(pyridin-4-yl)-3H-imidazo[4,5-b]pyridin-5-yl]-3-(methoxymethyl)piperazine |
| 157-S | (3S)-1-[2-(4-fluorophenyl)-3-(pyridin-4-yl)-3H-imidazo[4,5-b]pyridin-5-yl]-3-(methoxymethyl)piperazine |
| 158 | 6-[2-(4-fluorophenyl)-3-(pyridin-4-yl)-3H-imidazo[4,5-b]pyridin-5-yl]-1,6-diazaspiro[3.3]heptane |
| 159 | 1-{4-[2-(2,4-difluorophenyl)-3-(pyridin-4-yl)-3H-imidazo[4,5-b]pyridin-5-yl]piperazin-1-yl}ethan-1-one |
| 160 | 1-[2-(4-fluorophenyl)-3-(3-fluoropyridin-4-yl)-3H-imidazo[4,5-b]pyridin-5-yl]piperazine |
| 161 | 1-[2-(2,4-difluorophenyl)-3-(pyridin-4-yl)-3H-imidazo[4,5-b]pyridin-5-yl]-4-methanesulfonylpiperazine |
| 162 | 3-[2-(2,4-difluorophenyl)-3-(pyridin-4-yl)-3H-imidazo[4,5-b]pyridin-5-yl]-3,8-diazabicyclo[3.2.1]octane |
| 163 | 1-[2-(4-fluorophenyl)-3-(pyridin-4-yl)-3H-imidazo[4,5-b]pyridin-5-yl]-1,6-diazaspiro[3.3]heptane |
| 164 | 1-[2-(2,4-difluorophenyl)-3-(pyridin-4-yl)-3H-imidazo[4,5-b]pyridin-5-yl]-1,6-diazaspiro[3.3]heptane |
| 165 | 6-[2-(2,4-difluorophenyl)-3-(pyridin-4-yl)-3H-imidazo[4,5-b]pyridin-5-yl]-1,6-diazaspiro[3.3]heptane |
| 166 | 4-[5-(piperazin-1-yl)-2-(2,3,4-trifluorophenyl)-3H-imidazo[4,5-b]pyridin-3-yl]pyrimidine |
| 167 | 4-[5-(piperazin-1-yl)-2-(2,4,5-trifluorophenyl)-3H-imidazo[4,5-b]pyridin-3-yl]pyrimidine |
| 168 | 1-[2-(3,5-difluoropyridin-2-yl)-3-(pyridin-4-yl)-3H-imidazo[4,5-b]pyridin-5-yl]piperazine |
| 169 | 1-{3-[2-(difluoromethyl)pyridin-4-yl]-2-(4-fluorophenyl)-3H-imidazo[4,5-b]pyridin-5-yl}-2-methylpiperazine |
| 169-R | (2R)-1-{3-[2-(difluoromethyl)pyridin-4-yl]-2-(4-fluorophenyl)-3H-imidazo[4,5-b]pyridin-5-yl}-2-methylpiperazine |
| 169-S | (2S)-1-{3-[2-(difluoromethyl)pyridin-4-yl]-2-(4-fluorophenyl)-3H-imidazo[4,5-b]pyridin-5-yl}-2-methylpiperazine |
| 170 | 4-{5-[octahydropyrrolo[1,2-a]pyrazin-2-yl]-2-(4-fluorophenyl)-3H-imidazo[4,5-b]pyridin-3-yl}pyridine |
| 170-R | 4-{5-[(8aR)-octahydropyrrolo[1,2-a]pyrazin-2-yl]-2-(4-fluorophenyl)-3H-imidazo[4,5-b]pyridin-3-yl}pyridine |
| 170-S | 4-{5-[(8aS)-octahydropyrrolo[1,2-a]pyrazin-2-yl]-2-(4-fluorophenyl)-3H-imidazo[4,5-b]pyridin-3-yl}pyridine |
| 171 | 1-[2-(4-fluorophenyl)-3-(pyridin-4-yl)-3H-imidazo[4,5-b]pyridin-5-yl]-2,6-dimethylpiperazine |
| 171-RR | (2R,6R)-1-[2-(4-fluorophenyl)-3-(pyridin-4-yl)-3H-imidazo[4,5-b]pyridin-5-yl]-2,6-dimethylpiperazine |
| 172 | 1-[2-(4-fluorophenyl)-3-(pyridin-4-yl)-3H-imidazo[4,5-b]pyridin-5-yl]-N-methylpyrrolidin-3-amine |
| 172-S | (3S)-1-[2-(4-fluorophenyl)-3-(pyridin-4-yl)-3H-imidazo[4,5-b]pyridin-5-yl]-N-methylpyrrolidin-3-amine |
| 172-R | (3R)-1-[2-(4-fluorophenyl)-3-(pyridin-4-yl)-3H-imidazo[4,5-b]pyridin-5-yl]-N-methylpyrrolidin-3-amine |
| 173 | 8-[2-(4-fluorophenyl)-3-(pyridin-4-yl)-3H-imidazo[4,5-b]pyridin-5-yl]-5,8-diazaspiro[3.5]nonane |
| 174 | 1-[2-(2,4-difluorophenyl)-3-(pyridin-4-yl)-3H-imidazo[4,5-b]pyridin-5-yl]-N-methylpyrrolidin-3-amine |
| 174-S | (3S)-1-[2-(2,4-difluorophenyl)-3-(pyridin-4-yl)-3H-imidazo[4,5-b]pyridin-5-yl]-N-methylpyrrolidin-3-amine |
| 174-R | (3R)-1-[2-(2,4-difluorophenyl)-3-(pyridin-4-yl)-3H-imidazo[4,5-b]pyridin-5-yl]-N-methylpyrrolidin-3-amine |
| 175 | 1-{3-[2-(difluoromethyl)pyridin-4-yl]-2-(2,4-difluorophenyl)-3H-imidazo[4,5-b]pyridin-5-yl}-2-methylpiperazine |
| 175-R | (2R)-1-{3-[2-(difluoromethyl)pyridin-4-yl]-2-(2,4-difluorophenyl)-3H-imidazo[4,5-b]pyridin-5-yl}-2-methylpiperazine |
| 176 | 4-[2-(2,4-difluorophenyl)-5-[2-methylpiperazin-1-yl]-3H-imidazo[4,5-b]pyridin-3-yl]pyridazine |
| 176-R | 4-[2-(2,4-difluorophenyl)-5-[(2R)-2-methylpiperazin-1-yl]-3H-imidazo[4,5-b]pyridin-3-yl]pyridazine |
| 177 | 4-[2-(5-chloro-2-fluorophenyl)-5-[2-methylpiperazin-1-yl]-3H-imidazo[4,5-b]pyridin-3-yl]pyridazine |
| 177-R | 4-[2-(5-chloro-2-fluorophenyl)-5-[(2R)-2-methylpiperazin-1-yl]-3H-imidazo[4,5-b]pyridin-3-yl]pyridazine |
| 178 | 7-[2-(4-fluorophenyl)-3-(pyridin-4-yl)-3H-imidazo[4,5-b]pyridin-5-yl]-3-oxa-7,9-diazabicyclo[3.3.1]nonane |
| 179 | 2-(2,4-difluorophenyl)-6-(piperazin-1-yl)-1-(pyrimidin-4-yl)-1H-1,3-benzodiazole |
| 180 | 1-{3-[2-(difluoromethyl)pyridin-4-yl]-2-(5-fluoropyridin-2-yl)-3H-imidazo[4,5-b]pyridin-5-yl}-2-methylpiperazine |
| 180-R | (2R)-1-{3-[2-(difluoromethyl)pyridin-4-yl]-2-(5-fluoropyridin-2-yl)-3H-imidazo[4,5-b]pyridin-5-yl}-2-methylpiperazine |
| 181 | 1-{3-[2-(difluoromethyl)pyridin-4-yl]-2-phenyl-3H-imidazo[4,5-b]pyridin-5-yl}-2-methylpiperazine |
| 181-R | (2R)-1-{3-[2-(difluoromethyl)pyridin-4-yl]-2-phenyl-3H-imidazo[4,5-b]pyridin-5-yl}-2-methylpiperazine |
| 182 | 9-[2-(4-fluorophenyl)-3-(pyridin-4-yl)-3H-imidazo[4,5-b]pyridin-5-yl]-3-oxa-7,9-diazabicyclo[3.3.1]nonane |
| 182-RS | (1R,5S)-9-[2-(4-fluorophenyl)-3-(pyridin-4-yl)-3H-imidazo[4,5-b]pyridin-5-yl]-3-oxa-7,9-diazabicyclo[3.3.1]nonane |
| 183 | 4-[2-(4-fluorophenyl)-5-[2-methylpiperazin-1-yl]-3H-imidazo[4,5-b]pyridin-3-yl]pyrimidine |
| 183-R | 4-[2-(4-fluorophenyl)-5-[(2R)-2-methylpiperazin-1-yl]-3H-imidazo[4,5-b]pyridin-3-yl]pyrimidine |
| 184 | 4-[2-(2,4-difluorophenyl)-5-[2-methylpiperazin-1-yl]-3H-imidazo[4,5-b]pyridin-3-yl]pyrimidine |
| 184-R | 4-[2-(2,4-difluorophenyl)-5-[(2R)-2-methylpiperazin-1-yl]-3H-imidazo[4,5-b]pyridin-3-yl]pyrimidine |
| 184-S | 4-[2-(2,4-difluorophenyl)-5-[(2S)-2-methylpiperazin-1-yl]-3H-imidazo[4,5-b]pyridin-3-yl]pyrimidine |
| 185 | 6-[2,5-diazabicyclo[2.2.1]heptan-2-yl]-2-(4-fluorophenyl)-1-(pyrimidin-4-yl)-1H-1,3-benzodiazole |
| 185-RR | 6-[(1R,4R)-2,5-diazabicyclo[2.2.1]heptan-2-yl]-2-(4-fluorophenyl)-1-(pyrimidin-4-yl)-1H-1,3-benzodiazole |
| 186 | 2-(2,4-difluorophenyl)-6-[2-methylpiperazin-1-yl]-1-(pyrimidin-4-yl)-1H-1,3-benzodiazole |
| 186-R | 2-(2,4-difluorophenyl)-6-[(2R)-2-methylpiperazin-1-yl]-1-(pyrimidin-4-yl)-1H-1,3-benzodiazole |
| 187 | 1-[2-(2-fluoro-4-methoxyphenyl)-3-(pyridin-4-yl)-3H-imidazo[4,5-b]pyridin-5-yl]-2-methylpiperazine |
| 187-R | (2R)-1-[2-(2-fluoro-4-methoxyphenyl)-3-(pyridin-4-yl)-3H-imidazo[4,5-b]pyridin-5-yl]-2-methylpiperazine |
| 188 | 3-{5-[2-methylpiperazin-1-yl]-3-(pyridin-4-yl)-3H-imidazo[4,5-b]pyridin-2-yl}benzonitrile |
| 188-R | 3-{5-[(2R)-2-methylpiperazin-1-yl]-3-(pyridin-4-yl)-3H-imidazo[4,5-b]pyridin-2-yl}benzonitrile |
| 189 | 6-[2,5-diazabicyclo[2.2.2]octan-2-yl]-2-(4-fluorophenyl)-1-(pyrimidin-4-yl)-1H-1,3-benzodiazole |
| 189-RR | 6-[(1R,4R)-2,5-diazabicyclo[2.2.2]octan-2-yl]-2-(4-fluorophenyl)-1-(pyrimidin-4-yl)-1H-1,3-benzodiazole |
| 190 | 6-[2,5-diazabicyclo[2.2.2]octan-2-yl]-2-(2,4-difluorophenyl)-1-(pyrimidin-4-yl)-1H-1,3-benzodiazole |
| 190-RR | 6-[(1R,4R)-2,5-diazabicyclo[2.2.2]octan-2-yl]-2-(2,4-difluorophenyl)-1-(pyrimidin-4-yl)-1H-1,3-benzodiazole |
| 191 | 5-[2-(2,4-difluorophenyl)-3-(pyridin-4-yl)-3H-imidazo[4,5-b]pyridin-5-yl]-2-oxa-5-azabicyclo[2.2.1]heptane |
| 191-RR | (1R,4R)-5-[2-(2,4-difluorophenyl)-3-(pyridin-4-yl)-3H-imidazo[4,5-b]pyridin-5-yl]-2-oxa-5-azabicyclo[2.2.1]heptane |
| 192 | 1-[2-(3-chlorophenyl)-3-(pyridin-4-yl)-3H-imidazo[4,5-b]pyridin-5-yl]-2-methylpiperazine |
| 192-R | (2R)-1-[2-(3-chlorophenyl)-3-(pyridin-4-yl)-3H-imidazo[4,5-b]pyridin-5-yl]-2-methylpiperazine |
| 193 | 1-[2-(4-methoxyphenyl)-3-(pyridin-4-yl)-3H-imidazo[4,5-b]pyridin-5-yl]-2-methylpiperazine |
| 193-R | (2R)-1-[2-(4-methoxyphenyl)-3-(pyridin-4-yl)-3H-imidazo[4,5-b]pyridin-5-yl]-2-methylpiperazine |
| 194 | 1-[2-(2,4-difluorophenyl)-3-(pyridin-4-yl)-3H-imidazo[4,5-b]pyridin-5-yl]-3-(trifluoromethyl)piperazine |
| 195 | 1-[2-(2,4-difluorophenyl)-3-(pyridin-4-yl)-3H-imidazo[4,5-b]pyridin-5-yl]-6,6-difluoro-1,4-diazepane |
| 196 | 1-[2-(2,4-difluorophenyl)-3-(pyridin-4-yl)-3H-imidazo[4,5-b]pyridin-5-yl]-2-methylpiperazine |
| 196-R | (2R)-1-[2-(2,4-difluorophenyl)-3-(pyridin-4-yl)-3H-imidazo[4,5-b]pyridin-5-yl]-2-methylpiperazine |
| 197 | 4-[2-(2-fluorophenyl)-5-[2-methylpiperazin-1-yl]-3H-imidazo[4,5-b]pyridin-3-yl]pyrimidine |
| 197-R | 4-[2-(2-fluorophenyl)-5-[(2R)-2-methylpiperazin-1-yl]-3H-imidazo[4,5-b]pyridin-3-yl]pyrimidine |
| 198 | 4-[2-(3-chlorophenyl)-5-[2-methylpiperazin-1-yl]-3H-imidazo[4,5-b]pyridin-3-yl]pyrimidine |
| 198-R | 4-[2-(3-chlorophenyl)-5-[(2R)-2-methylpiperazin-1-yl]-3H-imidazo[4,5-b]pyridin-3-yl]pyrimidine |
| 199 | 1-[2-(2,4-difluorophenyl)-3-(pyridin-4-yl)-3H-imidazo[4,5-b]pyridin-5-yl]-7-methyl-1,4-diazepane |
| 200 | 4-[2-(2,4-difluorophenyl)-3-(pyridin-4-yl)-3H-imidazo[4,5-b]pyridin-5-yl]piperazine-2-carboxamide |
| 200-R | (2R)-4-[2-(2,4-difluorophenyl)-3-(pyridin-4-yl)-3H-imidazo[4,5-b]pyridin-5-yl]piperazine-2-carboxamide |
| 201 | 4-[2-(5-methyl-1,3-thiazol-2-yl)-5-[2-methylpiperazin-1-yl]-3H-imidazo[4,5-b]pyridin-3-yl]pyrimidine |
| 201-R | 4-[2-(5-methyl-1,3-thiazol-2-yl)-5-[(2R)-2-methylpiperazin-1-yl]-3H-imidazo[4,5-b]pyridin-3-yl]pyrimidine |
| 202 | 2-methyl-1-[2-(5-methyl-1,3-thiazol-2-yl)-3-(pyridin-4-yl)-3H-imidazo[4,5-b]pyridin-5-yl]piperazine |
| 202-R | (2R)-2-methyl-1-[2-(5-methyl-1,3-thiazol-2-yl)-3-(pyridin-4-yl)-3H-imidazo[4,5-b]pyridin-5-yl]piperazine |
| 203 | [1-[2-(2,4-difluorophenyl)-3-(pyridin-4-yl)-3H-imidazo[4,5-b]pyridin-5-yl]piperazin-2-yl]methanol |
| 203-S | [(2S)-1-[2-(2,4-difluorophenyl)-3-(pyridin-4-yl)-3H-imidazo[4,5-b]pyridin-5-yl]piperazin-2-yl]methanol |

Preferably, 8 is 8-SS. Preferably, 10 is 10-SS. Preferably, 11 is 11-SS. Preferably, 21 is 21-RR. Preferably, 21 is 21-SS. Preferably, 23 is 23-RS. Preferably, 24 is 24-RS. Preferably, 35 is 35-RR. Preferably, 35 is 35-SS. Preferably, 36 is 36-SS. Preferably, 36 is 36-RR. Preferably, 38 is 38-RS. Preferably, 41 is 41-RS. Preferably, 53 is 53-RS. Preferably, 56 is 56-RR. Preferably, 56 is 56-SS. Preferably, 58 is 58-RS. Preferably, 60 is 60-R. Preferably, 60 is 60-S. Preferably, 70 is 70-R. Preferably, 70 is 70-S. Preferably, 71 is 71-RR. Preferably, 72 is 72-RR. Preferably, 73 is 73-RR. Preferably, 77 is 77-RS. Preferably, 78 is 78-RS. Preferably, 79 is 79-RS. Preferably, 80 is 80-RS. Preferably, 81 is 81-RS. Preferably, 82 is 82-SS. Preferably, 82 is 82-RR. Preferably, 83 is 83-SS. Preferably, 83 is 83-RR. Preferably, 84 is 84-SS. Preferably, 86 is 86-RR. Preferably, 87 is 87-RR. Preferably, 89 is 89-RR. Preferably, 90 is 90-RR. Preferably, 93 is 93-RR. Preferably, 94 is 94-RR. Preferably, 96 is 96-RR. Preferably, 99 is 99-RR. Preferably, 104 is 104-RR. Preferably, 104 is 104-SS. Preferably, 105 is 105-RR. Preferably, 108 is 108-S. Preferably, 108 is 108-R. Preferably, 114 is 114-RR. Preferably, 115 is 115-RR. Preferably, 116 is 116-RR. Preferably, 117 is 117-RR. Preferably, 118 is 118-RR. Preferably, 119 is 119-RR. Preferably, 121 is 121-RR. Preferably, 122 is 122-RR. Preferably, 123 is 123-RR. Preferably, 124 is 124-RR. Preferably, 127 is 127-RR. Preferably, 128 is 128-RR. Preferably, 129 is 129-RR. Preferably, 130 is 130-RR. Preferably, 131 is 131-RS. Preferably, 132 is 132-RR. Preferably, 133 is 133-S. Preferably, 133 is 133-R. Preferably, 135 is 135-RS. Preferably, 135 is 135-SS. Preferably, 142 is 142-RR. Preferably, 143 is 143-RR. Preferably, 145 is 145-S. Preferably, 145 is 145-R. Preferably, 152 is 152-RR. Preferably, 154 is 154-SR. Preferably, 157 is 157-R. Preferably, 157 is 157-S. Preferably, 169 is 169-R. Preferably, 169 is 169-S. Preferably, 170 is 170-R. Preferably, 170 is 170-S. Preferably, 171 is 171-RR. Preferably, 172 is 172-S. Preferably, 172 is 172-R. Preferably, 174 is 174-S. Preferably, 174 is 174-R. Preferably, 175 is 175-R. Preferably, 176 is 176-R. Preferably, 177 is 177-R. Preferably, 180 is 180-R. Preferably, 181 is 181-R. Preferably, 182 is 182-RS. Preferably, 183 is 183-R. Preferably, 184 is 184-R. Preferably, 184 is 184-S. Preferably, 185 is 185-RR. Preferably, 186 is 186-R. Preferably, 187 is 187-R. Preferably, 188 is 188-R. Preferably, 189 is 189-RR. Preferably, 190 is 190-RR. Preferably, 191 is 191-RR. Preferably, 192 is 192-R. Preferably, 193 is 193-R. Preferably, 196 is 196-R. Preferably, 197 is 197-R. Preferably, 198 is 198-R. Preferably, 200 is 200-R. Preferably, 201 is 201-R. Preferably, 202 is 202-R. Preferably, 203 is 203-S.

In the context of the invention, a salt of a compound according to the invention is preferably a pharmaceutically acceptable salt. Such salts include salts derived from inorganic bases such as Li, Na, K, Ca, Mg, Fe, Cu, Zn and Mn; salts of organic bases such as N,N′-diacetylethylenediamine, glucamine, triethylamine, choline, dicyclohexylamine, benzylamine, trialkylamine, thiamine, guanidine, diethanolamine, alpha-phenylethylamine, piperidine, morpholine, pyridine, hydroxyethylpyrrolidine, hydroxyethylpiperidine, and the like. Such salts also include amino acid salts such as glycine, alanine, cystine, cysteine, lysine, arginine, phenylalanine, guanidine, etc. Such salts may include acid addition salts where appropriate, which are for example sulphates, nitrates, phosphates, perchlorates, borates, hydrohalides such as HCl or HBr salts, acetates, trifluoroacetates, tartrates, maleates, citrates, succinates, palmoates, methanesulphonates, tosylates, benzoates, salicylates, hydroxynaphthoates, benzenesulfonates, ascorbates, glycerophosphates, ketoglutarates and the like. Preferred salts are HCl salts, formic acid salts, acetic acid salts, and trifluoroacetic acid salts. More preferred salts are HCl salts, acetic acid salts and formic acid salts, most preferably HCl salts.

The compound according to the invention is preferably a hydrate or a solvate. In the context of the invention a hydrate refers to a solvate wherein the solvent is water. The term solvate, as used herein, refers to a crystal form of a substance which contains solvent. Solvates are preferably pharmaceutically acceptable solvates and may be hydrates or may comprise other solvents of crystallization such as alcohols, ether, and the like.

Each instance of acyl, alkyl, cycloalkyl, or heterocycloalkyl individually is optionally unsaturated, and optionally substituted with halogen, oxy, hydroxyl, methyl, ethyl, propyl, methoxy, ethoxy, trifluoromethyl, or optionally interrupted by one or more heteroatoms. A skilled person will understand that the valency of atoms is always to be fulfilled. In this context, heterocycloalkyl is to be interpreted as cycloalkyl that has been interrupted by one or more heteroatoms. In the context of this invention, acyl moieties are alkyl moieties wherein the proximal carbon atom is substituted by an oxo moiety (═O). In this context, haloalkyl is to be interpreted as alkyl that has been substituted with halogen. A preferred haloalkyl is a fluorinated alkyl, more preferably a perfluorinated alkyl, most preferably trifluoromehtyl. In the context of the invention, halogen is fluorine (F), chlorine (Cl), bromine (Br), or iodine (I). Preferred halogens for compounds according to the invention are fluorine, chlorine, and bromine, more preferred halogens are fluorine or chlorine, a most preferred halogen is fluorine.

In the context of this invention, the number of carbon atoms in a moiety such as alkyl, acyl, cycloalkyl, heterocycloalkyl, is indicated as for example C₁₋₆, in this non-limiting case indicating that from 1 to 6 carbon atoms are envisaged, such as 1, 2, 3, 4, 5, or 6 carbon atoms. Similarly C₂₋₄alkyl has 2, 3, or 4 carbon atoms. The number of carbon atoms can be expressed as the total number of carbon atoms not counting further substitutions, the total number of carbon atoms, or as the number of carbon atoms that can be found in the longest continuous internal sequence of carbon atoms. Preferably, the number of carbon atoms is expressed as the total number of carbon atoms not counting further substitutions.

In the context of this invention, a bridging moiety connects two sites. A bridging moiety is connected to a compound according to the invention on two locations. When a bridging moiety is asymmetric, it can be present in a compound according to the invention in both orientations; preferably, it is present in a compound according to the invention in the orientation in which it is presented, wherein the left side corresponds to the constituent substituent that is first named as forming the bridging moiety, and the right side corresponds to the constituent substituent that is last named as forming the bridging moiety.

In the context of this invention, unsubstituted alkyl groups have the general formula CₙH₂ₙ₊₁ and may be linear or branched. Unsubstituted alkyl groups may also contain a cyclic moiety, and thus have the concomitant general formula CₙH₂ₙ₋₁. Optionally, the alkyl groups are substituted by one or more substituents further specified in this document. Examples of suitable alkyl groups include, but are not limited to, —CH₃, —CH₂CH₃, —CH₂CH₂CH₃, —CH(CH₃)₂, —CH(CH₃)CH₂CH₃, —CH₂CH(CH₃)₂, —CH₂CH₂CH₂CH₃, —C(CH₃)₃, 1-hexyl and the like. Preferred alkyl groups are linear or branched, most preferably, linear. Cycloalkyl groups are cyclic alkyl groups; preferred cycloalkyl groups are cyclopropyl, cyclobutyl, cyclopentyl, and cyclohexyl, most preferably cyclopentyl. Heterocycloalkyl groups are cycloalkylgroups wherein at least one CH₂ moiety is replaced by a heteroatom. Preferred heteroatoms are S, O, and N. Preferred heterocycloalkyl groups are pyrrolidinyl, piperidinyl, oxiranyl, and oxolanyl. Preferred C₁₋₄alkyl groups are —CH₃, —CH₂CH₃, —CH₂CH₂CH₃, —CH(CH₃)₂, —CH(CH₃)CH₂CH₃, —CH₂CH(CH₃)₂, —CH₂CH₂CH₂CH₃, —C(CH₃)₃, cyclopropyl, and cyclobutyl, more preferably, —CH₃, —CH₂CH₃, —CH₂CH₂CH₃, —CH(CH₃)₂, —CH(CH₃)CH₂CH₃,, —CH₂CH(CH₃)₂, —CH₂CH₂CH₂CH₃, and —C(CH₃)₃.

Alkyl groups of the invention are optionally unsaturated. In preferred embodiments, alkyl is not unsaturated. Unsaturated alkyl groups are preferably alkenyl or alkynyl groups. In the context of this invention, unsubstituted alkenyl groups have the general formula CₙH₂ₙ₋₁, and may be linear or branched. Examples of suitable alkenyl groups include, but are not limited to, ethenyl, propenyl, isopropenyl, butenyl, pentenyl and the like. Unsubstituted alkenyl groups may also contain a cyclic moiety, and thus have the concomitant general formula CₙH₂ₙ₋₃. Preferred alkenyl groups are linear or branched, most preferably, linear. Highly preferred unsaturated cycloalkyl groups are aryl groups, such as phenyl.

In the context of this invention, unsubstituted alkynyl groups have the general formula CₙH₂ₙ₋₃ and may be linear or branched. Unsubstituted alkynyl groups may also contain a cyclic moiety, and thus have the concomitant general formula CₙH₂ₙ₋₅. Optionally, the alkynyl groups are substituted by one or more substituents further specified in this document. Examples of suitable alkynyl groups include, but are not limited to, ethynyl, propargyl, n-but-2-ynyl, n-but-3-ynyl, and octyne such as cyclooctyne. Preferred alkyl groups are linear or branched, most preferably linear.

In the context of this invention, aryl groups are aromatic and generally comprise at least six carbon atoms and may include monocyclic, bicyclic and polycyclic structures. Optionally, the aryl groups may be substituted by one or more substituents further specified in this document. Examples of aryl groups include groups such as phenyl, naphthyl, anthracyl and the like. A heteroaryl group is aromatic and comprises one to four heteroatoms selected from the group consisting of S, O, and N. Due to the heteroatoms it can have a smaller ring size than six.

In this invention, each instance of alkyl, acyl, cycloalkyl, and heterocycloalkyl is optionally substituted, preferably with one or more moieties selected from halogen, oxy, hydroxyl, methyl, ethyl, propyl, methoxy, ethoxy, trifluoromethyl, wherein each instance can also be interrupted by a heteroatom such as N, O, or S, and wherein each instance of alkyl, acyl, alkoxyl, cyclyl, and heterocyclyl is optionally unsaturated. Interruption by a heteroatom means interruption by one or more heteroatoms. In this context, preferably no more than 20, more preferably 1, 2, 3, 4, or 5 heteroatoms interrupt, even more preferably 1, 2, or 3, preferably 1 or 2, most preferably 1 heteroatom interrupts. Preferably all interrupting heteroatoms are of the same element. As a non-limiting example, the Csalkyl —CH₂—CH₂—CH₂—CH₂—CH₃ when interrupted by heteroatoms can be —CH₂—CH₂—O—CH₂—CH₂—O—CH₃. In preferred embodiments, there is no optional substitution. In preferred embodiments, there is both substitution and unsaturation.

In preferred embodiments, C₁₋₆alkyl when optionally unsaturated and optionally susbstituted can be C₁₋₆alkyl, C₁₋₆acyl, C₂₋₆alkenyl, C₂₋₆alkynyl, C₃₋₆cycloalkyl, C₃₋₆heterocycloalkyl, or C₅₋₆aryl, optionally substituted with one or more moieties selected from halogen, oxy, hydroxyl, methyl, ethyl, propyl, methoxy, ethoxy, and trifluoromethyl. In preferred embodiments, C₁₋₄alkyl when optionally unsaturated and optionally susbstituted can be C₁₋₄alkyl, C₁₋₄acyl, C₂₋₄alkenyl, C₂₋₄alkynyl, C₃₋₄cycloalkyl, or C₃₋₄heterocycloalkyl, optionally substituted with one or more moieties selected from halogen, oxy, hydroxyl, methyl, ethyl, propyl, methoxy, ethoxy, and trifluoromethyl.

Molecules provided in this invention can be optionally substituted. Suitable optional substitutions are replacement of —H by a halogen. Preferred halogens are F, Cl, Br, and I, most preferably F. Further suitable optional substitutions are substitutions of one or more —H by oxy, hydroxyl, methyl, ethyl, propyl, methoxy, ethoxy, and trifluoromethyl.

### Compositions and Combinations

In a further aspect, the invention provides a composition comprising at least one compound of general formula I, and a pharmaceutically acceptable excipient, preferably for use according to the invention (use is described elsewhere herein). Such a composition is referred to herein as a composition according to the invention. Preferred compositions according to the invention are pharmaceutical compositions. In preferred embodiments, the composition according to the invention is formulated for oral, sublingual, parenteral, intravascular, intravenous, subcutaneous, or transdermal administration, optionally for administration by inhalation; preferably for oral administration. More features and definitions of administration methods are provided in the section on formulation and administration.

The invention also provides combinations of compounds according to the invention with further measures known for treating or ameliorating diseases or conditions associated with DUX4, for example known for treatments of FSHD or cancer. In preferred embodiments of such combinations is provided a combination of a compound according to the invention and a chemotherapeutic agent. Chemotherapeutic agents are widely known. In another preferred combination, the compound according to the invention is combined with a p38 inhibitor, a β2 adrenergic receptor agonist, a CK1 inhibitor, and/or a BET inhibitor. In some preferred combinations the compound may be combined with clinical management, for example involving physical therapy, aerobic exercise, respiratory function therapy, or orthopedic interventions.

### Compound for Use

Following the central role of DUX4 in the consensus disease hypothesis for FSHD, a therapeutic approach with a disease-modifying potential is expected to rely on the inhibition of DUX4. The inventors have identified the compounds according to the invention as being able to achieve DUX4 repression in muscle cells. This invention has been made using primary FSHD patient-derived muscle cells. Because of the primate-specificity of the FSHD locus and questionable relevance of recombinant, immortalized, or tumorigenic cell or animal models to study endogenous DUX4 regulatory mechanisms, primary patient-derived muscle cells are the most relevant disease model. Assays based on immortalized cells bear the risk of altered epigenomes, thereby limiting their relevance in studying the endogenous regulation of DUX4 expression. Particularly the subtelomeric location of D4Z4 and the importance of the D4Z4 epigenome in the stability of DUX4 repression (Stadler et al., 2013, DOI: 10.1038/nsmb.2571) underscore the necessity of using primary muscle cells to discover physiologically relevant drug targets that regulate the expression of DUX4.

DUX4 has historically been regarded as being challenging to detect in FSHD muscle. Its expression in primary myoblasts from patients with FSHD has been shown to be stochastic. Studies have reported that only 1 in 1000 or 1 in 200 nuclei is DUX4 positive in proliferating FSHD myoblasts and during myoblast differentiation, respectively. Due to this particularly low abundance of DUX4, detection of DUX4 protein has been reported to be a technical challenge. While primary FSHD muscle cells have been used extensively in the FSHD literature, none of the reports appear to be applicable beyond a bench scale level. The limitations posed by using primary cells and the recognised complexity of detecting the low levels of endogenous DUX4 illustrate the challenges associated with applying primary FSHD muscle cells to higher throughput formats. Although DUX4 expression increases upon in vitro differentiation of proliferating FSHD myoblasts into multinucleated myotubes, the levels remain low and the dynamic variability is widely accepted to be extremely challenging for robust large-scale screening approaches (Campbell et al., 2017).

The invention thus provides compound according to the invention for use in the treatment of a disease or condition associated with (undue) DUX4 expression, wherein the compound reduces DUX4 expression. The invention provides a compound of general formula (I), or a composition according to the invention, for use as a medicament, wherein the medicament is preferably for use in the treatment of a disease or condition associated with DUX4 expression, and wherein the compound of general formula (I) reduces DUX4 expression, wherein more preferably said disease or condition associated with DUX4 expression is a muscular dystrophy or cancer, even more preferably wherein said disease or condition associated with DUX4 expression is a muscular dystrophy, most preferably facioscapulohumeral muscular dystrophy (FSHD). Such a compound is referred to herein as a compound for use according to the invention.

The medical use herein described is formulated as a compound for use as a medicament for treatment of the stated condition(s) (e.g. by administration of an effective amount of the compound), but could equally be formulated as i) a method of treatment of the stated condition(s) using a compound as defined herein comprising a step of administering to a subject an effective amount of the compound, ii) a compound as defined herein for use in the manufacture of a medicament to treat the stated condition(s), wherein preferably the compound is to be administered in an effective amount, and iii) use of a compound as defined herein for the treatment of the stated condition(s), preferably by administering an effective amount. Such medical uses are all envisaged by the present invention. Preferred subjects are subjects in need of treatment. Treatment preferably leads to delay, amelioration, alleviation, stabilization, cure, or prevention of a disease or condition. In other words, a compound for use according to the invention can be a compound for the treatment, delay, amelioration, alleviation, stabilization, cure, or prevention of the stated disease or condition.

The compound according to the invention reduces DUX4 expression. This DUX4 expression is preferably the overall DUX4 expression of the subject that is treated. DUX4 expression can be determined using methods known in the art or exemplified in the examples. As is known in the art, DUX4 expression can also be determined by determining the expression of its target genes. For example, DUX4 expression can be determined using PCR techniques such as RT-PCR, or using immunostaining, mass spectrometry, or ELISA, for example on a sample containing cells or cell extracts, preferably obtained from the subject. In this context, a reduction is preferably a reduction as compared to either a predetermined value, or to a reference value. A preferred reference value is a reference value obtained by determining DUX4 expression in an untreated sample containing cells or cell extracts. This untreated sample can be from the same subject or from a different and healthy subject, more preferably it is a sample that was obtained in the same way, thus containing the same type of cells. Conveniently, both the test sample and the reference sample can be part of a single larger sample that was obtained. Alternately, the test sample was obtained from the subject before treatment commenced. A highly preferred reference value is the expression level of DUX4 in a sample obtained from a subject prior to the first administration of the compound according to the invention. Another preferred reference value is a fixed value that represents an absence of DUX4 expression.

A reduction of DUX4 expression preferably means that expression is reduced by at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%. If expression of DUX4 is reduced by for example 100%, it may be that expression of DUX4 can no longer be detected. Reduction can be assessed at the protein level, for example through immunostaining, ELISA, or mass spectrometry, or it can be assessed at the mRNA level, for example through PCR techniques such as RT-PCR. In preferred embodiments, the invention provides a compound for use according to the invention, wherein the reduction of DUX4 expression is determined using PCR or immunostaining, wherein a preferred PCR technique is RT-PCR. In preferred embodiments the invention provides a compound for use according to the invention, wherein DUX4 expression is reduced by at least 20%, 40%, 60%, 80%, or more, more preferably by at least 30%, 40%, 60%, 80%, or more. In further preferred embodiments, DUX4 expression is reduced by at least 10%. In further preferred embodiments, DUX4 expression is reduced by at least 20%. In further preferred embodiments, DUX4 expression is reduced by at least 30%. In further preferred embodiments, DUX4 expression is reduced by at least 40%. In further preferred embodiments, DUX4 expression is reduced by at least 50%. In further preferred embodiments, DUX4 expression is reduced by at least 60%. In further preferred embodiments, DUX4 expression is reduced by at least 70%. In further preferred embodiments, DUX4 expression is reduced by at least 80%. In further preferred embodiments, DUX4 expression is reduced by at least 90%. In further preferred embodiments, DUX4 expression is reduced by at least 95%. In the most preferred embodiments, DUX4 expression is reduced by about 100%, preferably by 100%.

In preferred embodiments, the invention provides a compound for use according to the invention, wherein the compound reduces DUX4 expression in muscle cells, immune cells, or cancer cells, preferably in muscle cells or immune cells, most preferably in muscle cells. Preferred muscle cells are myoblasts, satellite cells, myotubes, and myofibers. Preferred immune cells are B cells, T cells, dendritic cells, neutrophils, natural killer cells, granulocytes, innate lymphoid cells, megakaryocytes, myeloid-derived suppressor cells, monocytes/ macrophages, and thymocytes, and optionally mast cells. Other preferred cells are platelets and red blood cells. In other embodiments, DUX4 expression is reduced in cancer cells.

In preferred embodiments, a compound according to the invention is for the treatment of patients suffering from both a DUX4-related condition and from muscle inflammation. Muscle inflammation contributes to the pathophysiology of muscular dystrophies such as FSHD. It precedes muscle destruction and fatty replacement, thereby representing an early marker for disease activity. Muscle inflammation can be identified using means known in the art. Preferably, muscle inflammation is identified by at least one of using biopsies and using MRI sequences with short TI inversion recovery (STIR), preferably using MRI with STIR. STIR hyperintensities (STIR+) visualize edema, which correlates with inflammation. A preferred inflamed muscle is a STIR+ muscle. A preferred muscle biopsy is a biopsy from a STIR+ muscle. A preferred muscle inflammation is MAPK-associated muscle inflammation, more preferably a muscle inflammation associated with the transcription and translation of inflammatory response-associated genes that encode proteins such as TNF-a, IL-1b, IL-6, and IL-8. Muscle inflammation predicts a faster fat replacement of muscle.

A preferred subject suffering from muscle inflammation has at least one inflamed muscle, more preferably at least 2, even more preferably at least 3, even more preferably at least 4, even more preferably at least 5, most preferably at least 6, 7, 8, 9, 10, or 11. Preferably the inflamed muscle is a skeletal muscle, more preferably it is a skeletal muscle of the face, scapula, or upper arms. A preferred subject suffering from muscle inflammation is a subject also suffering from muscular dystrophy, more preferably also suffering from FSHD. Preferably, such a subject suffering from FSHD has at least one inflamed muscle, more preferably at least one STIR+ muscle.

The invention provides a compound according to the invention for use in the treatment of a disease or condition associated with DUX4 expression in a subject, wherein the subject suffers from muscle inflammation. In preferred embodiments, the invention provides compound according to the invention for use in the treatment of FSHD, wherein the subject suffers from muscle inflammation. In preferred embodiments, the invention provides a compound according to the invention for use in the treatment of FSHD, wherein the subject has at least one inflamed muscle, preferably at least one inflamed skeletal muscle of the face, scapula, or upper arms. This muscle is preferably STIR+. Muscle inflammation is known to precede fatty infiltration. Accordingly, the invention provides a compound according to the invention for preventing or delaying fatty infiltration in a muscle of a subject suffering from FSHD.

In preferred embodiments, a compound according to the invention or a combination as defined herein is for the promotion of myogenic fusion and/or for the promotion of myogenic differentiation. The inventors have identified that compounds according to the invention promote both of these important characteristics of healthy or recovering muscles. The use in promoting myogenic fusion and/or myogenic differentiation aids with muscle regeneration.

Skeletal muscle is an example of a tissue that deploys a self-renewing stem cell, the satellite cell, to effect regeneration. These satellite cells remain adjacent to a skeletal muscle fiber, situated between the sarcolemma and the basement membrane of the endomysium (the connective tissue investment that divides the muscle fascicles into individual fibers). To activate myogenesis, the satellite cells must be stimulated to differentiate into new fibers. The satellite cells show asymmetric divisions to renew rare “immortal” stem cells and generate a clonal population of differentiation-competent myoblasts. The myoblast is thus a type of muscle progenitor cell that arises from myogenic satellite cells. Myoblasts differentiate to give rise to muscle cells. Differentiation is regulated by myogenic regulatory factors, including but not limited to MyoD, Myf5, myogenin, and MRF4. GATA4 and GATA6 also play a role in myocyte differentiation. Skeletal muscle fibers are made when myoblasts fuse together or to existing myofibers; muscle fibers therefore are cells with multiple nuclei, known as myonuclei. The myogenic fusion process is specific to skeletal muscle (e.g., biceps brachii) and not cardiac muscle or smooth muscle. The inventors have identified that compounds according to the invention promote this differentiation of satellite cells, thus ultimately promoting myotube formation and myogenesis.

The invention provides a compound according to the invention for use in the treatment of a disease or condition associated with DUX4 expression in a subject, wherein the compound is for promoting myogenic fusion and/or differentiation. Such promoted fusion and differentiation help reinstate healthy skeletal muscle biology. In preferred embodiments, the compound according to the invention is for promoting myogenic fusion. Myogenic fusion is quintessential to muscle formation and muscle regeneration, and it can be assessed using any known method. Preferably, it is assessed using image analysis, more preferably using high content image analysis. In preferred embodiments, the compound according to the invention for promoting myogenic fusion increases myogenic fusion with at least 1, 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 90, 95, 100% or more, preferably with at least 10% or more, more preferably with at least 30% or more, even more preferably with at least 50% or more. It can be that no myogenic fusion was present in a subject or in a muscle or in a sample. In such a case the compound according to the invention for promoting myogenic fusion preferably reinstates myogenic fusion, more preferably to at least 1%, 5%, 10%, 20%, 25%, 30%, 35%, 40%, 45%, 50% or more of a healthy control, even more preferably to at least 5% of a healthy control, more preferably still to at least 15%, most preferably to at least 25% of a healthy control.

In preferred embodiments the compound according to the invention is for promoting myogenic differentiation, which can be in vitro, in vivo, or ex vivo, preferably in vitro or ex vivo, more preferably in vitro. In these embodiments, a cell is preferably a primary cell. In these embodiments, a cell is preferably not an immortalized cell. Myogenic differentiation can be assessed using methods known in the art, such as quantification of myogenic differentiation markers such as MYH2, MyoD, Myf5, myogenin, and 15 MRF4, preferably such as myogenin or MYH2. In preferred embodiments, the compound according to the invention for promoting myogenic differentiation increases myogenic differentiation with at least 1, 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 90, 95, 100% or more, preferably with at least 10% or more, more preferably with at least 30% or more, even more preferably with at least 50% or more. It can be that no myogenic differentiation was present in a subject or in a muscle or in a sample. In such a case the compound according to the invention for promoting myogenic differentiation preferably reinstates myogenic differentiation, more preferably to at least 1%, 5%, 10%, 20%, 25%, 30%, 35%, 40%, 45%, 50% or more of a healthy control, even more preferably to at least 5% of a healthy control, more preferably still to at least 15%, most preferably to at least 25% of a healthy control.

In preferred embodiments, the compound according to the invention is for promoting myogenic fusion, wherein features and definitions are as defined elsewhere herein. In preferred embodiments the compound according to the invention is for promoting myogenic differentiation, wherein features and definitions are as defined elsewhere herein. In preferred embodiments, the compound according to the invention is for promoting myogenic fusion and/or differentiation, wherein features and definitions are as defined elsewhere herein.

In preferred embodiments the invention provides the compounds for use according to the invention, wherein said disease or condition associated with DUX4 expression is a muscular dystrophy or cancer or systemic cachexia, preferably wherein said disease or condition associated with DUX4 expression is a muscular dystrophy, most preferably facioscapulohumeral muscular dystrophy (FSHD). In other preferred embodiments, the compound according to the invention is for treating, ameliorating, or preventing systemic cachexia.

In this context, a preferred muscular dystrophy is FSHD; a preferred cancer is prostate cancer (WO2014081923), multiple myeloma (US20140221313), lung cancer (Lang et al., 2014, DOI: 10.14205/2310-8703.2014.02.01.1), colon cancer (Paz et al., 2003, DOI: 10.1093/hmg/ddg226) sarcoma, or leukemia; a preferred sarcoma is small round cell sarcoma (Oyama et al., 2017 DOI: 10.1038/s41598-017-04967-0 ; Bergerat et al., 2017, DOI: 10.1016/j.prp.2016.11.015 ; Chebib and Jo, 2016, DOI: 10.1002/cncy.21685); a preferred leukemia is acute lymphoblastic leukemia (ALL), more particularly B-cell precursor ALL (Yasuda et al., 2016, doi: 10.1038/ng.3535 ; Lilljebjörn & Fioretos, 2017, DOI: 10.1182/blood-2017-05-742643 ; Zhang et al., 2017, DOI:10.1038/ng.3691).

Accordingly, in preferred embodiments, the invention provides the compounds for use according to the invention, wherein said disease or condition associated with DUX4 expression is a muscular dystrophy or cancer, preferably wherein said disease or condition associated with DUX4 expression is FSHD, prostate cancer, multiple myeloma, lung cancer, colon cancer (preferably colorectal carcinoma), sarcoma (preferably small round cell sarcoma), leukemia (preferably acute lymphoblastic leukemia, more preferably B-cell precursor acute lymphoblastic leukemia), preferably said disease or condition associated with DUX4 expression is FSHD. In more preferred embodiments, the invention provides the compounds for use according to the invention, wherein said disease or condition associated with DUX4 expression is a muscular dystrophy or cancer, preferably wherein said disease or condition associated with DUX4 expression is FSHD or cancer, wherein cancer is preferably prostate cancer, multiple myeloma, lung cancer, colon cancer (preferably colorectal carcinoma), sarcoma (preferably small round cell sarcoma), leukemia (preferably acute lymphoblastic leukemia, more preferably B-cell precursor acute lymphoblastic leukemia), wherein cancer is more preferably sarcoma, most preferably small round cell sarcoma.

In a preferred embodiment, the invention provides the compounds for use according to the invention, wherein said disease or condition associated with DUX4 expression is cancer, wherein cancer is preferably prostate cancer, multiple myeloma, lung cancer, colon cancer (preferably colorectal carcinoma), sarcoma (preferably small round cell sarcoma), leukemia (preferably acute lymphoblastic leukemia, more preferably B-cell precursor acute lymphoblastic leukemia), wherein cancer is more preferably sarcoma, most preferably small round cell sarcoma.

Other DUX4 targets are known as “cancer testis antigens” (CTAs), which are genes that are normally expressed only in testis, but which are de-repressed in some cancers, eliciting an immune response. These observations imply that DUX4 de-repression in cancers mediates the activation of HSATII, CTAs and/or THE1B promoters (Young et al., 2013, doi:10.1371/journal.pgen.1003947). In line with this, Dmitriev et al. (2014, DOI: 10.1111/jcmm.12182) demonstrate a similarity between FSHD and cancer cell expression profiles, suggesting a common step in the pathogenesis of these diseases.

Expression of DUX4 is known to be associated with immune suppression in tumors (Guo-Liang Chew et al., 2019, Developmental Cell 50, 658-671, DOI: 10.1016/j.devcel.2019.06.011). DUX4 is re-expressed in many cancers, where it suppresses anti-cancer immune activity by blocking interferon-γ-mediated induction of MHC class I and is associated with reduced efficacy of immune checkpoint blockade therapy. DUX4-expressing cancers are characterized by low antitumor immune activity. DUX4 blocks interferon-γ-mediated induction of MHC class I and antigen presentation. As a result, DUX4 is significantly associated with failure to respond to anti-CTLA-4 therapy.

In preferred embodiments, a compound or composition according to the invention is for use in the treatment of cancer, wherein the compound or composition increases the immune response to cancer cells. This may mean that it initiates an immune response in cases where no immune response was present. In this application, a preferred cancer is a cancer with DUX4 expression, more preferably a cancer with reduced MHC class I expression.

In more preferred embodiments for increasing immune response, the compound or composition according to the invention is for increasing the production of immune system activating cytokines, such as interferon-y. Preferably, cytokine production is increased by 1%, 5%, 10%, 15%, 20%, 25%, 30%, 40%, 50%, 55%, 60%, 65%, 70% or 75%, or more, and is preferably detected through FACS. The increase in cytokines leads to increased immune suppression of cancers and can lead to immune-mediated suppression or partial immune-mediated suppression of cancers that would otherwise not be susceptible to immune-mediated suppression. In preferred embodiments, the compound or composition according to the invention is for increasing T-cell function, such as increasing production of interferon-y.

In preferred embodiments for increasing immune response, the compound or composition according to the invention is for increasing T-cell frequency. Preferably, such an increase is by 1%, 5%, 10%, 15%, 20%, 25%, 30%, 40%, 50%, 55%, 60%, 65%, 70% or 75%, or more. Such an increase can be determined by measuring CD8 or CD4. For example as described in Guo-Liang Chew et al. In other preferred embodiments for increasing immune response, the compound or composition according to the invention is for increasing specific T-cell subsets. Such subsets can be determined by TCR sequencing. In preferred embodiments for increasing immune response, the compound or composition according to the invention is for inducing T-cell function, preferably for inducing T-cell function by inducing IFNy production. Most preferably, the compound or composition according to the invention is for increasing T-cell frequency and simultaneously inducing T-cell function, preferably while simultaneously decreasing regulatory T cell population. Tumors with decreased Tregs and with increased CD8+ T effector cells are referred to as ‘hot’ tumors, which are tumors that do not have an immunosuppressed microenvironment. Conversely, tumors in an immunosuppressed microenvironment are referred to as ‘cold’ tumors.

Additionally, compounds and compositions according to the invention can reduce expression of immune suppressive target genes such as, but not limited to, CTLA-4 or PD-1 or PD-1L. Such a reduction is preferably by 1%, 5%, 10%, 15%, 20%, 25%, 30%, 40%, 50%, 55%, 60%, 65%, 70% or 75%, or more. Expression can be determined via qPCR. CTLA-4 and PD-1 are T cell inhibitory receptors on which immune checkpoint blockade therapies can act. Such therapy induces durable responses across diverse cancers in susceptible patients. In preferred embodiments, the compound or composition according to the invention is for reducing expression of CTLA-4 or of PD-1 or for reducing expression of CTLA-4 and PD-1.

Additionally, compounds and compositions according to the invention can be combined with compounds that inhibit immune checkpoints such as, but not limited to, CTLA-4, PD-1, or PD-L1. In preferred embodiments, a combination is provided comprising the compound or composition according to the invention and a further compound is for inhibiting CTLA-4, PD-1, or PD-L1. Examples of such further agents are pembrolizumab, spartalizumab, nivolumab (PD-1 inhibitors), and ipilimumab (CTLA-4 inhibitor). Such inhibition is preferably by 1%, 5%, 10%, 15%, 20%, 25%, 30%, 40%, 50%, 55%, 60%, 65%, 70% or 75%, or more. Inhibition can be determined via methods known in the art, such as described or referred to in Guo-Liang Chew et al., 2019.

The compounds of the present invention are also adapted to therapeutic use as antiproliferative agents (e.g., cancer), antitumor (e.g., effect against solid tumors) in mammals, particularly in humans. In particular, the compounds of the present invention are useful in the prevention and treatment of a variety of human hyperproliferative disorders including both malignant and benign abnormal cell growth. The compounds, compositions and methods provided herein are useful for the treatment of cancer and preparation of a medicament to treat cancer including but are not limited to cancer of:
the circulatory system, for example, heart (sarcoma [angiosarcoma, fibrosarcoma, rhabdomyosarcoma, liposarcoma], myxoma, rhabdomyoma, fibroma, lipoma and teratoma), mediastinum and pleura, and other intrathoracic organs, vascular tumors and tumor-associated vascular tissue;
respiratory tract, for example, nasal cavity and middle ear, accessory sinuses, larynx, trachea, bronchus and lung such as small cell lung cancer (SCLC), non-small cell lung cancer (NSCLC), bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; gastrointestinal, for example, esophagus (squamous cell carcinoma, adenocarcinoma, leiomyosarcoma, lymphoma), stomach (carcinoma, lymphoma, leiomyosarcoma), gastric, pancreas (ductal adenocarcinoma, insulinoma, glucagonoma, gastrinoma, carcinoid tumors, vipoma), small bowel (adenocarcinoma, lymphoma, carcinoid tumors, Karposi’s sarcoma, leiomyoma, hemangioma, lipoma, neurofibroma, fibroma), large bowel (adenocarcinoma, tubular adenoma, villous adenoma, hamartoma, leiomyoma);
genitourinary tract, for example, kidney (adenocarcinoma, Wilm’s tumor [nephroblastoma], lymphoma, leukemia), bladder and/or urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma);
liver, for example, hepatoma (hepatocellular carcinoma), cholangiocarcinoma, hepatoblastoma, angiosarcoma, hepatocellular adenoma, hemangioma, pancreatic endocrine tumors (such as pheochromocytoma, insulinoma, vasoactive intestinal peptide tumor, islet cell tumor and glucagonoma);
bone, for example, osteogenic sarcoma (osteosarcoma), fibrosarcoma, malignant fibrous histiocytoma, chondrosarcoma, Ewing’s sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors;
nervous system, for example, neoplasms of the central nervous system (CNS), primary CNS lymphoma, skull cancer (osteoma, hemangioma, granuloma, xanthoma, osteitis deformans), meninges (meningioma, meningiosarcoma, gliomatosis), brain cancer (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma);
reproductive system, for example, gynecological, uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminonna, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma) and other sites associated with female genital organs; placenta, penis, prostate, testis, and other sites associated with male genital organs;
hematologic, for example, blood (myeloid leukemia [acute and chronic], acute lymphoblastic leukemia, chronic lymphocytic leukemia, myeloproliferative diseases, multiple myeloma, myelodysplastic syndrome), Hodgkin’s disease, non-Hodgkin’s lymphoma [malignant lymphoma];
oral cavity, for example, lip, tongue, gum, floor of mouth, palate, and other parts of mouth, parotid gland, and other parts of the salivary glands, tonsil, oropharynx, nasopharynx, pyriform sinus, hypopharynx, and other sites in the lip, oral cavity and pharynx;
skin, for example, malignant melanoma, cutaneous melanoma, basal cell carcinoma, squamous cell carcinoma, Karposi’s sarcoma, moles dysplastic nevi, lipoma, angioma, dermatofibroma, and keloids;
adrenal glands: neuroblastoma; and
cancers involving other tissues including connective and soft tissue, retroperitoneum and peritoneum, eye, intraocular melanoma, and adnexa, breast, head or/and neck, anal region, thyroid, parathyroid, adrenal gland and other endocrine glands and related structures, secondary and unspecified malignant neoplasm of lymph nodes, secondary malignant neoplasm of respiratory and digestive systems and secondary malignant neoplasm of other sites.

More specifically, examples of “cancer” when used herein in connection with the present invention include cancer selected from lung cancer (NSCLC and SCLC), cancer of the head or neck, ovarian cancer, colon cancer, rectal cancer, cancer of the anal region, stomach cancer, breast cancer, cancer of the kidney or ureter, renal cell carcinoma, carcinoma of the renal pelvis, neoplasms of the central nervous system (CNS), primary CNS lymphoma, non-Hodgkins’s lymphoma, spinal axis tumors, or a combination of one or more of the foregoing cancers. Still more specifically, examples of “cancer” when used herein in connection with the present invention include cancer selected from lung cancer (NSCLC and SCLC), breast cancer, ovarian cancer, colon cancer, rectal cancer, cancer of the anal region, or a combination of one or more of the foregoing cancers. In one embodiment of the present invention the non-cancerous conditions include such hyperplastic conditions such as benign hyperplasia of the skin (e.g., psoriasis) and benign hyperplasia of the prostate (e.g., BPH).

In another embodiment the present invention provides a compound of general formula (I) for use in methods of treating neurological and psychiatric disorders comprising: administering to a mammal an amount of a compound of general formula (I) effective in treating such disorders, or a pharmaceutically acceptable salt thereof. Neurological and psychiatric disorders include but are not limited to: acute neurological and psychiatric disorders such as cerebral deficits subsequent to cardiac bypass surgery and grafting, stroke, cerebral ischemia, spinal cord trauma, head trauma, perinatal hypoxia, cardiac arrest, hypoglycemic neuronal damage, dementia, AIDS-induced dementia, vascular dementia, mixed dementias, age- associated memory impairment, Alzheimer’s disease, Huntington’s Chorea, amyotrophic lateral sclerosis, ocular damage, retinopathy, cognitive disorders, including cognitive disorders associated with schizophrenia and bipolar disorders, idiopathic and drug-induced Parkinson’s disease, muscular spasms and disorders associated with muscular spasticity including tremors, epilepsy, convulsions, migraine, migraine headache, urinary incontinence, substance tolerance, substance withdrawal, withdrawal from opiates, nicotine, tobacco products, alcohol, benzodiazepines, cocaine, sedatives, and hypnotics, psychosis, mild cognitive impairment, amnestic cognitive impairment, multi-domain cognitive impairment, obesity, schizophrenia, anxiety, generalized anxiety disorder, social anxiety disorder, panic disorder, post-traumatic stress disorder, obsessive compulsive disorder, mood disorders, depression, mania, bipolar disorders, trigeminal neuralgia, hearing loss, tinnitus, macular degeneration of the eye, emesis, brain edema, pain, acute and chronic pain states, severe pain, intractable pain, neuropathic pain, post-traumatic pain, tardive dyskinesia, sleep disorders, narcolepsy, attention deficit/hyperactivity disorder, autism, Asperger’s disease, and conduct disorder in a mammal. Accordingly, in one embodiment, the invention provides a method for treating a condition in a mammal, such as a human, selected from the conditions above, comprising administering a compound of general formula (I) to the mammal. The mammal is preferably a mammal in need of such treatment. As examples, the invention provides a compound of general formula (I) for use in method for treating or preparation of a medicament to treat attention deficit/hyperactivity disorder, schizophrenia and Alzheimer’s Disease.

The invention relates to a compound of general formula (I) for use in a method of treating a mood disorder selected from the group consisting of a depressive disorder and a bipolar disorder. In another embodiment of the invention, the depressive disorder is major depressive disorder. In a further embodiment of the invention, the mood disorder is a bipolar disorder. In another embodiment, the bipolar disorder is selected from the group consisting of bipolar I disorder and bipolar II disorder.

The compound of general formula (I) can also be for use in treating a condition selected from the group consisting of neurological and psychiatric disorders, including but not limited to: acute neurological and psychiatric disorders such as cerebral deficits subsequent to cardiac bypass surgery and grafting, stroke, cerebral ischemia, spinal cord trauma, head trauma, perinatal hypoxia, cardiac arrest, hypoglycemic neuronal damage, dementia, AIDS-induced dementia, vascular dementia, mixed dementias, age- associated memory impairment, Alzheimer’s disease, Huntington’s Chorea, amyotrophic lateral sclerosis, ocular damage, retinopathy, cognitive disorders, including cognitive disorders associated with schizophrenia and bipolar disorders, idiopathic and drug-induced Parkinson’s disease, muscular spasms and disorders associated with muscular spasticity including tremors, epilepsy, convulsions, migraine, migraine headache, urinary incontinence, substance tolerance, substance withdrawal, withdrawal from opiates, nicotine, tobacco products, alcohol, benzodiazepines, cocaine, sedatives, and hypnotics, psychosis, mild cognitive impairment, amnestic cognitive impairment, multi-domain cognitive impairment, obesity, schizophrenia, anxiety, generalized anxiety disorder, social anxiety disorder, panic disorder, post-traumatic stress disorder, obsessive compulsive disorder, mood disorders, depression, mania, bipolar disorders, trigeminal neuralgia, hearing loss, tinnitus, macular degeneration of the eye, emesis, brain edema, pain, acute and chronic pain states, severe pain, intractable pain, neuropathic pain, post-traumatic pain, tardive dyskinesia, sleep disorders, narcolepsy, attention deficit/hyperactivity disorder, autism, Asperger’s disease, and conduct disorder in a mammal, comprising administering an effective amount of a compound of general formula (I) or pharmaceutically acceptable salt thereof, and a pharmaceutically acceptable carrier. The composition optionally further comprises an atypical antipsychotic, a cholinesterase inhibitor, Dimebon, or NMDA receptor antagonist. Such atypical antipsychotics include, but are not limited to, ziprasidone, clozapine, olanzapine, risperidone, quetiapine, aripiprazole, paliperidone; such NMDA receptor antagonists include but are not limited to memantine; and such cholinesterase inhibitors include but are not limited to donepezil and galantamine.

Compounds according to the invention can also be used for treating auto-immune disorders. Particularly suitable disorders in this context are such as rheumatoid arthritis, asthma, psoriasis, chronic pulmonary inflammation, chronic obstructive pulmonary disease, asthma, glomerulonephritis, Crohn’s disease, ICF (immunodeficiency, centromeric region instability and facial anomalies), and myositis such as myositis ossificans, (idiopathic) inflammatory myopathies, dermatomyositis, juvenile dermatomyositis, polymyositis, inclusion body myositis, benign acute childhood myositis, statin-associated autoimmune myopathy, and pyomyositis. Preferred in this context are ICF and myositis, wherein myositis is most preferred.

Many targets are known to be associated with DUX4 repression. Examples are BET proteins (such as BRD2, BRD3, BRD4, BRDT) and β2-adrenergic receptor (Campbell et al., Skeletal Muscle. 2017 Sep 4; 7(1)); SMCHD1 (Balog et al., Epigenetics. 2015; 10(12): 1133-42); PARP1 (Sharma V et al., J. Genetic syndromes and Gene Therapy. 2016 Aug; 7(4)); WNT signalling proteins (such as WNT1-16, Axin, beta-catenin, Frizzled, and GSK3) and tankyrase (Block et al., Hum Mol Genet. 2013 Dec 1;22(23):4661-72) PRC2/EZH2 and SUV39H1 (Haynes et al., Epigenetics & Chromatin. 2018, 11 (47)); MBD2/NuRD complex, MBD1/CAF-1, TRIM28, SETDB1, KDM1A, SIN3 complex (Campbell et al., eLife. 2018, 7:e31023); ASH1L, BAP1, BAZ1A, BAZ1B, BAZ2A, BPTF, BRD2, BRD3, BRD4, BRDT, BRPF1, BRPF3, CARM1, KDM4A, KDM4B, KDM4C, KDM4D, KDM6A, KDM6B, KMT2A, KMT2C, KMT2E, MYSM1, NEK6, PHF2, PRMT1, SETD1A, SETD1B, SF3B1, SMARCA5, SMARCB1, SMYD3, UFL1, USP3, USP7, USP16 (Himeda et al., Molecular Therapy. 2018 Apr 20, 26 (7)); Src family (such as Src, Yes, Fyn, and Fgr, Lck, Hck, Blk, Lyn, Frk, WO2019084499); Syk family (such as Syk, WO2019084499); Abl family (such as Abl1, WO2019084499); Tie family (such as Tie1, Tie2, TEK, WO2019084499); Flt family (such as VEGFR1, WO2019084499); CK1 (such as CK1d, CK1e, WO2019115711); ErbB family (such as Her1 (EGFR, ErbB1), Her2 (Neu, ErbB2), Her3 (ErbB3), and Her4 (ErbB4), WO2019084499); p38 (WO2019071147); Trk family (such as TrkA, TrkB, TrkC, WO2019084499); and PI3K family (such as ATM, ATR, PRKDC, mTOR, SMG1, TRRAP, WO2019084499).

In light of the above, in preferred embodiments the compound is for use in modulating BET protein activity; in other preferred embodiments the compound is for use in modulating β2-adrenergic receptor activity; in other preferred embodiments the compound is for use in modulating SMCHD1 activity; in other preferred embodiments the compound is for use in modulating PARP1 activity; in other preferred embodiments the compound is for use in modulating WNT signaling activity; in other preferred embodiments the compound is for use in modulating tankyrase activity; in other preferred embodiments the compound is for use in modulating PRC2/EZH2 activity; in other preferred embodiments the compound is for use in modulating SUV39H1 activity; in other preferred embodiments the compound is for use in modulating MBD2/NuRD complex activity; in other preferred embodiments the compound is for use in modulating MBD1/CAF-1 activity; in other preferred embodiments the compound is for use in modulating TRIM28 activity; in other preferred embodiments the compound is for use in modulating SETDB1 activity; in other preferred embodiments the compound is for use in modulating KDM1A activity; in other preferred embodiments the compound is for use in modulating SIN3 complex activity; in other preferred embodiments the compound is for use in modulating ASH1L activity; in other preferred embodiments the compound is for use in modulating BAP1 activity; in other preferred embodiments the compound is for use in modulating BAZ1A activity; in other preferred embodiments the compound is for use in modulating BAZ1B activity; in other preferred embodiments the compound is for use in modulating BAZ2A activity; in other preferred embodiments the compound is for use in modulating BPTF activity; in other preferred embodiments the compound is for use in modulating BRD2 activity; in other preferred embodiments the compound is for use in modulating BRD3 activity; in other preferred embodiments the compound is for use in modulating BRD4 activity; in other preferred embodiments the compound is for use in modulating BRDT activity; in other preferred embodiments the compound is for use in modulating BRPF1 activity; in other preferred embodiments the compound is for use in modulating BRPF3 activity; in other preferred embodiments the compound is for use in modulating CARM1 activity; in other preferred embodiments the compound is for use in modulating KDM4A activity; in other preferred embodiments the compound is for use in modulating KDM4B activity; in other preferred embodiments the compound is for use in modulating KDM4C activity; in other preferred embodiments the compound is for use in modulating KDM4D activity; in other preferred embodiments the compound is for use in modulating KDM6A activity; in other preferred embodiments the compound is for use in modulating KDM6B activity; in other preferred embodiments the compound is for use in modulating KMT2A activity; in other preferred embodiments the compound is for use in modulating KMT2C activity; in other preferred embodiments the compound is for use in modulating KMT2E activity; in other preferred embodiments the compound is for use in modulating MYSM1 activity; in other preferred embodiments the compound is for use in modulating NEK6 activity; in other preferred embodiments the compound is for use in modulating PHF2 activity; in other preferred embodiments the compound is for use in modulating PRMT1 activity; in other preferred embodiments the compound is for use in modulating SETD1A activity; in other preferred embodiments the compound is for use in modulating SETD1B activity; in other preferred embodiments the compound is for use in modulating SF3B1 activity; in other preferred embodiments the compound is for use in modulating SMARCA5 activity; in other preferred embodiments the compound is for use in modulating SMARCB1 activity; in other preferred embodiments the compound is for use in modulating SMYD3 activity; in other preferred embodiments the compound is for use in modulating UFL1 activity; in other preferred embodiments the compound is for use in modulating USP3 activity; in other preferred embodiments the compound is for use in modulating USP7 activity; in other preferred embodiments the compound is for use in modulating USP16 activity; in other preferred embodiments the compound is for use in modulating Src family activity; in other preferred embodiments the compound is for use in modulating Syk family activity; in other preferred embodiments the compound is for use in modulating Abl family activity; in other preferred embodiments the compound is for use in modulating Tie family activity; in other preferred embodiments the compound is for use in modulating Flt family activity; in other preferred embodiments the compound is for use in modulating CK1 activity; in other preferred embodiments the compound is for use in modulating ErbB family activity; in other preferred embodiments the compound is for use in modulating p38 activity; in other preferred embodiments the compound is for use in modulating Trk family activity; in other preferred embodiments the compound is for use in modulating PI3K family activity. In this context, modulation of activity is preferably inhibition of activity. Modulation and inhibition can be assayed as described in the respective sources cited above.

### Formulation and Administration

The compositions comprising the compounds as described above, can be prepared as a medicinal or cosmetic preparation or in various other media, such as foods for humans or animals, including medical foods and dietary supplements. A “medical food” is a product that is intended for the specific dietary management of a disease or condition for which distinctive nutritional requirements exist. By way of example, not limitation, medical foods may include vitamin and mineral formulations fed through a feeding tube (referred to as enteral administration). A “dietary supplement” shall mean a product that is intended to supplement the human diet and is typically provided in the form of a pill, capsule, tablet or like formulation. By way of example, not limitation, a dietary supplement may include one or more of the following ingredients: vitamins, minerals, herbs, botanicals; amino acids, dietary substances intended to supplement the diet by increasing total dietary intake, and concentrates, metabolites, constituents, extracts or combinations of any of the foregoing. Dietary supplements may also be incorporated into food, including, but not limited to, food bars, beverages, powders, cereals, cooked foods, food additives and candies; or other functional foods designed to promote health or to prevent or halt the progression of a degenerative disease associated with DUX4 expression.

The subject compounds and compositions may be compounded with other physiologically acceptable materials that can be ingested including, but not limited to, foods. In addition, or alternatively, the compositions as described herein may be administered orally in combination with (the separate) administration of food.

The compositions or compound according to the invention may be administered alone or in combination with other pharmaceutical or cosmetic agents and can be combined with a physiologically acceptable carrier thereof. In particular, the compounds described herein can be formulated as pharmaceutical or cosmetic compositions by formulation with additives such as pharmaceutically or physiologically acceptable excipients carriers, and vehicles. Suitable pharmaceutically or physiologically acceptable excipients, carriers and vehicles include processing agents and drug delivery modifiers and enhancers, such as, for example, calcium phosphate, magnesium stearate, talc, monosaccharides, disaccharides, starch, gelatin, cellulose, methyl cellulose, sodium carboxymethyl cellulose, dextrose, hydroxypropyl-P-cyclodextrin, polyvinylpyrrolidinone, low melting waxes, ion exchange resins, and the like, as well as combinations of any two or more thereof. Other suitable pharmaceutically acceptable excipients are described in “Remington’s Pharmaceutical Sciences, “ Mack Pub. Co., New Jersey (1991), and “Remington: The Science and Practice of Pharmacy, “ Lippincott Williams & Wilkins, Philadelphia, 20th edition (2003), 21^{st} edition (2005) and 22^{nd} edition (2012), incorporated herein by reference.

Compositions for use according to the invention may be manufactured by processes well known in the art; e.g., by means of conventional mixing, dissolving, granulating, dragee-making, levigating, emulsifying, encapsulating, entrapping or lyophilizing processes, which may result in liposomal formulations, coacervates, oil-in-water emulsions, nanoparticulate/microparticulate powders, or any other shape or form. Compositions for use in accordance with the invention thus may be formulated in a conventional manner using one or more physiologically acceptable carriers comprising excipients and auxiliaries that facilitate processing of the active compounds into preparations which can be used pharmaceutically. Proper formulation is dependent on the route of administration chosen.

For injection, the compounds and compositions for use according to the invention may be formulated in aqueous solutions, preferably in physiologically compatible buffers such as Hanks’s solution, Ringer’s solution, or physiological saline buffer. For transmucosal administration, penetrants appropriate to the barrier to be permeated are used in the formulation. Such penetrants are generally known in the art.

Oral and parenteral administration may be used where the compounds and compositions for use are formulated by combining them with pharmaceutically acceptable carriers well known in the art, or by using them as a food additive. Such strategies enable the compounds and compositions for use according to the invention to be formulated as tablets, pills, dragees, capsules, liquids, gels, syrups, slurries, suspensions and the like, for oral ingestion by a subject to be treated. Preparations or pharmacological preparations for oral use may be made with the use of a solid excipient, optionally grinding the resulting mixture, and processing the mixture of granules, after adding suitable auxiliaries, if desired, to obtain tablets or dragée cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as crosslinked polyvinyl pyrrolidone, agar, or alginic acid or a salt thereof such as sodium alginate. Additionally, coformulations may be made with uptake enhancers known in the art.

Dragée cores are provided with suitable coatings. For this purpose, concentrated sugar solutions may be used, which may optionally contain gum arabic, talc, PVP, carbopol gel, polyethylene glycol, and/or titanium dioxide, lacquer solution, and suitable organic solvents or solvent mixtures. Polymethacrylates can be used to provide pH-responsive release profiles so as to pass the stomach. Dyestuffs or pigments may be added to the tablets or dragée coatings for identification or to characterize different combinations of active compound doses.

Compounds and compositions which can be administered orally include push-fit capsules made of gelatin, as well as soft, sealed capsules made of gelatin and a plasticizer, such as glycerol or sorbitol. The push-fit capsules may contain the active ingredients in admixture with a filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compounds and compositions for use according to the invention may be administered in the form of tablets or lozenges formulated in a conventional manner.

The compounds and compositions for use according to the invention may be formulated for parenteral administration by injection, e.g., by bolus injection or continuous infusion. In this way it is also possible to target a particular organ, tissue, tumor site, site of inflammation, etc. Formulations for infection may be presented in unit dosage form, e.g., in ampoules or in multi-dose container, with an added preservative. The compositions may take such forms as suspensions, solutions or emulsions in oily or aqueous vehicles, and may contain formulatory agents such as suspending, stabilizing and/or dispersing agents. This formulation is preferred because it enables specific targeting of muscle tissue.

Compositions for parenteral administration include aqueous solutions of the compositions in water soluble form. Additionally, suspensions may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compositions to allow for the preparation of highly concentrated solutions.

Alternatively, one or more components of the composition may be in powder form for constitution with a suitable vehicle, e.g., sterile pyrogen-free water, before use.

The compositions for use according to the invention may also be formulated in rectal compositions such as suppositories or retention enemas, e.g., containing conventional suppository bases such as cocoa butter or other glycerides.

In addition to the formulations described previously, the compounds and compositions for use according to the invention may also be formulated as a depot preparation. Such long acting formulations may be administered by implantation (for example subcutaneously or intramuscularly) or by intramuscular injection. Thus, for example, they may be formulated with suitable polymeric or hydrophobic materials (for example as an emulsion in an acceptable oil), or as part of a solid or semi-solid implant that may or may not be auto-degrading in the body, or ion exchange resins, or one or more components of the composition can be formulated as sparingly soluble derivatives, for example, as a sparingly soluble salt. Examples of suitable polymeric materials are known to the person skilled in the art and include PLGA and polylactones such as polycaproic acid.

The compositions for use according to the invention also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

The compositions for use according to the invention may also be comprised in a transdermal patch. Preferred transdermal patches for use according to the invention are selected from single-layer drug-in-adhesive patch, or multi-layer drug-in-adhesive patch, or reservoir patch, or matrix patch, or vapour patch.

Compositions for use according to the invention include compounds and compositions wherein the active ingredients are contained in an amount effective to achieve their intended purposes. More specifically, a therapeutically effective amount means an amount of compound effective to prevent, stabilize, alleviate, revert, or ameliorate causes or symptoms of disease, or prolong the survival, mobility, or independence of the subject being treated. Determination of a therapeutically effective amount is within the capability of those skilled in the art, especially in light of the detailed disclosure provided herein. For any compounds and compositions used in the invention, the therapeutically effective amount or dose can be estimated initially from cell culture assays, for example as exemplified herein. Dosage may vary within this range depending upon the dosage form employed and the route of administration utilized. The exact formulation, route of administration and dosage can be chosen by the individual physician in view of the patient’s condition. (See e.g., Fingl, et al., 1975, in “The Pharmacological Basis of Therapeutics” Ch. 1 p. 1). The amount of compound and compositions administered will, of course, be dependent on the subject being treated, on the subject’s weight, the severity of the affliction, the manner of administration and the judgment of the prescribing physician.

A composition for use according to the invention may be supplied such that a compound for use according to the invention and one or more of the other components as defined herein are in the same container, either in solution, in suspension, or in powder form. A composition for use according to the invention may also be provided with all components provided separately from one another, for example to be mixed with one another prior to administration, or for separate or sequential administration. Various packaging options are possible and known to the ones skilled in the art, depending, among others, on the route and mechanism of administration. In light of the methods of administration described above, the invention provides a compound for use according to the invention, or a composition for use according to the invention, characterized in that it is administered orally, sublingually, intravascularly, intravenously, subcutaneously, transdermally, or optionally by inhalation; preferably orally.

An “effective amount” of a compound or composition is an amount which, when administered to a subject, is sufficient to reduce or eliminate either one or more symptoms of a disease, or to retard the progression of one or more symptoms of a disease, or to reduce the severity of one or more symptoms of a disease, or to suppress the manifestation of a disease, or to suppress the manifestation of adverse symptoms of a disease. An effective amount can be given in one or more administrations.

The “effective amount” of that may be combined with the carrier materials to produce a single dosage form will vary depending upon the host to which the active ingredient is administered and the particular mode of administration. The unit dosage chosen is usually fabricated and administered to provide a desired final concentration of the compound in the blood.

The effective amount (i.e. the effective total daily dose), preferably for adults, is herein defined as a total daily dose of about 0.01 to 2000 mg, or about 0.01 to 1000 mg, or about 0.01 to 500 mg, or about 5 to 1000 mg, or about 20 to 800 mg, or about 30 to 800 mg or about 30 to 700 mg, or about 20 to 700 mg or about 20 to 600 mg, or about 30 to 600 mg, or about 30 to 500 mg, about 30 to 450 mg or about 30 to 400 mg, or about 30 to 350 mg or about 30 to 300 mg or about 50 to 600 mg, or about 50 to 500 mg, or about 50 to 450 mg, or about 50 to 400 mg or about 50 to 300 mg, or about 50 to 250 mg, or about 100 to 250 mg or about 150 to 250 mg. In the most preferred embodiment, the effective amount is about 200 mg. In preferred embodiments, the invention provides a compound for use according to the invention, or a composition for use according to the invention, characterized in that it is administered to a subject in an amount ranging from 0.1 to 1500 mg/day, preferably from 0.1 to 1000 mg/day, more preferably from 0.1 to 400 mg/day, still more preferably from 0.25 to 150 mg/day, such as about 100 mg/day.

Alternatively, the effective amount of the compound, preferably for adults, preferably is administered per kg body weight. The total daily dose, preferably for adults, is therefore about 0.05 to about 40 mg/kg, about 0.1 to about 20 mg/kg, about 0.2 mg/kg to about 15 mg/kg, or about 0.3 mg/kg to about 15 mg/kg or about 0.4 mg/kg to about 15 mg/kg or about 0.5 mg/kg to about 14 mg/kg or about 0.3 mg/kg to about 14 mg/kg or about 0.3 mg/kg to about 13 mg/kg or about 0.5 mg/kg to about 13 mg/kg or about 0.5 mg/kg to about 11 mg/kg.

The total daily dose for children is preferably at most 200 mg. More preferably the total daily dose is about 0.1 to 200 mg, about 1 to 200 mg, about 5 to 200 mg about 20 to 200 mg about 40 to 200 mg, or about 50 to 200 mg. Preferably, the total daily dose for children is about 0.1 to 150 mg, about 1 to 150 mg, about 5 to 150 mg about 10 to 150 mg about 40 to 150 mg, or about 50 to 150 mg. More preferably, the total daily dose is about 5 to 100 mg, about 10 to 100 mg, about 20 to 100 mg about 30 to 100 mg about 40 to 100 mg, or about 50 to 100 mg. Even more preferably, the total daily dose is about 5 to 75 mg, about 10 to 75 mg, about 20 to 75 mg about 30 to 75 mg about 40 to 75 mg, or about 50 to 75 mg.

Alternative examples of dosages which can be used are an effective amount of the compounds for use according to the invention within the dosage range of about 0.1 µg /kg to about 300 mg/kg, or within about 1.0 µg /kg to about 40 mg/kg body weight, or within about 1.0 µg/kg to about 20 mg/kg body weight, or within about 1.0 µg /kg to about 10 mg/kg body weight, or within about 10.0 µg /kg to about 10 mg/kg body weight, or within about 100 µg/kg to about 10 mg/kg body weight, or within about 1.0 mg/kg to about 10 mg/kg body weight, or within about 10 mg/kg to about 100 mg/kg body weight, or within about 50 mg/kg to about 150 mg/kg body weight, or within about 100 mg/kg to about 200 mg/kg body weight, or within about 150 mg/kg to about 250 mg/kg body weight, or within about 200 mg/kg to about 300 mg/kg body weight, or within about 250 mg/kg to about 300 mg/kg body weight. Other dosages which can be used are about 0.01 mg/kg body weight, about 0.1 mg/kg body weight, about 1 mg/kg body weight, about 10 mg/kg body weight, about 20 mg/kg body weight, about 30 mg/kg body weight, about 40 mg/kg body weight, about 50 mg/kg body weight, about 75 mg/kg body weight, about 100 mg/kg body weight, about 125 mg/kg body weight, about 150 mg/kg body weight, about 175 mg/kg body weight, about 200 mg/kg body weight, about 225 mg/kg body weight, about 250 mg/kg body weight, about 275 mg/kg body weight, or about 300 mg/kg body weight.

Compounds or compositions for use according to the present invention may be administered in a single daily dose, or the total daily dosage may be administered in divided dosage of two, three or four times daily.

In a preferred embodiment of the invention, “subject”, “individual”, or “patient” is understood to be an individual organism, preferably a vertebrate, more preferably a mammal, even more preferably a primate and most preferably a human.

In a further preferred embodiment of the invention, the human is an adult, e.g. a person that is 18 years or older. In addition, it is herein understood that the average weight of an adult person is 62 kg, although the average weight is known to vary between countries. In another embodiment of the invention the average weight of an adult person is therefore between about 50-90 kg. It is herein understood that the effective dose as defined herein is not confined to subjects having an average weight. Preferably, the subject has a BMI (Body Mass Index) between 18.0 to 40.0 kg/m², and more preferably a BMI between 18.0 to 30.0 kg/m².

Alternatively, the subject to be treated is a child, e.g. a person that is 17 years or younger. In addition, the subject to be treated may be a person between birth and puberty or between puberty and adulthood. It is herein understood that puberty starts for females at the age of 10-11 years and for males at the age of 11-12 year. Furthermore, the subject to be treated may be a neonate (first 28 days after birth), an infant (0-1 year), a toddler (1-3 years), a preschooler (3-5 years); a school-aged child (5-12 years) or an adolescent (13-18 years).

To maintain an effective range during treatment, the compound or composition may be administered once a day, or once every two, three, four, or five days. However preferably, the compound may be administered at least once a day. Hence in a preferred embodiment, the invention pertains to a compound for use according to the invention, or a composition for use according to the invention, characterized in that it is administered to a subject 4, 3, 2, or 1 times per day or less, preferably 1 time per day. The total daily dose may be administered as a single daily dose. Alternatively, the compound is administered at least twice daily. Hence, the compound as defined herein may be administered once, twice, three, four or five times a day. As such, the total daily dose may be divided over the several doses (units) resulting in the administration of the total daily dose as defined herein. In a preferred embodiment, the compound is administered twice daily. It is further understood that the terms “twice daily”, “bid” and “bis in die” can be used interchangeable herein.

In a preferred embodiment, the total daily dose is divided over several doses per day. These separate doses may differ in amount. For example, for each total daily dose, the first dose may have a larger amount of the compound than the second dose or vice versa. However preferably, the compound is administered in similar or equal doses. Therefore, in a most preferred embodiment, the compound is administered twice daily in two similar or equal doses.

In a further preferred embodiment of the invention, the total daily dose of the compound as defined herein above is administered in at least two separate doses. The interval between the administration of the at least two separate doses is at least about 0.5, 1, 2, 3, 4, 5, 6, 7, 8, 9, 10, 11 or 12 hours, preferably the interval between the at least two separate doses is at least about 4, 5, 6, 7, 8, 9, 10, 11 or 12 hours and more preferably the interval between the at least two separate doses is at least about 8, 9, 10, 11 or 12 hours.

### Use

In one aspect of the invention, the use is provided of either a compound of general formula I, or of a composition according to the invention. Said use is for the treatment of a disease or condition associated with DUX4 expression of a subject in need thereof, and comprises administration to the subject of an effective dose of a compound of general formula I or composition according to the invention, wherein the compound of general formula I or composition are as defined earlier herein.

In one embodiment of this aspect, the use is provided of either a compound of general formula I, or of a composition according to the invention. Said use is for the treatment of muscular dystrophy or cancer in a subject in need thereof, and comprises administration to the subject of an effective dose of a compound of general formula I or composition according to the invention, wherein the compound of general formula I or composition are as defined earlier herein. Further features and definitions are preferably as defined elsewhere herein, particularly for diseases or conditions to be treated, or for uses such as use of the compounds for the promotion of myogenic fusion and/or for the promotion of myogenic differentiation, which can be in vitro, in vivo, or ex vivo.

### Method

One aspect of the invention provides an in vivo, in vitro, or ex vivo method for reducing DUX4 expression, the method comprising the step of contacting a cell with a compound of general formula I as defined earlier herein, or with a composition as defined earlier herein. Preferably, said method is for treating a disease or condition associated with DUX4 expression, such as a muscular dystrophy or cancer, most preferably said disease or condition is facioscapulohumeral muscular dystrophy (FSHD). The method preferably comprises use as defined earlier herein. Preferred methods comprise contacting a cell with a compound of general formula I or composition as defined earlier herein. In the context of the invention, contacting a cell with a compound of general formula I or a composition can comprise adding such a compound of general formula I or composition to a medium in which a cell is cultured. Contacting a cell with a compound of general formula I or a composition can also comprise adding such a compound of general formula I or composition to a medium, buffer, or solution in which a cell is suspended, or which covers a cell. Other preferred methods of contacting a cell comprise injecting a cell with a compound of general formula I or composition, or exposing a cell to a material comprising a compound of general formula I or composition according to the invention. Further methods for administration are defined elsewhere herein. Preferred cells are cells known to express DUX4, cells suspected of expressing DUX4, or cells known to be affected by a disease or condition as defined earlier herein.

In one embodiment of this aspect, the method is an in vitro method. In a further embodiment of this aspect, the method is an ex vivo method. In a further embodiment of this aspect, the method is an in vivo method. In a preferred embodiment of this aspect, the method is an in vitro or an ex vivo method.

Within the embodiments of this aspect, the cell may be a cell from a sample obtained from a subject. Such a sample may be a sample that has been previously obtained from a subject. Within the embodiments of this aspect, samples may have been previously obtained from a human subject. Within the embodiments of this aspect, samples may have been obtained from a non-human subject. In a preferred embodiment of this aspect, obtaining the sample is not part of the method according to the invention.

In preferred embodiments, the method according to the invention is a method for reducing DUX4 expression in a subject in need thereof, the method comprising the step of administering an effective amount of a compound of general formula I as defined earlier herein, or a composition as defined earlier herein. In more preferred embodiments, the method is for the treatment of a disease or condition associated with DUX4 expression, preferably a muscular dystrophy or cancer, most preferably said disease or condition is facioscapulohumeral muscular dystrophy (FSHD). Further features and definitions are preferably as defined elsewhere herein. The method can be for any use, preferably for any non-medical use as described herein, such as for the promotion of myogenic fusion and/or for the promotion of myogenic differentiation, which can be in vitro, in vivo, or ex vivo.

### General Definitions

In this document and in its claims, the verb “to comprise” and its conjugations is used in its non-limiting sense to mean that items following the word are included, but items not specifically mentioned are not excluded. In addition, the verb “to consist” may be replaced by “to consist essentially of” meaning that a combination or a composition as defined herein may comprise additional component(s) than the ones specifically identified, said additional component(s) not altering the unique characteristic of the invention. In addition, reference to an element by the indefinite article “a” or “an” does not exclude the possibility that more than one of the element is present, unless the context clearly requires that there be one and only one of the elements. The indefinite article “a” or “an” thus usually means “at least one”.

When a structural formula or chemical name is understood by the skilled person to have chiral centers, yet no chirality is indicated, for each chiral center individual reference is made to all three of either the racemic mixture, the pure R enantiomer, or the pure S enantiomer.

Whenever a parameter of a substance is discussed in the context of this invention, it is assumed that unless otherwise specified, the parameter is determined, measured, or manifested under physiological conditions. Physiological conditions are known to a person skilled in the art, and comprise aqueous solvent systems, atmospheric pressure, pH-values between 6 and 8, a temperature ranging from room temperature to about 37° C. (from about 20° C. to about 40° C.), and a suitable concentration of buffer salts or other components.

The use of a substance as a medicament as described in this document can also be interpreted as the use of said substance in the manufacture of a medicament. Similarly, whenever a substance is used for treatment or as a medicament, it can also be used for the manufacture of a medicament for treatment. Products for use as a medicament described herein can be used in methods of treatments, wherein such methods of treatment comprise the administration of the product for use. compound of general formula I or compositions according to this invention are preferably for use in methods or uses according to this invention.

Throughout this application, expression is considered to be the transcription of a gene into functional mRNA, leading to a polypeptide such as an enzyme or transcription factor or for example DUX4 polypeptide. A polypeptide can assert an effect or have an activity. In this context, increased or decreased expression or activity of a polypeptide can be considered an increased or decreased level of mRNA encoding said polypeptide, an increased or decreased level or amount of polypeptide molecules, or an increased or decreased total activity of said polypeptide molecules. Preferably, an increased or decreased expression of a polypeptide results in an increased or decreased activity of said polypeptide, respectively, which can be caused by increased or decreased levels or amounts of polypeptide molecules. More preferably, a reduction of DUX4 expression is a reduction of transcription of a DUX4 gene, destabilisation or degradation of DUX4 mRNA, reduction of the amount of DUX4 polypeptide molecules, reduction of DUX4 polypeptides molecule activity, destabilisation or degradation of DUX4 polypeptide, or combinations thereof. A destabilized mRNA leads to lower expression of its encoded polypeptide, possibly it cannot lead to such expression. A degraded mRNA is destroyed and cannot lead to expression of its encoded polypeptide. A destabilized polypeptide asserts less of an effect or has lower activity than the same polypeptide that has not been destabilized, possibly it asserts no effect or has no activity. A destabilized polypeptide can be denatured or misfolded. A degraded polypeptide is destroyed and does not assert an effect or have an activity.

In the context of this invention, a decrease or increase of a parameter to be assessed means a change of at least 5% of the value corresponding to that parameter. More preferably, a decrease or increase of the value means a change of at least 10%, even more preferably at least 20%, at least 30%, at least 40%, at least 50%, at least 70%, at least 90%, or 100%. In this latter case, it can be the case that there is no longer a detectable value associated with the parameter.

The word “about” or “approximately” when used in association with a numerical value (e.g. about 10) preferably means that the value may be the given value (of 10) more or less 5% of the value.

Each embodiment as identified herein may be combined together unless otherwise indicated. The invention has been described above with reference to a number of embodiments. A skilled person could envision trivial variations for some elements of the embodiments. These are included in the scope of protection as defined in the appended claims. All patent and literature references cited are hereby incorporated by reference in their entirety.

### EXAMPLES

### Example 1 - Synthesis of Compounds of General Formula (I)

### 1.1 - General Methods

All reagents, for which the synthesis is not described in the experimental part, are either commercially available, or are known compounds or may be formed from known compounds by known methods.

The compounds and intermediates produced according to the methods of the invention may require purification. Purification of organic compounds is well known to a person skilled in the art and there may be several ways of purifying the same compound. In some cases, no purification may be necessary. In some cases, the compounds may be purified by crystallization. In some cases, impurities may be stirred out using a suitable solvent. In some cases, the compounds may be purified by chromatography, particularly flash column chromatography, using prepacked silica gel cartridges, e.g. Biotage SNAP cartidges KP-Sil® or KP-NH® in combination with a Biotage autopurifier system (SP4® or Isolera Four®) and eluents such as gradients of hexane/EtOAc or DCM/MeOH. In some cases, the compounds may be purified by preparative HPLC using methods as described.

Purification methods as described herein may provide compounds of the present invention which possess a sufficiently basic or acidic functionality in the form of a salt, such as, in the case of a compound of the present invention which is sufficiently basic, a trifluoroacetate or formate salt, or, in the case of a compound of the present invention which is sufficiently acidic, an ammonium salt. A salt of this type can either be transformed into its free base or free acid form, respectively, by various methods known to a person skilled in the art, or be used as salts in subsequent biological assays. It is to be understood that the specific form of a compound of the present invention as isolated and as described herein is not necessarily the only form in which said compound can be applied to a biological assay in order to quantify the specific biological activity.

All the starting materials and reagents are commercially available and were used as is. ¹H Nuclear magnetic resonance (NMR) spectroscopy was carried out using a Bruker instrument operating at 400 MHz or 500 MHz as specified, using the stated solvent at around room temperature unless otherwise stated. In all cases, NMR data were consistent with the proposed structures. Characteristic chemical shifts (δ) are given in parts-per-million using conventional abbreviations for designation of major peaks: e.g. s, singlet; d, doublet; t, triplet; q, quartet; dd, doublet of doublets; dt, doublet of triplets; m, multiplet; br, broad. Preparative HPLC purification was performed by reverse phase HPLC using a Waters Fractionlynx preparative HPLC system (2525 pump, 2996/2998 UV/VIS detector, 2767 liquid handler) or an equivalent HPLC system such as a Gilson Trilution UV directed system. The Waters 2767 liquid handler acted as both auto-sampler and fraction collector. The columns used for the preparative purification of the compounds were a Waters Sunfire OBD Phenomenex Luna Phenyl Hexyl (10 µm21.2 × 150 mm, 10 µm) or Waters Xbridge Phenyl (10 µm 19 × 150 mm, 5 µm). Appropriate focused gradients were selected based on acetonitrile and methanol solvent systems under either acidic or basic conditions. The modifiers used under acidic/basic conditions were formic acid (0.1% V/V) and ammonium bicarbonate (10 mM) respectively. The purification was controlled by Waters Fractionlynx software through monitoring at 210-400 nm, and triggered a threshold collection value at 260 nm and, when using the Fractionlynx, the presence of target molecular ion as observed under APi conditions. Collected fractions were analysed by LCMS (Waters Acquity systems with Waters SQD). Normal phase flash column chromatography was performed utilizing a Biotage Isolera system. The silica gel columns were purchased from either Interchim or Biotage. The mobile phase was either ethyl acetate in hexanes or methanol in dichloromethane with various ratios, and the fraction collection was triggered by UV absorbance at 254 nm. Analytical high-performance liquid chromatography-mass spectrometry (HPLC-MS) was performed utilizing HP or Waters DAD + Micromass ZQ, single quadrupole LC-MS or Quattro Micro LC-MS-MS. Method 1: The RP-HPLC column was Phenomenex Luna 5 µm C18 (2), (100 × 4.6 mm). Mobile phase 5-95% acetonitrile in water (0.1% formic acid) gradient, flow rate 2.0 mL/min, and 6.5 min run time. Method 2: The RP-HPLC column was Waters Xterra MS 5 µm C18, 100 × 4.6 mm. Mobile phase 5-95% acetonitrile in water (10 mM ammonium bicarbonate (ammonium hydrogen carbonate)).

Chemical names were generated using the JChem for Excel naming software (Version 16.7.1800.1000) by Chem Axon Ltd. In some cases, generally accepted names of commercially available reagents were used in place of names generated by the naming software.

### Analytical LC-MS Methods: Method A

Column: Phenomenex Kinetix-XB C18 1.2 × 100 mm, 1.7 µm; eluent A: water + 0.1 vol% formic acid, eluent B: acetonitrile + 0.1 vol% formic acid; gradient: 0-5.3 min5-100% B, 5.3-5.8 min 100% B, 5.8-5.82 min100-5% B, 5.82-7.00 min5% B; flow 0.6 mL/min; injection volume 1 µL; temperature: 40° C.; UV scan: 215 nm; PDA Spectrum range: 200-400 nm step: 1 nm; MSD signal settings- scan pos: 150-850.

Method B Column: Waters UPLC®BEH™ C18 2.1 × 100 mm, 1.7 µm; eluent A: 2 mM ammonium bicarbonate, buffered to pH10, eluent B: acetonitrile; gradient: 0-5.3 min5-100% B, 5.3-5.8 min 100% B, 5.8-5.82 min100-5% B, 5.8-7.0 min5% B; flow 0.6 mL/min; injection volume 2 µL; temperature: 40° C.; UV scan: 215 nm; PDA Spectrum range: 200-400 nm step: 1 nm; MSD signal settings- scan pos: 150-850.

Method C Column: Phenomenex Gemini -NX C18 2.01 × 100 mm, 3 µm; eluent A: 2 mM ammonium bicarbonate, buffered to pH10, eluent B: acetonitrile; gradient: 0-5.5 min5-100% B, 5.5-5.9 min 100% B, 5.9-5.92 min100-5% B, 5.92-7.00 min5% B; flow 0.6 mL/min; injection volume 3 µL; temperature: 40° C.; UV scan: 215 nm; PDA Spectrum range: 210-400 nm step: 1 nm; MSD signal settings- scan pos: 150-850.

Method D Column: Waters Atlantis dC18 2.1 × 100 mm, 3 µm eluent A: water + 0.1 vol% formic acid, eluent B: acetonitrile + 0.1 vol% formic acid; gradient: 0-5.0 min5-100% B, 5.0-5.4 min 100% B, 5.4-5.42 min100-5% B, 5.42-7.00 min5% B; flow 0.6 mL/min; injection volume 3 µL; temperature: 40° C.; UV scan: 215 nm; PDA Spectrum range: 200-400 nm step: 1 nm; MSD signal settings- scan pos: 150-1000.

Method E Column: Kinetex Core-Shell C18 2.1 × 50 mm, 5 µm eluent A: water + 0.1 vol% formic acid, eluent B: acetonitrile + 0.1 vol% formic acid; gradient: 0 - 1.2 min5 - 100% B, 1.3 - 1.3 min 100% B, 1.3 - 1.31 min100 - 5% B, 1.31 - 1.65 min5% B; flow 1.2 mL/min; injection volume 3 µL; temperature: 40° C.; UV scan: 215 nm; PDA Spectrum range: 210-420 nm step: 1 nm; MSD signal settings- scan pos: 100-1000.

Method F Column: Waters UPLC®CSH™ C18 2.1 × 100 mm, 1.7 µm; eluent A: water + 0.1 vol% formic acid, eluent B: acetonitrile + 0.1 vol% formic acid; gradient: 0 - 1.1 min5 - 100% B, 1.1 -1.35 min100% B, 1.35 - 1.4 min100 - 5% B, 1.4 - 1.5 min5% B; flow 0.9 mL/min; injection volume 2 µL; temperature: 40° C.; UV scan: 215 nm; PDA Spectrum range: 200-400 nm step: 1 nm; MSD signal settings- scan pos: 150-850.

Method G Column: Phenomenex Gemini-NX C18 2.0 × 50 mm, 3 µm; eluent A: 2 mM ammonium hydroxide, buffered to pH10, eluent B: acetonitrile; gradient: 0 - 1.8 min1 - 100% B, 1.8 - 2.1 min 100% B, 2.1 - 2.3 min100 - 1% B; flow 1 mL/min; injection volume 3 µL; temperature: 40° C.; UV scan: 215 nm; PDA Spectrum range: 210-420 nm step: 1 nm; MSD signal settings- scan pos: 150-850.

Method H Column: Waters UPLC®BEH™ C18 2.1 × 30 mm, 1.7 µm; eluent A: 2 mM ammonium bicarbonate, buffered to pH10, eluent B: acetonitrile; gradient: 0 - 0.75 min5 - 100% B, 0.75 - 0.85 min100% B, 0.85 - 0.9 min100 - 5% B, 0.9 - 1.0 min5% B; flow 1 mL/min; injection volume 2 µL; temperature: 40° C.; UV scan: 215 nm; PDA Spectrum range: 200-400 nm step: 1 nm; MSD signal settings- scan pos: 100-1000.

Method I Column: Waters UPLC® BEHTM C18 2.1 × 50 mm, 1.7 µm; eluent A: water + 0.1 vol% formic acid, eluent B: acetonitrile + 0.1 vol% formic acid; gradient: 0 - 1.1 min5 - 100% B, 1.1 -1.35 min100% B, 1.35 - 1.4 min100 - 5% B, 1.4 - 1.5 min5% B; flow 0.9 mL/min; injection volume 1 µL; temperature: 40° C.; UV scan: 215 nm; PDA Spectrum range: 200-400 nm step: 1 nm; MSD signal settings- scan pos: 100-1000.

Method J Column: Waters UPLC® CORTECSTM C8 2.1 × 100 mm, 1.6 µm; eluent A: water + 0.1 vol% formic acid, eluent B: acetonitrile + 0.1 vol% formic acid; gradient: 0 - 1.1 min5 - 100% B, 1.1 - 1.40 min100% B, 1.40 - 1.42 min100 - 5% B, 1.42 - 1.70 min5% B; flow 0.9 mL/min; injection volume 1 µL; temperature: 40° C.; UV scan: 215 nm; PDA Spectrum range: 200-400 nm step: 1 nm; MSD signal settings- scan pos: 100-1000.

Method K Column: Waters UPLC® BEHTM C18 2.1 × 30 mm, 1.7 µm; eluent A: 2 mM ammonium bicarbonate, buffered to pH10, eluent B: acetonitrile; gradient: 0 - 1.1 min1 - 100% B, 1.1 - 1.35 min100% B, 1.35 - 1.40 min100 - 1% B, 1.40 - 1.8 min1% B; flow 1 mL/min; injection volume 1 µL; temperature: 40° C.; UV scan: 215 nm; PDA Spectrum range: 200-400 nm step: 1 nm; MSD signal settings- scan pos: 100-1000.

### Purification Methods:

Biotage Isolera™ chromatography system (see www.biotage.com/product-area/flash-purification) using pre-packed silica and pre-packed modified silica cartridges.

### Preparative HPLC, Method A1:

Instrument: pump: Gilson 331 & 332; auto injector: Gilson GX281; UV detector: Gilson 159; collector: Gilson GX281 or pump: Gilson 333 & 334; auto injector: Gilson GX281; UV detector: Gilson 155; collector: Gilson GX281; Column: Waters Xbridge C18 30 × 100 mm, 10 µm; eluent A: water + 0.2 vol% ammonium hydroxide, eluent B: acetonitrile + 0.2 vol% ammonium hydroxide; gradient: 0 - 0.8 min10% B, 0.8 - 14. 5 min10 - 95% B, 14.5 - 16.7 min 95% B; flow 40 mL/min; injection volume 1500 µL; temperature: 25° C.; UV scan: 215 nm.

### Preparative HPLC, Method A2:

Instrument: pump: Gilson 331 & 332; auto injector: Gilson GX281; UV detector: Gilson 159; collector: Gilson GX281 or pump: Gilson 333 & 334; auto injector: Gilson GX281; UV detector: Gilson 155; collector: Gilson GX281; Column: Waters Xbridge C18 30 × 100 mm, 10 µm; eluent A: water + 0.2 vol% ammonium hydroxide, eluent B: acetonitrile + 0.2 vol% ammonium hydroxide; gradient: 0 - 1.1 min30% B, 1.1 - 10.05 min30 - 95% B, 10.05 - 11.5 min 95% B; flow 40 mL/min; injection volume 1500 µL; temperature: 25° C.; UV scan: 215 nm.

### Preparative HPLC, Method B1:

Instrument pump: Gilson 331 & 332; auto injector: Gilson GX281; UV detector: Gilson 159; collector: Gilson GX281; Column: Waters Sunfire C18 30 × 100 mm, 10 µm; eluent A: water + 0.1 vol% formic acid, eluent B: acetonitrile + 0.1 vol% formic acid; gradient: 0 - 0.8 min10% B, 0.8 - 14.5 min5 - 95% B, 14.5 - 16.7 min95% B; flow 40 mL/min; injection volume 1500 µL; temperature: 25° C.; UV scan: 215 nm.

### Preparative HPLC, Method B2:

Instrument pump: Gilson 331 & 332; auto injector: Gilson GX281; UV detector: Gilson 159; collector: Gilson GX281; Column: Waters Sunfire C18 30 × 100 mm, 10 µm; eluent A: water + 0.1 vol% formic acid, eluent B: acetonitrile + 0.1 vol% formic acid; gradient: 0 - 1.1 min30% B, 1.1 - 10.05 min30 - 95% B, 10.05 - 11.5 min95% B; flow 40 mL/min; injection volume 1500 µL; temperature: 25° C.; UV scan: 215 nm.

### 1.2 - Synthesis of Intermediates

### Synthesis of N-(5-fluoro-2-nitrophenyl)pyridin-4-amine / Intermediate 1-1

KO^{t}Bu (2.05 g, 18.2 mmol) was added to an ice-cold solution of 4-aminopyridine (0.86 g, 9.11 mmol) in THF (10 mL). The reaction was stirred for 15 min then a solution of 2,4-difluoro-1-nitro-benzene (1.0 mL, 9.11 mmol) in THF (10 mL) was added. The reaction was stirred for 45 min, then quenched into sat. NH₄Cl (aq). The aqueous layer was extracted into EtOAc (2×), the combined organics washed with brine, dried over MgSO₄ and concentrated in vacuo. The residue was purified by flash chromatography (50 g, silica) eluting with 0-10% MeOH/DCM to afford the title compound (950 mg, 44% yield). 1H NMR (400 MHz, DMSO-d6) δ 9.41 (s, 1H), 8.42 - 8.37 (m, 2H), 8.23 (dd, J = 9.3, 6.0 Hz, 1H), 7.36 (dd, J = 11.0, 2.7 Hz, 1H), 7.24 - 7.19 (m, 2H), 7.01 (ddd, J = 9.4, 7.5, 2.7 Hz, 1H). LCMS (Analytical Method E) Rt = 0.62 min, MS (ESIpos): m/z 234.0 [M+H]+, Purity = 100%.

### Synthesis of Tert-butyl 4-{4-nitro-3-[(pyridin-4-yl)amino]phenyl}piperazine-1-carboxylate / Intermediate 1-2

A solution of N-(5-fluoro-2-nitro-phenyl)pyridin-4-amine (Intermediate 1-1) (0.29 g, 1.24 mmol), N-Boc-piperazine (255 mg, 1.37 mmol) and DIPEA (0.33 mL, 1.87 mmol) in THF (10 mL) was heated to 65° C. for 24 h. Additional N-boc-piperazine (100 mg, 0.53 mmol) and DIPEA (0.12 mL, 0.68 mmol) were added and heating continued for 24 h. The reaction was cooled and quenched into sat. NaHCOs (aq). The aqueous layer was extracted into EtOAc (2×) and the combined organics washed with brine, dried over MgSO₄ and concentrated in vacuo. The residue was purified by flash chromatography (25 g, silica) eluting with 30-100% EtOAc/heptane to yield the title compound (310 mg, 62% yield). 1H NMR (400 MHz, DMSO-d6) δ 9.48 (s, 1H), 8.38 - 8.33 (m, 2H), 8.05 (d, J = 9.6 Hz, 1H), 7.27 - 7.19 (m, 2H), 6.78 (d, J = 2.6 Hz, 1H), 6.72 (dd, J = 9.6, 2.6 Hz, 1H), 3.50 - 3.42 (m, 8H), 1.41 (s, 9H). LCMS (Analytical Method E) Rt = 0.98 min, MS (ESIpos): m/z 400.2 [M+H]+, Purity = 100%.

### Synthesis of Tert-butyl 4-{4-amino-3-[(pyridin-4-yl)amino]phenyl}piperazine-1-carboxylate / Intermediate 1-3

A suspension of tert-butyl 4-[4-nitro-3-(4-pyridylamino)phenyl]piperazine-1-carboxylate (Intermediate 1-2) (155 mg, 0.388 mmol), iron (108 mg, 1.94 mmol) and NH₄Cl (166 mg, 3.10 mmol) in MeOH (7 mL) and water (3 mL) was heated to 80° C. for 3 h. The mixture was cooled and filtered through celite, then concentrated in vacuo. The residue was taken up in DCM/MeOH and loaded onto an SCX-2 ion exchange cartridge. The cartridge was washed with MeOH, and then the compound was eluted with 2 M NH₃ in MeOH, and concentratd in vacuo. The residue was purified by flash chromatography (10 g, silica) eluting with 0-25% MeOH/DCM to yield the title compound (96 mg, 60% yield). 1H NMR (400 MHz, DMSO-d6) δ 8.50 (s, 1H), 8.10 (d, J = 6.3 Hz, 2H), 6.72 (s, 2H), 6.65 (d, J = 1.9 Hz, 1H), 6.61 (d, J = 5.5 Hz, 2H), 4.52 (s, 2H), 3.44 - 3.40 (m, 4H), 2.90 - 2.84 (m, 4H), 1.41 (s, 9H). LCMS (Analytical Method E) Rt = 0.82 min, MS (ESIpos): m/z 370.1 [M+H]+, Purity = 89%.

### Synthesis of Tert-butyl 4-[2-(4-fluorophenyl)-1-(pyridin-4-yl)-1H-1,3-benzodiazol-6-yllpiperazine-1-carboxylate / Intermediate 1

4-fluorobenzaldehyde (16 µL, 0.149 mmol), CAN (7.4 mg, 0.0135 mmol) and hydrogen peroxide (35%, 47 µL, 0.541 mmol) were added sequentially to a suspension of tert-butyl 4-[4-amino-3-(4-pyridylamino)phenyl]piperazine-1-carboxylate (Intermediate 1-3) (50 mg, 0.135 mmol) in EtOH (2 mL). The reaction was heated to 45° C. for 2 h, then cooled and quenched into water. The aqueous layer was extracted into EtOAc (2×), the combined organics washed with brine, dried over MgSO₄ and concentrated in vacuo. The residue was purified by flash chromatography (10 g, silica) eluting with 30-100% EtOAc/heptane to yield the title compound (29 mg, 45% yield). 1H NMR (400 MHz, Chloroform-d) δ 8.82 - 8.74 (m, 2H), 7.77 (d, J = 8.8 Hz, 1H), 7.48 (dd, J = 8.8, 5.3 Hz, 2H), 7.25 - 7.23 (m, 2H), 7.08 (dd, J = 8.8, 2.1 Hz, 1H), 7.03 (t, J = 8.6 Hz, 2H), 6.78 (s, 1H), 3.65 - 3.57 (m, 4H), 3.17 - 3.06 (m, 4H), 1.48 (s, 9H). LCMS (Analytical Method E) Rt = 1.16 min, MS (ESIpos): m/z 474.1 [M+H]+, Purity = 100%.

### Synthesis of N-(5-fluoro-2-nitrophenyl)-2-methylpyridin-4-amine / Intermediate 2-1

KO^{t}Bu (818 mg, 7.29 mmol) was added to an ice-cold solution of 2-methylpyridin-4-amine (395 mg, 3.65 mmol) in THF (6 mL). The reaction was stirred for 15 min then a solution of 2,4-difluoro-1-nitro-benzene (400 µL, 3.65 mmol) in THF (6 mL) was added. The mixture was stirred for 1.5 h then quenched with sat. NH₄Cl solution and extracted with EtOAc (2×). The organics were combined, dried over MgSO₄ and concentrated in vacuo. The residue was purified by flash chromatography (10 g, silica) eluting with 0-10% MeOH/DCM to yield the title compound (621 mg, 69% yield). 1H NMR (500 MHz, DMSO-d6) δ 9.35 (s, 1H), 8.27 (d, J = 5.6 Hz, 1H), 8.22 (dd, J = 9.3, 6.0 Hz, 1H), 7.32 (dd, J = 11.0, 2.7 Hz, 1H), 7.08 (d, J = 1.8 Hz, 1H), 7.04 (dd, J = 5.6, 2.1 Hz, 1H), 6.98 (ddd, J = 9.9, 7.5, 2.7 Hz, 1H), 2.40 (s, 3H). LCMS (Analytical Method E) Rt = 0.71 min, MS (ESIpos): m/z 247.9 [M+H]+, Purity = 100%.

### Synthesis of Tert-butyl 4-{3-[(2-methylpyridin-4-yl)amino]-4-nitrophenyl}piperazine-1-carboxylate / Intermediate 2-2

A solution of N-(5-fluoro-2-nitro-phenyl)-2-methyl-pyridin-4-amine (Intermediate 2-1) (621 mg, 2.51 mmol), N-boc-piperazine (700 mg, 3.76 mmol) and DIPEA (700 µL, 4.01 mmol) in MeCN (10 mL) was stirred at 80° C. for 20 h. The mixture was diluted with water and extracted with DCM. The organics were dried over MgSO₄ and concentrated in vacuo. The residue was purified by flash chromatography (50 g, silica) eluting with 0-100% EtOAc/heptane, then 0-40% MeOH/EtOAc to yield the title compound (876 mg, 83% yield)._1H NMR (500 MHz, DMSO-d6) δ 9.45 (s, 1H), 8.24 (d, J = 5.6 Hz, 1H), 8.04 (d, J = 9.6 Hz, 1H), 7.10 (d, J = 2.1 Hz, 1H), 7.08 (dd, J = 5.6, 2.2 Hz, 1H), 6.75 (d, J = 2.6 Hz, 1H), 6.70 (dd, J = 9.7, 2.6 Hz, 1H), 3.48 - 3.39 (m, 8H), 2.39 (s, 3H), 1.41 (s, 9H). LCMS (Analytical Method E) Rt = 0.98 min, MS (ESIpos): m/z 414.2 [M+H]+, Purity = 100%.

### Synthesis of Tert-butyl 4-{4-amino-3-[(2-methylpyridin-4-yl)amino]phenyl}piperazine-1-carboxylate / Intermediate 2-3

To a suspension of tert-butyl 4-[3-[(2-methyl-4-pyridyl)amino]-4-nitrophenyl]piperazine-1-carboxylate (Intermediate 2-2) (870 mg, 2.06 mmol) in de-gassed EtOH (10 mL), 10% Pd/C (80 mg, 0.625 mmol) was added, and the mixture was stirred under a hydrogen atmosphere for 5 h. The hydrogen was removed under vacuum and the reaction mixture was filtered through celite. The filtrate was concentrated in vacuo to yield the title compound (706 mg, 47% yield), which was used in the next step without further purification. 1H NMR (400 MHz, DMSO-d6) δ 8.0 - 7.9 (m, 1H), 7.9 (s, 1H), 6.7 - 6.6 (m, 3H), 6.4 (d, J = 4.8 Hz, 2H), 4.4 (s, 2H), 3.4 (s, 4H), 2.9 - 2.8 (m, 4H), 2.3 (s, 3H), 1.4 (s, 9H). LCMS (Analytical Method E) Rt = 0.84 min, MS (ESIpos): m/z 384.2 [M+H]+, Purity = 90%.

### Synthesis of Tert-butyl 4-[2-(4-fluorophenyl)-1-(2-methylpyridin-4-yl)-1H-1,3-benzodiazol-6-yl]piperazine-1-carboxylate / Intermediate 2

To a solution of tert-butyl 4-[4-amino-3-[(2-methyl-4-pyridyl)amino]phenyl]piperazine-1-carboxylate (Intermediate 2-3) (130 mg, 0.305 mmol) in EtOH (3 mL), 4-fluorobenzaldehyde (36 µL, 0.336 mmol), CAN (17 mg, 0.0311 mmol) and hydrogen peroxide (35%, 107 µL, 1.22 mmol) were added sequentially. The reaction was heated at 30° C. for 1 h then at 45° C. for another h. The reaction was cooled to RT, diluted with water and extracted with EtOAc (2×). The organics were combined, dried over MgSO₄ and concentrated in vacuo. The residue was purified by flash chromatography (10 g, silica) eluting with 0-100% EtOAc/heptane, then 0-20% MeOH/EtOAc to yield the title compound (68 mg, 39% yield). 1H NMR (500 MHz, Chloroform-d) δ 8.65 (d, J = 5.3 Hz, 1H), 7.74 (d, J = 8.8 Hz, 1H), 7.50 - 7.45 (m, 2H), 7.08 - 6.99 (m, 5H), 6.73 (d, J = 2.2 Hz, 1H), 3.63 -3.54 (m, 4H), 3.13 - 3.07 (m, 4H), 2.61 (s, 3H), 1.47 (s, 9H). LCMS (Analytical Method E) Rt = 1.15 min, MS (ESIpos): m/z 488.2 [M+H]+, Purity = 99%.

### Synthesis of N-(5-fluoro-2-nitrophenyl)pyrimidin-4-amine / Intermediate 3-1

NaH (60%, 566 mg, 14.1 mmol) was added to an ice-cold solution of 4-aminopyrimidine (0.90 g, 9.43 mmol) in DMF (20 mL). The reaction was stirred for 10 min then 2,4-difluoro-1-nitro-benzene (1.0 mL, 9.43 mmol) was added dropwise and the reaction stirred for 1 h. The reaction was quenched by dropwise addition of water. The aqueous layer was extracted into EtOAc (2×), the combined organics washed with brine, dried over MgSO₄ and concentrated in vacuo. The residue was purified by flash chromatography (100 g, silica) eluting with 0-85% EtOAc/heptane to yield the title compound (600 mg, 16% yield). 1H NMR (400 MHz, DMSO-d6) δ 10.07 (s, 1H), 8.68 - 8.65 (m, 1H), 8.45 (d, J = 5.8 Hz, 1H), 8.18 (dd, J = 9.2, 5.9 Hz, 1H), 8.01 (dd, J = 11.1, 2.8 Hz, 1H), 7.17 (ddd, J = 9.2, 7.5, 2.8 Hz, 1H), 7.08 (dd, J = 5.9, 1.2 Hz, 1H). LCMS (Analytical Method E) Rt = 0.85 min, MS (ESIpos): m/z 235.0 [M+H]+, Purity = 60%.

### Synthesis of Tert-butyl 4-{4-nitro-3-[(pyrimidin-4-yl)amino]phenyl}piperazine-1-carboxylate / Intermediate 3-2

A solution of N-(5-fluoro-2-nitro-phenyl)pyrimidin-4-amine (Intermediate 3-1) (800 mg, 2.56 mmol), N-boc-piperazine (1.43 g, 7.69 mmol) and DIPEA (1.8 mL, 10.2 mmol) in THF (25 mL) was heated to 65° C. for 18 h. The reaction was cooled and quenched into water. The aqueous layer was extracted into EtOAc (2×) and the combined organics washed with brine, dried over MgSO₄ and concentrated in vacuo. The residue was purified by flash chromatography (50 g, silica) eluting with 0-100% EtOAc/heptane to yield the title compound (292 mg, 29% yield). 1H NMR (500 MHz, DMSO-d6) δ 10.17 (s, 1H), 8.70 - 8.66 (m, 1H), 8.40 (d, J = 5.9 Hz, 1H), 8.04 (d, J = 9.6 Hz, 1H), 7.76 (d, J = 2.7 Hz, 1H), 7.09 (dd, J = 5.9, 1.2 Hz, 1H), 6.79 (dd, J = 9.6, 2.8 Hz, 1H), 3.48 (s, 8H), 1.43 (s, 9H). LCMS (Analytical Method E) Rt = 1.07 min, MS (ESIpos): m/z 401.1 [M+H]+, Purity = 100%.

### Synthesis of Tert-butyl 4-{4-amino-3-[(pyrimidin-4-yl)amino]phenyl}piperazine-1-carboxylate / Intermediate 3-3

EtOH (5 mL) was added to a flask containing tert-butyl 4-[4-nitro-3-(pyrimidin-4-ylamino)phenyl]piperazine-1-carboxylate (Intermediate 3-2) (150 mg, 0.375 mmol) and Pd/C (10%, 14 mg, 0.112 mmol). The reaction was stirred under an atmosphere of hydrogen for 18 h. The hydrogen was removed under vacuum and the mixture was filtered through a pad of celite, washing with MeOH, then concentrated in vacuo to afford the title compound pure (130 mg, 87% yield). 1H NMR (500 MHz, DMSO-d6) δ 8.70 (s, 1H), 8.48 (s, 1H), 8.15 (d, J = 6.0 Hz, 1H), 6.81 (d, J = 1.9 Hz, 1H), 6.74 - 6.67 (m, 2H), 6.47 (d, J = 5.6 Hz, 1H), 3.45 - 3.41 (m, 4H), 2.90 - 2.84 (m, 4H), 1.42 (s, 9H). LCMS (Analytical Method E) Rt = 0.79 min, MS (ESIpos): m/z 371.1 [M+H]+, Purity = 93%.

### Synthesis of Tert-butyl 4-[2-(4-fluorophenyl)-1-(pyrimidin-4-yl)-1H-1,3-benzodiazol-6-yl]piperazine-1-carboxylate / Intermediate 3

CAN (cerium ammonium nitrate, 10 mg, 0.0175 mmol) and hydrogen peroxide (35%, 61 µL, 0.702 mmol) were added sequentially to a solution of 4-fluorobenzaldehyde (21 µL, 0.193 mmol) and tert-butyl 4-[4-amino-3-(pyrimidin-4-ylamino)phenyl]piperazine-1-carboxylate (Intermediate 3-3) (65 mg, 0.175 mmol) in EtOH (2 mL). The reaction was heated to 40° C. for 1 h, then cooled and quenched into water. The aqueous layer was extracted into EtOAc (2×) and the combined organics washed with brine, dried over MgSO₄ and concentrated in vacuo. The residue was purified by flash chromatography (10 g, silica) eluting with 20-90% EtOAc/heptane to yield the title compound (48 mg, 58% yield). 1HNMR (500 MHz, DMSO-d6) δ 9.27 (d, J = 1.0 Hz, 1H), 8.95 (d, J = 5.4 Hz, 1H), 7.67 (d, J = 8.8 Hz, 1H), 7.55 - 7.50 (m, 2H), 7.48 (dd, J = 5.4, 1.2 Hz, 1H), 7.27 (t, J = 8.9 Hz, 2H), 7.23 (d, J = 2.2 Hz, 1H), 7.13 (dd, J = 8.9, 2.2 Hz, 1H), 3.49 (s, 4H), 3.14 - 3.08 (m, 4H), 1.43 (s, 9H). LCMS (Analytical Method E) Rt = 1.18 min, MS (ESIpos): m/z 475.1 [M+H]+, Purity = 100%.

### Synthesis of Tert-butyl 4-(6-amino-5-nitropyridin-2-yl)piperazine-1-carboxylate / Intermediate 4-1

A suspension of 6-chloro-3-nitro-pyridin-2-amine (2.50 g, 14.4 mmol), N-boc-piperazine (2.95 g, 15.8 mmol) and DIPEA (5.0 mL, 28.8 mmol) in MeCN (50 mL) was heated to 70° C. for 18 h. The reaction was cooled and partitioned between water and EtOAc. The organic layer was separated and the aqueous layer was extracted into EtOAc (2×). The combined organics were washed with brine, dried over MgSO₄ and concentrated in vacuo. The residue was triturated with EtOAc and collected by filtration, washing with EtOAc, and dried in vacuo to yield the title compound (4.49 g, 94% yield). 1H NMR (400 MHz, DMSO-d6) δ 8.09 (d, J = 9.5 Hz, 1H), 8.03 - 7.62 (m, 2H), 6.33 (d, J = 9.5 Hz, 1H), 3.80 - 3.65 (m, 4H), 3.47 - 3.37 (m, 4H), 1.43 (s, 9H). LCMS (Analytical Method E) Rt = 1.13 min, MS (ESIpos): m/z 324.1 [M+H]+, Purity = 100%.

### Synthesis of Tert-butyl 4-{5-nitro-6-[(pyridin-4-yl)amino]pyridin-2-yl}piperazine-1-carboxylate/ Intermediate 4

A mixture of tert-butyl 4-(6-amino-5-nitro-2-pyridyl)piperazine-1-carboxylate (Intermediate 4-1) (1.30 g, 4.02 mmol), 4-iodopyridine (824 mg, 4.02 mmol), Pd₂(dba)₃ (92 mg, 0.101 mmol), xantphos (116 mg, 0.201 mmol) and Cs₂CO₃ (2.62 g, 8.04 mmol) in 1,4-dioxane (13 mL) was degassed by sparging with nitrogen. The reaction was heated to 100° C. for 14 h. The reaction was cooled and the solid material removed by filtration, washing with 1,4-dioxane. The filtrate was concentrated in vacuo. The residue was purified by flash chromatography (50 g, silica) eluting with 0-7% MeOH/DCM to yield the title compound (1.1 g, 58% yield). 1H NMR (500 MHz, DMSO-d6) δ 10.63 (s, 1H), 8.48 (dd, J = 4.9, 1.4 Hz, 2H), 8.28 (d, J = 9.6 Hz, 1H), 7.72 - 7.66 (m, 2H), 6.59 (d, J = 9.6 Hz, 1H), 3.82 - 3.72 (m, 4H), 3.52 - 3.45 (m, 4H), 1.43 (s, 9H). LCMS (Analytical Method E) Rt = 0.72 min, MS (ESIpos): m/z 401.2 [M+H]+, Purity = 84%.

### Synthesis of Tert-butyl 4-{6-[(2-methylpyridin-4-yl)amino]-5-nitropyridin-2-yl}piperazine-1-carboxylate / Intermediate 5

A mixture of tert-butyl 4-(6-amino-5-nitro-2-pyridyl)piperazine-1-carboxylate (Intermediate 4-1) (0.50 g, 1.55 mmol), 4-bromo-2-methylpyridine (266 mg, 1.55 mmol), Pd₂(dba)₃ (35 mg, 0.0387 mmol), xantphos (45 mg, 0.0773 mmol) and Cs₂CO₃ (1.01 g, 3.09 mmol) in 1,4-dioxane (5 mL) was degassed by sparging with nitrogen. The reaction was heated to 100° C. for 16 h. The reaction was cooled and partitioned between EtOAc and water, and the aqueous layer extracted into EtOAc. The combined organics were washed with brine, dried over MgSO₄ and concentrated in vacuo. The residue was purified by flash chromatography (25 g, silica) eluting with 0-10% MeOH/DCM to yield the title compound (1.54 g, 100% yield). 1H NMR (500 MHz, Chloroform-d) δ 10.76 (s, 1H), 8.42 (d, J = 5.6 Hz, 1H), 8.34 (d, J = 9.5 Hz, 1H), 7.44 - 7.39 (m, 2H), 6.23 (d, J = 9.5 Hz, 1H), 3.79 (s, 4H), 3.63 - 3.54 (m, 4H), 2.55 (s, 3H), 1.50 (s, 9H). LCMS (Analytical Method E) Rt = 0.73 min, MS (ESIpos): m/z 415.3 [M+H]+, Purity = 98%.

### Synthesis of Tert-butyl 4-{6-[(2-methoxypyridin-4-yl)amino]-5-nitropyridin-2-yl}piperazine-1-carboxylate / Intermediate 6

A mixture of tert-butyl 4-(6-amino-5-nitro-2-pyridyl)piperazine-1-carboxylate (Intermediate 4-1) (0.50 g, 1.55 mmol), 4-bromo-2-methoxypyridine (291 mg, 1.55 mmol), Pd₂(dba)₃ (35 mg, 0.0387 mmol), Xantphos (45 mg, 0.0773 mmol) and Cs₂CO₃ (1.01 g, 3.09 mmol) in 1,4-dioxane (5 mL) was degassed by sparging with nitrogen. The reaction was heated to 100° C. for 16 h. The reaction was cooled and partitioned between EtOAc and water, and the aqueous layer extracted into EtOAc. The combined organics were washed with brine, dried over MgSO₄ and concentrated in vacuo. The residue was purified by flash chromatography (25 g, silica) eluting with 0-70% EtOAc/heptane to yield the title compound (600 mg, 88% yield). 1H NMR (500 MHz, Chloroform-d) δ 10.79 (s, 1H), 8.34 (d, J = 9.5 Hz, 1H), 8.07 (d, J = 5.7 Hz, 1H), 7.21 (d, J = 1.7 Hz, 1H), 6.99 (dd, J = 5.7, 1.9 Hz, 1H), 6.22 (d, J = 9.5 Hz, 1H), 3.95 (s, 3H), 3.79 (s, 4H), 3.64 - 3.53 (m, 4H), 1.50 (s, 9H). LCMS (Analytical Method E) Rt = 0.98 min, MS (ESIpos): m/z 431.3 [M+H]+, Purity = 98%.

### Synthesis of Tert-butyl 4-(6-{[2-(difluoromethyl)pyridin-4-yl]amino}-5-nitropyridin-2-yl)piperazine-1-carboxylate / Intermediate 7

A mixture of tert-butyl 4-(6-amino-5-nitro-2-pyridyl)piperazine-1-carboxylate (Intermediate 4-1) (0.25 g, 0.773 mmol), Pd₂(dba)₃ (18 mg, 0.0193 mmol), xantphos (22 mg, 0.0387 mmol), Cs₂CO₃ (0.50 g, 1.55 mmol) and 4-bromo-2-(difluoromethyl)pyridine (161 mg, 0.773 mmol) in 1,4-dioxane (2.5 mL) was degassed by sparging with nitrogen. The reaction was heated to 100° C. for 16 h. The reaction was cooled and the mixture was partitioned between EtOAc and water and the aqueous layer extracted into EtOAc. The combined organics were washed with brine, dried over MgSO₄ and concentrated in vacuo. The residue was purified by flash chromatography (25 g, silica) eluting with 0-75% EtOAc/heptane to yield the title compound (330 mg, 95% yield). 1H NMR (500 MHz, DMSO-d6) δ 10.71 (s, 1H), 8.55 (d, J = 5.5 Hz, 1H), 8.30 (d, J = 9.5 Hz, 1H), 8.24 (d, J = 1.9 Hz, 1H), 7.72 (dd, J = 5.5, 2.0 Hz, 1H), 6.93 (t, J = 55.1 Hz, 1H), 6.62 (d, J = 9.6 Hz, 1H), 3.77 (s, 4H), 3.53 - 3.42 (m, 4H), 1.43 (s, 9H). LCMS (Analytical Method F) Rt = 1.05 min, MS (ESIpos): m/z 451.2 [M+H]+, Purity = 100%.

### Synthesis of Tert-butyl 5-(6-amino-5-nitropyridin-2-yl)-octahydropyrrolo[3,4-c]pyrrole-2-carboxylate / Intermediate 8-1

A suspension of tert-butyl -hexahydropyrrolo[3,4-c]pyrrole-2(1H)-carboxylate (500 mg, 2.35 mmol) and 6-chloro-3-nitro-pyridin-2-amine (379 mg, 2.14 mmol) in MeCN (10 mL) was heated at 70° C. for 1 h. The reaction was cooled and the precipitate was collected by filtration and washed with MeCN to yield the title compound (677 mg, 91% yield). 1H NMR (500 MHz, DMSO-d6) δ 8.07 (d, J = 9.4 Hz, 1H), 8.01 (s, 1H), 7.66 (s, 1H), 6.02 (d, J = 9.4 Hz, 1H), 3.88 - 3.62 (m, 2H), 3.61 - 3.48 (m, 2H), 3.48 - 3.34 (m, 2H), 3.15 (s, 2H), 2.98 (m, 2H), 1.39 (s, 9H). LCMS (Analytical Method F) Rt = 0.89 min, MS (ESIpos): m/z 350.2 [M+H]+, Purity = 100%.

### Synthesis of Tert-butyl 5-{5-nitro-6-[(pyridin-4-yl)amino]pyridin-2-yl}-octahydropyrrolo[3,4-c]pyrrole-2-carboxylate / Intermediate 8

To a mixture of tert-butyl 5-(6-amino-5-nitropyridin-2-yl)-octahydropyrrolo[3,4-c]pyrrole-2-carboxylate (Intermediate 8-1) (100 mg, 0.234 mmol) and Na₂S₂O₄ (124 mg, 0.703 mmol) in DMSO (1 mL) and EtOH (0.2 mL) was added 4-fluorobenzaldehyde (38 µL, 0.352 mmol) and the reaction stirred at 100° C. for 20 h. The reaction was cooled and quenched into water. The aqueous layer was extracted with EtOAc then once with DCM. The combined organics were washed with brine then passed through a phase separating filter and concentrated in vacuo. The crude product was purified flash chromatography (10 g, silica) eluting with 5-30% MeOH/DCM to yield the title compound (28 mg, 29% yield). 1H NMR (400 MHz, DMSO-d6) δ 8.75 - 8.65 (m, 2H), 7.93 (d, J = 8.8 Hz, 1H), 7.57 - 7.47 (m, 2H), 7.47 - 7.40 (m, 2H), 7.26 (t, J = 8.9 Hz, 2H), 6.58 (d, J = 8.8 Hz, 1H), 3.61 (dd, J = 10.7, 7.9 Hz, 2H), 3.21 (dd, J = 10.8, 3.5 Hz, 2H), 2.92 (dd, J = 10.6, 6.6 Hz, 2H), 2.81 (s, 2H), 2.66 - 2.59 (m, 2H), 2.36 - 2.31 (m, 1H). LCMS (Analytical Method B) Rt = 1.38 min, MS (ESIpos): m/z 401.3 [M+H]+, Purity = 97%.

### Synthesis of Tert-butyl 5-{6-[(2-methylpyridin-4-yl)amino]-5-nitropyridin-2-yl}-octahydropyrrolo[3,4-c]pyrrole-2-carboxylate / Intermediate 9

A mixture of tert-butyl 2-(6-amino-5-nitro-2-pyridyl)-1,3,3a,4,6,6a-hexahydropyrrolo[3,4-c]pyrrole-5-carboxylate (Intermediate 8-1) (200 mg, 0.572 mmol), 4-bromo-2-methylpyridine (100 mg, 0.572 mmol), Pd₂(dba)₃ (13 mg, 0.0143 mmol), xantphos (17 mg, 0.0286 mmol) and Cs₂CO₃ (0.37 g, 1.14 mmol) in 1,4-dioxane (1.8 mL) was degassed by sparging with nitrogen. The reaction was stirred at 100° C. for 20 h. The reaction was cooled and the solid material removed by filtration, washing with 1,4-dioxane followed by DCM. The filtrate was concentrated in vacuo and the crude product was purified by flash chromatorgaphy (25 g, silica) eluting with 0-10% MeOH/DCM to yield the title compound (207 mg, 80% yield). 1H NMR (400 MHz, DMSO-d6) δ 10.76 (s, 1H), 8.34 (d, J = 5.8 Hz, 1H), 8.24 (d, J = 9.5 Hz, 1H), 7.75 - 7.61 (m, 2H), 6.25 (d, J = 9.5 Hz, 1H), 4.00 - 3.89 (m, 1H), 3.83 - 3.70 (m, 1H), 3.65 - 3.49 (m, 3H), 3.49 - 3.38 (m, 1H), 3.27 - 3.18 (m, 2H), 3.11 - 3.01 (m, 2H), 2.44 (s, 3H), 1.40 (s, 9H). LCMS (Analytical Method F) Rt = 0.84 min, MS (ESIpos): m/z 441.3 [M+H]+, Purity = 100%.

### Synthesis of Tert-butyl 4-{5-nitro-6-[(pyridazin-4-yl)amino]pyridin-2-yl}piperazine-1-carboxylate / Intermediate 10

tert-Butyl 4-(6-amino-5-nitro-2-pyridyl)piperazine-1-carboxylate (Intermediate 4-1) (250 mg, 0.773 mmol), 4-bromopyridazine (125 mg, 0.786 mmol), xantphos (50 mg, 0.0864 mmol) and Cs₂CO₃ (500 mg, 1.53 mmol) were suspended in 1,4-dioxane (4 mL) and the mixture was degassed with nitrogen for 5 min, then Pd₂(dba)₃ (40 mg, 0.0437 mmol) was added. The mixture was degassed for 5 min then sealed and stirred at 100° C. for 4 h under microwave irradiation. The reaction was retreated with 4-bromopyridazine (80 mg, 0.503 mmol), Pd₂(dba)₃ (40 mg, 0.0437 mmol) and Cs₂CO₃ (250 mg, 0.767 mmol) and stirred at 100° C. for 4 h under microwave irradiation. The mixture was quenched with water and extracted with EtOAc. The organics were combined and concentrated in vacuo and the residue was purified via flash cropatography (25 g, silica) eluting with 0-10% MeOH/DCM. The product was triturated with Et₂O and the solid collected by filtration to yield the title compound (318 mg, 87% yield). 1H NMR (500 MHz, DMSO-d6) δ 10.60 (s, 1H), 9.48 (dd, J = 2.8, 0.9 Hz, 1H), 9.07 (dd, J = 5.9, 0.7 Hz, 1H), 8.30 (d, J = 9.6 Hz, 1H), 8.03 (dd, J = 5.9, 2.8 Hz, 1H), 6.64 (d, J = 9.6 Hz, 1H), 3.79 - 3.73 (m, 4H), 3.52 - 3.45 (m, 4H), 1.43 (s, 9H). LCMS (Analytical Method F) Rt = 0.77 min, MS (ESIpos): m/z 402.3 [M+H]i+, Purity = 85%.

### Synthesis of Tert-butyl 3-(6-amino-5-nitropyridin-2-yl)-3,6-diazabicyclo[3.1.1]heptane-6-carboxylate / Intermediate 11-1

A suspension of tert-butyl 3,6-diazabicyclo[3.1.1]heptane-6-carboxylate (616 mg, 3.11 mmol) and 6-chloro-3-nitro-pyridin-2-amine (500 mg, 2.82 mmol) in MeCN (13.2 mL) was heated at 70° C. for 2 h. The reaction was cooled and the solvent removed in vacuo to yield the title compound as a yellow solid (1.14 g, quant. yield). 1H NMR (400 MHz, DMSO-d6) δ 8.12 (d, J = 9.4 Hz, 1H), 7.93 (s, 2H), 6.22 (d, J = 9.4 Hz, 1H), 4.19 (d, J = 6.1 Hz, 2H), 3.64 - 3.49 (m, 4H), 3.18 -3.06 (m, 2H), 1.29 (s, 9H). LCMS (Analytical Method H) Rt = 0.57 min, MS (ESIpos): m/z 336.3 [M+H]+, Purity = 90%.

### Synthesis of Tert-butyl 3-{5-nitro-6-[(pyridin-4-yl)amino]pyridin-2-yl}-3,6-diazabicyclo[3.1.1]heptane-6-carboxylate / Intermediate 11

A mixture of tert-butyl 3-(6-amino-5-nitro-2-pyridyl)-3,6-diazabicyclo[3.1.1]heptane-6-carboxylate (Intermediate 11-1) (500 mg, 1.34 mmol), 4-iodopyridine (289 mg, 1.41 mmol), Pd₂(dba)₃ (31 mg, 0.034 mmol), xantphos (39 mg, 0.067 mmol) and Cs₂CO₃ (874 mg, 2.68 mmol) in 1,4-dioxane (4 mL) was degassed by sparging with nitrogen. The reaction was stirred at 100° C. for 18 h. The reaction was cooled and the solid material removed by filtration. The filtrate was concentrated in vacuo and the residue was purified by flash chromatography (25 g, silica) eluting with 0-10% MeOH/DCM to yield the title compound as a yellow solid (457 mg, 76% yield). 1H NMR (500 MHz, DMSO-d6) δ 10.71 (s, 1H), 8.51 - 8.45 (m, 2H), 8.34 (d, J = 9.5 Hz, 1H), 7.82 (dd, J = 4.9, 1.5 Hz, 2H), 6.49 (d, J = 9.4 Hz, 1H), 4.32 - 4.18 (m, 3H), 3.80 - 3.57 (m, 2H), 3.18 (d, J = 5.2 Hz, 2H), 2.59 (d, J = 8.2 Hz, 1H), 1.27 (s, 9H). LCMS (Analytical Method F) Rt = 0.73 min, MS (ESIpos): m/z 413.3 [M+H]+, Purity = 92%.

### Synthesis of Tert-butyl 5-(6-amino-5-nitropyridin-2-yl)-2,5-diazabicyclo[2.2.2]octane-2-carboxylate / Intermediate 12-1

A suspension of tert-butyl 2,5-diazabicyclo[2.2.2]octane-2-carboxylate (396 mg, 1.86 mmol) and 6-chloro-3-nitro-pyridin-2-amine (300 mg, 1.69 mmol) in MeCN (8 mL) was heated at 70° C. for 1 h. The reaction was cooled and the solvent removed in vacuo. The residue was disolved in DCM, washed with water (3×) and brine, filtered through a Telos phase separator and evaporated in vacuo to yield the title compound as a yellow solid (548 mg, 91% yield). 1H NMR (400 MHz, Chloroform-d) δ 8.15 (d, J = 9.2 Hz, 1H), 5.77 (d, J = 9.0 Hz, 1H), 5.14 (d, J = 13.8 Hz, 1H), 4.32 (d, J = 63.5 Hz, 1H), 3.71 - 3.32 (m, 4H), 2.13 - 1.87 (m, 2H), 1.83 - 1.69 (m, 2H), 1.39 (s, 9H). LCMS (Analytical Method F) Rt = 0.94 min, MS (ESIpos): m/z 350.2 [M+H]+, Purity = 98%.

### Synthesis of Tert-butyl 5-{5-nitro-6-[(pyridin-4-yl)amino]pyridin-2-yl}-2,5-diazabicyclo[2.2.2]octane-2-carboxylate / Intermediate 12

A mixture of tert-butyl 5-(6-amino-5-nitro-2-pyridyl)-2,5-diazabicyclo[2.2.2]octane-2-carboxylate (Intermediate 12-1) (200 mg, 0.57 mmol), 4-iodopyridine (123 mg, 0.601 mmol), Pd₂(dba)₃(13 mg, 0.014 mmol), xantphos (17 mg, 0.029 mmol) and Cs₂CO₃ (373 mg, 1.14 mmol) in 1,4-dioxane (1.7 mL) was degassed by sparging with nitrogen. The reaction was stirred at 100° C. for 16 h. The reaction was cooled and the solid material removed by filtration washing with MeOH. The filtrate was concentrated in vacuo and the residue was purified by flash chromatography (25 g, silica) eluting with 0-5% MeOH/DCM to yield the title compound as a yellow solid (223 mg, 89% yield). 1H NMR (500 MHz, DMSO-d6) δ 10.88 - 10.52 (m, 1H), 8.52 - 8.41 (m, 2H), 8.35 - 8.19 (m, 1H), 7.81 (d, J = 5.1 Hz, 1H), 7.68 (d, J = 6.1 Hz, 1H), 6.70 - 6.18 (m, 1H), 5.04 - 4.50 (m, 1H), 4.41 - 4.21 (m, 1H), 3.81 (s, 1H), 3.76 - 3.59 (m, 1H), 3.59 - 3.45 (m, 2H), 2.03 - 1.77 (m, 4H), 1.49 - 1.37 (m, 9H). LCMS (Analytical Method F) Rt = 0.73 min, MS (ESIpos): m/z 413.3 [M+H]+, Purity = 92%.

### Synthesis of Tert-butyl 4-{6-[(2-benzamidopyridin-4-yl)amino]-5-nitropyridin-2-yl}piperazine-1-carboxylate / Intermediate 13

A mixture of tert-butyl 4-(6-amino-5-nitro-2-pyridyl)piperazine-1-carboxylate (Intermediate 4-1) (0.50 g, 1.55 mmol), N-(4-bromopyridin-2-yl)benzamide (628 mg, 1.70 mmol), and Cs₂CO₃ (1.01 g, 3.09 mmol) in 1,4-dioxane (5 mL) was degassed by sparging with nitrogen for 1 min. Then, Pd₂(dba)₃ (35 mg, 0.0387 mmol) and xantphos (45 mg, 0.0773 mmol) were added, and the reaction was heated to 100° C. for 2 h in a sealed tube. The reaction was cooled, diluted with water (10 mL) and extracted with EtOAc (3 × 20 mL). The organic extracts were combined, washed with brine (10 mL), dried over Na₂SO₄, filtered, and concentrated in vacuo. The residue was purified by flash chromatography (55 g, KP-NH) eluting with 0-100% TBME/heptane to afford the title compound as a yellow solid (112 mg, 14% yield). 1H NMR (500 MHz, DMSO-d6) δ 10.85 (s, 1H), 10.74 (s, 1H), 8.87 (s, 1H), 8.35 - 8.23 (m, 2H), 8.07 - 8.03 (m, 2H), 7.61 (t, J = 7.4 Hz, 1H), 7.52 (t, J = 7.7 Hz, 2H), 7.27 (d, J = 4.8 Hz, 1H), 6.62 (d, J = 9.6 Hz, 1H), 3.86 (br s, 4H), 3.51 (br s, 4H), 1.40 (s, 9H). LCMS (Analytical Method E) Rt = 1.25 min, MS (ESIpos): m/z 520.1 [M+H]+, Purity = 89%.

### Synthesis of N-(4-bromopyridin-2-yl)-4-fluorobenzamide / Intermediate 14-1

4-Fluorobenzoyl chloride (0.40 mL, 3.40 mmol) was added to a solution of 4-bromopyridin-2-amine (300 mg, 1.70 mmol) and DIPEA (0.59 mL, 3.40 mmol) in anhydrous DCM (3 mL), and the reaction mixture was stirred at RT for 18 h. MeOH (3 mL) and 2 M NaOH (3.0 mL, 6.00 mmol) were added and the reaction stirred at RT for 3.5 h. The mixture was diluted with water (3 mL) and extracted with DCM (3× 20 mL). The organic extracts were combined, dried over Na₂SO₄, filtered and concentrated in vacuo. The residue was purified by flash chromatography (25 g, silica) eluting with 0-40% EtOAc/heptane to provide the title compound as a white solid (442 mg, 88% yield). 1H NMR (400 MHz, Chloroform-d) δ 8.66 (d, J = 1.5 Hz, 1H), 8.58 (s, 1H), 8.13 (d, J = 5.3 Hz, 1H), 8.00 - 7.91 (m, 2H), 7.27 (dd, J = 5.4, 1.8 Hz, 1H), 7.25 - 7.18 (m, 2H). LCMS (Analytical Method F) Rt = 0.95 min, MS (ESIpos): m/z 294.9 [M+H]+, Purity = 100%.

### Synthesis of Tert-butyl 4-(6-{[2-(4-fluorobenzamido)pyridin-4-yl]amino}-5-nitropyridin-2-yl)piperazine-1-carboxylate / Intermediate 14

A mixture of tert-butyl 4-(6-amino-5-nitro-2-pyridyl)piperazine-1-carboxylate (Intermediate 4-1) (300 mg, 0.928 mmol), N-(4-bromopyridin-2-yl)-4-fluorobenzamide (Intermediate 14-1) (279 mg, 0.946 mmol), xantphos (54 mg, 0.0928 mmol) and Cs₂CO₃ (605 mg, 1.86 mmol) in 1,4-dioxane (5 mL) was degassed with nitrogen for 5 min. Then Pd₂(dba)₃ (42 mg, 0.0464 mmol) was added and the reaction was sealed under nitrogen and stirred at 100° C. for 2 h. The reaction was quenched with water and extracted with EtOAc (2×). The organic extracts were combined, dried over Na₂SO₄, filtered, and concentrated in vacuo. The residue was triturated with MeCN to afford the title compound as a yellow solid (480 mg, 77% yield), which was used in the next step without further purification. 1H NMR (500 MHz, DMSO-d6) δ 10.80 (s, 2H), 8.75 (s, 1H), 8.31 - 8.21 (m, 2H), 8.18 - 8.07 (m, 2H), 7.35 - 7.29 (m, 2H), 7.23 - 7.17 (m, 1H), 6.57 (d, J = 9.5 Hz, 1H), 3.88 - 3.72 (m, 4H), 3.50 - 3.44 (m, 4H), 1.39 (s, 9H). LCMS (Analytical Method H) Rt = 0.73 min, MS (ESIpos): m/z 538.3 [M+H]+, Purity = 80%.

### Synthesis of N-(4-bromopyridin-2-yl)pyridine-3-carboxamide / Intermediate 15-1

HATU (850 mg, 2.24 mmol) was added to a stirred solution of nicotinic acid (250 mg, 2.03 mmol) and DIPEA (1.0 mL, 5.73 mmol) in DMF (5 mL). After stirring at RT for 10 min, 4-bromopyridin-2-amine (370 mg, 2.10 mmol) was added and the reaction was stirred at RT for 16 h. The reaction was quenched with water, extracted with EtOAc, dried over Na₂SO₄, filtered, and concentrated in vacuo. The residue was purified by flash chromatography (25 g, silica) eluting with 0-100% EtOAc/heptane. The resulting product was triturated with MeOH to yield the title compound as a white solid (145 mg, 25% yield). 1H NMR (400 MHz, DMSO-d6) δ 11.31 (s, 1H), 9.12 (d, J = 2.2 Hz, 1H), 8.76 (dd, J = 4.8, 1.6 Hz, 1H), 8.46 (d, J = 1.7 Hz, 1H), 8.36 - 8.30 (m, 2H), 7.55 (dd, J = 8.0, 4.8 Hz, 1H), 7.47 (dd, J = 5.3, 1.8 Hz, 1H). LCMS (Analytical Method E) Rt = 0.97 min, MS (ESIpos): m/z 277.95, 279.95 [M+H]+, Purity = 99%.

### Synthesis of Tert-butyl 4-(5-nitro-6-{[2-(pyridine-3-amido)pyridin-4-yl]amino}pyridin-2-yl)piperazine-1-carboxylate / Intermediate 15-2

A mixture of tert-butyl 4-(6-amino-5-nitro-2-pyridyl)piperazine-1-carboxylate (Intermediate 4-1) (150 mg, 0.464 mmol), N-(4-bromopyridin-2-yl)pyridine-3-carboxamide (Intermediate 15-1) (145 mg, 0.521 mmol), xantphos (28 mg, 0.0484 mmol) and Cs₂CO₃ (305 mg, 0.936 mmol) in 1,4-dioxane (3 mL) was degassed with nitrogen for 5 min. Then Pd₂(dba)₃ (22 mg, 0.0240 mmol) was added and the reaction was sealed under nitrogen and stirred at 100° C. for 3 h under microwave irradiation. Additional tert-butyl 4-(6-amino-5-nitro-2-pyridyl)piperazine-1-carboxylate (Intermediate 4-1) (50 mg, 0.464 mmol) and Pd₂(dba)₃ (425 mg, 0.464 mmol) were added and the mixture was stirred at 100° C. for 1 h under microwave irradiation. The reaction was quenched with water and extracted with EtOAc (2×). The organic extracts were combined, dried over Na₂SO₄, filtered, and concentrated in vacuo. The residue was purified by flash chromatography (25 g, silica) eluting with 0-5% MeOH/DCM to afford the title compound as a yellow solid (265 mg, 98% yield). 1H NMR (500 MHz, DMSO-d6) δ 11.05 (s, 1H), 10.85 (s, 1H), 9.15 (d, J = 1.6 Hz, 1H), 8.86 (s, 1H), 8.76 (dd, J = 4.8, 1.6 Hz, 1H), 8.37 (dt, J = 8.0, 1.9 Hz, 1H), 8.32 - 8.27 (m, 2H), 7.54 (dd, J = 7.9, 4.8 Hz, 1H), 7.28 (d, J = 4.1 Hz, 1H), 6.61 (d, J = 9.6 Hz, 1H), 3.97 -3.75 (m, 4H), 3.53 - 3.46 (m, 4H), 1.39 (s, 9H). LCMS (Analytical Method H) Rt = 0.62 min, MS (ESIpos): m/z 521.4 [M+H]+, Purity = 91%.

### Synthesis of Tert-butyl 4-[2-(4-fluorophenyl)-3-[2-(pyridine-3-amido)pyridin-4-yl]-3H-imidazo[4,5-blpyridin-5-yllpiperazine-1-carboxylate / Intermediate 15

Na₂S₂O₄ (275 mg, 1.56 mmol) was added to a stirred solution of tert-butyl 4-(5-nitro-6-{[2-(pyridine-3-amido)pyridin-4-yl]amino}pyridin-2-yl)piperazine-1-carboxylate (Intermediate 15-2) (300 mg, 0.519 mmol) and 4-fluorobenzaldehyde (70 µL, 0.653 mmol) in DMSO (5 mL) and EtOH (1 mL) and the mixture was heated at 100° C. for 16 h in a sealed vial. The reaction was cooled to RT and quenched with sat. NaHCOs, extracted with EtOAc (2×), dried over Na₂SO₄, filtered, and concentrated in vacuo. The residue was purified by flash chromatography (25 g, silica) eluting with 0-8% MeOH/DCM, followed by preparative HPLC (Method A1) to provide the title compound as a yellow solid (94 mg, 27% yield). 1H NMR (500 MHz, DMSO-d6) δ 11.34 (s, 1H), 9.13 - 9.11 (m, 1H), 8.76 (dd, J = 4.8, 1.6 Hz, 1H), 8.54 (d, J = 1.6 Hz, 1H), 8.46 (d, J = 5.3 Hz, 1H), 8.35 - 8.31 (m, 1H), 8.01 (d, J = 8.9 Hz, 1H), 7.61 - 7.57 (m, 2H), 7.57 - 7.52 (m, 1H), 7.32 - 7.25 (m, 2H), 7.03 (dd, J = 5.4, 1.9 Hz, 1H), 6.95 (d, J = 9.0 Hz, 1H), 3.59 - 3.54 (m, 4H), 3.47 - 3.39 (m, 4H), 1.40 (s, 9H). LCMS (Analytical Method F) Rt = 0.98 min, MS (ESIpos): m/z 595.3 [M+H]+, Purity = 93%.

### Synthesis of N-(4-bromopyridin-2-yl)oxane-3-carboxamide / Intermediate 16-1

To a stirred solution of tetrahydropyran-3-carboxylic acid (285 mg, 2.12 mmol) and 4-bromopyridin-2-amine (250 mg, 1.42 mmol) in DMF (2 mL), DIPEA (742 µL, 4.25 mmol) and HATU (592 mg, 1.56 mmol) were added, and the mixture was stirred at RT for 72 h. The reaction was quenched with water (15 mL) and extracted with TBME (3 × 20 mL). The organic extracts were combined, washed with water (3 × 15 mL) and brine (15 mL), dried over Na₂SO₄, filtered and concentrated in vacuo. The residue was purified by flash chromatography (25 g, silica) eluting with 0-100% EtOAc/heptane to afford the title compound as a colourless oil (192.7 mg, 45% yield). 1H NMR (400 MHz, DMSO-d6) δ 10.74 (s, 1H), 8.32 (d, J = 1.7 Hz, 1H), 8.22 (d, J = 5.3 Hz, 1H), 7.36 (dd, J = 5.3, 1.8 Hz, 1H), 3.97 - 3.90 (m, 1H), 3.84 - 3.75 (m, 1H), 3.45 - 3.38 (m, 2H), 2.86 - 2.71 (m, 1H), 1.95 - 1.89 (m, 1H), 1.75 -1.45 (m, 3H). LCMS (Analytical Method E) Rt = 1.03 min, MS (ESIpos): m/z 284.8, 286.8 [M+H]+, Purity = 98%.

### Synthesis of Tert-butyl 4-(5-nitro-6-{[2-(oxane-3-amido)pyridin-4-yl]amino}pyridin-2-yl)piperazine-1 -carboxylate / Intermediate 16

A mixture of tert-butyl 4-(6-amino-5-nitro-2-pyridyl)piperazine-1-carboxylate (Intermediate 4-1) (215 mg, 0.666 mmol), N-(4-bromopyridin-2-yl)oxane-3-carboxamide (Intermediate 16-1) (190 mg, 0.666 mmol) and Cs₂CO₃ (434 mg, 1.33 mmol) in 1,4-dioxane (2.2 mL) was degassed with nitrogen for 1 min. Then Pd₂(dba)₃ (15 mg, 0.0167 mmol) and xantphos (19 mg, 0.0333 mmol) were added, and the reaction was heated at 100° C. for 2 h in a sealed tube. The reaction was diluted with water and extracted with EtOAc (3 × 20 mL). The organic extracts were combined, washed with brine (10 mL), dried over Na₂SO₄, filtered, and concentrated in vacuo. The residue was purified by flash chromatography (25 g, silica) eluting with 0-50% IPA/DCM, then by preparative HPLC (Method A2) to afford the title compound as a yellow solid (125.6 mg, 36% yield). 1HNMR(500 MHz, DMSO-d6) δ 10.79 (s, 1H), 10.48 (s, 1H), 8.76 (s, 1H), 8.29 (d, J = 9.6 Hz, 1H), 8.19 (d, J = 5.5 Hz, 1H), 7.19 - 7.07 (m, 1H), 6.60 (d, J = 9.7 Hz, 1H), 4.00 - 3.93 (m, 1H), 3.90 - 3.71 (m, 3H), 3.50 (s, 8H), 2.86 - 2.76 (m, 1H), 1.99 - 1.91 (m, 1H), 1.75 -1.66 (m, 1H), 1.65 - 1.59 (m, 1H), 1.58 - 1.50 (m, 1H), 1.43 (s, 9H). LCMS (Analytical Method E) Rt = 1.16 min, MS (ESIpos): m/z 528.35 [M+H]+, Purity = 100%.

### Synthesis of N-(4-bromopyridin-2-yl)cyclopropanecarboxamide / Intermediate 17-1

To a stirred solution of 4-bromopyridin-2-amine (300 mg, 1.70 mmol) in dry DCM (3 mL), DIPEA (0.59 mL, 3.40 mmol) was added, followed by cyclopropanecarbonyl chloride (0.31 mL, 3.40 mmol), and the resulting mixture was allowed to stir at RT overnight. MeOH (3 mL) and 2 M NaOH (3.0 mL, 6.00 mmol) were added and the mixture stirred at RT for 3.5 h. The mixture was diluted with water (3 mL), and extracted with DCM (3×20 mL). The organic extracts were combined, dried over Na₂SO₄, filtered and concentrated in vacuo. The residue was purified by flash chromatography (25 g, silica) eluting with 0-40% EtOAc/heptane to provide the title compound as a white solid (426 mg, quat. yield). 1H NMR (400 MHz, Chloroform-d) δ 8.49 (d, J = 1.6 Hz, 1H), 8.34 (s, 1H), 8.10 (d, J = 5.4 Hz, 1H), 7.20 (dd, J = 5.4, 1.7 Hz, 1H), 1.62 - 1.51 (m, 1H), 1.17 - 1.11 (m, 2H), 0.97 - 0.90 (m, 2H). LCMS (Analytical Method F) Rt = 0.77 min, MS (ESIpos): m/z 241.0 [M+H]+, Purity = 100%.

### Synthesis of Tert-butyl 4-{6-[(2-cyclopropaneamidopyridin-4-yl)amino]-5-nitropyridin-2-yl}piperazine-1-carboxylate / Intermediate 17

A mixture of tert-butyl 4-(6-amino-5-nitro-2-pyridyl)piperazine-1-carboxylate (Intermediate 4-1) (300 mg, 0.928 mmol), N-(4-bromopyridin-2-yl)cyclopropanecarboxamide (Intermediate 17-1) (228 mg, 0.946 mmol), xantphos (54 mg, 0.0928 mmol) and Cs₂CO₃ (605 mg, 1.86 mmol) in 1,4-dioxane (5 mL) was degassed with nitrogen for 5 min. Then Pd₂(dba)₃ (42 mg, 0.0464 mmol) was added and the reaction was sealed under nitrogen and stirred at 100° C. for 2 h. The reaction was quenched with water and extracted with EtOAc (2×). The organic extracts were combined, dried over Na₂SO₄, filtered, and concentrated in vacuo. The residue was purified by flash chromatography (25 g, silica) eluting with 0-10% MeOH/DCM to provide the title compound as an orange solid (440 mg, 88% yield). 1H NMR (500 MHz, DMSO-d6) δ 10.77 (d, J = 17.2 Hz, 2H), 8.75 (s, 1H), 8.27 (d, J = 9.6 Hz, 1H), 8.18 (d, J = 5.6 Hz, 1H), 7.10 (d, J = 4.3 Hz, 1H), 6.57 (d, J = 9.6 Hz, 1H), 3.79 (s, 4H), 3.46 (s, 4H), 2.05 - 1.99 (m, 1H), 1.44 (s, 9H), 0.83 - 0.82 (m, 2H), 0.82 - 0.80 (m, 2H). LCMS (Analytical Method F) Rt = 0.92 min, MS (ESIpos): m/z 484.2 [M+H]+, Purity = 90%.

### Synthesis of N-(4-bromopyridin-2-yl)acetamide / Intermediate 18-1

To an ice-cold solution of 4-bromopyridin-2-amine (450 mg, 2.55 mmol) in THF (8 mL), DIPEA (1.1 mL, 6.44 mmol) was added, followed by acetyl chloride (324 µL, 5.54 mmol). The mixture was stirred at RT for 1 h, then concentrated in vacuo. The residue was dissolved in MeOH (3 mL), and 2 M NaOH (1.5 mL, 3.00 mmol) was added and the reaction was stirred for 1 h before being quenched with 2 M HCI (1.5 mL). The mixture was diluted with water and extracted with DCM (2×). The organic extracts were combined and concentrated in vacuo, and the residue was purified by flash chromatography (25 g, silica) eluting with 0-100% EtOAc/heptane to provide the title compound as a white solid (595 mg, 97% yield). _1H NMR (500 MHz, DMSO-d6) δ 10.71 (s, 1H), 8.32 (d, J = 1.4 Hz, 1H), 8.21 (d, J = 5.3 Hz, 1H), 7.34 (dd, J = 5.3, 1.8 Hz, 1H), 2.10 (s, 3H). LCMS (Analytical Method F) Rt = 0.62 min, MS (ESIpos): m/z 215.0, 217.0 [M+H]+, Purity = 89%.

### Synthesis of Tert-butyl 4-{6-[(2-acetamidopyridin-4-yl)amino]-5-nitropyridin-2-yl}piperazine-1-carboxylate / Intermediate 18

A mixture of tert-butyl 4-(6-amino-5-nitro-2-pyridyl)piperazine-1-carboxylate (Intermediate 4-1) (350 mg, 1.08 mmol), N-(4-bromopyridin-2-yl)acetamide (Intermediate 18-1) (250 mg, 1.16 mmol), xantphos (63 mg, 0.108 mmol) and Cs₂CO₃ (705 mg, 2.16 mmol) in 1,4-dioxane (5 mL) was degassed with nitrogen for 5 min. Then Pd₂(dba)₃ (50 mg, 0.0541 mmol) was added and the reaction was heated at 100° C. for 2 h under microwave irradiation. The reaction was quenched with water and extracted with EtOAc (2×). The organic extracts were combined, dried over Na₂SO₄, filtered, and concentrated in vacuo. The residue was purified by flash chromatography (25 g, silica) eluting with 0-10% MeOH/DCM to provide the title compound as a yellow solid (303 mg, 54% yield). 1H NMR (400 MHz, DMSO-d6) δ 10.74 (s, 1H), 10.43 (s, 1H), 8.59 (d, J = 1.5 Hz, 1H), 8.27 (d, J = 9.6 Hz, 1H), 8.19 (d, J = 5.6 Hz, 1H), 7.22 (dd, J = 5.6, 2.0 Hz, 1H), 6.58 (d, J = 9.6 Hz, 1H), 3.86 - 3.74 (m, 4H), 3.51 - 3.43 (m, 4H), 2.09 (s, 3H), 1.43 (s, 9H). LCMS (Analytical Method F) Rt = 0.80 min, MS (ESIpos): m/z 458.3 [M+H]+, Purity = 89%.

### Synthesis of Tert-butyl (1S,4S)-5-(6-amino-5-nitropyridin-2-yl)-2,5-diazabicyclo[2.2.1]heptane-2-carboxylate / Intermediate 19-1

A suspension of 6-chloro-3-nitro-pyridin-2-amine (0.73 g, 4.19 mmol), tert-butyl (1S, 4S)-2,5-diazabicyclo[2.2.1]heptane-2-carboxylate (0.83 g, 4.19 mmol) and DIPEA (1.5 mL, 8.39 mmol) in MeCN (15 mL) was heated to 70° C. for 18 h. The reaction was cooled, and the precipitate was collected by filtration and washed with EtOAc to yield the title compound as a bright-yellow solid (1.29 g, 92% yield), which was used in the next step without further purification._1H NMR (400 MHz, DMSO-d6) δ 8.07 (d, J = 9.3 Hz, 1H), 7.62 (s, 2H), 6.07 (s, 1H), 4.96 (s, 1H), 4.51 (s, 1H), 3.57 (dd, J = 10.4, 1.9 Hz, 1H), 3.48 - 3.37 (m, 2H), 3.21 (d, J = 9.9 Hz, 1H), 1.99 - 1.88 (m, 2H), 1.42 (s, 9H). LCMS (Analytical Method F) Rt = 0.86 min, MS (ESIpos): m/z 336.2 [M+H]+, Purity = 100%.

### Synthesis of Tert-butyl (1S,4S)-5-{6-[(2-benzamidopyridin-4-yl)amino]-5-nitropyridin-2-yl}-2,5-diazabicyclo[2.2.1]heptane-2-carboxylate / Intermediate 19

A mixture of tert-butyl (1S, 4S)-5-(6-amino-5-nitropyridin-2-yl)-2,5-diazabicyclo[2.2.1]heptane-2-carboxylate (Intermediate 19-1) (250 mg, 0.745 mmol), N-(4-bromopyridin-2-yl)benzamide (211 mg, 0.760 mmol), xantphos (22 mg, 0.0373 mmol), Pd₂(dba)₃ (17 mg, 0.0186 mmol) and Cs₂CO₃ (486 mg, 1.49 mmol) in 1,4-dioxane (7.2 mL) was degassed with nitrogen for 5 min. The reaction was heated at 100° C. for 3 h under microwave irradiation. The reaction was diluted with water and extracted with EtOAc (3×). The organic extracts were combined, dried over Na₂SO₄, filtered, and concentrated in vacuo. The residue was purified by flash chromatography (25 g, silica) eluting with 0-100% EtOAc/heptane to provide the title compound as a bright-yellow solid (332 mg, 84% yield). LCMS (Analytical Method F) Rt = 0.98 min, MS (ESIpos): m/z 532.2 [M+H]+, Purity = 100%.

### Synthesis of Tert-butyl (1S,4S)-5-(6-{[2-(4-fluorobenzamido)pyridin-4-yl]amino}-5-nitropyridin-2-yl)-2,5-diazabicyclo[2.2.1]heptane-2-carboxylate / Intermediate 20

A mixture of tert-butyl (1S, 4S)-5-(6-amino-5-nitropyridin-2-yl)-2,5-diazabicyclo[2.2.1]heptane-2-carboxylate (Intermediate 19-1) (200 mg, 0.596 mmol), N-(4-bromopyridin-2-yl)-4-fluorobenzamide (Intermediate 14-1) (180 mg, 0.608 mmol), xantphos (17 mg, 0.0298 mmol), Pd₂(dba)₃ (14 mg, 0.0149 mmol) and Cs₂CO₃ (389 mg, 1.19 mmol) in 1,4-dioxane (5.8 mL) was degassed with nitrogen for 5 min. The reaction was heated at 100° C. for 3 h using a sealed tube. The reaction was diluted with water and extracted with EtOAc (3×). The organic extracts were combined, dried over Na₂SO₄, filtered, and concentrated in vacuo. The residue was triturated with MeCN to provide the title compound as a yellow solid (260 mg, 76% yield), which was used in the next step without further purification. LCMS (Analytical Method F) Rt = 1.01 min, MS (ESIpos): m/z 550.2 [M+H]+, Purity = 96%.

### Synthesis of Tert-butyl (1S,4S)-5-{6-[(2-cyclopropaneamidopyridin-4-yl)amino]-5-nitropyridin-2-yl}-2,5-diazabicyclo[2.2.1]heptane-2-carboxylate / Intermediate 21

A mixture of tert-butyl (1S, 4S)-5-(6-amino-5-nitropyridin-2-yl)-2,5-diazabicyclo[2.2.1]heptane-2-carboxylate (Intermediate 19-1) (200 mg, 0.596 mmol), N-(4-bromopyridin-2-yl)cyclopropanecarboxamide (Intermediate 17-1) (147 mg, 0.608 mmol), xantphos (17 mg, 0.0298 mmol), Pd₂(dba)₃ (14 mg, 0.0149 mmol) and Cs₂CO₃ (389 mg, 1.19 mmol) in 1,4-dioxane (5.8 mL) was degassed with nitrogen for 5 min. The reaction was heated at 100° C. for 3 h using a sealed tube. The reaction was diluted with water and extracted with EtOAc (3×). The organic extracts were combined, dried over Na₂SO₄, filtered, and concentrated in vacuo. The residue was triturated with MeCN to provide the title compound as a yellow solid (165 mg, 55% yield), which was used in the next step without further purification. LCMS (Analytical Method F) Rt = 0.84 min, MS (ESIpos): m/z 496.2 [M+H]+, Purity = 99%.

### Synthesis of Tert-butyl 4-(6-{[2-({[(tert-butoxy)carbonyl](methyl)amino}methyl)pyridin-4-yl]amino}-5-nitropyridin-2-yl)piperazine-1-carboxylate / Intermediate 22

A mixture of tert-butyl 4-(6-amino-5-nitro-2-pyridyl)piperazine-1-carboxylate (Intermediate 4-1) (450 mg, 1.39 mmol), tert-butyl N-[(4-bromopyridin-2-yl)methyl]-N-methylcarbamate (Intermediate 22-2) (428 mg, 1.42 mmol), xantphos (40 mg, 0.0696 mmol), Pd₂(dba)₃ (32 mg, 0.0348 mmol) and Cs₂CO₃ (907 mg, 2.78 mmol) in 1,4-dioxane (13.5 mL) was degassed with nitrogen for 5 min. The reaction was heated at 100° C. for 6 h under microwave irradiation. The reaction was diluted with water and extracted with EtOAc (3×). The organic extracts were combined, dried over Na₂SO₄, filtered, and concentrated in vacuo. The residue was purified by flash chromatography (25 g, silica) eluting with 0-100% EtOAc/heptane to provide the title compound as a bright-yellow solid (656 mg, 87% yield). 1H NMR (400 MHz, DMSO-d6) δ 10.67 (s, 1H), 8.42 (d, J = 5.6 Hz, 1H), 8.29 (d, J = 9.6 Hz, 1H), 7.66 - 7.47 (m, 2H), 6.60 (d, J = 9.6 Hz, 1H), 4.44 (s, 2H), 3.77 (s, 4H), 3.50 (s, 4H), 2.88 (s, 3H), 1.44 - 1.31 (m, 18H). LCMS (Analytical Method F) Rt = 0.89 min, MS (ESIpos): m/z 544.3 [M+H]+, Purity = 100%.

### Synthesis of N-(4-bromopyridin-2-yl)morpholine-4-carboxamide / Intermediate 23-1

To a solution of 4-bromopyridin-2-amine (250 mg, 1.42 mmol) and pyridine (0.13 mL, 1.56 mmol) in THF (2 mL), (4-nitrophenyl) carbonochloridate (314 mg, 1.56 mmol) was added and the reaction mixture stirred at RT for 1 h. Morpholine (0.22 mL, 1.84 mmol) and DIPEA (0.37 mL, 2.12 mmol) in THF (1 mL) were added and the reaction mixture stirred at RT for 1 h. The mixture was concentrated in vacuo. The residue was purified by flash chromatography (25 g, silica) eluting with 0-100% EtOAc/heptane to yield the title compound (332 mg, 65% yield). 1H NMR (400 MHz, DMSO-d6) δ 9.46 (s, 1H), 8.17 - 8.13 (m, 1H), 8.05 (d, J = 1.4 Hz, 1H), 7.23 (dd, J = 5.3, 1.8 Hz, 1H), 3.64 - 3.53 (m, 4H), 3.47 -3.43 (m, 4H). LCMS (Analytical Method E) Rt = 0.86 min, MS (ESIpos): m/z 285.8, 287.7 [M+H]+, Purity = 99%.

### Synthesis of Tert-butyl 4-[6-({2-[(morpholine-4-carbonyl)amino]pyridin-4-yl}amino)-5-nitropyridin-2-yllpiperazine-1-carboxylate / Intermediate 23

A mixture of tert-butyl 4-(6-amino-5-nitro-2-pyridyl)piperazine-1-carboxylate (Intermediate 4-1) (289 mg, 0.895 mmol), N-(4-bromo-2-pyridyl)morpholine-4-carboxamide (Intermediate 23-1) (320 mg, 0.895 mmol) and Cs₂CO₃ (583 mg, 1.79 mmol) in 1,4-dioxane (3 mL) was degassed by sparging with nitrogen for 1 min. Pd₂(dba)₃ (20 mg, 0.0224 mmol) and xantphos (26 mg, 0.0447 mmol) were then added and the reaction mixture degassed with nitrogen for 1 min before it was stirred at 100° C. for 18 h. The reaction was retreated with Pd₂(dba)₃ (20 mg, 0.0224 mmol) and xantphos (26 mg, 0.0447 mmol) and the reaction mixture degassed with nitrogen for 1 min. The mixture was stirred at 100° C. for 2 h. The reaction was cooled, diluted with water (10 mL) and extracted with EtOAc (2×). The organic extracts were combined, washed with brine, dried over Na₂SO₄, filtered and concentrated in vacuo. The residue was purified by flash chromatography (25 g, silica) eluting with 0-50% IPA/DCM. The resulting product was further purified by preparative HPLC (Method A2) to yield the title compound (58 mg, 12% yield). 1H NMR (500 MHz, DMSO-d6) δ 10.77 (s, 1H), 9.18 (s, 1H), 8.48 (s, 1H), 8.28 (d, J = 9.6 Hz, 1H), 8.13 (d, J = 5.5 Hz, 1H), 7.06 (d, J = 5.0 Hz, 1H), 6.59 (d, J = 9.7 Hz, 1H), 3.81 (s, 4H), 3.64 - 3.57 (m, 4H), 3.51 - 3.46 (m, 8H), 1.43 (s, 9H). LCMS (Analytical Method E) Rt = 1.04 min, MS (ESIpos): m/z 529.4 [M+H]+, Purity = 96%.

### Synthesis of N-(4-bromopyridin-2-yl)piperidine-1-carboxamide / Intermediate 24-1

To a solution of 4-bromopyridin-2-amine (250 mg, 1.42 mmol) and pyridine (0.13 mL, 1.56 mmol) in THF (2 mL), (4-nitrophenyl)carbonochloridate (314 mg, 1.56 mmol) was added and the reaction mixture stirred at RT for 15 min. Piperidine (0.18 mL, 1.84 mmol) and DIPEA (0.37 mL, 2.12 mmol) in THF (1 mL) were added and the reaction mixture stirred at RT for 1 h. The mixture was concentrated in vacuo and the residue was purified by flash chromatography (25 g, silica), eluting with 0-100% EtOAc/heptane to yield the title compound (263 mg, 52% yield)._1H NMR (400 MHz, DMSO-d6) δ 9.34 (s, 1H), 8.12 - 8.10 (m, 1H), 8.04 (d, J = 1.5 Hz, 1H), 7.20 (dd, J = 5.4, 1.8 Hz, 1H), 3.46 -3.40 (m, 4H), 1.63 - 1.52 (m, 2H), 1.50 - 1.43 (m, 4H)._LCMS (Analytical Method E) Rt = 1.00 min, MS (ESIpos): m/z 283.8, 285.7 [M+H]+, Purity = 87%.

### Synthesis of Tert-butyl 4-[5-nitro-6-({2-[(piperidine-1-carbonyl)amino]pyridin-4-yl}amino)pyridin-2-yl]piperazine-1-carboxylate / Intermediate 24-2

A mixture of tert-butyl 4-(6-amino-5-nitro-2-pyridyl)piperazine-1-carboxylate (Intermediate 4-1) (237 mg, 0.732 mmol), N-(4-bromo-2-pyridyl)piperidine-1-carboxamide (Intermediate 24-1) (260 mg, 0.732 mmol) and Cs₂CO₃ (477 mg, 1.46 mmol) in 1,4-dioxane (2.4 mL) was degassed by sparging with nitrogen for 1 min. Pd₂(dba)₃ (17 mg, 0.0183 mmol) and xantphos (21 mg, 0.0366 mmol) were then added and the reaction mixture degassed with nitrogen for 1 min before it was stirred at 100° C. for 18 h. The reaction was retreated with Pd₂(dba)₃ (17 mg, 0.0183 mmol) and xantphos (21 mg, 0.0366 mmol) and the reaction mixture degassed with nitrogen for 1 min. The mixture was stirred at 100° C. for 2 h. The reaction was cooled, diluted with water (10 mL) and extracted with EtOAc (3×). The organic extracts were combined, washed with brine, dried over Na₂SO₄, filtered and concentrated in vacuo. The residue was purified by flash chromatography (25 g, silica) eluting with 0-50% IPA/DCM. The resulting product was further purified by preparative HPLC (Method B2) to yield the title compound as a yellow solid (52 mg, 12% yield). 1H NMR (500 MHz, DMSO-d6) δ 10.78 (s, 1H), 9.03 (s, 1H), 8.48 (s, 1H), 8.28 (d, J = 9.6 Hz, 1H), 8.11 (d, J = 5.5 Hz, 1H), 7.02 (d, J = 4.5 Hz, 1H), 6.58 (d, J = 9.6 Hz, 1H), 3.89 - 3.74 (m, 2H), 3.52 - 3.48 (m, 4H), 3.46 - 3.44 (m, 6H), 1.62 - 1.54 (m, 2H), 1.53 - 1.45 (m, 4H), 1.43 (s, 9H). LCMS (Analytical Method E) Rt = 1.13 min, MS (ESIpos): m/z 527.4 [M+H]+, Purity = 86%.

### Synthesis of Tert-butyl 4-[2-(4-fluorophenyl)-3-{2-[(piperidine-1-carbonyl)amino]pyridin-4-yl}-3H-imidazo[4,5-b]pyridin-5-yl]piperazine-1-carboxylate/ Intermediate 24

4-fluorobenzaldehyde (36 mg, 0.291 mmol) was added to a solution of tert-butyl 4-[5-nitro-6-[[2-(piperidine-1-carbonylamino)-4-pyridyl]amino]-2-pyridyl]piperazine-1-carboxylate (Intermediate 24-2) (59 mg, 0.0968 mmol) in EtOH (0.15 mL) and DMSO (1 mL). The reaction was stirred for 5 min then Na₂S₂O₄ (102 mg, 0.581 mmol) was added and the reaction was heated at 100° C. for 18 h. Additional piperidine (0.20 mL, 2.02 mmol) was added and the reaction heated at 120° C. for 18 h. The reaction was then separated between DCM (2 × 5 mL) and NaHCOs (5 mL) and filtered through a Telos phase separator. The filtrate was concentrated in vacuo and then purified by preparative HPLC (Method B1) to afford the title compound as a brown oil (29 mg, 47% yield), which was used in the next step without further purification. LCMS (Analytical Method F) Rt = 0.98 min, MS (ESIpos): m/z 601.5 [M+H]+, Purity = 95%.

### Synthesis of 4-bromo-2-(methoxymethyl)pyridine / Intermediate 25-1

NaH (60%, 48 mg, 1.20 mmol) was adde to an ice-cold solution of (4-bromopyridin-2-yl)methanol (150 mg, 0.798 mmol) in anhydrous THF (3.7 mL), and the mixture was allowed to warm up to RT and stirred for 1 h. The solution was then cooled to 0° C., and iodomethane (74 µL, 1.20 mmol) was added and the solution stirred at RT for 2 h. lodomethane (10 µL, 0.16 mmol) was added again and the solution allowed to stir for a further 3 h. The mixture was filtered-off washing with THF, and the filtrate was evaporated in vacuo. The residue was purified by flash chromatography (10 g, silica), eluting with 0-55% EtOAc/heptane to yield the title compound as a pale-yellow volatile oil (108 mg, 67% yield). 1H NMR (400 MHz, Chloroform-d) δ 8.29 (d, J = 5.3 Hz, 1H), 7.76 - 7.52 (m, 1H), 7.30 (dd, J = 5.3, 1.9 Hz, 1H), 4.49 (s, 2H), 3.42 (s, 3H). LCMS (Analytical Method F) Rt = 0.65 min, MS (ESIpos): m/z 202.0 [M+H]+, Purity = 100%.

### Synthesis of Tert-butyl 4-(6-{[2-(methoxymethyl)pyridin-4-yl]amino}-5-nitropyridin-2-yl)piperazine-1-carboxylate / Intermediate 25

A mixture of tert-butyl 4-(6-amino-5-nitro-2-pyridyl)piperazine-1-carboxylate (Intermediate 4-1) (150 mg, 0.464 mmol), 4-bromo-2-(methoxymethyl)pyridine (Intermediate 25-1) (94 mg, 0.464 mmol), Pd₂(dba)₃ (11 mg, 0.0116 mmol), xantphos (13 mg, 0.0232 mmol) and Cs₂CO₃ (0.300 g, 0.928 mmol) in 1,4-dioxane (1.5 mL) was degassed by sparging with nitrogen. The reaction was heated to 100° C. for 20 h. The reaction was cooled and the solid material removed by filtration, washing with 1,4-dioxane and DCM. The filtrate was concentrated in vacuo and the residue was purified by flash chromatography (25 g, silica), eluting with 0-10% MeOH/DCM to yield the title compound as a yellow solid (140 mg, 24% yield). 1H NMR (400 MHz, DMSO-d6) δ 10.69 (s, 1H), 8.40 (d, J = 5.5 Hz, 1H), 8.09 (d, J = 9.5 Hz, 1H), 8.06 - 8.01 (m, 1H), 7.44 - 7.39 (m, 1H), 6.33 (d, J = 9.5 Hz, 1H), 4.50 (s, 2H), 3.80 (s, 4H), 3.52 - 3.47 (m, 4H), 2.90 (s, 3H), 1.44 (s, 9H). LCMS (Analytical Method F) Rt = 0.84 min, MS (ESIpos): m/z 445.2 [M+H]+, Purity = 36%.

### Synthesis of N-(6-chloro-3-nitropyridin-2-yl)pyrimidin-4-amine / Intermediate 26-1

NaH (60%, 155 mg, 3.89 mmol) was added to an ice-cold solution of pyrimidin-4-amine (370 mg, 3.89 mmol) in anhydrous DMF (5.2 mL). After stirring for 10 min, 2,6-dichloro-3-nitropyridine (500 mg, 2.59 mmol) was added dropwise and the reaction stirred at 0° C. for2 h. The reaction was quenched by addition of sat. NH₄Cl (20 mL). The aqueous layer was extracted with EtOAc (3 × 20 mL), the combined organics washed with brine (15 mL), dried over Na₂SO₄ and concentrated in vacuo. The residue was purified by flash chromatography (25 g, silica) eluting with 0-100% TBME/heptane to provide the title compound as yellow solid (188 mg, 28% yield). 1H NMR (400 MHz, DMSO-d6) δ 10.63 (s, 1H), 8.81 (d, J = 1.0 Hz, 1H), 8.69 (d, J = 5.9 Hz, 1H), 8.62 (d, J = 8.6 Hz, 1H), 7.89 (dd, J = 5.8, 1.3 Hz, 1H), 7.39 (d, J = 8.6 Hz, 1H). LCMS (Analytical Method E) Rt = 1.03 min, MS (ESIpos): m/z 251.8 [M+H]+, Purity = 97%.

### Synthesis of Tert-butyl 4-{5-nitro-6-[(pyrimidin-4-yl)amino]pyridin-2-yl}piperazine-1-carboxylate / Intermediate 26

To a stirred solution of N-(6-chloro-3-nitropyridin-2-yl)pyrimidin-4-amine (Intermediate 26-1) (94 mg, 0.374 mmol) and tert-butyl piperazine-1-carboxylate (213 mg, 1.12 mmol) in IPA (0.5 mL), DIPEA (0.20 mL, 1.12 mmol) was added, and the resulting mixture was stirred at 100° C. for 1.5 h in a sealed tube. The solvent was concentrated in vacuo and the residue was purified by flash chromatography (25 g, silica) eluting with 0-50% MeOH/TBME to afford the title compound as a yellow solid (91 mg, 58% yield). 1H NMR (500 MHz, DMSO-d6) δ 11.04 (s, 1H), 8.88 (d, J = 0.9 Hz, 1H), 8.74 (d, J = 5.8 Hz, 1H), 8.33 (d, J = 9.6 Hz, 1H), 8.22 (dd, J = 5.8, 1.3 Hz, 1H), 6.69 (d, J = 9.6 Hz, 1H), 3.87 - 3.78 (m, 4H), 3.55 - 3.50 (m, 4H), 1.44 (s, 9H). LCMS (Analytical Method E) Rt = 1.17 min, MS (ESIpos): m/z 402.05 [M+H]+, Purity = 100%.

### Example 1.3 - Synthesis of Compounds

### 2-Fluorophenyl)-6-(piperazin-1-yl)-1-(pyridin-4-yl)-1H-1,3-benzodiazole / Compound 1-1 (#28 from Table 1)

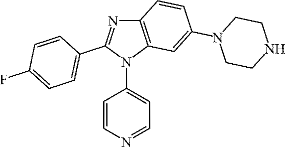 [SMILES: Fc1ccc(-c2nc3ccc(N4CCNCC4)cc3n2-c2ccncc2)cc1]

4 M HCI in 1,4-dioxane (507 µL, 2.03 mmol) was added to a suspension of tert-butyl 4-[2-(4-fluorophenyl)-3-(4-pyridyl)benzimidazol-5-yl]piperazine-1-carboxylate (Intermediate 1) (48 mg, 0.101 mmol) in 1,4-dioxane (1 mL). The reaction was stirred for 1 h then the precipitate collected by filtration, washed with 1,4-dioxane and dried in vacuo. The residue was purified by preparative HPLC (Method A1) to afford the title compound (18 mg, 48% yield). 1H NMR (400 MHz, DMSO-d6) δ 8.78 - 8.73 (m, 2H), 7.63 (d, J = 8.9 Hz, 1H), 7.53 -7.43 (m, 4H), 7.24 (t, J = 8.9 Hz, 2H), 7.07 (dd, J = 8.9, 2.3 Hz, 1H), 6.70 (d, J = 2.1 Hz, 1H), 3.05 - 2.99 (m, 4H), 2.86 - 2.79 (m, 4H). LCMS (Analytical Method A) Rt = 1.39 min, MS (ESIpos): m/z 374.2 [M+H]+, Purity = 100%.

### 2-Fluorophenyl)-1-(2-methylpyridin-4-yl)-6-(piperazin-1-yl)-1H-1,3-benzodiazole / Compound 1-2 (#29 From Table 1)

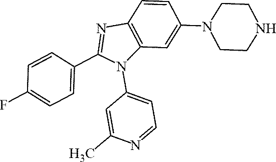 [SMILES: Cc1cc(-n2c(-c3ccc(F)cc3)nc3ccc(N4CCNCC4)cc32)ccn1]

tert-Butyl 4-[2-(4-fluorophenyl)-3-(2-methyl-4-pyridyl)benzimidazol-5-yl]piperazine-1-carboxylate (Intermediate 2) (65 mg, 0.113 mmol) was suspended in 4 M HCl in 1,4-dioxane (2 mL) and stirred at RT for 10 min. MeOH (1 mL) was added and the reaction was stirred for 1 h. The mixture was concentrated in vacuo and the residue loaded to an SCX-2 ion exchange cartridge. The cartridge was washed with DCM/MeOH then the product was eluted with 7N NH₃ in MeOH, concentrated in vacuo and lyophilised overnight to afford the title compound (40 mg, 91% yield). 1H NMR (400 MHz, DMSO-d6) δ 8.61 (d, J = 5.3 Hz, 1H), 7.64 (d, J = 8.9 Hz, 1H), 7.53 - 7.47 (m, 2H), 7.35 (d, J = 1.7 Hz, 1H), 7.27 - 7.20 (m, 3H), 7.07 (dd, J = 8.9, 2.1 Hz, 1H), 6.70 (d, J = 2.0 Hz, 1H), 3.15 - 3.06 (m, 4H), 2.98 - 2.89 (m, 4H), 2.52 (s, 3H). LCMS (Analytical Method A) Rt = 1.30 min, MS (ESIpos): m/z 388.2 [M+H]+, Purity = 100%.

### 2-Fluorophenyl)-6-(piperazin-1-yl)-1-(pyrimidin-4-yl)-1H-1,3-benzodiazole / Compound 1-3 (#26 of Table 1)

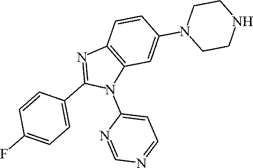 [SMILES: Fc1ccc(-c2nc3ccc(N4CCNCC4)cc3n2-c2ccncn2)cc1]

TFA (150 µL, 2.02 mmol) was added to a solution of tert-butyl 4-[2-(4-fluorophenyl)-3-pyrimidin-4-yl-benzimidazol-5-yl]piperazine-1-carboxylate (Intermediate 3) (48 mg, 0.101 mmol) in DCM (1 mL) and the reaction stirred for 1 h then concentrated in vacuo. The residue was purified by preparative HPLC (Method A1) to afford the title compound (26 mg, 67% yield). 1H NMR (400 MHz, DMSO-d6) δ 9.27 (d, J = 1.0 Hz, 1H), 8.94 (d, J = 5.5 Hz, 1H), 7.63 (d, J = 8.9 Hz, 1H), 7.51 (dd, J = 8.9, 5.4 Hz, 2H), 7.46 (dd, J = 5.4, 1.3 Hz, 1H), 7.26 (t, J = 8.9 Hz, 2H), 7.17 (d, J = 2.2 Hz, 1H), 7.09 (dd, J = 8.9, 2.3 Hz, 1H), 3.08 - 3.02 (m, 4H), 2.87 - 2.82 (m, 4H). LCMS (Analytical Method A) Rt = 1.36 min, MS (ESIpos): m/z 375.2 [M+H]+, Purity = 98%.

### N-{4-(4-fluorophenyl)-5-(piperazin-1-yl)-3H-imidazo[4,5-b]pyridin-3-yl]pyridin-2-yl}pyridine-3-carboxamide / Compound 1-4 (#2 in Table 1)

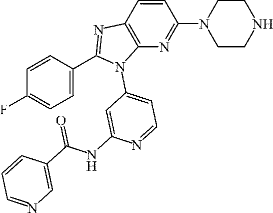 [SMILES: O=C(Nc1cc(-n2c(-c3ccc(F)cc3)nc3ccc(N4CCNCC4)nc32)ccn1)c1cccnc1]

tert-Butyl 4-[2-(4-fluorophenyl)-3-[2-(pyridine-3-carbonylamino)-4-pyridyl]imidazo[4,5-b]pyridin-5-yl]piperazine-1-carboxylate (Intermediate 15) (94 mg, 0.142 mmol) was dissolved in 4 M HCl in 1,4-dioxane (3 mL) and stirred for 1 h. The mixture was concentrated in vacuo and the residue purified by preparative HPLC (Method A1) to afford the title compound (41 mg, 56% yield). 1H NMR (400 MHz, DMSO-d6) δ 11.30 (s, 1H), 9.11 (dd, J = 2.3, 0.8 Hz, 1H), 8.76 (dd, J = 4.8, 1.6 Hz, 1H), 8.50 - 8.45 (m, 2H), 8.32 (m, 1H), 7.96 (d, J = 8.9 Hz, 1H), 7.62 - 7.53 (m, 3H), 7.31 - 7.24 (m, 2H), 7.08 (dd, J = 5.3, 2.0 Hz, 1H), 6.89 (d, J = 9.0 Hz, 1H), 3.47 - 3.43 (m, 4H), 2.80 - 2.74 (m, 4H). LCMS (Analytical Method B) Rt = 2.59 min, MS (ESIpos): m/z 495.4 [M+H]+, Purity = 97%.

### N-{4-(4-fluorophenyl)-5-(piperazin-1-yl)-3H-imidazo[4,5-b]pyridin-3-yl]pyridin-2-yl)piperidine-1-carboxamide / Compound 1-5 (#33 of Table 1)

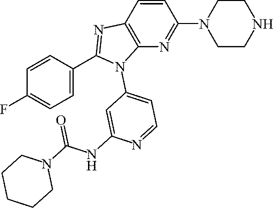 [SMILES: O=C(Nc1cc(-n2c(-c3ccc(F)cc3)nc3ccc(N4CCNCC4)nc32)ccn1)N1CCCCC1]

To a stirred solution of tert-butyl 4-[2-(4-fluorophenyl)-3-[2-(piperidine-1-carbonylamino)-4-pyridyl]imidazo[4,5-b]pyridin-5-yl]piperazine-1-carboxylate (Intermediate 24) (29 mg, 0.0479 mmol) in DCM (0.8 mL) was added TFA (0.2 mL, 2.62 mmol) and the mixture stirred at RT for 1 h. The mixture was concentrated in vacuo and the residue purified by preparative HPLC (Method A1) to afford the title compound (2 mg, 10% yield). 1H NMR (500 MHz, DMSO-d6) δ 9.30 (s, 1H), 8.22 (d, J = 5.4 Hz, 1H), 7.91 (d, J = 1.7 Hz, 1H), 7.87 (d, J = 8.9 Hz, 1H), 7.54 -7.43 (m, 2H), 7.19 (t, J = 8.9 Hz, 2H), 6.80 (d, J = 9.0 Hz, 1H), 6.78 (dd, J = 5.4, 1.9 Hz, 1H), 3.36 - 3.34 (m, 8H), 2.73 - 2.65 (m, 4H), 1.55 - 1.46 (m, 2H), 1.44 - 1.34 (m, 4H). LCMS (Analytical Method A) Rt = 1.80 min, MS (ESIpos): m/z 501.4 [M+H]+, Purity = 99%.

### 1-(4-Fluorophenyl)-3-(pyridin-4-yl)-3H-imidazo[4,5-b]pyridin-5-yl]piperazine / Compound 2-1 (#17 of Table 1)

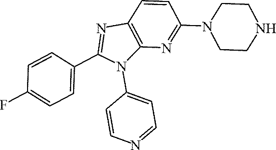 [SMILES: Fc1ccc(-c2nc3ccc(N4CCNCC4)nc3n2-c2ccncc2)cc1]

A mixture of tert-butyl 4-[5-nitro-6-(4-pyridylamino)-2-pyridyl]piperazine-1-carboxylate (Intermediate 4) (100 mg, 0.250 mmol) and Na₂S₂O₄ (132 mg, 0.749 mmol) in DMSO (1 mL) and EtOH (0.2 mL) was gently warmed for 30 s. 4-Fluorobenzaldehyde (40 µL, 0.375 mmol) was added and the reaction heated to 100° C. for 20 h. The reaction was cooled and quenched into water. The aqueous layer was washed with EtOAc then basified with NaHCOs (aq.). The aqueous layer was extracted into EtOAc (3×), the combined organics washed with brine, dried over MgSO₄ and concentrated in vacuo. The residue was purified by preparative HPLC (Method A1) to afford the title compound (13 mg, 14% yield). 1H NMR (500 MHz, DMSO-d6) δ 8.72 - 8.68 (m, 2H), 7.95 (d, J = 8.9 Hz, 1H), 7.50 (dd, J = 8.9, 5.4 Hz, 2H), 7.45 -7.41 (m, 2H), 7.25 (t, J = 8.9 Hz, 2H), 6.89 (d, J = 9.0 Hz, 1H), 3.42 - 3.38 (m, 4H), 2.79 - 2.74 (m, 4H). LCMS (Analytical Method A) Rt = 1.34 min, MS (ESIpos): m/z 375.3 [M+H]+, Purity = 98%.

### Synthesis of 1-[2-(4-Chlorophenyl)-3-(pyridin-4-yl)-3H-imidazo[4,5-b]pyridin-5-yl]piperazine / Compound 2-2 (#18 of Table 1)

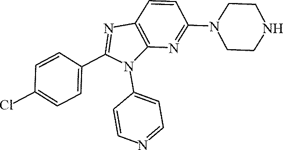 [SMILES: Clc1ccc(-c2nc3ccc(N4CCNCC4)nc3n2-c2ccncc2)cc1]

Na₂S₂O₄ (132 mg, 0.749 mmol) was added to a suspension of tert-butyl 4-[5-nitro-6-(4-pyridylamino)-2-pyridyl]piperazine-1-carboxylate (Intermediate 4) (100 mg, 0.250 mmol) and 4-chlorobenzaldehyde (54 mg, 0.375 mmol) in EtOH (0.2 mL) and DMSO (1 mL). The reaction was heated to 100° C. for 18 h then cooled and diluted with water. The aqueous layer was washed with 1:1 THF/EtOAc (3 × 20 mL) then neutralised with sat. aq. NaHCO₃ and then extracted into EtOAc (3×). The organic extracts were combined, dried over Na₂SO₄ and concentrated in vacuo. The residue was purified by preparative HPLC (Method B1) to afford the title compound (10 mg, 10% yield). 1H NMR (400 MHz, DMSO-d6) δ 8.75 - 8.69 (m, 2H), 8.24 (s, 1H), 7.97 (d, J = 8.9 Hz, 1H), 7.47 (s, 4H), 7.45 - 7.43 (m, 2H), 6.91 (d, J = 9.0 Hz, 1H), 3.46 - 3.42 (m, 4H), 2.85 - 2.78 (m, 4H). LCMS (Analytical Method A) Rt = 1.51 min, MS (ESIpos): m/z 391.3 [M+H]+, Purity = 99%.

### 1-(4-Fluorophenyl)-3-(2-methylpyridin-4-yl)-3H-imidazo[4,5-b]pyridin-5-yl]piperazine / Compound 2-3 (#13 of Table 1)

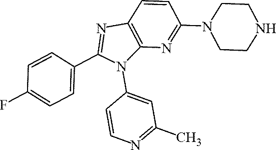 [SMILES: Cc1cc(-n2c(-c3ccc(F)cc3)nc3ccc(N4CCNCC4)nc32)ccn1]

A mixture of Na₂S₂O₄ (127 mg, 0.724 mmol) and tert-butyl 4-[6-[(2-methyl-4-pyridyl)amino]-5-nitro-2-pyridyl]piperazine-1-carboxylate (Intermediate 5) (100 mg, 0.241 mmol) in DMSO (1 mL) and EtOH (0.2 mL) was gently heated for 30 s. 4-Fluorobenzaldehyde (39 µL, 0.362 mmol) was added and the reaction heated to 100° C. for 20 h. The reaction was cooled and quenched into water. The aqueous layer was washed with EtOAc (2×) then basified with NaHCOs (aq). The aqueous layer was extracted into EtOAc (3×), the combined organics washed with brine, dried over MgSO₄ and concentrated in vacuo. The residue was purified by preparative HPLC (Method A1) to afford the title compound (26 mg, 26% yield). 1H NMR (500 MHz, Chloroform-d) δ 8.58 (d, J = 5.4 Hz, 1H), 7.92 (d, J = 8.9 Hz, 1H), 7.51 (dd, J = 8.9, 5.3 Hz, 2H), 7.21 (d, J = 1.8 Hz, 1H), 7.14 (dd, J = 5.4, 1.9 Hz, 1H), 7.05 (t, J = 8.7 Hz, 2H), 6.73 (d, J = 8.9 Hz, 1H), 3.55 - 3.48 (m, 4H), 3.02 - 2.96 (m, 4H), 2.58 (s, 3H). LCMS (Analytical Method B) Rt = 2.61 min, MS (ESIpos): m/z 389.3 [M+H]+, Purity = 95%.

### 1-(4-Fluorophenyl)-3-(2-methoxypyridin-4-yl)-3H-imidazo[4,5-b]pyridin-5-yl]piperazine / Compound 2-4 (#32 of Table 1)

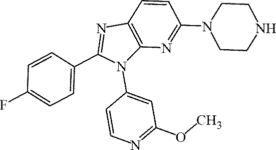 [SMILES: COc1cc(-n2c(-c3ccc(F)cc3)nc3ccc(N4CCNCC4)nc32)ccn1]

A mixture of Na₂S₂O₄ (123 mg, 0.697 mmol) and tert-butyl 4-[6-[(2-methoxy-4-pyridyl)amino]-5-nitro-2-pyridyl]piperazine-1-carboxylate (Intermediate 6) (100 mg, 0.232 mmol) in DMSO (1 mL) and EtOH (0.12 mL) was gently warmed for 30 s. 4-Fluorobenzaldehyde (37 µL, 0.348 mmol) was added and the reaction heated to 100° C. for 20 h. The reaction was cooled and quenched into water. The aqueous layer was washed with EtOAc (2×) then basified with NaHCO₃ (aq.). The aqueous layer was extracted into EtOAc (3×), the combined organics washed with brine, dried over MgSO4 and concentrated in vacuo. The residue was purified by preparative HPLC (Method A1) to afford the title compound (10 mg, 11% yield). 1H NMR (500 MHz, DMSO-d6) δ 8.26 (d, J = 5.5 Hz, 1H), 7.94 (d, J = 8.9 Hz, 1H), 7.53 (dd, J = 8.9, 5.5 Hz, 2H), 7.26 (t, J = 8.9 Hz, 2H), 6.96 (dd, J = 5.5, 1.7 Hz, 1H), 6.91 (d, J = 1.4 Hz, 1H), 6.88 (d, J = 9.0 Hz, 1H), 3.89 (s, 3H), 3.42 - 3.37 (m, 4H), 2.79 - 2.73 (m, 4H). LCMS (Analytical Method A) Rt = 1.83 min, MS (ESlpos): m/z 405.4 [M+H]+, Purity = 100%.

### 1-{3-(Difluoromethyl)pyridin-4-yl]-2-(4-fluorophenyl)-3H-imidazo[4,5-b]pyridin-5-yl}piperazine / Compound 2-5 (#16 of Table 1)

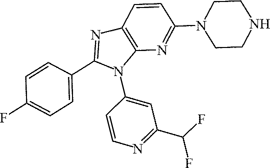 [SMILES: Fc1ccc(-c2nc3ccc(N4CCNCC4)nc3n2-c2ccnc(C(F)F)c2)cc1]

A mixture of tert-butyl 4-[6-[[2-(difluoromethyl)-4-pyridyl]amino]-5-nitro-2-pyridyl]piperazine-1-carboxylate (Intermediate 7) (65 mg, 0.144 mmol) and Na₂S₂O₄ (76 mg, 0.433 mmol) in DMSO (0.6 mL) and EtOH (0.12 mL) was gently heated for 30 s. 4-Fluorobenzaldehyde (23 µL, 0.216 mmol) was added and the reaction heated to 100° C. for 20 h. The reaction was cooled and quenched into water. The aqueous layer was washed with EtOAc (2×) then basified with NaHCO₃ (aq). The aqueous layer was extracted into EtOAc (3×), the combined organics washed with brine, dried over MgSO₄ and concentrated in vacuo. The residue was purified by preparative HPLC (Method A1) to afford the title compound (27 mg, 44% yield). 1H NMR (500 MHz, DMSO-d6) δ 8.78 (d, J = 5.3 Hz, 1H), 7.97 (d, J = 8.9 Hz, 1H), 7.82 (d, J = 1.8 Hz, 1H), 7.56 - 7.50 (m, 3H), 7.28 (t, J = 8.9 Hz, 2H), 7.01 (t, J = 55 Hz, 1H) 6.92 (s, 1H), 3.44 - 3.39 (m, 4H), 2.80 - 2.73 (m, 4H). LCMS (Analytical Method A) Rt = 1.83 min, MS (ESlpos): m/z 425.3 [M+H]+, Purity = 99%.

### 4-(4-fluorophenyl)-5-{octahydropyrrolo[3,4-c]pyrrol-2-yl}-3H-imidazo[4,5-b]pyridin-3-yl]pyridine / Compound 2-6 (#23 in Table 1)

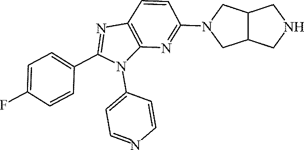 [SMILES: Fc1ccc(-c2nc3ccc(N4CC5CNCC5C4)nc3n2-c2ccncc2)cc1]

A mixture of tert-butyl 2-[5-nitro-6-(4-pyridylamino)-2-pyridyl]-1,3,3a,4,6,6a-hexahydropyrrolo[3,4-c]pyrrole-5-carboxylate (Intermediate 8) (100 mg, 0.234 mmol) and Na₂S₂O₄ (124 mg, 0.703 mmol) in DMSO (1 mL) and EtOH (0.2 mL) was gently heated for 30 s. 4-Fluorobenzaldehyde (38 µL, 0.352 mmol) was added and the reaction heated to 100° C. for 20 h. The reaction was cooled and quenched into water. The aqueous layer was extracted into EtOAc (3×) then into DCM. The combined organic extracts were washed with brine, dried and concentrated in vacuo. The residue was purified by flash chromatography (25 g, silica) eluting with 0-8% MeOH/DCM to afford the title compound (28 mg, 29% yield). 1H NMR (400 MHz, DMSO-d6) δ 8.75 - 8.65 (m, 2H), 7.93 (d, J = 8.8 Hz, 1H), 7.57 - 7.47 (m, 2H), 7.47 - 7.40 (m, 2H), 7.26 (t, J = 8.9 Hz, 2H), 6.58 (d, J = 8.8 Hz, 1H), 3.61 (dd, J = 10.7, 7.9 Hz, 2H), 3.21 (dd, J = 10.8, 3.5 Hz, 2H), 2.92 (dd, J = 10.6, 6.6 Hz, 2H), 2.85 - 2.76 (m, 2H), 2.66 - 2.59 (m, 2H). LCMS (Analytical Method B) Rt = 1.38 min, MS (ESlpos): m/z 401.3 [M+H]+, Purity = 97%.

### 2-fluorophenyl)-1-(2-methylpyridin-4-yl)-6-{octahydropyrrolo[3,4-c]pyrrol-2-yl}-1H-1,3-benzodiazole / Compound 2-7 (#24 in Table 1)

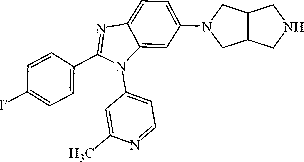 [SMILES: Cc1cc(-n2c(-c3ccc(F)cc3)nc3ccc(N4CC5CNCC5C4)cc32)ccn1]

A mixture of tert-butyl 2-[6-[(2-methyl-4-pyridyl)amino]-5-nitro-2-pyridyl]-1,3,3a,4,6,6a-hexahydropyrrolo[3,4-c]pyrrole-5-carboxylate (Intermediate 9) (100 mg, 0.227 mmol) and Na₂S₂O₄ (120 mg, 0.681 mmol) in DMSO (1 mL) and EtOH (0.2 mL) was gently heated for 30 s. 4-Fluorobenzaldehyde (37 µL, 0.341 mmol) was added and the reaction heated to 100° C. for 22 h. The reaction was cooled and the solution diluted with MeCN and water and heated until fully dissolved. The solution was cooled and the solid material removed by filtration and the filtrate concentrated in vacuo. The residue was purified by preparative HPLC (Method A2) to afford the title compound (20 mg, 21% yield). 1H NMR (400 MHz, DMSO-d6) δ 8.56 (d, J = 5.4 Hz, 1H), 7.92 (d, J = 8.8 Hz, 1H), 7.55 - 7.46 (m, 2H), 7.38 - 7.31 (m, 1H), 7.31 - 7.16 (m, 3H), 6.57 (d, J = 8.8 Hz, 1H), 3.68 - 3.55 (m, 2H), 3.20 (dd, J = 10.7, 3.5 Hz, 2H), 2.96 - 2.88 (m, 2H), 2.85 - 2.76 (m, 2H), 2.65 - 2.60 (m, 2H). LCMS (Analytical Method A) Rt = 1.37 min, MS (ESlpos): m/z 415.4 [M+H]+, Purity = 100%.

### 1-(2-fluorophenyl)-3-(pyridin-4-yl)-3H-imidazo[4,5-b]pyridin-5-yl]piperazine / Compound 2-8 (#20 in Table 1)

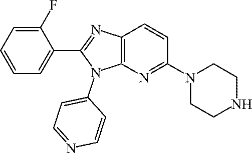 [SMILES: Fc1ccccc1-c1nc2ccc(N3CCNCC3)nc2n1-c1ccncc1]

A mixture of tert-butyl 4-[5-nitro-6-(4-pyridylamino)-2-pyridyl]piperazine-1-carboxylate (Intermediate 4) (100 mg, 0.250 mmol) and Na₂S₂O₄ (132 mg, 0.749 mmol) in DMSO (1 mL) and EtOH (0.2 mL) was gently heated for 30 s. 2-Fluorobenzaldehyde (40 µL, 0.375 mmol) was added and the reaction heated to 100° C. for 20 h. The reaction was cooled and quenched into water. The aqueous layer was extracted into EtOAc (3×), the combined organics washed with brine, dried over MgSO₄ and concentrated in vacuo. The residue was purified by flash chromatography (25 g, silica) eluting with 0-8% MeOH/DCM to afford the title compound (30 mg, 26% yield). 1H NMR (400 MHz, DMSO-d6) δ 8.74 - 8.60 (m, 2H), 7.99 (d, J = 9.0 Hz, 1H), 7.73 (td, J = 7.5, 1.8 Hz, 1H), 7.61 - 7.49 (m, 1H), 7.42 - 7.32 (m, 3H), 7.30 - 7.14 (m, 1H), 6.94 (d, J = 9.0 Hz, 1H), 3.49 - 3.39 (m, 4H), 2.87 - 2.72 (m, 4H). LCMS (Analytical Method A) Rt = 1.20 min, MS (ESlpos): m/z 375.2 [M+H]+, Purity = 96%.

### 4-(4-fluorophenyl)-5-(piperazin-1-yl)-3H-imidazo[4,5-b]pyridin-3-yl]pyridazine / Compound 2-9 (#22 in Table 1)

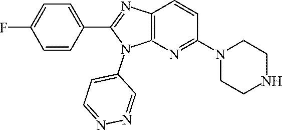 [SMILES: Fc1ccc(-c2nc3ccc(N4CCNCC4)nc3n2-c2ccnnc2)cc1]

Na₂S₂O₄ (335 mg, 2.99 mmol) was added to a solution of tert-butyl 4-[5-nitro-6-(pyridazin-4-ylamino)-2-pyridyl]piperazine-1-carboxylate (Intermediate 10) (300 mg, 0.635 mmol) and 4-fluorobenzaldehyde (85 µL, 0.792 mmol) in DMSO (6 mL) and EtOH (1 mL), and the reaction heated to 100° C. for 16 h. The reaction was cooled and quenched with sat. NaHCO₃. The aqueous layer was extracted into EtOAc (3×), the combined organics washed with brine, dried over MgSO₄ and concentrated in vacuo. The residue was purified by flash chromatography (25 g, silica) eluting with 0-10% 7N NH₃ in MeOH/DCM followed by preparative HPLC (Method A1) to afford the title compound (17 mg, 7% yield). 1H NMR (400 MHz, DMSO-d6) δ 9.38 (dd, J = 5.6 Hz, 1.0 1H), 9.34 (dd, J = 2.6, 1.0 Hz, 1H), 7.98 (d, J = 8.9 Hz, 1H), 7.74 (dd, J = 5.6, 2.7 Hz, 1H), 7.57 - 7.51 (m, 2H), 7.33 - 7.25 (m, 2H), 6.92 (d, J = 9.0 Hz, 1H), 3.45 - 3.40 (m, 4H), 2.82 - 2.75 (m, 4H). LCMS (Analytical Method A) Rt = 1.40 min, MS (ESlpos): m/z 376.3 [M+H]+, Purity = 100%.

### 2-(4-fluorophenyl)-3-(pyridin-4-yl)-3H-imidazo[4,5-b]pyridin-5-yl]-2,5-diazabicyclo[2.2.2]octane / Compound 2-10 (#21 in Table 1)

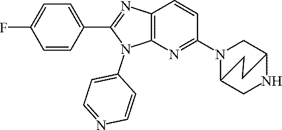 [SMILES: Fc1ccc(-c2nc3ccc(N4CC5CCC4CN5)nc3n2-c2ccncc2)cc1]

A mixture of tert-butyl 5-[5-nitro-6-(4-pyridylamino)-2-pyridyl]-2,5-diazabicyclo[2.2.2]octane-2-carboxylate (Intermediate 12) (100 mg, 0.234 mmol) and Na₂S₂O₄ (124 mg, 0.703 mmol) in DMSO (0.94 mL) and EtOH (0.19 mL) was gently warmed for 30 s. 4-Fluorobenzaldehyde (38 µL, 0.352 mmol) was added and the reaction heated to 100° C. for 20 h. The reaction was cooled and quenched into water. The aqueous layer was extracted into EtOAc (3×), the combined organics washed with brine, dried over MgSO₄ and concentrated in vacuo. The residue was purified by flash chromatography (10 g, silica) eluting with 5-30% MeOH/DCM to afford the title compound (41 mg, 43% yield) as a yellow solid. 1H NMR (400 MHz, DMSO-d6) δ 8.66 - 8.58 (m, 2H), 7.85 (d, J = 8.9 Hz, 1H), 7.45 - 7.39 (m, 2H), 7.38 - 7.30 (m, 2H), 7.21 - 7.14 (m, 2H), 6.52 (d, J = 8.9 Hz, 1H), 4.36 (s, 1H), 3.48 (d, J = 10.4 Hz, 1H), 3.38 (dd, J = 10.3, 1.8 Hz, 1H), 3.00 (d, J = 10.9 Hz, 2H), 2.93 (dd, J = 10.6, 1.8 Hz, 1H), 1.77 (d, J = 13.1 Hz, 4H), 1.60 (d, J = 10.5 Hz, 1H). LCMS (Analytical Method A) Rt = 1.48 min, MS (ESlpos): m/z 401.3 [M+H]+, Purity = 100%.

### 3-(4-fluorophenyl)-3-(pyridin-4-yl)-3H-imidazo[4,5-b]pyridin-5-yl]-3,6-diazabicyclo[3.1.1]heptane / Compound 2-11 (#30 in Table 1)

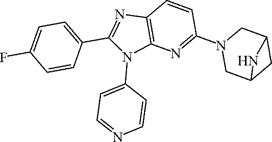 [SMILES: Fc1ccc(-c2nc3ccc(N4CC5CC(C4)N5)nc3n2-c2ccncc2)cc1]

A mixture of tert-butyl 3-[5-nitro-6-(4-pyridylamino)-2-pyridyl]-3,6-diazabicyclo[3.1.1]heptane-6-carboxylate (Intermediate 11) (200 mg, 0.446 mmol) and Na₂S₂O₄ (236 mg, 1.34 mmol) in DMSO (2 mL) and EtOH (0.4 mL) was gently warmed for 30 s. 4-Fluorobenzaldehyde (72 µL, 0.669 mmol) was added and the reaction heated to 100° C. for 18 h. The reaction was cooled and quenched into water. The aqueous layer was extracted into EtOAc (3×), the combined organics washed with brine, dried over MgSO₄ and concentrated in vacuo. The residue was purified by flash chromatography (10 g, silica) eluting with 0-100% MeOH/DCM to afford the title compound (42 mg, 23% yield) as a tan solid. 1H NMR (500 MHz, DMSO-d6) δ 8.73 - 8.67 (m, 2H), 7.99 (d, J = 8.9 Hz, 1H), 7.56 -7.49 (m, 2H), 7.47 - 7.41 (m, 2H), 7.31 - 7.22 (m, 2H), 6.70 (d, J = 8.9 Hz, 1H), 3.82 - 3.44 (m, 7H), 1.68 (s, 1H), 1.45 (d, J = 8.4 Hz, 1H). LCMS (Analytical Method A) Rt = 1.30 min, MS (ESlpos): m/z 387.2 [M+H]+, Purity = 94%.

### N-{4-(4-fluorophenyl)-5-(piperazin-1-yl)-3H-imidazo[4,5-b]pyridin-3-yl]pyridin-2-yl}benzamide / Compound 2-12 (#5 in Table 1)

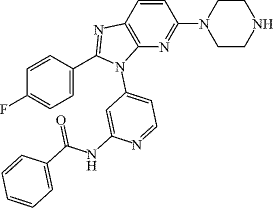 [SMILES: O=C(Nc1cc(-n2c(-c3ccc(F)cc3)nc3ccc(N4CCNCC4)nc32)ccn1)c1ccccc1]

4-Fluorobenzaldehyde (40 mg, 0.323 mmol) was added to a solution of tert-butyl 4-[6-[(2-benzamido-4-pyridyl)amino]-5-nitro-2-pyridyl]piperazine-1-carboxylate (Intermediate 13) (112 mg, 0.216 mmol) in EtOH (0.15 mL) and DMSO (1 mL). The reaction was stirred for 5 min then Na₂S₂O₄ (114 mg, 0.647 mmol) was added and the reaction was heated to 100° C. for 18 h. The reaction was cooled and quenched into water. The aqueous layer was neutralised with NaHCO₃ and extracted into (1:1) EtOAc/THF (3×). The combined organics were washed with brine, dried over Na₂SO₄, and concentrated in vacuo. The residue was purified by preparative HPLC (Method A2) to afford the title compound (14 mg, 13% yield). 1H NMR (500 MHz, DMSO-d6) δ 11.03 (s, 1H), 8.52 - 8.43 (m, 2H), 8.02 - 7.99 (m, 2H), 7.97 (d, J = 8.9 Hz, 1H), 7.59 (m, 3H), 7.52 (t, J = 7.6 Hz, 2H), 7.28 (t, J = 8.9 Hz, 2H), 7.07 (dd, J = 5.5, 1.7 Hz, 1H), 6.90 (d, J = 9.0 Hz, 1H), 3.48 - 3.42 (m, 4H), 2.80 - 2.73 (m, 4H). LCMS (Analytical Method A) Rt = 1.99 min, MS (ESlpos): m/z 494.3 [M+H]+, Purity = 99%.

### N-{5-[(1S,4S)-2,5-diazabicyclo[2.2.1]heptan-2-yl]-2-fluorophenyl)-3H-imidazo[4,5-b]pyridin-3-yl}pyridin-2-yl)benzamide / Compound 2-13 (#8 in Table 1)

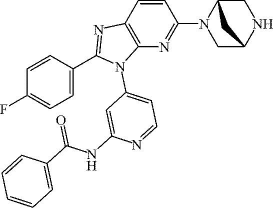 [SMILES: O=C(Nc1cc(-n2c(-c3ccc(F)cc3)nc3ccc(N4C[C@@H]5C[C@H]4CN5)nc32)ccn1)c1ccccc1]

Na₂S₂O₄ (159 mg, 0.903 mmol) was added to a suspension of tert-butyl (1S,4S)-5-[6-[(2-benzamido-4-pyridyl)amino]-5-nitro-2-pyridyl]-2,5-diazabicyclo[2.2.1]heptane-2-carboxylate (Intermediate 19) (160 mg, 0.301 mmol) in EtOH (0.25 mL) and DMSO (1.2 mL). The reaction was gently warmed then 4-fluorobenzaldehyde (48 µL, 0.452 mmol) was added and the reaction heated to 100° C. for 18 h. The reaction was cooled and quenched into NaHCO₃ (aq.). The aqueous layer was extracted into EtOAc (3×), the combined organics dried over MgSO₄ and concentrated in vacuo. The residue was purified by preparative HPLC (Method A1). The residue was further purified by preparative HPLC (Method B1) to afford the title compound (23 mg, 15% yield). 1H NMR (500 MHz, DMSO-d6) δ 11.02 (s, 1H), 8.48 (d, J = 1.6 Hz, 1H), 8.46 (d, J = 5.3 Hz, 1H), 8.04 - 7.98 (m, 2H), 7.92 (d, J = 8.8 Hz, 1H), 7.64 - 7.48 (m, 5H), 7.32 - 7.23 (m, 2H), 7.07 (dd, J = 5.3, 1.9 Hz, 1H), 6.56 (d, J = 8.8 Hz, 1H), 4.69 (s, 1H), 3.62 (s, 1H), 3.46 (dd, J = 9.3, 1.9 Hz, 1H), 3.21 (d, J = 9.2 Hz, 1H), 2.90 - 2.85 (m, 1H), 2.82 (d, J = 9.6 Hz, 1H), 1.74 (d, J = 8.7 Hz, 1H), 1.63 (d, J = 8.9 Hz, 1H). LCMS (Analytical Method A) Rt = 2.08 min, MS (ESlpos): m/z 506.3 [M+H]+, Purity = 97%.

### N-{5-[(1S,4S)-2,5-diazabicyclo[2.2.1]heptan-2-yl]-2-fluorophenyl)-3H-imidazo[4,5-b]pyridin-3-yl}pyridin-2-yl)cyclopropanecarboxamide / Compound 2-14 (#11 In table 1)

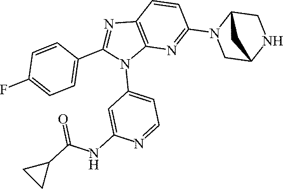 [SMILES: O=C(Nc1cc(-n2c(-c3ccc(F)cc3)nc3ccc(N4C[C@@H]5C[C@H]4CN5)nc32)ccn1)C1CC1]

Na₂S₂O₄ (174 mg, 0.989 mmol) was added to a suspension of tert-butyl (1S,4S)-5-[6-[[2-(cyclopropanecarbonylamino)-4-pyridyl]amino]-5-nitro-2-pyridyl]-2,5-diazabicyclo[2.2.1]heptane-2-carboxylate (Intermediate 21) (165 mg, 0.330 mmol) in EtOH (0.26 mL) and DMSO (1.32 mL). The reaction was gently warmed then 4-fluorobenzaldehyde (53 µL, 0.494 mmol) was added and the reaction heated to 100° C. for 18 h. The reaction was cooled and quenched into NaHCO₃ (aq.). The aqueous layer was extracted into EtOAc (3×), the combined organics dried over MgSO₄ and concentrated in vacuo. The residue was purified by preparative HPLC (Method A2) to afford the title compound (85 mg, 55% yield). 1H NMR (500 MHz, DMSO-d6) δ 11.02 (s, 1H), 8.37 (d, J = 5.4 Hz, 1H), 8.34 (d, J = 1.5 Hz, 1H), 7.89 (d, J = 8.8 Hz, 1H), 7.56 - 7.49 (m, 2H), 7.29 - 7.21 (m, 2H), 6.96 (dd, J = 5.4, 1.9 Hz, 1H), 6.53 (d, J = 8.8 Hz, 1H), 4.64 (s, 1H), 3.62 (s, 1H), 3.43 (dd, J = 9.3, 1.8 Hz, 1H), 3.18 (d, J = 9.3 Hz, 1H), 2.85 (dd, J = 9.5, 1.3 Hz, 1H), 2.78 (d, J = 9.6 Hz, 1H), 2.06 - 1.97 (m, 1H), 1.73 (d, J = 8.9 Hz, 1H), 1.62 (d, J = 9.0 Hz, 1H), 0.85 - 0.73 (m, 4H). LCMS (Analytical Method A) Rt = 1.77 min, MS (ESlpos): m/z 470.4 [M+H]+, Purity = 100%.

### N-{5-[(1S,4S)-2,5-diazabicyclo[2.2.1]heptan-2-yl]-2-fluorophenyl)-3H-imidazo[4,5-b]pyridin-3-yl}pyridin-2-yl)-4-fluorobenzamide / Compound 2-15 (#10 in Table 1)

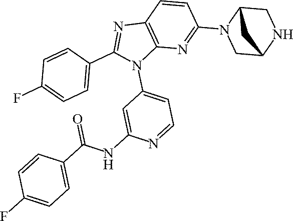 [SMILES: O=C(Nc1cc(-n2c(-c3ccc(F)cc3)nc3ccc(N4C[C@@H]5C[C@H]4CN5)nc32)ccn1)c1ccc(F)cc1]

Na₂S₂O₄ (148 mg, 0.839 mmol) was added to a suspension of tert-butyl (1S,4S)-5-[6-[[2-[(4-fluorobenzoyl)amino]-4-pyridyl]amino]-5-nitro-2-pyridyl]-2,5-diazabicyclo[2.2.1]heptane-2-carboxylate (Intermediate 20) (160 mg, 0.280 mmol) in EtOH (0.2 mL) and DMSO (1.1 mL). The reaction was gently warmed then 4-fluorobenzaldehyde (45 µL, 0.419 mmol) was added and the reaction heated to 100° C. for 18 h. The reaction was cooled and quenched into NaHCO₃ (aq.). The aqueous layer was extracted into EtOAc (3×), the combined organics dried over MgSO₄ and concentrated in vacuo. The residue was purified by preparative HPLC (Method A2) to afford the title compound (76 mg, 52% yield). 1H NMR (500 MHz, DMSO-d6) δ 11.08 (s, 1H), 8.49 - 8.43 (m, 2H), 8.13 - 8.06 (m, 2H), 7.92 (d, J = 8.8 Hz, 1H), 7.60 - 7.52 (m, 2H), 7.39 - 7.32 (m, 2H), 7.31 - 7.23 (m, 2H), 7.07 (dd, J = 5.5, 1.8 Hz, 1H), 6.55 (d, J = 8.8 Hz, 1H), 4.69 (s, 1H), 3.62 (s, 1H), 3.46 (dd, J = 9.3, 1.8 Hz, 1H), 3.21 (d, J = 9.2 Hz, 1H), 2.88 (dd, J = 9.6, 1.4 Hz, 1H), 2.82 (d, J = 9.6 Hz, 1H), 1.74 (d, J = 8.9 Hz, 1H), 1.63 (d, J = 8.9 Hz, 1H). LCMS (Analytical Method A) Rt = 2.12 min, MS (ESlpos): m/z 524.4 [M+H]+, Purity = 100%.

### 4-fluoro-N-{4-[2-(4-fluorophenyl)-5-(piperazin-1-yl)-3H-imidazo[4,5-b]pyridin-3-yl]pyridin-2-yl}benzamide / Compound 2-16 (#6 in Table 1)

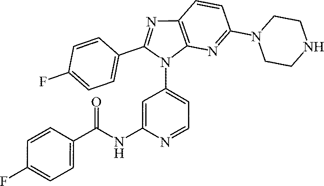 [SMILES: O=C(Nc1cc(-n2c(-c3ccc(F)cc3)nc3ccc(N4CCNCC4)nc32)ccn1)c1ccc(F)cc1]

tert-Butyl 4-[6-[[2-[(4-fluorobenzoyl)amino]-4-pyridyl]amino]-5-nitro-2-pyridyl]piperazine-1-carboxylate (Intermediate 14) (250 mg, 0.372 mmol) and Na₂S₂O₄ (200 mg, 1.14 mmol) were suspended in EtOH (1 mL) and DMSO (4 mL), then 4-fluorobenzaldehyde (61 µL, 0.564 mmol) was added. The mixture was heated to 100° C. for 3 h. The reaction was quenched with NaHCO₃ (aq.) and extracted with EtOAc, dried over MgSO₄ and concentrated in vacuo. The residue was purified by preparative HPLC (Method A2). The residue was further purified by preparative HPLC (Method B1) to afford the title compound (22 mg, 11% yield). 1H NMR (400 MHz, DMSO-d6) δ 11.1 (s, 1H), 8.5 - 8.4 (m, 2H), 8.1 - 8.1 (m, 2H), 8.0 (d, J = 8.9 Hz, 1H), 7.6 - 7.5 (m, 2H), 7.4 - 7.3 (m, 2H), 7.3 - 7.2 (m, 2H), 7.0 (dd, J = 5.3, 1.9 Hz, 1H), 6.9 (d, J = 9.0 Hz, 1H), 3.6 - 3.5 (m, 4H), 2.9 - 2.8 (m, 4H). LCMS (Analytical Method A) Rt = 2.13 min, MS (ESlpos): m/z 512.3 [M+H]+, Purity = 100%.

### N-{4-(4-fluorophenyl)-5-(piperazin-1-yl)-3H-imidazo[4,5-b]pyridin-3-yl]pyridin-2-yl}oxane-3-carboxamide / Compound 2-17 (#1 in Table 1)

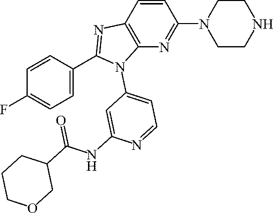 [SMILES: O=C(Nc1cc(-n2c(-c3ccc(F)cc3)nc3ccc(N4CCNCC4)nc32)ccn1)C1CCCOC1]

4-Fluorobenzaldehyde (42 mg, 0.341 mmol) was added to a solution of tert-butyl 4-[5-nitro-6-[[2-(tetrahydropyran-3-carbonylamino)-4-pyridyl]amino]-2-pyridyl]piperazine-1-carboxylate (Intermediate 16) (120 mg, 0.227 mmol) in DMSO (1 mL) and EtOH (0.15 mL). The reaction was stirred for 5 min then Na₂S₂O₄ (120 mg, 0.682 mmol) was added and the reaction was heated to 100° C. for 18 h. The mixture was neutralised with NaHCO₃ (aq.) and then extracted with (1:1) EtOAc/THF. The organics were washed with brine, dried over Na₂SO₄, and concentrated in vacuo. The residue was purified by preparative HPLC (Method A1). The residue was loaded onto an SCX-2 ion exchange cartridge primed with MeOH. The cartridge was washed swith MeOH then the product was eluted with 2 M NH₃ in MeOH and concentrated in vacuo. The residue was further purified by preparative HPLC (Method B1) to afford the title compound (9 mg, 8% yield). 1H NMR (400 MHz, DMSO-d6) δ 10.75 (s, 1H), 8.41 (d, J = 1.6 Hz, 1H), 8.35 (d, J = 5.4 Hz, 1H), 8.26 (s, 1H), 7.96 (d, J = 8.9 Hz, 1H), 7.56 (dd, J = 8.8, 5.4 Hz, 2H), 7.27 (t, J = 8.9 Hz, 2H), 6.96 - 6.82 (m, 2H), 3.95 - 3.89 (m, 1H), 3.79 (d, J = 11.0 Hz, 1H), 3.51 - 3.45 (m, 6H), 2.89 - 2.81 (m, 4H), 2.81 - 2.74 (m, 1H), 1.92 (d, J = 9.6 Hz, 1H), 1.72 -1.58 (m, 2H), 1.58 - 1.45 (m, 1H). LCMS (Analytical Method A) Rt = 1.81 min, MS (ESlpos): m/z 502.4 [M+H]+, Purity = 97%.

### N-{4-(4-fluorophenyl)-5-(piperazin-1-yl)-3H-imidazo[4,5-b]pyridin-3-yl]pyridin-2-yl}acetamide / Compound 2-18 (#4 in Table 1)

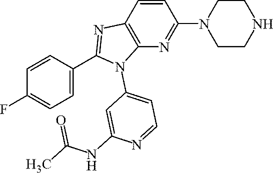 [SMILES: CC(=O)Nc1cc(-n2c(-c3ccc(F)cc3)nc3ccc(N4CCNCC4)nc32)ccn1]

tert-Butyl 4-[6-[(2-acetamido-4-pyridyl)amino]-5-nitro-2-pyridyl]piperazine-1-carboxylate (Intermediate 18) (200 mg, 0.437 mmol) and 4-fluorobenzaldehyde (47 µL, 0.437 mmol) were dissolved in DMSO (5 mL), then Na₂S₂O₄ (232 mg, 1.32 mmol) was added. The mixture was heated to 100° C. for 16 h. The reaction was cooled to RT and quenched into NaHCO₃ (aq.) and extracted into DCM. The organics were concentrated in vacuo and purified by preparative HPLC (Method A2) to afford the title compound (55 mg, 28% yield). 1H NMR (500 MHz, DMSO-d6) δ 10.71 (s, 1H), 8.38 (d, J = 5.4 Hz, 1H), 8.24 (s, 1H), 7.94 (d, J = 8.9 Hz, 1H), 7.56 - 7.50 (m, 2H), 7.29 - 7.22 (m, 2H), 7.02 (dd, J = 5.4, 1.9 Hz, 1H), 6.88 (d, J = 9.0 Hz, 1H), 3.43 - 3.39 (m, 4H), 2.79 -2.73 (m, 4H), 2.08 (s, 3H). LCMS (Analytical Method B) Rt = 2.31 min, MS (ESlpos): m/z 432.4 [M+H]+, Purity = 96%.

### N-{4-(4-fluorophenyl)-5-(piperazin-1-yl)-3H-imidazo[4,5-b]pyridin-3-yl]pyridin-2-yl}morpholine-4-carboxamide / Compound 2-19 (#9 in Table 1)

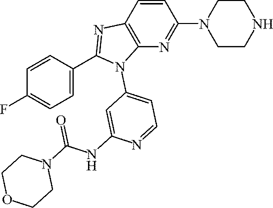 [SMILES: O=C(Nc1cc(-n2c(-c3ccc(F)cc3)nc3ccc(N4CCNCC4)nc32)ccn1)N1CCOCC1]

4-Fluorobenzaldehyde (33 mg, 0.270 mmol) was added to a solution of tert-butyl 4-[6-[[2-(morpholine-4-carbonylamino)-4-pyridyl]amino]-5-nitro-2-pyridyl]piperazine-1-carboxylate (Intermediate 23) (57 mg, 0.108 mmol) in EtOH (0.15 mL) and DMSO (1 mL). The reaction was stirred for 5 min then Na₂S₂O₄ (95 mg, 0.539 mmol) was added and the reaction was heated to 100° C. for 18 h. Morpholine (0.20 mL, 1.65 mmol) was then added and the mixture heated to 120° C. for 18 h. The reaction was cooled and partitioned between DCM and NaHCO₃ (aq.). The aqueous layer was extracted into DCM and the combined organics passed through a hydrophobic frit and concentrated in vacuo. The residue was purified by preparative HPLC (Method B1) to afford the title compound (5 mg, 9% yield). 1H NMR (500 MHz, DMSO-d6) δ 9.50 (s, 1H), 8.31 (d, J = 5.4 Hz, 1H), 8.03 (d, J = 1.7 Hz, 1H), 7.96 (d, J = 8.9 Hz, 1H), 7.58 - 7.52 (m, 2H), 7.27 (t, J = 8.9 Hz, 2H), 6.90 (d, J = 9.0 Hz, 1H), 6.87 (dd, J = 5.4, 1.9 Hz, 1H), 3.75 - 3.49 (m, 12H), 2.89 - 2.82 (m, 4H). LCMS (Analytical Method A) Rt = 1.54 min, MS (ESlpos): m/z 503.3 [M+H]+, Purity = 91%.

### 1-(4-fluorophenyl)-3-(methoxymethyl)pyridin-4-yl]-3H-imidazo[4,5-b]pyridin-5-yl]piperazine / Compound 2-20 (#12 in Table 1)

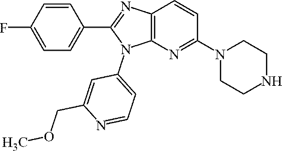 [SMILES: COCc1cc(-n2c(-c3ccc(F)cc3)nc3ccc(N4CCNCC4)nc32)ccn1]

A mixture of tert-butyl 4-[6-[[2-(methoxymethyl)-4-pyridyl]amino]-5-nitro-2-pyridyl]piperazine-1-carboxylate (Intermediate 25) (140 mg, 0.271 mmol) and Na₂S₂O₄ (143 mg, 0.813 mmol) in DMSO (1 mL) and EtOH (0.2 mL) was gently warmed for 30 s. 4-Fluorobenzaldehyde (44 µL, 0.406 mmol) was added and the reaction heated to 100° C. for 20 h. The reaction was cooled and quenched into water. The aqueous layer was extracted into EtOAc (3×), the combined organics washed with brine, dried over MgSO₄ and concentrated in vacuo. The residue was purified by flash chromatography (25 g, silica) eluting with 5-30% MeOH/DCM followed by preparative HPLC (high pH, custom method) to afford the title compound (6 mg, 5% yield). 1H NMR (500 MHz, Methanol-d4) δ 8.48 (d, J = 5.4 Hz, 1H), 7.81 (d, J = 9.0 Hz, 1H), 7.51 - 7.48 (m, 1H), 7.48 - 7.42 (m, 2H), 7.24 (dd, J = 5.4, 2.1 Hz, 1H), 7.11 - 7.04 (m, 2H), 6.82 (d, J = 9.0 Hz, 1H), 4.47 (s, 2H), 3.50 - 3.42 (m, 4H), 3.27 (s, 3H), 2.85 - 2.77 (m, 4H). LCMS (Analytical Method A) Rt = 1.58 min, MS (ESlpos): m/z 419.3 [M+H]+, Purity = 99%.

### 4-(4-fluorophenyl)-5-(piperazin-1-yl)-3H-imidazo[4,5-b]pyridin-3-yl]pyrimidine / Compound 2-21 (#27 in Table 1)

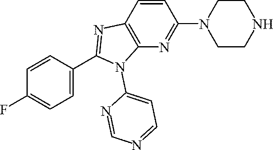 [SMILES: Fc1ccc(-c2nc3ccc(N4CCNCC4)nc3n2-c2ccncn2)cc1]

4-Fluorobenzaldehyde (40 mg, 0.324 mmol) was added to a solution of tert-butyl 4-[5-nitro-6-(pyrimidin-4-ylamino)-2-pyridyl]piperazine-1-carboxylate (Intermediate 26) (91 mg, 0.216 mmol) in EtOH (0.15 mL) and DMSO (1 mL). The reaction was stirred for 5 min then Na₂S₂O₄ (114 mg, 0.648 mmol) was added and the reaction was heated to 100° C. for 18 h. The reaction was quenched into water and the aqueous was then neutralised with NaHCO₃ (aq.). The aqueous layer was extracted with (1:1) EtOAc/THF (3×). The combined organics were washed with brine, dried over Na₂SO₄ and concentrated in vacuo. The residue was purified by preparative HPLC (Method A2) to afford the title compound (3 mg, 4% yield). 1H NMR (500 MHz, DMSO-d6) δ 9.13 (d, J = 5.4 Hz, 1H), 9.03 (d, J = 0.9 Hz, 1H), 8.15 (dd, J = 5.4, 1.2 Hz, 1H), 7.98 (d, J = 8.9 Hz, 1H), 7.55 - 7.47 (m, 2H), 7.26 - 7.19 (m, 2H), 6.93 (d, J = 9.0 Hz, 1H), 3.46 - 3.43 (m, 4H), 2.82 - 2.75 (m, 4H). LCMS (Analytical Method A) Rt = 1.43 min, MS (ESlpos): m/z 376.2 [M+H]+, Purity = 97%.

### N-{4-(4-fluorophenyl)-5-(piperazin-1-yl)-3H-imidazo[4,5-b]pyridin-3-yl]pyridin-2-yl}cyclopropanecarboxamide / Compound 3-1 (#3 in Table 1)

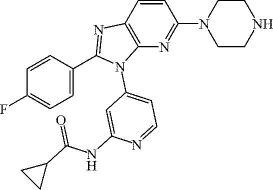 [SMILES: O=C(Nc1cc(-n2c(-c3ccc(F)cc3)nc3ccc(N4CCNCC4)nc32)ccn1)C1CC1]

tert-Butyl 4-[6-[[2-(cyclopropanecarbonylamino)-4-pyridyl]amino]-5-nitro-2-pyridyl]piperazine-1-carboxylate (Intermediate 17) (195 mg, 0.363 mmol) and Na₂S₂O₄ (195 mg, 1.11 mmol) were suspended in EtOH (1 mL) and DMSO (3 mL), then 4-fluorobenzaldehyde (50 µL, 0.466 mmol) was added. The mixture was heated to 100° C. for 12 h then cooled to RT. 4 M HCl in 1,4-dioxane (1 mL) was added and the reaction was stirred for 2 h. The mixture was quenched with 2 M NaOH and extracted into DCM. The organics were combined and concentrated in vacuo and the residue was purified via flash chromatography (25 g, silica) eluting with 0-10% MeOH/DCM. The residue was further purified by preparative HPLC (Method A1) to afford the title compound (80 mg, 46% yield). 1H NMR (400 MHz, DMSO-d6) δ 11.02 (s, 1H), 8.38 - 8.34 (m, 2H), 7.93 (d, J = 8.9 Hz, 1H), 7.57 - 7.50 (m, 2H), 7.29 - 7.21 (m, 2H), 6.94 (dd, J = 5.4, 1.9 Hz, 1H), 6.87 (d, J = 9.0 Hz, 1H), 3.44 - 3.37 (m, 4H), 2.78 - 2.71 (m, 4H), 2.04 - 1.98 (m, 1H), 0.84 - 0.72 (m, 4H). LCMS (Analytical Method B) Rt = 2.67 min, MS (ESlpos): m/z 458.3 [M+H]+, Purity = 95%.

### 4-(4-fluorophenyl)-5-(piperazin-1-yl)-3H-imidazo[4,5-b]pyridin-3-yl]pyridin-2-amine / Compound 3-2 (#15 in Table 1)

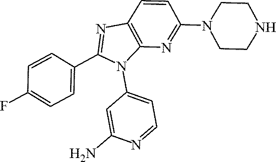 [SMILES: Nc1cc(-n2c(-c3ccc(F)cc3)nc3ccc(N4CCNCC4)nc32)ccn1]

tert-Butyl 4-[6-[(2-acetamido-4-pyridyl)amino]-5-nitro-2-pyridyl]piperazine-1-carboxylate (Intermediate 18) (300 mg, 0.479 mmol) and Na₂S₂O₄ (257 mg, 1.46 mmol) were suspended in EtOH (1 mL) and DMSO (5 mL), then 4-fluorobenzaldehyde a(78 µL, 0.726 mmol) was added. The mixture was heated to 100° C. for 16 h. The reaction was cooled to RT and 4 M HCl in 1,4-dioxane (2 mL) was added and the reaction was stirred for 2 h. The reaction was concentrated in vacuo. The residue was partitioned between 2 M NaOH and DCM. The organics were concentrated in vacuo and the residue was purified by preparative HPLC (Method A2) to afford the title compound (50 mg, 24% yield). 1H NMR (400 MHz, DMSO-d6) δ 8.00 - 7.98 (m, 1H), 7.91 (d, J = 8.9 Hz, 1H), 7.60 - 7.53 (m, 2H), 7.30 - 7.23 (m, 2H), 6.85 (d, J = 9.0 Hz, 1H), 6.46 - 6.43 (m, 1H), 6.43 - 6.40 (m, 1H), 6.22 (s, 2H), 3.41 - 3.38 (m, 4H), 2.80 - 2.74 (m, 4H). LCMS (Analytical Method B) Rt = 2.27 min, MS (ESlpos): m/z 390.3 [M+H]+, Purity = 90%.

### ({4-(4-fluorophenyl)-5-(piperazin-1-yl)-3H-imidazo[4,5-b]pyridin-3-yl]pyridin-2-yl}methyl)(methyl)amine / Compound 3-3 (#25 in Table 1)

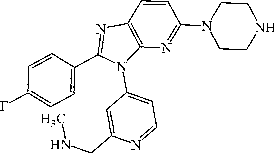 [SMILES: CNCc1cc(-n2c(-c3ccc(F)cc3)nc3ccc(N4CCNCC4)nc32)ccn1]

A mixture of tert-butyl 4-[6-[[2-[[tert-butoxycarbonyl(methyl)amino]methyl]-4-pyridyl]amino]-5-nitro-2-pyridyl]piperazine-1-carboxylate (Intermediate 22) (150 mg, 0.276 mmol) and Na₂S₂O₄ (146 mg, 0.828 mmol) in DMSO (1.1 mL ) and EtOH (0.2 mL) was gently warmed for 30 s. 4-Fluorobenzaldehyde (45 µL, 0.414 mmol) was added and the reaction heated to 100° C. for 18 h. The reaction was cooled and quenched into water. The aqueous layer was extracted into EtOAc (3×), the combined organics washed with brine, dried over MgSO₄ and concentrated in vacuo. The residue dissolved in DCM (3 mL), treated with 4 M HCl (1.4 mL, 5.50 mmol), and the resulting mixture stirred at RT overnight. The solvent was evaporated under reduced pressure and the residue was purified by preparative HPLC (Method A1) to yield the title compound as a tan solid (21 mg, 18% yield). 1H NMR (400 MHz, DMSO-d6) δ 8.61 (d, J = 5.3 Hz, 1H), 7.95 (d, J = 8.9 Hz, 1H), 7.55 - 7.46 (m, 3H), 7.30 - 7.21 (m, 3H), 6.89 (d, J = 9.0 Hz, 1H), 3.78 (s, 2H), 3.45 - 3.37 (m, 4H), 2.81 -2.72 (m, 4H), 2.18 (s, 3H). LCMS (Analytical Method B) Rt = 2.32 min, MS (ESlpos): m/z 418.3 [M+H]+, Purity = 97%.

### 4-fluoro-N-{4-[2-(4-fluorophenyl)-5-(4-methylpiperazin-1-yl)-3H-imidazo[4,5-b]pyridin-3-yl]pyridin-2-yl}benzamide / Compound 4-1 (#7 in Table 1)

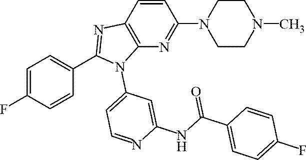 [SMILES: CN1CCN(c2ccc3nc(-c4ccc(F)cc4)n(-c4ccnc(NC(=O)c5ccc(F)cc5)c4)c3n2)CC1]

4-Fluoro-N-[4-[2-(4-fluorophenyl)-5-piperazin-1-yl-imidazo[4,5-b]pyridin-3-yl]-2-pyridyl]benzamide (30 mg, 0.0584 mmol) and 13 M formaldehyde (5.4 µL, 0.0701 mmol) were dissolved in DCM (0.6231 mL) and stirred for 10 min, then NaBH(OAc)₃ (22 mg, 0.105 mmol) was added. The reaction was stirred for 1 h. The reaction was quenched with NaHCO₃ (aq) and extracted with DCM. The organics were passed through a hydrophobic frit and concentrated in vacuo. The reside was purified by flash chromatography (10 g, silica) eluting with 0-7% MeOH/DCM to afford the title compound (6 mg, 19% yield). 1H NMR (400 MHz, Chloroform-d) δ 8.81 (s, 1H), 8.66 (d, J = 1.6 Hz, 1H), 8.31 (d, J = 5.4 Hz, 1H), 7.97 - 7.91 (m, 3H), 7.60 - 7.53 (m, 2H), 7.22 - 7.16 (m, 2H), 7.11 - 7.04 (m, 2H), 6.99 (dd, J = 5.5, 1.9 Hz, 1H), 6.74 (d, J = 8.9 Hz, 1H), 3.69 - 3.61 (m, 4H), 2.60 - 2.54 (m, 4H), 2.36 (s, 3H). LCMS (Analytical Method B) Rt = 3.47 min, MS (ESlpos): m/z 526.3 [M+H]+, Purity = 98%.

### 1-(4-fluorophenyl)-3-(pyridin-4-yl)-3H-imidazo[4,5-b]pyridin-5-yl]-4-methylpiperazine / Compound 4-2 (#14 in Table 1)

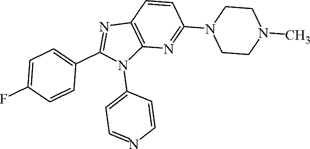 [SMILES: CN1CCN(c2ccc3nc(-c4ccc(F)cc4)n(-c4ccncc4)c3n2)CC1]

Formaldehyde (37%, 64 mg, 0.785 mmol) was added to a solution of 2-(4-fluorophenyl)-5-piperazin-1-yl-3-(4-pyridyl)imidazo[4,5-b]pyridine (Compound 17 of Table 1) (30 mg, 0.0785 mmol) in DCM (1 mL), MeOH (0.2 mL) and acetic acid (0.05 mL) and the mixture was stirred for 3 hours. NaBH(OAc)₃ (166 mg, 0.785 mmol) was then added and the reaction stirred for 20 hours. The reaction was quenched into water. The aqueous layer was extracted into EtOAc (3×), the combined organics washed with brine, dried over MgSO₄ and concentrated in vacuo. The residue was purified by preparative HPLC (Method A1) to yield the title compound as a white solid (12 mg, 37% yield). 1H NMR (500 MHz, Chloroform-d) δ 8.72 - 8.69 (m, 2H), 7.92 (d, J = 8.9 Hz, 1H), 7.50 (dd, J = 8.9, 5.3 Hz, 2H), 7.36 - 7.33 (m, 2H), 7.06 (t, J = 8.7 Hz, 2H), 6.74 (d, J = 8.9 Hz, 1H), 3.62 - 3.54 (m, 4H), 2.56 - 2.50 (m, 4H), 2.35 (s, 3H). LCMS (Analytical Method B) Rt = 2.78 min, MS (ESlpos): m/z 389.3 [M+H]+, Purity = 96%.

### Example 1.4 - Synthesis of Further Intermediates

### Synthesis of 4-bromo-2-(bromomethyl)pyridine / Intermediate 22-1

To a stirred solution of (4-bromopyridin-2-yl)methanol (1.00 g, 5.32 mmol) and carbon tetrabromide (2.82 g, 8.51 mmol) in DCM (20 mL) at 0° C., triphenylphosphine (1.67 g, 6.38 mmol) was added portion-wise, and the mixture was allowed to stir at 0° C. for 1 h, then at RT overnight. The solvent was evaporated under reduced pressure and the residue was purified by flash chromatography (100 g, silica) eluting with 0-100% EtOAc/heptane to yield the title compound as a dark purple liquid (829 mg, 50% yield). 1H NMR (400 MHz, Chloroform-d) δ 8.40 (d, J = 5.3 Hz, 1H), 7.63 (d, J = 1.7 Hz, 1H), 7.40 (dd, J = 5.3, 1.8 Hz, 1H), 4.50 (s, 2H). LCMS (Analytical Method F) Rt = 0.81 min, MS (ESlpos): m/z 249.9 [M+H]+, Purity = 58%.

### Synthesis of Tert-butyl N-[(4-bromopyridin-2-yl)methyl]-N-methylcarbamate / Intermediate 22-2

NaH (69 mg, 2.88 mmol) was added portionwise to an ice-cold solution of tert-butyl methylcarbamate (377 mg, 2.88 mmol) in THF (13 mL), and the mixture was allowed to stir at RT for 1 h. Then, the mixture was cooled down to 0° C. and a solution of 4-bromo-2-(bromomethyl)pyridine (Intermediate 22-1) (820 mg, 2.61 mmol) in THF (13 mL) was added dropwise and the reaction stirred at RT overnight. The mixture was carefully quenched with water, extracted with EtOAc (2×), dried over MgSO₄, filtered and evaporated under reduced pressure. The residue was purified by flash chromatography (25 g, silica) eluting with 0-40% EtOAc/heptane to yield the title compound as a yellow oil (439 mg, 52% yield) 1H NMR (500 MHz, Chloroform-d) δ 8.35 (d, J = 5.3 Hz, 1H), 7.43 -7.32 (m, 2H), 4.59 - 4.45 (m, 2H), 3.03 - 2.84 (m, 3H), 1.55 - 1.36 (m, 9H). LCMS (Analytical Method F) Rt = 0.94 min, MS (ESlpos): m/z 301 [M+H]+, Purity = 94%.

### Synthesis of Tert-butyl 4-(6-amino-5-nitro-2-pyridyl)-1,4-diazepane-1-carboxylate / Intermediate 27-1

A suspension of tert-butyl 1,4-diazepane-1-carboxylate (98% purity, 618 mg, 3.02 mmol), 6-chloro-3-nitropyridin-2-amine (500 mg, 2.88 mmol) and DIPEA (1.5 mL, 8.64 mmol) in MeCN (5 mL) was heated to 100° C. for 2 h. The reaction was cooled and concentrated in vacuo. The residue was taken up in DCM (5 mL) and washed with water (2 × 5 mL). The combined organics were passed through a phase separator and concentrated in vacuo to yield the title compound (1.05 g, 99% yield) as a yellow solid. 1H NMR (400 MHz, Chloroform-d) δ 8.11 (d, J = 9.4 Hz, 1H), 5.95 (d, J = 9.4 Hz, 1H), 3.90 - 3.49 (m, 4H), 3.52 - 3.45 (m, 2H), 3.30 (t, J = 5.8 Hz, 1H), 3.22 (t, J = 6.1 Hz, 1H), 1.91 - 1.82 (m, 2H), 1.40 - 1.29 (m, 9H). LCMS (Analytical Method I) Rt = 0.86 min, MS (ESlpos): m/z 338.2 [M+H]+, Purity = 100%.

Each of intermediates 28-1 through 70-1 as listed in Table 1.4.1 were prepared according to the method of intermediate 27-1 using the intermediates listed in the “Synthesis” column. The intermediates were purified by flash chromatography, SCX or preparative HPLC Methods, A1, A2, B1, B2 as required.

TABLE 1.4.1

| | | | |
|---|---|---|---|
| Intermediate | Synthesis | Structure/Name | Data |
| Intermediate 28-1 | tert-butyl 2-aza-6-azoniaspiro[3.3]heptane-2-carboxylate;2-hydroxy-2-oxo-acetate & 6-chloro-3-nitro-pyridin-2-amine (94% Yield) | tert-butyl 6-(6-amino-5-nitropyridin-2-yl)-2,6-diazaspiro[3.3]heptane-2-carboxylate | 1H NMR (500 MHz, DMSO-d6) δ 8.05 (d, J = 9.3 Hz, 1H), 7.84 (s, 2H), 5.81 (d, J = 9.3 Hz, 1H), 4.23 (s, 4H), 4.04 (s, 4H), 1.38 (s, 9H). LCMS (Analytical Method I) Rt = 0.86 min, MS (ESlpos): m/z 336.2 [M+H]+, Purity = 100%. |
| Intermediate 29-1 | tert-butyl-(1S,5R)-3,6-diazabicyclo[3.1.1]heptane-3-carboxylate & 6-chloro-3-nitro-pyridin-2-amine (75% Yield) | tert-butyl (1R,5S)-6-(6-amino-5-nitropyridin-2-yl)-3,6-diazabicyclo[3.1.1]heptane-3-carboxylate | 1H NMR (400 MHz, DMSO-d6) δ 8.09 (d, J = 9.2 Hz, 1H), 7.89 (s, 2H), 6.05 (d, J = 9.2 Hz, 1H), 4.63 - 4.32 (m, 2H), 4.09 - 3.79 (m, 1H), 3.75 - 3.58 (m, 1H), 3.48 - 3.35 (m, 2H), 2.77 -2.59 (m, 1H), 1.59 (d, J = 8.8 Hz, 1H), 1.35 (s, 9H). LCMS (Analytical Method I) Rt = 1.01 min, MS (ESIpos): m/z 336.2 [M+H]+, Purity = 100%. |
| Intermediate 30-1 | tert-butyl 4,7-diazaspiro[2.5]octane-4-carboxylate & 6-chloro-3-nitro-pyridin-2-amine (99% Yield) | tert-butyl 7-(6-amino-5-nitropyridin-2-yl)-4,7-diazaspiro[2.5]octane-4-carboxylate | 1H NMR (400 MHz, DMSO-d6) δ 8.06 (d, J = 9.5 Hz, 1H), 7.58 (s, 2H), 6.28 (d, J = 9.5 Hz, 1H), 3.79 - 3.71 (m, 2H), 3.58 (s, 2H), 3.55 - 3.49 (m, 2H), 1.44 (s, 9H), 0.99 - 0.93 (m, 2H), 0.85 - 0.80 (m, 2H). LCMS (Analytical Method I) Rt = 0.95 min, MS (ESIpos): m/z 350 [M+H]+, Purity = 100%. |
| Intermediate 31-1 | tert-butyl 1,6-diazaspiro[3.3]heptane-1-carboxylate & 6-chloro-3-nitro-pyridin-2-amine (78% Yield) | tert-butyl 6-(6-amino-5-nitropyridin-2-yl)-1,6-diazaspiro[3.3]heptane-1-carboxylate | 1H NMR (400 MHz, DMSO-d6) δ 8.06 (d, J = 9.2 Hz, 1H), 7.67 (s, 2H), 5.85 (d, J = 9.2 Hz, 1H), 4.49 (d, J = 10.2 Hz, 2H), 4.26 - 4.14 (m, 2H), 3.73 (t, J = 7.2 Hz, 2H), 2.49 - 2.44 (m, 2H), 1.33 (s, 9H). LCMS (Analytical Method I) Rt = 0.78 min, MS (ESlpos): m/z 336 [M+H]+, Purity = 100%. |
| Intermediate 32-1 | 6-chloro-3-nitropyridin-2-amine & tert-butyl(1R,5S)-3,8-diazabicyclo[3.2.1]octane-8-carboxylate (100% Yield) | tert-butyl (1R,5S)-3-(6-amino-5-nitropyridin-2-yl)-3,8-diazabicyclo[3.2.1]octane-8-carboxylate | 1H NMR (400 MHz, DMSO-d6) δ 8.08 (d, J = 9.5 Hz, 1H), 7.99 - 7.47 (m, 2H), 6.28 (d, J = 9.6 Hz, 1H), 4.29 -4.06 (m, 2H), 3.34 - 3.31 (m, 2H), 3.21 - 2.95 (m, 2H), 1.95 - 1.74 (m, 2H), 1.66 - 1.50 (m, 2H), 1.43 (s, 9H). LCMS (Analytical Method I) Rt = 1.00 min, MS (ESIpos): m/z 350.2 [M+H]+, Purity = 100%. |
| Intermediate 33-1 | oxalic acid tert-butyl1,6-diazaspiro[3.3]heptane-6-carboxylate & 6-chloro-3-nitro-pyridin-2-amine (93% Yield) | tert-butyl 1-(6-amino-5-nitropyridin-2-yl)-1,6-diazaspiro[3.3]heptane-6-carboxylate | 1H NMR (400 MHz, DMSO-d6) δ 8.12 (d, J = 9.2 Hz, 1H), 7.42 (s, 2H), 5.87 (d, J = 9.2 Hz, 1H), 4.54 (d, J = 9.1 Hz, 2H), 4.00 (d, J = 9.6 Hz, 2H), 3.97 -3.88 (m, 2H), 2.61 - 2.54 (m, 2H), 1.44 (s, 9H). LCMS (Analytical Method I) Rt = 0.88 min, MS (ESIpos): m/z 336 [M+H]+, Purity = 100%. |
| Intermediate 34-1 | tert-butyl (3R)-3-methylpiperazine-1-carboxylate & 6-chloro-4-methoxy-3-nitro-pyridin-2-amine (97% Yield) | tert-butyl (3R)-4-(6-amino-5-nitropyridin-2-yl)-3-methylpiperazine-1-carboxylate | 1H NMR (500 MHz, CDCl3) δ 8.20 (d, J = 9.5 Hz, 1H), 6.05 (d, J = 9.5 Hz, 1H), 4.66 - 4.49 (m, 1H), 4.27 - 3.80 (m, 3H), 3.30 - 3.20 (m, 1H), 3.20 -3.10 (m, 1H), 3.11 - 2.84 (m, 1H), 1.49 (s, 9H), 1.22 (d, J = 6.7 Hz, 3H). LCMS (Analytical Method I) Rt = 0.92 min, MS (ESIpos): m/z 338.3 [M+H]+, Purity = 98%. |
| Intermediate 35-1 | tert-butyl-(1R,5S)-3,8-diazabicyclo[3.2.1]octane-3-carboxylate & 6-chloro-3-nitro-pyridin-2-amine (91% Yield) | tert-butyl (1R,5S)-8-(6-amino-5-nitropyridin-2-yl)-3,8-diazabicyclo[3.2.1]octane-3-carboxylate | 1H NMR (400 MHz, DMSO-d6) δ 8.08 (d, J = 9.4 Hz, 1H), 8.01 - 7.50 (m, 2H), 6.28 (d, J = 9.5 Hz, 1H), 4.68 (s, 2H), 3.87 - 3.59 (m, 2H), 3.14 - 2.79 (m, 2H), 2.04 - 1.80 (m, 2H), 1.80 -1.62 (m, 2H), 1.41 (s, 9H). LCMS (Analytical Method I) Rt = 0.97 min, MS (ESIpos): m/z 350.2 [M+H]+, Purity = 98%. |
| Intermediate 36-1 | tert-butyl 2,7-diazaspiro[3.5]nonane-7-carboxylate & 6-chloro-3-nitropyridin-2-amine (89% Yield) | tert-butyl 2-(6-amino-5-nitropyridin-2-yl)-2,7-diazaspiro[3.5]nonane-7-carboxylate | 1H NMR (400 MHz, DMSO-d6) δ 8.04 (d, J = 9.3 Hz, 1H), 5.82 (d, J = 9.3 Hz, 1H), 3.82 (s, 4H), 3.31 - 3.26 (m, 4H), 1.72 - 1.63 (m, 4H), 1.39 (s, 9H). LCMS (Analytical Method I) Rt = 0.88 min, MS (ESIpos): m/z 364.3 [M+H]+, Purity = 100%. |
| Intermediate 37-1 | tert-butyl (3R)-3-aminopyrrolidine-1-carboxylate & 6-chloro-3-nitro-pyridin-2-amine (84% Yield) | tert-butyl (3R)-3-[(6-amino-5-nitropyridin-2-yl)amino]pyrrolidine-1-carboxylate | 1H NMR (400 MHz, DMSO-d6) δ 8.10 (s, 2H), 7.96 (d, J = 9.2 Hz, 1H), 7.80 (s, 1H), 5.97 (d, J = 9.2 Hz, 1H), 4.58 - 4.27 (m, 1H), 3.64 - 3.57 (m, 1H), 3.45 - 3.36 (m, 2H), 3.16 - 3.04 (m, 1H), 2.18 - 2.08 (m, 1H), 1.84 (d, J = 5.3 Hz, 1H), 1.40 (s, 9H). LCMS (Analytical Method I) Rt = 0.88 min, MS (ESlpos): m/z 324.2 [M+H]+, Purity = 100%. |
| Intermediate 38-1 | tert-butyl (1R,4R)-2,5-diazabicyclo[2.2.1]heptane-2-carboxylate & 6-chloro-3-nitro-pyridin-2-amine (89% Yield) | tert-butyl (1 R,4R)-5-(6-amino-5-nitropyridin-2-yl)-2,5-diazabicyclo[2.2.1]heptane-2-carboxylate | 1H NMR (500 MHz, DMSO-d6) δ 8.12 - 7.91 (m, 2H), 7.72 (s, 1H), 6.39 -5.85 (m, 1H), 5.11 - 4.75 (m, 1H), 4.60 - 4.36 (m, 1H), 3.67 - 3.39 (m, 2H), 3.23 - 3.09 (m, 1H), 2.05 - 1.77 (m, 2H), 1.39 (m, 9H). LCMS (Analytical Method I) Rt = 0.91 min, MS (ESIpos): m/z 336.2 [M+H]+, Purity = 99%. |
| Intermediate 39-1 | tert-butyl (1S,4S)-2,5-diazabicyclo[2.2.2]octane-2-carboxylate & 6-chloro-3-nitro-pyridin-2-amine (99% Yield) | tert-butyl (1S,4S)-5-(6-amino-5-nitropyridin-2-yl)-2,5-diazabicyclo[2.2.2]octane-2-carboxylate | 1H NMR (500 MHz, Chloroform-d) δ 8.22 (d, J = 9.3 Hz, 1H), 5.90 - 5.77 (m, 1H), 5.31 - 5.13 (m, 1H), 4.59 -4.25 (m, 1H), 3.90 - 3.40 (m, 4H), 2.17 - 1.94 (m, 2H), 1.94 - 1.73 (m, 2H), 1.52 - 1.41 (m, 9H). LCMS (Analytical Method I) Rt = 0.91 min, MS (ESIpos): m/z 350 [M+H]+, Purity = 100%. |
| Intermediate 40-1 | tert-butyl (1R,4R)-2,5-diazabicyclo[2.2.2]octane-2-carboxylate & 6-chloro-3-nitro-pyridin-2-amine (100% Yield) | tert-butyl (1R,4R)-5-(6-amino-5-nitropyridin-2-yl)-2,5-diazabicyclo[2.2.2]octane-2-carboxylate | 1H NMR (400 MHz, Chloroform-d) δ 8.22 (d, J = 9.3 Hz, 1H), 5.92 - 5.69 (m, 1H), 5.32 - 5.02 (m, 1H), 4.39 (m, 1H), 3.91 - 3.31 (m, 4H), 2.24 - 1.69 (m, 4H), 1.52 - 1.34 (m, 9H). LCMS (Analytical Method I) Rt = 0.90 min, MS (ESIpos): m/z 350 [M+H]+, Purity = 100%. |
| Intermediate 41-1 | tert-butyl (3S)-3-aminopyrrolidine-1-carboxylate & 6-chloro-3-nitro-pyridin-2-amine (78% Yield) | tert-butyl (3S)-3-[(6-amino-5-nitropyridin-2-yl)amino]pyrrolidine-1-carboxylate | 1H NMR (400 MHz, DMSO-d6) δ 8.10 (s, 2H), 7.96 (d, J = 9.2 Hz, 1H), 7.79 (s, 1H), 5.97 (d, J = 9.2 Hz, 1H), 4.57 - 4.36 (m, 1H), 3.67 - 3.53 (m, 1H), 3.45 - 3.36 (m, 1H), 3.18 - 3.05 (m, 1H), 2.25 - 2.06 (m, 1H), 1.96 - 1.74 (m, 1H), 1.40 (s, 9H). LCMS (Analytical Method I) Rt = 0.89 min, MS (ESIpos): m/z 324.2 [M+H]+, Purity = 96%. |
| Intermediate 42-1 | tert-butyl 2,7-diazaspiro[4.4]nonane-2-carboxylate & 6-chloro-3-nitropyridin-2-amine (98% Yield) | tert-butyl 7-(6-amino-5-nitropyridin-2-yl)-2,7-diazaspiro[4.4]nonane-2-carboxylate | 1H NMR (500 MHz, DMSO-d6) δ 8.06 (m, 1H), 7.69 (s, 1H), 6.04 (m, 1H), 3.69 - 3.33 (m, 6H), 3.23 (d, J = 8.5 Hz, 2H), 2.07 - 1.77 (m, 4H), 1.40 (m, 9H). LCMS (Analytical Method I) Rt = 0.9 min, MS (ESIpos): m/z 364.3 [M+H]+, Purity = 95%. |
| Intermediate 43-1 | tert-butyl (2S)-2-methylpiperazine-1-carboxylate & 6-chloro-3-nitro-pyridin-2-amine (99% Yield) | tert-butyl (2S)-4-(6-amino-5-nitropyridin-2-yl)-2-methylpiperazine-1-carboxylate | 1H NMR (400 MHz, DMSO-d6) δ 8.09 (d, J = 9.5 Hz, 1H), 7.60 (s, 2H), 6.29 (d, J = 9.5 Hz, 1H), 4.34 - 4.10 (m, 3H), 3.88 - 3.73 (m, 1H), 3.40 (dd, J = 13.5, 4.1 Hz, 1H), 3.24 - 3.09 (m, 2H), 1.48 - 1.38 (m, 9H), 1.08 (d, J = 6.6 Hz, 3H). LCMS (Analytical Method I) Rt = 0.90 min, MS (ESIpos): m/z 338 [M+H]+, Purity = 100%. |
| Intermediate 44-1 | tert-butyl (2R)-2-methylpiperazine-1-carboxylate & 6-chloro-3-nitro-pyridin-2-amine (99% Yield) | tert-butyl (2R)-4-(6-amino-5-nitropyridin-2-yl)-2-methylpiperazine-1-carboxylate | 1H NMR (400 MHz, DMSO-d6) δ 8.09 (d, J = 9.5 Hz, 1H), 7.60 (s, 2H), 6.29 (d, J = 9.5 Hz, 1H), 4.34 - 4.10 (m, 3H), 3.87 - 3.73 (m, 1H), 3.40 (dd, J = 13.5, 4.0 Hz, 1H), 3.25 - 3.09 (m, 2H), 1.44 (s, 9H), 1.08 (d, J = 6.6 Hz, 3H). LCMS (Analytical Method I) Rt = 0.90 min, MS (ESIpos): m/z 338 [M+H]+, Purity = 100%. |
| Intermediate 45-1 | tert-butyl (1S,6R)-3,9-diazabicyclo[4.2.1]nonane-9-carboxylate & 6-chloro-3-nitropyridin-2-amine (72% Yield) | tert-butyl (1S,6R)-3-(6-amino-5-nitropyridin-2-yl)-3,9-diazabicyclo[4.2.1]nonane-9-carboxylate | 1H NMR (400 MHz, Chloroform-d) δ 8.12 (d, J = 9.4 Hz, 1H), 6.00 (d, J = 9.4 Hz, 1H), 4.99 - 4.73 (m, 1H), 4.42 - 4.12 (m, 2H), 3.93 - 3.65 (m, 1H), 3.25 - 2.94 (m, 2H), 2.34 - 2.00 (m, 2H), 1.94 - 1.67 (m, 1H), 1.64 - 1.51 (m, 1H), 1.44 - 1.38 (m, 9H), 1.36 -1.18 (m, 2H). LCMS (Analytical Method I) Rt = 0.63 min, MS (ESIpos): m/z 364.4 [M+H]+, Purity = 96%. |
| Intermediate 46-1 | tert-butyl (2R,6S)-2,6-dimethylpiperazine-1-carboxylate & 6-chloro-3-nitro-pyridin-2-amine (99% Yield) | tert-butyl (2R,6S)-4-(6-amino-5-nitropyridin-2-yl)-2,6-dimethylpiperazine-1-carboxylate | 1H NMR (400 MHz, DMSO-d6) δ 8.09 (d, J = 9.5 Hz, 1H), 7.60 (s, 2H), 6.36 (d, J = 9.5 Hz, 1H), 4.33 (d, J = 13.5 Hz, 2H), 4.26 - 4.11 (m, 2H), 3.24 (dd, J = 13.4, 4.7 Hz, 2H), 1.44 (s, 9H), 1.14 (d, J = 6.8 Hz, 6H). LCMS (Analytical Method I) Rt = 0.97 min, MS (ESIpos): m/z 352 [M+H]+, Purity = 100%. |
| Intermediate 47-1 | tert-butyl 2,2-dimethylpiperazine-1-carboxylate & 6-chloro-3-nitro-pyridin-2-amine (93% Yield) | tert-butyl 4-(6-amino-5-nitropyridin-2-yl)-2,2-dimethylpiperazine-1-carboxylate | 1H NMR (400 MHz, DMSO-d6) δ 8.10 (d, J = 9.4 Hz, 1H), 7.60 (s, 2H), 6.20 (d, J = 9.4 Hz, 1H), 3.85 (s, 2H), 3.79 - 3.71 (m, 2H), 3.66 - 3.54 (m, 2H), 1.44 (s, 9H), 1.36 (s, 6H). LCMS (Analytical Method I) Rt = 0.95 min, MS (ESIpos): m/z 352 [M+H]+, Purity = 100%. |
| Intermediate 48-1 | tert-butyl (2S,6S)-2,6-dimethylpiperazine-1-carboxylate & 6-chloro-3-nitro-pyridin-2-amine (95% Yield) | tert-butyl (2S,6S)-4-(6-amino-5-nitropyridin-2-yl)-2,6-dimethylpiperazine-1-carboxylate | 1H NMR (400 MHz, DMSO-d6) δ 8.12 (d, J = 9.4 Hz, 1H), 7.61 (s, 2H), 6.17 (d,J = 9.5 Hz, 1H), 4.19 - 4.10 (m, 2H), 3.95 (s, 2H), 3.68 (dd, J = 13.5, 4.0 Hz, 2H), 1.45 (s, 9H), 1.20 (d, J = 6.6 Hz, 6H). LCMS (Analytical Method I) Rt = 0.96 min, MS (ESIpos): m/z 352 [M+H]+, Purity = 100%. |
| Intermediate 49-1 | tert-butyl (3S)-3-methylpiperazine-1-carboxylate & 6-chloro-3-nitro-pyridin-2-amine (100% Yield) | tert-butyl (3S)-4-(6-amino-5-nitropyridin-2-yl)-3-methylpiperazine-1-carboxylate | 1H NMR (400 MHz, DMSO-d6) δ 8.09 (d, J = 9.5 Hz, 1H), 7.59 (s, 2H), 6.27 (d, J = 9.5 Hz, 1H), 4.75 - 4.55 (m, 1H), 4.24 (dt, J = 13.6, 3.3 Hz, 1H), 3.90 (d, J = 13.3 Hz, 1H), 3.84 - 3.70 (m, 1H), 3.32 - 3.13 (m, 2H), 3.04 -2.95 (m, 1H), 1.44 (s, 9H), 1.16 (d, J = 6.7 Hz, 3H). LCMS (Analytical Method I) Rt = 0.93 min, MS (ESIpos): m/z 338 [M+H]+, Purity = 100%. |
| Intermediate 50-1 | tert-butyl 3,6-diazabicyclo[3.2.1]octane-3-carboxylate & 6-chloro-3-nitropyridin-2-amine (98% Yield) | tert-butyl 6-(6-amino-5-nitropyridin-2-yl)-3,6-diazabicyclo[3.2.1]octane-3-carboxylate | 1H NMR (400 MHz, DMSO-d6) δ 8.06 (d, J = 9.4 Hz, 1H), 7.60 (s, 2H), 6.24 - 5.93 (m, 1H), 4.60 - 4.06 (m, 2H), 4.01 - 3.87 (m, 1H), 3.56 - 3.49 (m, 1H), 3.36 - 3.15 (m, 1H), 3.01 - 2.86 (m, 2H), 2.62 - 2.54 (m, 1H), 2.05 -1.95 (m, 1H), 1.92 - 1.81 (m, 1H), 1.22 (m, 9H). LCMS (Analytical Method H) Rt = 0.55 min, MS (ESIpos): m/z 350.4 [M+H]+, Purity = 96%. |
| Intermediate 51-1 | N-boc-piperazine & 6-chloro-5-methyl-3-nitro-pyridin-2-amine (92% Yield) | tert-butyl 4-(6-amino-3-methyl-5-nitropyridin-2-yl)piperazine-1-carboxylate | 1H NMR (400 MHz, Chloroform-d) δ 8.06 - 7.94 (m, 1H), 3.46 (m, 4H), 3.36 (m, 4H), 2.16 - 2.07 (m, 3H), 1.41 (s, 9H). LCMS (Analytical Method I) Rt = 1.12 min, MS (ESIpos): m/z 338.3 [M+H]+, Purity = 97%. |
| Intermediate 52-1 | tert-butyl (2R)-2-(methoxymethyl)piperazine-1-carboxylate & 6-chloro-3-nitro-pyridin-2-amine (85% Yield) | tert-butyl (2R)-4-(6-amino-5-nitropyridin-2-yl)-2-(methoxymethyl)piperazine-1-carboxylate | 1H NMR (400 MHz, DMSO-d6) δ 8.10 (d, J = 9.5 Hz, 1H), 7.61 (s, 2H), 6.24 (d, J = 9.5 Hz, 1H), 4.38 - 4.11 (m, 3H), 3.90 - 3.73 (m, 1H), 3.42 - 3.31 (m, 3H), 3.25 (s, 3H), 3.22 - 3.08 (m, 2H), 1.44 (s, 9H). LCMS (Analytical Method I) Rt = 0.88 min, MS (ESIpos): m/z 368 [M+H]+, Purity = 99%. |
| Intermediate 53-1 | tert-butyl (2S)-2-(methoxymethyl)piperazine-1-carboxylate & 6-chloro-3-nitro-pyridin-2-amine (96% Yield) | tert-butyl (2S)-4-(6-amino-5-nitropyridin-2-yl)-2-(methoxymethyl)piperazine-1-carboxylate | 1H NMR (400 MHz, DMSO-d6) δ 8.10 (d, J = 9.5 Hz, 1H), 7.61 (s, 2H), 6.24 (d, J = 9.5 Hz, 1H), 4.36 - 4.13 (m, 3H), 3.89 - 3.78 (m, 1H), 3.42 - 3.31 (m, 3H), 3.25 (s, 3H), 3.22 - 3.09 (m, 2H), 1.44 (s, 9H). LCMS (Analytical Method I) Rt = 0.88 min, MS (ESIpos): m/z 368 [M+H]+, Purity = 100%. |
| Intermediate 54-1 | tert-butyl (3R,5R)-3,5-dimethylpiperazine-1-carboxylate & 6-chloro-3-nitro-pyridin-2-amine (40% Yield) | tert-butyl (3R,5R)-4-(6-amino-5-nitropyridin-2-yl)-3,5-dimethylpiperazine-1-carboxylate | 1H NMR (400 MHz, DMSO) δ 8.10 (d, J = 9.5 Hz, 1H), 7.58 (s, 2H), 6.15 (d, J = 9.5 Hz, 1H), 4.46 (s, 2H), 3.72 -3.49 (m, 4H), 1.46 (s, 9H), 1.28 (d, J = 6.6 Hz, 6H). LCMS (Analytical Method I) Rt = 0.95 min, MS (ESIpos): m/z 352 [M+H]+, Purity = 80%. |
| Intermediate 55-1 | tert-butyl N-methyl-N-[(3S)-pyrrolidin-3-yl]carbamate;hydrochloride & 6-chloro-3-nitro-pyridin-2-amine (84% Yield) | tert-butyl N-[(3S)-1-(6-amino-5-nitropyridin-2-yl)pyrrolidin-3-yl]-N-methylcarbamate | 1H NMR (400 MHz, DMSO) δ 8.07 (d, J = 9.4 Hz, 1H), 7.60 (s, 2H), 6.04 (d, J = 9.4 Hz, 1H), 4.67 (p, J = 7.6 Hz, 1H), 3.81 - 3.65 (m, 2H), 3.55 - 3.38 (m, 2H), 2.77 (s, 3H), 2.25 (m, 2H), 1.44 (s, 9H). LCMS (Analytical Method I) Rt = 0.89 min, MS (ESIpos): m/z 338 [M+H]+, Purity = 100%. |
| Intermediate 56-1 | tert-butyl 5,8-diazaspiro[3.5]nonane-5-carboxylate & 6-chloro-3-nitropyridin-2-amine (95% Yield) | tert-butyl 8-(6-amino-5-nitropyridin-2-yl)-5,8-diazaspiro[3.5]nonane-5-carboxylate | 1H NMR (400 MHz, DMSO-d6) δ 8.09 (d, J = 9.5 Hz, 1H), 8.05 - 7.67 (m, 2H), 6.33 (s, 1H), 3.92 (s, 2H), 3.56 (s, 2H), 3.45 (s, 2H), 2.48 - 2.38 (m, 2H), 1.90 - 1.70 (m, 4H), 1.44 (s, 9H). LCMS (Analytical Method I) Rt = 1.02 min, MS (ESIpos): m/z 364.3 [M+H]+, Purity = 100%. |
| Intermediate 57-1 | tert-butyl methyl[(3R)-pyrrolidin-3-yl]carbamate & 6-chloro-3-nitro-pyridin-2-amine (95% Yield) | tert-butyl N-[(3R)-1-(6-amino-5-nitropyridin-2-yl)pyrrolidin-3-yl]-N-methylcarbamate | 1H NMR (400 MHz, DMSO) δ 8.07 (d, J = 9.4 Hz, 1H), 7.60 (s, 2H), 6.04 (d, J = 9.4 Hz, 1H), 4.75 - 4.57 (m, 1H), 3.82 - 3.65 (m, 2H), 3.57 - 3.34 (m, 2H), 2.77 (s, 3H), 2.21 - 2.04 (m, 2H), 1.44 (s, 9H). LCMS (Analytical Method I) Rt = 0.89 min, MS (ESIpos): m/z 338 [M+H]+, Purity = 100%. |
| Intermediate 58-1 | tert-butyl 3-oxa-7,9-diazabicyclo[3.3.1]nonane-9-carboxylate & 6-chloro-3-nitropyridin-2-amine (99% Yield) | tert-butyl 7-(6-amino-5-nitropyridin-2-yl)-3-oxa-7,9-diazabicyclo[3.3.1]nonane-9-carboxylate | 1H NMR (400 MHz, DMSO) δ 8.07 (d, J = 9.6 Hz, 1H), 8.01 - 7.46 (m, 2H), 6.33 (d, J = 9.6 Hz, 1H), 5.06 - 4.12 (m, 2H), 4.08 - 3.92 (m, 2H), 3.92 -3.71 (m, 2H), 3.67 - 3.54 (m, 2H), 1.45 (s, 9H). 2 protons obscured. LCMS (Analytical Method I) Rt = 0.82 min, MS (ESIpos): m/z 366.3 [M+H]+, Purity = 100%. |
| Intermediate 59-1 | tert-butyl 3-oxa-7,9-diazabicyclo[3.3.1]nonane-7-carboxylate & 6-chloro-3-nitropyridin-2-amine (63% Yield) | tert-butyl 9-(6-amino-5-nitropyridin-2-yl)-3-oxa-7,9-diazabicyclo[3.3.1]nonane-7-carboxylate | 1H NMR (500 MHz, DMSO) δ 8.13 (d, J = 9.5 Hz, 1H), 8.08 - 7.63 (m, 2H), 6.33 (d, J = 9.5 Hz, 1H), 4.91 - 4.75 (m, 1H), 4.31 - 4.20 (m, 2H), 4.14 (d, J = 13.6 Hz, 1H), 4.04 - 3.91 (m, 2H), 3.79 - 3.55 (m, 2H), 3.21 (d, J = 13.3 Hz, 1H), 3.13 - 2.95 (m, 1H), 1.41 (s, 9H). LCMS (Analytical Method I) Rt = 0.79 min, MS (ESIpos): m/z 366.3 [M+H]+, Purity = 88%. |
| Intermediate 60-1 | (1R,4R)-2-oxa-5-azabicyclo[2.2.1]heptane & 6-chloro-3-nitropyridin-2-amine (66% Yield) | 3-nitro-6-[(1R,4R)-2-oxa-5-azabicyclo[2.2.1]heptan-5-yl]pyridin-2-amine | 1H NMR (400 MHz, DMSO) δ 8.33 -7.45 (m, 3H), 6.36 - 5.81 (m, 1H), 5.23 - 4.83 (m, 1H), 4.80 - 4.64 (m, 1H), 3.88 - 3.74 (m, 1H), 3.74 - 3.60 (m, 1H), 3.55 - 3.33 (m, 2H), 2.06 -1.79 (m, 2H). LCMS (Analytical Method I) Rt = 0.49 min, MS (ESIpos): m/z 237.1 [M+H]+, Purity = 77%. |
| Intermediate 61-1 | 2-trifluoromethyl-piperazine-1-carboxylic acid tert-butyl ester & 6-chloro-3-nitropyridin-2-amine (99% Yield) | tert-butyl 4-(6-amino-5-nitropyridin-2-yl)-2-(trifluoromethyl)piperazine-1-carboxylate | 1H NMR (400 MHz, DMSO) δ 8.11 (d, J = 9.5 Hz, 1H), 8.04 - 7.59 (m, 2H), 6.33 (d, J = 9.5 Hz, 1H), 5.08 - 4.59 (m, 2H), 4.61 - 4.14 (m, 1H), 3.99 -3.89 (m, 1H), 3.51 (d, J = 10.4 Hz, 1H), 3.25 - 3.08 (m, 1H), 3.09 - 2.96 (m, 1H), 1.44 (s, 9H). LCMS (Analytical Method I) Rt = 0.96 min, MS (ESIpos): m/z 392 [M+H]+, Purity = 100%. |
| Intermediate 62-1 | tert-butyl 6,6-difluoro-1,4-diazepane-1-carboxylate & 6-chloro-3-nitropyridin-2-amine (53% Yield) | tert-butyl 4-(6-amino-5-nitropyrid in-2-yl)-6,6-difluoro-1,4-diazepane-1-carboxylate | 1H NMR (500 MHz, DMSO) δ 8.13 (d, J = 9.5 Hz, 1H), 7.94 (s, 2H), 6.37 (d, J = 9.4 Hz, 1H), 4.41 - 4.15 (m, 2H), 3.95 - 3.75 (m, 4H), 3.71 - 3.56 (m, 2H), 1.46 - 1.30 (m, 9H). LCMS (Analytical Method I) Rt = 0.91 min, MS (ESIpos): m/z 374.2 (M+H]+, Purity = 95% |
| Intermediate 63-1 | tert-butyl-5-methyl-1,4-diazepane-1-carboxylate & 6-chloro-3-nitropyridin-2-amine (79% Yield) | tert-butyl 4-(6-amino-5-nitropyridin-2-yl)-5-methyl-1,4-diazepane-1-carboxylate | 1H NMR (500 MHz, DMSO) δ 8.05 (d, J = 9.4 Hz, 1H), 7.96 (s, 1H), 7.68 (s, 1H), 6.28 (d, J = 9.6 Hz, 1H), 5.20 -4.83 (m, 1H), 4.60 - 4.32 (m, 1H), 4.31 - 4.02 (m, 1H), 3.90 - 3.74 (m, 1H), 3.74 - 3.57 (m, 1H), 3.27 - 3.07 (m, 1H), 2.95 - 2.76 (m, 1H), 2.18 -2.00 (m, 1H), 1.59 - 1.48 (m, 1H), 1.42 - 1.28 (m, 9H), 1.16 (d, J = 6.5 Hz, 3H). LCMS (Analytical Method I) Rt = 0.94 min, MS (ESIpos): m/z 352.3 [M+H]+, Purity = 93%. |
| Intermediate 64-1 | tert-butyl (3S)-3-(hydroxymethyl)piperazine-1-carboxylate & 6-chloro-3-nitropyridin-2-amine (93% Yield) | tert-butyl (3S)-4-(6-amino-5-nitropyridin-2-yl)-3-(hydroxymethyl)piperazine-1-carboxylate | 1H NMR (500 MHz, MeOD) δ 8.18 (d, J = 9.5 Hz, 1H), 6.30 (d, J = 9.6 Hz, 1H), 4.60 (s, 1H), 4.50 - 4.28 (m, 1H), 4.25 - 4.15 (m, 1H), 4.03 - 3.90 (m, 1H), 3.71 - 3.62 (m, 2H), 3.29 - 2.98 (m, 3H), 1.51 (s, 9H). LCMS (Analytical Method I) Rt = 0.75 min, MS (ESIpos): m/z 354.3 [M+H]+, Purity = 100%. |
| Intermediate 65-1 | N-boc-piperazine & 6-chloro-4-methyl-3-nitropyridin-2-amine (85% Yield) | tert-butyl 4-(6-amino-4-methyl-5-nitropyridin-2-yl)piperazine-1-carboxylate | 1H NMR (500 MHz, DMSO-d6) δ 7.68 (s, 2H), 6.20 (s, 1H), 3.74 - 3.58 (m, 4H), 3.42 - 3.36 (m, 4H), 2.46 (s, 3H), 1.42 (s, 9H). LCMS (Analytical Method I) Rt = 0.99 min, MS (ESIpos): m/z 338.2 [M+H]+, Purity = 100%. |
| Intermediate 66-1 | tert-butyl (1R,5S)-3,8-diazabicyclo[3.2.1]octane-8-carboxylate & 6-chloro-4-methyl-3-nitropyridin-2-amine (94% Yield) | tert-butyl (1R,5S)-3-(6-amino-4-methyl-5-nitropyridin-2-yl)-3,8-diazabicyclo[3.2.1]octane-8-carboxylate | 1H NMR (500 MHz, Chloroform-d) δ 5.84 (s, 1H), 4.50 - 3.79 (m, 4H), 3.14 (br s, 2H), 2.57 (s, 3H), 2.00 - 1.87 (m, 2H), 1.72 - 1.60 (m, 2H), 1.48 (s, 9H). LCMS (Analytical Method I) Rt = 1.10 min, MS (ESIpos): m/z 364.3 [M+H]+, Purity = 100%. |
| Intermediate 67-1 | tert-butyl (1S,4S)-2,5-diazabicyclo[2.2.1]heptane-2-carboxylate & 6-chloro-4-methyl-3-nitropyridin-2-amine (91% Yield) | tert-butyl (1S,4S)-5-(6-amino-4-methyl-5-nitropyridin-2-yl)-2,5-diazabicyclo[2.2.1]heptane-2-carboxylate | LCMS (Analytical Method I) Rt = 1.00 min, MS (ESIpos): m/z 350.2 [M+H]+, Purity = 99%. |
| Intermediate 68-1 | tert-butyl (1R,4R)-2-aza-5-azoniabicyclo[2.2.1]heptane-2-carboxylate;chloride & 6-chloro-4-methyl-3-nitropyridin-2-amine (95% Yield) | tert-butyl (1R,4R)-5-(6-amino-4-methyl-5-nitropyridin-2-yl)-2,5-diazabicyclo[2.2.1]heptane-2-carboxylate | 1H NMR (400 MHz, DMSO-d6) δ 7.40 (s, 2H), 5.91 (s, 1H), 4.91 (s, 1H), 4.48 (s, 1H), 3.52 (dd, J = 10.3, 1.9 Hz, 1H), 3.44 - 3.33 (m, 2H), 3.19 (d, J = 9.9 Hz, 1H), 2.47 (s, 3H), 1.90 (q, J = 9.9 Hz, 2H), 1.41 (s, 9H). LCMS (Analytical Method I) Rt = 0.89 min, MS (ESIpos): m/z 350.2 [M+H]+, Purity = 100%. |
| Intermediate 69-1 | O1-tert-butyl O2-methyl (2R)-piperazine-1,2-dicarboxylate & 6-chloro-3-nitropyridin-2-amine (91% Yield) | 1-tert-butyl 2-methyl(2R)-4-(6-amino-5-nitropyridin-2-yl)piperazine-1,2-dicarboxylate | 1H NMR (400 MHz, DMSO) δ 8.09 (d, J = 9.5 Hz, 1H), 7.90 (s, 2H), 6.26 (d, J = 9.2 Hz, 1H), 5.22 - 4.50 (m, 2H), 4.29 (s, 1H), 3.79 (dd, J = 9.6, 3.3 Hz, 1H), 3.73 - 3.40 (m, 4H), 3.28 - 3.03 (m, 2H), 1.40 (m, 9H). LCMS (Analytical Method I) Rt = 0.87 min, MS (ESIpos): m/z 382.3 [M+H]+, Purity = 100%. |
| Intermediate 70-1 | tert-butyl (1R,6S)-3,8-diazabicyclo[4.2.0]octane-8-carboxylate & 6-chloro-3-nitropyridin-2-amine (99% Yield) | tert-butyl (1S,6R)-3-(6-amino-5-nitropyridin-2-yl)-3,8-diazabicyclo[4.2.0]oc-tane-8-carboxylate | 1HNMR(400 MHz, DMSO-d6) δ 8.15 - 8.04 (m, 1H), 8.02 - 7.48 (m, 2H), 6.20 (d,J = 9.5 Hz, 1H), 4.50 - 4.40 (m, 1H), 4.08 - 3.36 (m, 6H), 2.81 -2.70 (m, 1H), 2.17 - 1.99 (m, 1H), 1.90 - 1.78 (m, 1H), 1.29 (s, 9H) LCMS (Analytical Method H) Rt = 0.55 min, MS (ESIpos): m/z 350.4 [M+H]+, Purity = 97%. |

### Synthesis of Tert-butyl 4-{5-nitro-6-[(pyridin-4-yl)amino]pyridin-2-yl}-1,4-diazepane-1-carboxylate / intermediate 27

To a degassed solution of cesium carbonate (2.03 g, 6.22 mmol), (5-diphenylphosphanyl-9,9-dimethyl-xanthen-4-yl)-diphenyl-phosphane (90 mg, 0.156 mmol), 4-iodopyridine (638 mg, 3.11 mmol), tert-butyl 4-(6-amino-5-nitro-2-pyridyl)-1,4-diazepane-1-carboxylate (Intermediate 27-1) (1.05 g, 3.11 mmol) in 1,4-dioxane (5.4 mL) was added (1{E},4{E})-1,5-diphenylpenta-1,4-dien-3-one;palladium (71 mg, 0.0778 mmol) and the solution sparged with nitrogen. The mixture was heated to 100° C. for 19 h. The reaction was cooled and the supernatant liquid decanted and concentrated in vacuo. The product was purified by flash chromatography (50 g, silica), eluting with 0-20% MeOH/DCM to yield the title compound (1.03 g, 76% yield) as a yellow solid. 1H NMR (500 MHz, Chloroform-d) δ 10.77 - 10.61 (m, 1H), 8.47 (d, J = 5.4 Hz, 2H), 8.24 (d, J = 9.2 Hz, 1H), 7.73 (d, J = 5.9 Hz, 2H), 6.57 - 6.43 (m, 1H), 4.04 - 3.65 (m, 4H), 3.65 - 3.46 (m, 3H), 3.32 - 3.23 (m, 1H), 1.91 - 1.65 (m, 2H), 1.32 - 1.08 (m, 9H). LCMS (Analytical Method I) Rt = 0.67 min, MS (ESIpos): m/z 415.3 [M+H]+, Purity = 98%.

Each of Intermediates 28-2 through 82-1 as listed in Table 1.4.2 were prepared according to the method of intermediate 27 using the intermediates listed in the “Synthesis” column. The intermediates were purified by flash chromatography, SCX or preparative HPLC Methods, A1, A2, B1, B2 as required.

TABLE 1.4.2

| | | | |
|---|---|---|---|
| Intermediate | Synthesis | Structure/Name | Data |
| Intermediate 28-2 | Intermediate 28-1 & 4-bromo-2-methylpyridine (91% Yield) | tert-butyl 6-{6-[(2-methylpyridin-4-yl)amino]-5-nitropyridin-2-yl}-2,6-diazaspiro[3.3]heptane-2-carboxylate | 1H NMR (400 MHz, Chloroform-d) δ 10.71 (s, 1H), 8.28 (d, J = 5.7 Hz, 1H), 8.05 (d, J = 9.3 Hz, 1H), 7.43 (dd, J = 5.7, 2.0 Hz, 1H), 5.65 (d, J = 9.3 Hz, 1H), 4.24 (s, 4H), 4.13 (s, 4H), 2.46 (s, 3H), 1.41 (s, 9H). LCMS (Analytical Method I) Rt = 0.75 min, MS (ESIpos): m/z 427.4 [M+H]+, Purity = 98%. |
| Intermediate 29 | Intermediate 29-1 & 4-iodopyridine (97% Yield) | tert-butyl 6-{5-nitro-6-[(pyridin-4-yl)amino]pyridin-2-yl}-3,6-diazabicyclo[3.1.1]heptane-3-carboxylate | 1H NMR (400 MHz, DMSO-d6) δ 10.67 (s, 1H), 8.46 (d, J = 6.3 Hz, 2H), 8.30 (d, J = 9.2 Hz, 1H), 7.79 (d, J = 6.2 Hz, 2H), 6.34 (d, J = 9.2 Hz, 1H), 4.77 - 4.54 (m, 2H), 3.96 - 3.69 (m, 2H), 3.55 - 3.42 (m, 2H), 2.82 - 2.74 (m, 1H), 1.74 - 1.63 (m, 1H), 1.39 - 1.24 (m, 9H). LCMS (Analytical Method G) Rt = 1.62 min, MS (ESIpos): m/z 413.2 [M+H]+, Purity = 91%. |
| Intermediate 30 | Intermediate 30-1 & 4-iodopyridine (96% Yield) | tert-butyl 7-{5-nitro-6-[(pyridin-4-yl)amino]pyridin-2-yl}-4,7-diazaspiro[2.5]octane-4-carboxylate | 1H NMR (400 MHz, DMSO-d6) δ 10.53 (s, 1H), 8.48 (dd, J = 4.8, 1.5 Hz, 2H), 8.25 (d, J = 9.5 Hz, 1H), 7.61 (dd, J = 4.8, 1.6 Hz, 2H), 6.55 (d, J = 9.6 Hz, 1H), 3.85 - 3.74 (m, 2H), 3.66 - 3.53 (m, 4H), 1.45 (s, 9H), 1.04 - 0.94 (m, 2H), 0.87 - 0.78 (m, 2H). LCMS (Analytical Method I) Rt = 0.73 min, MS (ESIpos): m/z 427 [M+H]+, Purity = 100%. |
| Intermediate 31-2 | Intermediate 31-1 & 4-iodopyridine (98% Yield) | tert-butyl 6-{5-nitro-6-[(pyridin-4-yl)amino]pyrid in-2-yl}-1,6-diazaspiro[3.3]heptane-1-carboxylate | 1H NMR (400 MHz, DMSO-d6) δ 10.70 (s, 1H), 8.47 (dd, J = 4.9, 1.4 Hz, 2H), 8.26 (d, J = 9.3 Hz, 1H), 7.81 (dd, J = 4.8, 1.5 Hz, 2H), 6.13 (d, J = 9.3 Hz, 1H), 4.62 (d, J = 10.0 Hz, 2H), 4.38 (d, J = 10.5 Hz, 2H), 3.77 (t, J = 7.1 Hz, 2H), 1.33 (s, 9H). 2 protons obscured. LCMS (Analytical Method I) Rt = 0.68 min, MS (ESIpos): m/z 413 [M+H]+, Purity = 100%. |
| Intermediate 32 | Intermediate 32-1 & 4-iodopyridine (64% Yield) | tert-butyl (1R,5S)-3-{5-nitro-6-[(pyrid in-4-yl)amino]pyridin-2-yl}-3,8-diazabicyclo[3.2.1]octane-8-carboxylate | 1H NMR (500 MHz, DMSO-d6) δ 10.61 (s, 1H), 8.49 (d, J = 6.2 Hz, 2H), 8.28 (d, J = 9.6 Hz, 1H), 7.78 - 7.57 (m, 2H), 6.55 (d, J = 9.6 Hz, 1H), 4.42 - 4.18 (m, H), 3.26 - 3.16 (m, 2H), 1.92 - 1.76 (m, 2H), 1.74 - 1.52 (m, 2H), 1.44 (s, 9H). 1 signal obscured. LCMS (Analytical Method I) Rt = 0.64 min, MS (ESIpos): m/z 427.4 [M+H]+, Purity = 86%. |
| Intermediate 33-2 | Intermediate 33-1 & 4-iodopyridine (76% Yield) | tert-butyl 1-{5-nitro-6-[(pyridin-4-yl)amino]pyridin-2-yl}-1,6-diazaspiro[3.3]heptane-6-carboxylate | 1H NMR (400 MHz, DMSO-d6) δ 10.74 (s, 1H), 8.41 (dd, J = 4.9, 1.4 Hz, 2H), 8.31 (d, J = 9.3 Hz, 1H), 7.81 - 7.67 (m, 2H), 6.12 (d, J = 8.5 Hz, 1H), 4.55 (s, 2H), 4.17 - 3.94 (m, 4H), 2.73 - 2.61 (m, 2H), 1.42 (s, 9H). LCMS (Analytical Method A) Rt = 2.05 min, MS (ESIpos): m/z 413 [M+H]+, Purity = 97%. |
| Intermediate 34-2 | Intermediate 34-1 & 4-bromopyridazine (93% Yield) | tert-butyl (3R)-3-methyl-4-{5-nitro-6-[(pyridazin-4-yl)amino]pyridin-2-yl}piperazine-1-carboxylate | 1H NMR (400 MHz, DMSO) δ 10.65 (s, 1H), 9.54 (d, J = 1.9 Hz, 1H), 9.14 (d, J = 5.9 Hz, 1H), 8.37 (d, J = 9.6 Hz, 1H), 8.14 - 8.06 (m, 1H), 6.67 (d, J = 9.6 Hz, 1H), 4.79 - 4.55 (m, 1H), 4.33 - 4.10 (m, 1H), 4.05 - 3.77 (m, 3H), 3.33 - 3.18 (m, 2H), 1.49 (s, 9H), 1.24 (d, J = 6.6 Hz, 3H). LCMS (Analytical Method I) Rt = 0.92 min, MS (ESIpos): m/z 416.3 [M+H]+, Purity = 68%. |
| Intermediate 35 | Intermediate 35-1 & 4-iodopyridine (67% Yield) | tert-butyl (1R,5S)-8-{5-nitro-6-[(pyridin-4-yl)amino]pyridin-2-yl}-3,8-diazabicyclo[3.2.1]octane-3-carboxylate | 1H NMR (400 MHz, Methanol-d4) δ 8.37 - 8.32 (m, 2H), 8.24 (d, J = 9.5 Hz, 1H), 7.71 - 7.62 (m, 2H), 6.36 (d, J = 9.5 Hz, 1H), 3.93 - 3.78 (m, 2H), 3.16 - 2.92 (m, 2H), 2.08 - 1.93 (m, 2H), 1.87 - 1.74 (m, 2H), 1.39 (s, 9H). LCMS (Analytical Method I) Rt = 0.75 min, MS (ESIpos): m/z 427.3 [M+H]+, Purity = 95%. |
| Intermediate 36 | Intermediate 36-1 & 4-iodopyridine (92% Yield) | tert-butyl 2-{5-nitro-6-[(pyridin-4-yl)amino]pyridin-2-yl}-2,7-diazaspiro[3.5]nonane-7-carboxylate | 1H NMR (500 MHz, Methanol-d4) δ 8.41 - 8.36 (m, 2H), 8.27 (d, J = 9.3 Hz, 1H), 7.91 - 7.86 (m, 2H), 6.04 (d, J = 9.3 Hz, 1H), 4.12 - 3.90 (m, 4H), 3.57 - 3.35 (m, 4H), 1.84 (t, J = 5.6 Hz, 4H), 1.47 (s, 9H). LCMS (Analytical Method I) Rt = 0.88 min, MS (ESIpos): m/z 441.3 [M+H]+, Purity = 100%. |
| Intermediate 37 | Intermediate 37-1 & 4-iodopyridine (99% Yield) | tert-butyl (3R)-3-({5-nitro-6-[(pyridin-4-yl)amino]pyridin-2-yl}amino)pyrrolidine-1-carboxylate | 1H NMR (500 MHz, Methanol-d4) δ 8.44 (d, J = 6.0 Hz, 2H), 8.24 (d, J = 8.7 Hz, 1H), 7.89 (m, 2H), 6.23 (d, J = 9.0 Hz, 1H), 4.62 (s, 1H), 3.79 - 3.71 (m, 1H), 3.58 - 3.47 (m, 2H), 3.45 - 3.36 (m, 1H), 2.37 - 2.25 (m, 1H), 2.06 (d, J = 7.2 Hz, 1H), 1.53 - 1.41 (m, 9H). LCMS (Analytical Method I) Rt = 0.76 min, MS (ESIpos): m/z 401.3 [M+H]+, Purity = 87%. |
| Intermediate 38 | Intermediate 38-1 & 4-iodopyridine (64% Yield) | tert-butyl (1R,4R)-5-{5-nitro-6-[(pyridin-4-yl)amino]pyridin-2-yl}-2,5-diazabicyclo[2.2.1]heptane-2-carboxylate | 1H NMR (400 MHz, DMSO-d6) δ 10.85 -10.62 (m, 1H), 8.47 (m, 2H), 8.25 (m, 1H), 7.78 (m, 2H), 6.65 - 6.14 (m, 1H), 5.10 -4.87 (m, 1H), 4.63 - 4.43 (m, 1H), 3.85 -3.39 (m, 3H), 3.26 - 3.08 (m, 1H), 2.10 -1.91 (m, 2H), 1.52 - 1.27 (m, 9H). LCMS (Analytical Method I) Rt = 0.79 min, MS (ESIpos): m/z 413.3 [M+H]+, Purity = 98%. |
| Intermediate 39-2 | Intermediate 39-1 & 4-iodopyridine (87% Yield) | tert-butyl (1S,4S)-5-{5-nitro-6-[(pyridin-4-yl)amino]pyridin-2-yl}-2,5-diazabicyclo[2.2.2]octane-2-carboxylate | 1H NMR (400 MHz, DMSO-d6) δ 10.72 (m, 1H), 8.47 (s, 2H), 8.27 (m, 1H), 7.74 (m, 2H), 6.67 - 6.14 (m, 1H), 5.02 - 4.46 (m, 1H), 4.41 - 4.17 (m, 1H), 3.85 - 3.42 (m, 4H), 2.10 - 1.59 (m, 4H), 1.63 - 1.06 (m, 9H). LCMS (Analytical Method I) Rt = 0.73 min, MS (ESIpos): m/z 427 [M+H]+, Purity = 100%. |
| Intermediate 40 | Intermediate 40-1 & 4-iodopyridine (94% Yield) | tert-butyl (1R,4R)-5-{5-nitro-6-[(pyridin-4-yl)amino]pyridin-2-yl}-2,5-diazabicyclo[2.2.2]octane-2-carboxylate | 1H NMR (500 MHz, DMSO-d6) δ 10.72 (m, 1H), 8.47 (m, 2H), 8.27 (m, 1H), 7.74 (m, 2H), 6.69 - 6.13 (m, 1H), 5.02 - 4.47 (m, 1H), 4.39 - 4.19 (m, 1H), 3.84 - 3.43 (m, 4H), 1.99 - 1.74 (m, 4H), 1.47 - 1.33 (m, 9H). LCMS (Analytical Method 1) Rt = 0.73 min, MS (ESIpos): m/z 427 [M+H]+, Purity = 100%. |
| Intermediate 41 | Intermediate 41-1 & 4-iodopyridine (65% Yield) | tert-butyl (3S)-3-({5-nitro-6-[(pyrid in-4-yl)amino]pyridin-2-yl}amino)pyrrolidine-1-carboxylate | 1H NMR (400 MHz, DMSO-d6) δ 10.91 -10.75 (m, 1H), 8.57 (d, J = 5.7 Hz, 1H), 8.46 (d, J = 6.2 Hz, 2H), 8.17 (d, J = 9.3 Hz, 1H), 7.86 - 7.72 (m, 2H), 6.25 (d, J = 9.3 Hz, 1H), 4.58 - 4.36 (m, 1H), 3.74 -3.51 (m, 1H), 3.48 - 3.37 (m, 2H), 3.25 -3.16 (m, 1H), 2.32 - 2.13 (m, 1H), 2.05 -1.86 (m, 1H), 1.40 (m, 9H). LCMS (Analytical Method 1) Rt = 0.78 min, MS (ESIpos): m/z 401.3 [M+H]+, Purity = 92%. |
| Intermediate 42 | Intermediate 42-1 & 4-iodopyridine (91% Yield) | tert-butyl 7-{5-nitro-6-[(pyridin-4-yl)amino]pyrid in-2-yl}-2,7-diazaspiro[4.4]nonane-2-carboxylate | 1H NMR (500 MHz, DMSO-d6) δ 10.84 -10.72 (m, 1H), 8.51 - 8.41 (m, 2H), 8.25 (m, 1H), 7.89 - 7.77 (m, 2H), 6.28 (m, 1H), 3.84 - 3.72 (m, 2H), 3.72 - 3.58 (m, 2H), 3.55 - 3.45 (m, 2H), 3.32 - 3.21 (m, 2H), 2.10 - 1.95 (m, 2H), 1.95 - 1.80 (m, 2H), 1.42 - 1.37 (m, 9H). LCMS (Analytical Method 1) Rt = 0.70 min, MS (ESlpos): m/z 441.3 [M+H]+, Purity = 97%. |
| Intermediate 43 | Intermediate 43-1 & 4-iodopyridine (97% Yield) | tert-butyl (2S)-2-methyl-4-{5-nitro-6-[(pyridin-4-yl)amino]pyridin-2-yl}piperazine-1-carboxylate | 1H NMR (400 MHz, DMSO-d6) δ 10.57 (s, 1H), 8.49 (dd, J = 4.7, 1.6 Hz, 2H), 8.27 (d, J = 9.5 Hz, 1H), 7.71 - 7.61 (m, 2H), 6.55 (d, J = 9.6 Hz, 1H), 4.33 - 4.09 (m, 3H), 3.90 - 3.79 (m, 1H), 3.51 (dd, J = 13.6, 4.1 Hz, 1H), 3.38 - 3.17 (m, 2H), 1.44 (s, 9H), 1.11 (d, J = 6.6 Hz, 3H). LCMS (Analytical Method 1) Rt = 0.69 min, MS (ESlpos): m/z 415 [M+H]+, Purity = 100%. |
| Intermediate 44 | Intermediate 44-1 & 4-iodopyridine (97% Yield) | tert-butyl (2R)-2-methyl-4-{5-nitro-6-[(pyridin-4-yl)amino]pyridin-2-yl}piperazine-1-carboxylate | 1H NMR (400 MHz, DMSO-d6) δ 10.57 (s, 1H), 8.49 (dd, J = 4.7, 1.6 Hz, 2H), 8.27 (d, J = 9.5 Hz, 1H), 7.74 - 7.56 (m, 2H), 6.55 (d, J = 9.6 Hz, 1H), 4.32 - 4.09 (m, 3H), 3.91 - 3.79 (m, 1H), 3.51 (dd, J = 13.6, 4.1 Hz, 1H), 3.38 - 3.18 (m, 2H), 1.44 (s, 9H), 1.11 (d, J = 6.6 Hz, 3H). LCMS (Analytical Method 1) Rt = 0.70 min, MS (ESlpos): m/z 415 [M+H]+, Purity = 100%. |
| Intermediate 45 | Intermediate 45-1 & 4-iodopyridine (88% Yield) | tert-butyl (1R,6S)-3-{5-nitro-6-[(pyridin-4-yl)amino]pyridin-2-yl}-3,9-diazabicyclo[4.2.1]nonane-9-carboxylate | 1H NMR (500 MHz, Chloroform-d) δ 11.18 - 10.79 (m, 1H), 8.58 - 8.37 (m, 2H), 8.37 - 8.15 (m, 1H), 7.71 - 7.46 (m, 2H), 6.37 - 6.02 (m, 1H), 4.78 - 4.12 (m, 3H), 3.87 - 3.13 (m, 3H), 2.41 - 1.56 (m, 6H), 1.39 (m, 9H). LCMS (Analytical Method 1) Rt = 0.73 min, MS (ESlpos): m/z 441.3 [M+H]+, Purity = 100%. |
| Intermediate 46 | Intermediate 46-1 & 4-iodopyridine (90% Yield) | tert-butyl (2R,6S)-2,6-dimethyl-4-{5-nitro-6-[(pyridin-4-yl)amino]pyridin-2-yl}piperazine-1-carboxylate | 1H NMR (400 MHz, DMSO-d6) δ 10.58 (s, 1H), 8.50 (dd, J = 4.8, 1.5 Hz, 2H), 8.28 (d, J = 9.5 Hz, 1H), 7.66 (dd, J = 4.7, 1.6 Hz, 2H), 6.63 (d, J = 9.6 Hz, 1H), 4.38 - 4.18 (m, 4H), 3.35 (dd, J = 13.5, 4.6 Hz, 2H), 1.45 (s, 9H), 1.16 (d, J = 6.8 Hz, 6H). LCMS (Analytical Method 1) Rt = 0.74 min, MS (ESlpos): m/z 429 [M+H]+, Purity = 98%. |
| Intermediate 47 | Intermediate 47-1 & 4-iodopyridine (97% Yield) | tert-butyl 2,2-dimethyl-4-{5-nitro-6-[(pyridin-4-yl)amino]pyridin-2-yl}piperazine-1-carboxylate | 1H NMR (400 MHz, DMSO-d6) δ 10.60 (s, 1H), 8.48 (dd, J = 4.7, 1.5 Hz, 2H), 8.29 (d, J = 9.5 Hz, 1H), 7.70 (dd, J = 4.8, 1.5 Hz, 2H), 6.49 (d, J = 9.6 Hz, 1H), 3.86 (s, 2H), 3.84 - 3.78 (m, 2H), 3.74 - 3.66 (m, 2H), 1.45 (s, 9H), 1.38 (s, 6H). LCMS (Analytical Method 1) Rt = 0.75 min, MS (ESlpos): m/z 429 [M+H]+, Purity = 100%. |
| Intermediate 48 | Intermediate 48-1 & 4-iodopyridine (87% Yield) | tert-butyl (2S,6S)-2,6-dimethyl-4-{5-nitro-6-[(pyridin-4-yl)amino]pyridin-2-yl}piperazine-1-carboxylate | 1H NMR (400 MHz, DMSO-d6) δ 10.62 (s, 1H), 8.48 (dd, J = 4.7, 1.6 Hz, 2H), 8.31 (d, J = 9.5 Hz, 1H), 7.80 - 7.64 (m, 2H), 6.43 (d, J = 9.5 Hz, 1H), 4.29 - 4.13 (m, 2H), 4.03 - 3.73 (m, 4H), 1.47 (s, 9H), 1.23 (d, J = 6.7 Hz, 6H). LCMS (Analytical Method 1) Rt = 0.74 min, MS (ESlpos): m/z 429 [M+H]+, Purity = 100% |
| Intermediate 49 | Intermediate 49-1 & 4-iodopyridine (99% Yield) | tert-butyl (3S)-3-methyl-4-{5-nitro-6-[(pyridin-4-yl)amino]pyridin-2-yl}piperazine-1-carboxylate | 1H NMR (400 MHz, DMSO-d6) δ 10.55 (s, 1H), 8.49 (dd, J = 4.8, 1.5 Hz, 2H), 8.28 (d, J = 9.5 Hz, 1H), 7.66 (dd, J = 4.8, 1.5 Hz, 2H), 6.54 (d, J = 9.6 Hz, 1H), 4.71 - 4.55 (m, 1H), 4.16 (dt, J = 13.6, 3.6 Hz, 1H), 3.99 - 3.88 (m, 1H), 3.87 - 3.77 (m, 1H), 3.46 - 3.32 (m, 1H), 3.25 (dd, J = 13.5, 3.9 Hz, 1H), 3.16 - 3.06 (m, 1H), 1.45 (s, 9H), 1.22 (d, J = 6.7 Hz, 3H). LCMS (Analytical Method 1) Rt = 0.71 min, MS (ESlpos): m/z 415 [M+H]+, Purity = 94%. |
| Intermediate 50 | Intermediate 50-1 & 4-iodopyridine (84% Yield) | tert-butyl 6-{5-nitro-6-[(pyridin-4-yl)amino]pyridin-2-yl}-3,6-diazabicyclo[3.2.1]octane-3-carboxylate | 1HNMR(400 MHz, DMSO-d6) δ 10.78 -10.65 (m, 1H), 8.51 - 8.44 (m, 2H), 8.26 (d,J = 9.1 Hz, 1H), 7.82 - 7.68 (m, 2H), 6.53 - 6.18 (m, 1H), 4.58 - 4.33 (m, 1H), 4.28 - 4.04 (m, 1H), 4.02 - 3.93 (m, 1H), 3.75 - 3.52 (m, 2H), 3.34 - 3.23 (m, 1H), 3.00 - 2.92 (m, 1H), 2.64 - 2.58 (m, 1H), 2.14 - 2.02 (m, 1H), 2.00 - 1.87 (m, 1H), 1.20 (s, 9H). LCMS (Analytical Method H) Rt = 0.58 min, MS (ESlpos): m/z 427 [M+H]+, Purity = 89%. |
| Intermediate 51 | Intermediate 51-1 & 4-iodopyridine (62% Yield) | tert-butyl 4-{3-methyl-5-nitro-6-[(pyridin-4-yl)amino]pyridin-2-yl}piperazine-1-carboxylate | 1H NMR (400 MHz, DMSO-d6) δ 10.39 (s, 1H), 8.49 - 8.41 (m, 2H), 8.21 (s, 1H), 7.79 - 7.65 (m, 2H), 3.58 - 3.52 (m, 4H), 3.51 - 3.46 (m, 4H), 2.28 (s, 3H), 1.43 (s, 9H). LCMS (Analytical Method 1) Rt = 0.93 min, MS (ESlpos): m/z 415.3 [M+H]+, Purity = 88%. |
| Intermediate 52 | Intermediate 52-1 & 4-iodopyridine (100% Yield) | tert-butyl (2R)-2-(methoxymethyl)-4-{5-nitro-6-[(pyridin-4-yl)amino]pyridin-2-yl}piperazine-1-carboxylate | 1H NMR (400 MHz, DMSO-d6) δ 10.57 (s, 1H), 8.47 (dd, J = 4.8, 1.5 Hz, 2H), 8.29 (d, J = 9.5 Hz, 1H), 7.69 (dd, J = 4.8, 1.6 Hz, 2H), 6.51 (d, J= 9.6 Hz, 1H), 4.40 (d, J = 13.4 Hz, 1H), 4.30 - 4.21 (m, 1H), 4.17 (d, J = 12.6 Hz, 1H), 3.88 (dt, J = 13.3, 3.6 Hz, 1H), 3.48 (dd, J = 13.8, 4.2 Hz, 1H), 3.36 (d, J = 7.2 Hz, 2H), 3.35 -3.18 (m, 5H), 1.45 (s, 9H). LCMS (Analytical Method A) Rt = 2.14 min, MS (ESlpos): m/z 445 [M+H]+, Purity = 100%. |
| Intermediate 53 | Intermediate 53-1 & 4-iodopyridine (100% Yield) | tert-butyl (2S)-2-(methoxymethyl)-4-{5-nitro-6-[(pyridin-4-yl)amino]pyridin-2-yl}piperazine-1-carboxylate | 1H NMR (400 MHz, DMSO-d6) δ 10.57 (s, 1H), 8.48 (d, J = 6.2 Hz, 2H), 8.29 (d, J = 9.5 Hz, 1H), 7.69 (dd, J = 4.8, 1.5 Hz, 2H), 6.51 (d, J = 9.6 Hz, 1H), 4.47 - 4.33 (m, 1H), 4.31 - 4.20 (m, 1H), 4.21 -4.10 (m, 1H), 3.88 (dt, J = 13.3, 3.6 Hz, 1H), 3.48 (dd, J = 13.8, 4.2 Hz, 1H), 3.36 (d, J = 7.2 Hz, 2H), 3.35 - 3.17 (m, 5H), 1.45 (s, 9H). LCMS (Analytical Method 1) Rt = 0.74 min, MS (ESlpos): m/z 445 [M+H]+, Purity = 99%. |
| Intermediate 54 | Intermediate 54-1 & 4-iodopyridine (52% Yield) | tert-butyl (3R,5R)-3,5-dimethyl-4-{5-nitro-6-[(pyridin-4-yl)amino]pyridin-2-yl}piperazine-1-carboxylate | 1H NMR (400 MHz, DMSO) δ 10.64 (s, 1H), 8.48 (dd, J = 4.7, 1.5 Hz, 2H), 8.29 (d, J = 9.6 Hz, 1H), 7.74 - 7.68 (m, 2H), 6.41 (d, J = 9.6 Hz, 1H), 4.55 - 4.43 (m, 2H), 3.78 - 3.66 (m, 2H), 3.68 - 3.53 (m, 2H), 1.47 (s, 9H), 1.32 (d, J = 6.6 Hz, 6H). LCMS (Analytical Method 1) Rt = 0.70 min, MS (ESlpos): m/z 429 [M+H]+, Purity = 82%. |
| Intermediate 55 | Intermediate 55-1 & 4-iodopyridine (95% Yield) | tert-butyl N-methyl-N-[(3S)-1-{5-nitro-6-[(pyridin-4-yl)amino]pyridin-2-yl}pyrrolidin-3-yl]carbamate | 1H NMR (400 MHz, DMSO) δ 10.70 (s, 1H), 8.45 (dd, J = 4.9, 1.4 Hz, 2H), 8.26 (d, J = 9.4 Hz, 1H), 7.78 (dd, J = 4.9, 1.4 Hz, 2H), 6.30 (d, J = 9.4 Hz, 1H), 4.79 -4.66 (m, 1H), 3.91 - 3.75 (m, 2H), 3.67 -3.49 (m, 2H), 2.80 (s, 3H), 2.28- 2.11 (m, 2H), 1.44 (s, 9H). LCMS (Analytical Method 1) Rt = 0.75 min, MS (ESlpos): m/z 415 [M+H]+, Purity = 100%. |
| Intermediate 56 | Intermediate 56-1 & 4-iodopyridine (92% Yield) | tert-butyl 8-{5-nitro-6-[(pyridin-4-yl)amino]pyridin-2-yl}-5,8-diazaspiro[3.5]nonane-5-carboxylate | 1H NMR (500 MHz, DMSO-d6) δ 10.63 (s, 1H), 8.49 (dd, J = 4.8, 1.4 Hz, 2H), 8.28 (d, J = 9.6 Hz, 1H), 7.74 - 7.68 (m, 2H), 6.54 (s, 1H), 3.97 (s, 2H), 3.68 -3.49 (m, 4H), 2.48 - 2.44 (m, 2H), 1.90 -1.81 (m, 2H), 1.78 - 1.68 (m, 2H), 1.44 (s, 9H). LCMS (Analytical Method 1) Rt = 0.79 min, MS (ESlpos): m/z 441.4 [M+H]+, Purity = 93%. |
| Intermediate 57 | Intermediate 57-1 & 4-iodopyridine (97% Yield) | tert-butyl N-methyl-N-[(3R)-1-{5-nitro-6-[(pyridin-4-yl)amino]pyridin-2-yl}pyrrolidin-3-yl]carbamate | 1H NMR (400 MHz, DMSO) δ 10.70 (s, 1H), 8.45 (dd, J = 4.8, 1.5 Hz, 2H), 8.26 (d, J = 9.4 Hz, 1H), 7.78 (dd, J = 4.8, 1.6 Hz, 2H), 6.30 (d, J = 9.4 Hz, 1H), 4.79 -4.67 (m, 1H), 3.84 (s, 2H), 3.67 - 3.48 (m, 2H), 2.80 (s, 3H), 2.28 - 2.07 (m, 2H), 1.44 (s, 9H). LCMS (Analytical Method 1) Rt = 0.73 min, MS (ESlpos): m/z 415 [M+H]+, Purity = 99%. |
| Intermediate 58 | Intermediate 58-1 & 4-iodopyridine (85% Yield) | tert-butyl 7-{5-nitro-6-[(pyridin-4-yl)amino]pyridin-2-yl}-3-oxa-7,9-diazabicyclo[3.3.1]nonane-9-carboxylate | 1H NMR (500 MHz, DMSO) δ 10.68 (s, 1H), 8.60 - 8.44 (m, 2H), 8.28 (d, J = 9.6 Hz, 1H), 7.71 (d, J = 6.2 Hz, 2H), 6.60 (d, J = 9.6 Hz, 1H), 4.89 - 4.68 (m, 1H), 4.42 - 4.22 (m, 1H), 4.22 - 4.01 (m, 3H), 3.97 - 3.81 (m, 3H), 3.67 - 3.57 (m, 2H), 1.45 (s, 9H). LCMS (Analytical Method 1) Rt = 0.66 min, MS (ESlpos): m/z 443.4 [M+H]+, Purity = 100%. |
| Intermediate 59 | Intermediate 59-1 & 4-iodopyridine (97% Yield) | tert-butyl 9-{5-nitro-6-[(pyridin-4-yl)amino]pyridin-2-yl}-3-oxa-7,9-diazabicyclo[3.3.1]nonane-7-carboxylate | 1H NMR (400 MHz, DMSO) δ 10.60 (s, 1H), 8.55 - 8.43 (m, 2H), 8.39 - 8.28 (m, 1H), 7.87 - 7.80 (m, 1H), 7.69 - 7.59 (m, 2H), 4.64 (s, 1H), 4.37 - 4.14 (m, 3H), 4.07 - 3.91 (m, 2H), 3.72 (t, J = 10.6 Hz, 2H), 1.41 (s, 9H). 1 signal obscured. LCMS (Analytical Method 1) Rt = 0.66 min, MS (ESlpos): m/z 443.4 [M+H]+, Purity = 95%. |
| Intermediate 60 | Intermediate 60-1 & 4-iodopyridine (84% Yield) | 3-nitro-6-[(1R,4R)-2-oxa-5-azabicyclo[2.2.1]heptan-5-yl]-N-(pyridin-4-yl)pyridin-2-amine | 1H NMR (400 MHz, DMSO) δ 10.75 -10.64 (m, 1H), 8.54 - 8.42 (m, 2H), 8.26 (d, J = 9.4 Hz, 1H), 7.74 (d, J = 5.5 Hz, 2H), 6.2 - 6.5 (m, 1H), 5.10 - 4.95 (m, 1H), 4.79 - 4.73 (m, 1H), 3.88 (d, J = 7.6 Hz, 1H), 3.76 (d, J = 7.5 Hz, 1H), 3.72 -3.61 (m, 1H), 3.57 - 3.43 (m, 1H), 2.05 -1.94 (m, 2H). LCMS (Analytical Method B) Rt = 2.47 min, MS (ESlpos): m/z 314.2 [M+H]+, Purity = 85%. |
| Intermediate 61 | Intermediate 61-1 & 4-iodopyridine (96% Yield) | tert-butyl 4-{5-nitro-6-[(pyridin-4-yl)amino]pyridin-2-yl}-2-(trifluoromethyl)piperazine-1-carboxylate | 1H NMR (400 MHz, DMSO) δ 10.56 (s, 1H), 8.82 - 8.39 (m, 2H), 8.32 (d, J = 9.5 Hz, 1H), 7.88 - 7.47 (m, 2H), 6.62 (d, J = 9.5 Hz, 1H), 4.93 (s, 3H), 4.28 (s, 1H), 4.02 (s, 1H), 3.57 (s, 1H), 3.12 (s, 1H), 1.44 (s, 9H). LCMS (Analytical Method 1) Rt = 0.74 min, MS (ESlpos): m/z 469.3 [M+H]+, Purity = 100%. |
| Intermediate 62 | Intermediate 62-1 & 4-iodopyridine (70% Yield) | tert-butyl 6,6-difluoro-4-{5-nitro-6-[(pyridin-4-yl)amino]pyridin-2-yl}-1,4-diazepane-1-carboxylate | 1H NMR (500 MHz, DMSO) δ 10.52 (s, 1H), 8.52 - 8.41 (m, 2H), 8.36 - 8.29 (m, 1H), 7.75 - 7.66 (m, 2H), 6.65 (s, 1H), 4.29 (s, 2H), 4.05 - 3.79 (m, 4H), 3.75 -3.64 (m, 2H), 1.45 - 1.17 (m, 9H). LCMS (Analytical Method 1) Rt = 0.69 min, MS (ESlpos): m/z 451.4 [M+H]+, Purity = 90%. |
| Intermediate 63 | Intermediate 63-1 & 4-iodopyridine (49% Yield) | tert-butyl 5-methyl-4-{5-nitro-6-[(pyridin-4-yl)amino]pyridin-2-yl}-1,4-diazepane-1-carboxylate | 1H NMR (500 MHz, MeOD) δ 8.44 (d, J = 5.2 Hz, 2H), 8.36 (d, J = 9.6 Hz, 1H), 7.80 (d, J = 6.1 Hz, 2H), 6.59 - 6.39 (m, 1H), 3.96 - 3.81 (m, 2H), 3.70 - 3.64 (m, 1H), 3.11 - 2.90 (m, 2H), 2.38 - 2.19 (m, 2H), 1.81 - 1.61 (m, 2H), 1.51 - 1.35 (m, 9H), 1.32 (d, J = 6.6 Hz, 3H). LCMS (Analytical Method 1) Rt = 0.71 min, MS (ESlpos): m/z 429.4 [M+H]+, Purity = 89%. |
| Intermediate 64 | Intermediate 64-1 & 4-iodopyridine (76% Yield) | tert-butyl (3S)-3-(hydroxymethyl)-4-{5-nitro-6-[(pyridin-4-yl)amino]pyridin-2-yl}piperazine-1-carboxylate | 1H NMR (500 MHz, MeOD) δ 8.44 (d, J = 6.4 Hz, 2H), 8.36 (d, J = 9.5 Hz, 1H), 7.79 (d, J = 5.7 Hz, 2H), 6.56 (d, J = 9.6 Hz, 1H), 4.76 - 4.50 (m, 1H), 4.48 - 4.15 (m, 2H), 4.09 - 4.00 (m, 1H), 3.79 - 3.72 (m, 2H), 3.46 - 3.37 (m, 1H), 3.30 - 3.09 (m, 2H), 1.52 (s, 9H). LCMS (Analytical Method 1) Rt = 0.62 min, MS (ESlpos): m/z 431.4 [M+H]+, Purity = 99%. |
| Intermediate 65 | Intermediate 65-1 & 4-iodopyridine (91% Yield) | tert-butyl 4-{4-methyl-5-nitro-6-[(pyridin-4-yl)amino]pyridin-2-yl}piperazine-1-carboxylate | 1H NMR (400 MHz, Chloroform-d) δ 10.74 (s, 1H), 8.57 - 8.42 (m, 2H), 7.54 -7.45 (m, 2H), 6.07 (s, 1H), 3.80 - 3.68 (m, 4H), 3.61 - 3.49 (m, 4H), 2.61 (s, 3H), 1.49 (s, 9H). LCMS (Analytical Method 1) Rt = 0.79 min, MS (ESlpos): m/z 415.3 [M+H]+, Purity = 95%. |
| Intermediate 66 | Intermediate 66-1 & 4-iodopyridine (94% Yield) | tert-butyl (1 R,5S)-3-{4-methyl-5-nitro-6-[(pyridin-4-yl)amino]pyridin-2-yl}-3,8-diazabicyclo[3.2.1]octane-8-carboxylate | 1H NMR (400 MHz, Chloroform-d) δ 10.74 (s, 1H), 8.57 - 8.41 (m, 2H), 7.56 -7.41 (m, 2H), 6.02 (s, 1H), 4.40 (s, 2H), 4.07 (s, 2H), 3.27 (s, 2H), 2.60 (s, 3H), 2.05 - 1.90 (m, 2H), 1.77 - 1.55 (m, 2H), 1.50 (s, 9H). LCMS (Analytical Method 1) Rt = 0.94 min, MS (ESlpos): m/z 441.3 [M+H]+, Purity = 100%. |
| Intermediate 67 | Intermediate 67-1 & 4-iodopyridine (99% Yield) | tert-butyl (1S,4S)-5-{4-methyl-5-nitro-6-[(pyridin-4-yl)amino]pyridin-2-yl}-2,5-diazabicyclo[2.2.1]heptane-2-carboxylate | LCMS (Analytical Method 1) Rt = 0.88 min, MS (ESlpos): m/z 427.3 [M+H]+, Purity = 99%. |
| Intermediate 68 | Intermediate 68-1 & 4-iodopyridine (76% Yield) | tert-butyl (1R,4R)-5-{4-methyl-5-nitro-6-[(pyridin-4-yl)amino]pyridin-2-yl}-2,5-diazabicyclo[2.2.1]heptane-2-carboxylate | 1H NMR (400 MHz, DMSO-d6) δ 10.32 (s, 1H), 8.45 - 8.39 (m, 2H), 7.68 - 7.62 (m, 2H), 6.21 (s, 1H), 4.90 (s, 1H), 4.54 (s, 1H), 3.64 (dd, J = 10.3, 1.8 Hz, 1H), 3.50 - 3.41 (m, 2H), 3.26 (d, J = 10.0 Hz, 1H), 2.01 - 1.92 (m, 2H), 1.41 (s, 9H). LCMS (Analytical Method 1) Rt = 0.66 min, MS (ESlpos): m/z 427.3 [M+H]+, Purity = 93%. |
| Intermediate 69-2 | Intermediate 69-1 & 4-iodopyridine (72% Yield) | 1-tert-butyl 2-methyl(2R)-4-{5-nitro-6-[(pyridin-4-yl)amino]pyridin-2-yl}piperazine-1,2-dicarboxylate | 1H NMR (500 MHz, DMSO) δ 10.58 (s, 1H), 8.61 - 8.40 (m, 2H), 8.30 (d, J = 9.5 Hz, 1H), 7.70 (dd, J = 4.9, 1.4 Hz, 2H), 6.55 (dd, J = 9.5, 4.4 Hz, 1H), 4.84 (s, 1H), 4.76 (m, 2H), 4.18 (s, 1H), 3.85 (s, 1H), 3.61 (d, J = 10.3 Hz, 1H), 3.53 (s, 3H), 3.28 (s, 1H), 1.40 (m, 9H). LCMS (Analytical Method 1) Rt = 0.70 min, MS (ESlpos): m/z 459.4 [M+H]+, Purity = 100%. |
| Intermediate 70 | Intermediate 70-1 & 4-iodopyridine (96% Yield) | tert-butyl (1S,6R)-3-{5-nitro-6-[(pyridin-4-yl)amino]pyridin-2-yl}-3,8-diazabicyclo[4.2.0]octane-8-carboxylate | 1H NMR (400 MHz, DMSO-d6) δ 10.63 (s, 1H), 8.48 - 8.44 (m, 2H), 8.28 (d, J = 9.5 Hz, 1H), 7.77 - 7.72 (m, 2H), 6.44 (d, J = 9.6 Hz, 1H), 4.53 - 4.48 (m, 1H), 3.94 - 3.86 (m, 2H), 3.82 - 3.70 (m, 2H), 3.69 - 3.65 (m, 1H), 3.60 - 3.54 (m, 1H), 2.87 - 2.78 (m, 1H), 2.20 - 2.10 (m, 1H), 1.96 - 1.89 (m, 1H), 1.24 (s, 9H). LCMS (Analytical Method H) Rt = 0.57 min, MS (ESlpos): m/z 427.4 [M+H]+, Purity = 94%. |
| Intermediate 71 | Intermediate 19-1 & 4-iodopyridine (26% Yield) | tert-butyl (1S,4S)-5-{5-nitro-6-[(pyridin-4-yl)amino]pyridin-2-yl}-2,5-diazabicyclo[2.2.1]heptane-2-carboxylate | 1H NMR (400 MHz, DMSO-d6) δ 10.75 (m, 1H), 8.49 (d, J = 6.4 Hz, 2H), 8.26 (m, 1H), 7.87 - 7.76 (m, 2H), 6.39 (m, 1H), 5.18 - 4.41 (m, 2H), 3.81 - 3.45 (m, 4H), 1.99 (m, 2H), 1.39 (m, 9H). LCMS (Analytical Method 1) Rt = 0.79 min, MS (ESlpos): m/z 413.2 [M+H]+, Purity = 87%. |
| Intermediate 72 | Intermediate 40-1 & 4-bromo-2-methylpyridine (87% Yield) | tert-butyl (1R,4R)-5-{6-[(2-methylpyridin-4-yl)amino]-5-nitropyridin-2-yl}-2,5-diazabicyclo[2.2.2]octane-2-carboxylate | 1H NMR (400 MHz, DMSO-d6) δ 10.57 (br s, 1H), 8.34 (d, J = 5.6 Hz, 1H), 8.26 (d, J = 9.3 Hz, 1H), 7.60 (s, 1H), 7.49 (br s, 1H), 6.67 - 6.05 (m, 1H), 5.15 - 4.41 (m, 1H), 4.32 (s, 1H), 3.89 - 3.62 (m, 2H), 3.61 - 3.47 (m, 2H), 2.45 (s, 3H), 2.05 -1.79 (m, 4H), 1.43 (s, 9H). LCMS (Analytical Method 1) Rt = 0.71 min, MS (ESlpos): m/z 441.3 [M+H]+, Purity = 96%. |
| Intermediate 73 | Intermediate 4-1 & 4-bromo-3-methyl-pyridin-1-ium;chloride (70% Yield) | tert-butyl 4-{6-[(3-methylpyridin-4-yl)amino]-5-nitropyridin-2-yl}piperazine-1-carboxylate | 1H NMR (400 MHz, DMSO-d6) δ 10.75 (s, 1H), 8.43 - 8.39 (m, 2H), 8.29 (d, J = 9.6 Hz, 1H), 8.18 (d, J = 5.6 Hz, 1H), 6.60 (d, J = 9.6 Hz, 1H), 3.85 - 3.69 (m, 4H), 3.52 - 3.42 (m, 4H), 2.31 (s, 3H), 1.43 (s, 9H). LCMS (Analytical Method 1) Rt = 0.70 min, MS (ESlpos): m/z 415.3 [M+H]+, Purity = 99%. |
| Intermediate 74 | Intermediate 38-1 & 4-bromo-2-methylpyridine (96% Yield) | tert-butyl (1R,4R)-5-{6-[(2-methylpyridin-4-yl)amino]-5-nitropyridin-2-yl}-2,5-diazabicyclo[2.2.1]heptane-2-carboxylate | 1H NMR (500 MHz, Chloroform-d) δ 11.13 - 10.76 (m, 1H), 8.32 (m, 1H), 8.26 (d, J = 9.3 Hz, 1H), 7.64 - 7.33 (m, 2H), 6.20 - 5.77 (m, 1H), 5.12 - 5.00 (m, 1H), 4.77 - 4.47 (m, 1H), 3.81 - 3.67 (m, 1H), 3.54 - 3.42 (m, 2H), 3.42 - 3.24 (m, 1H), 2.52 (s, 3H), 2.10 - 1.87 (m, 2H), 1.43 -1.29 (m, 9H). LCMS (Analytical Method 1) Rt = 0.66 min, MS (ESlpos): m/z 427.3 [M+H]+, Purity = 97%. |
| Intermediate 75 | Intermediate 43-1 & 4-bromo-2-methylpyridine (100% Yield) | tert-butyl (2S)-2-methyl-4-{6-[(2-methylpyridin-4-yl)amino]-5-nitropyridin-2-yl}piperazine-1-carboxylate | 1H NMR (400 MHz, DMSO-d6) δ 10.53 (s, 1H), 8.36 (d, J = 5.6 Hz, 1H), 8.27 (d, J = 9.5 Hz, 1H), 7.56 (d, J = 2.0 Hz, 1H), 7.49 (dd, J = 5.6, 2.1 Hz, 1H), 6.54 (d, J = 9.6 Hz, 1H), 4.34 - 4.08 (m, 3H), 3.90 - 3.80 (m, 1H), 3.54 (dd, J = 13.6, 4.2 Hz, 1H), 3.37 - 3.22 (m, 2H), 2.47 (s, 3H), 1.44 (s, 9H), 1.11 (d, J = 6.6 Hz, 3H). LCMS (Analytical Method 1) Rt = 0.83 min, MS (ESlpos): m/z 429 [M+H]+, Purity = 100%. |
| Intermediate 76 | Intermediate 30-1 & 4-bromo-2-methylpyridine (98% Yield) | tert-butyl 7-{6-[(2-methylpyridin-4-yl)amino]-5-nitropyridin-2-yl}-4,7-diazaspiro[2.5]octane-4-carboxylate | 1H NMR (400 MHz, DMSO-d6) δ 10.50 (s, 1H), 8.34 (d, J = 5.6 Hz, 1H), 8.25 (d, J = 9.5 Hz, 1H), 7.54 (d, J = 2.0 Hz, 1H), 7.40 (dd, J = 5.5, 2.0 Hz, 1H), 6.54 (d, J = 9.6 Hz, 1H), 3.84 - 3.72 (m, 2H), 3.63 (s, 2H), 3.60 (dd, J = 6.2, 4.3 Hz, 2H), 2.46 (s, 3H), 1.45 (s, 9H), 1.03 - 0.96 (m, 2H), 0.87 - 0.80 (m, 2H). LCMS (Analytical Method 1) Rt = 0.87 min, MS (ESlpos): m/z 441 [M+H]+, Purity = 100%. |
| Intermediate 77 | Intermediate 44-1 & 4-bromo-2-methylpyridine (99% Yield) | tert-butyl (2R)-2-methyl-4-{6-[(2-methylpyridin-4-yl)amino]-5-nitropyridin-2-yl}piperazine-1-carboxylate | 1H NMR (400 MHz, DMSO-d6) δ 10.61 (s, 1H), 8.35 (d, J = 5.6 Hz, 1H), 8.27 (d, J = 9.6 Hz, 1H), 7.57 (s, 1H), 7.53 - 7.49 (m, 1H), 6.58 (d, J = 9.6 Hz, 1H), 4.23 (s, 3H), 3.88 - 3.77 (m, 1H), 3.49 (dd, J = 13.6, 3.9 Hz, 1H), 3.29 - 3.20 (m, 2H), 2.45 (s, 3H), 1.42 (s, 9H), 1.07 (d, J = 6.6 Hz, 3H). LCMS (Analytical Method 1) Rt = 0.80 min, MS (ESlpos): m/z 429 [M+H]+, Purity = 100%. |
| Intermediate 78 | Intermediate 38-1 & 4-bromo-2-(difluoromethyl)pyridine (100% Yield) | tert-butyl (1R,4R)-5-(6-{[2-(difluoromethyl)pyridin-4-yl]amino}-5-nitropyridin-2-yl)-2,5-diazabicyclo[2.2.1]heptane-2-carboxylate | 1H NMR (400 MHz, DMSO-d6) δ 10.78 (s, 1H), 8.54 (d, J = 5.5 Hz, 1H), 8.36 (br s, 1H), 8.27 (d, J = 9.4 Hz, 1H), 7.72 (d, J = 4.2 Hz, 1H), 6.86 (t, J = 55.3 Hz, 1H), 6.68 - 6.10 (m, 1H), 4.96 (br s, 1H), 4.56 (s, 1H), 3.70 (d, J = 10.4 Hz, 1H), 3.53 (br s, 1H), 3.44 (dd, J = 10.0, 1.7 Hz, 1H), 3.30 (d, J = 9.7 Hz, 1H), 2.01 (s, 2H), 1.41 (s, 9H). LCMS (Analytical Method 1) Rt = 0.98 min, MS (ESlpos): m/z 463.3 [M+H]+, Purity = 99%. |
| Intermediate 79 | Intermediate 34-1 & 4-iodopyridine (99% Yield) | tert-butyl (3R)-3-methyl-4-{5-nitro-6-[(pyridin-4-yl)amino]pyridin-2-yl}piperazine-1-carboxylate | 1H NMR (400 MHz, DMSO-d6) δ 10.55 (s, 1H), 8.49 (dd, J = 4.8, 1.5 Hz, 2H), 8.28 (d, J = 9.5 Hz, 1H), 7.66 (dd, J = 4.8, 1.6 Hz, 2H), 6.54 (d, J = 9.6 Hz, 1H), 4.71 - 4.51 (m, 1H), 4.16 (dt, J = 13.6, 3.6 Hz, 1H), 3.97 - 3.89 (m, 1H), 3.85 - 3.79 (m, 1H), 3.44 - 3.34 (m, 1H), 3.25 (dd, J = 13.5, 4.0 Hz, 1H), 3.15 - 3.07 (m, 1H), 1.45 (s, 9H), 1.22 (d, J = 6.7 Hz, 3H). LCMS (Analytical Method 1) Rt = 0.70 min, MS (ESlpos): m/z 415 [M+H]+, Purity = 97%. |
| Intermediate 80 | Intermediate 34-1 & 4-bromo-2-(difluoromethyl)pyridine (98% Yield) | tert-butyl (3R)-4-(6-{[2-(difluoromethyl)pyridin-4-yl]amino}-5-nitropyridin-2-yl)-3-methylpiperazine-1-carboxylate | 1H NMR (400 MHz, DMSO) δ 10.66 (s, 1H), 8.56 (d, J = 5.5 Hz, 1H), 8.30 (d, J = 9.5 Hz, 1H), 8.20 (d, J = 2.0 Hz, 1H), 7.76 - 7.62 (m, 1H), 6.87 (t, J = 55.2 Hz, 1H), 6.57 (d, J = 9.6 Hz, 1H), 4.71 - 4.58 (m, 1H), 4.22 - 4.08 (m, 1H), 3.96 - 3.88 (m, 1H), 3.84 - 3.75 (m, 1H), 3.46 - 3.34 (m, 1H), 3.26 (dd, J = 13.5, 4.0 Hz, 1H), 3.15 - 3.07 (m, 1H), 1.45 (s, 9H), 1.21 (d, J = 6.7 Hz, 3H). LCMS (Analytical Method H) Rt = 0.71 min, MS (ESlpos): m/z 465 [M+H]+, Purity = 98%. |
| Intermediate 81 | Intermediate 49-1 & 4-bromo-2-(difluoromethyl)pyridine (91% Yield) | tert-butyl (3S)-4-(6-{[2-(difluoromethyl)pyridin-4-yl]amino}-5-nitropyridin-2-yl)-3-methylpiperazine-1-carboxylate | 1H NMR (400 MHz, DMSO) δ 10.66 (s, 1H), 8.55 (d, J = 5.5 Hz, 1H), 8.30 (d, J = 9.5 Hz, 1H), 8.20 (d, J = 2.0 Hz, 1H), 7.75 - 7.63 (m, 1H), 6.87 (t, J = 55.2 Hz, 1H), 6.57 (d, J = 9.6 Hz, 1H), 4.71 - 4.58 (m, 1H), 4.17 (dt, J = 13.7, 3.4 Hz, 1H), 3.92 (d, J = 13.3 Hz, 1H), 3.86 - 3.76 (m, 1H), 3.44 - 3.33 (m, 1H), 3.26 (dd, J = 13.5, 4.0 Hz, 1H), 3.15 - 3.06 (m, 1H), 1.45 (s, 9H), 1.21 (d, J = 6.7 Hz, 3H). LCMS (Analytical Method H) Rt = 0.71 min, MS (ESlpos): m/z 465 [M+H]+, Purity = 99%. |
| Intermediate 82-1 | Intermediate 4-1 & 4-bromo-3-fluoropyridine (84% Yield) | tert-butyl 4-{6-[(3-fluoropyridin-4-yl)amino]-5-nitropyridin-2-yl}piperazine-1-carboxylate | 1H NMR (500 MHz, Chloroform-d) δ 11.21 (s, 1H), 8.45 - 8.35 (m, 2H), 8.35 -8.27 (m, 2H), 6.27 (d, J = 9.5 Hz, 1H), 3.81 - 3.62 (m, 4H), 3.62 - 3.47 (m, 4H), 1.44 (s, 9H). LCMS (Analytical Method I) Rt = 0.88 min, MS (ESlpos): m/z 419.3 [M+H]+, Purity = 94%. |

Synthesis of tert-butyl 7-{5-amino-6-[(pyridin-4-yl)aminolpyridin-2-yl}-4,7-diazaspiro[2.5]octane-4-carboxylate / intermediate 83 To a suspension of tert-butyl 7-{5-nitro-6-[(pyridin-4-yl)amino]pyridin-2-yl}-4,7-diazaspiro[2.5]octane-4-carboxylate (Intermediate 30) (1.1 g, 2.55 mmol) in EtOH (20 mL) was added Pd/C (10%, 135 mg, 0.127 mmol). The mixture was stirred under an atmosphere of hydrogen for 20 h. The solution was filtered through a pad of celite and concentrated in vacuo to yield the title compound (993 mg, 90% yield) as a golden yellow solid. 1H NMR (500 MHz, Chloroform-d) δ 8.34 - 8.24 (m, 2H), 7.32 - 7.25 (m, 2H), 7.04 (s, 1H), 7.01 (d, J = 8.4 Hz, 1H), 6.04 (d, J = 8.4 Hz, 1H), 3.66 - 3.59 (m, 2H), 3.45 - 3.30 (m, 2H), 1.65 (s, 2H), 1.41 (s, 9H), 1.01 -0.91 (m, 2H), 0.82 - 0.72 (m, 2H). LCMS (Analytical Method I) Rt = 0.68 min, MS (ESlpos): m/z 397.3 [M+H]+, Purity = 91%.

Each of Intermediates 28 through 86 as listed in Table 1.4.3 were prepared according to the method of intermediate 94 using the intermediate listed in the “Synthesis” column. The intermediates were purified by flash chromatography, SCX or preparative HPLC Methods, A1, A2, B1, B2 as required.

TABLE 1.4.3

| | | | |
|---|---|---|---|
| Intermediate | Synthesis | Structure/Name | Data |
| Intermediate 28 | Intermediate 28-2 (92% Yield) | tert-butyl 6-{5-amino-6-[(2-methylpyridin-4-yl)amino]pyridin-2-yl}-2,6-diazaspiro[3.3]heptane-2-carboxylate | 1H NMR (400 MHz, Chloroform-d) δ 9.77 (s, 1H), 7.93 (s, 1H), 7.82 (s, 2H), 6.98 (d, J = 8.3 Hz, 1H), 5.97 (d, J = 8.3 Hz, 1H), 4.02 (d, J = 3.9 Hz, 4H), 3.95 (s, 4H), 2.56 - 2.45 (m, 3H), 1.38 (s, 9H). LCMS (Analytical Method I) Rt = 0.66 min, MS (ESIpos): m/z 397.4 [M+H]+, Purity = 54%. |
| Intermediate 31 | Intermediate 31-2 (99% Yield) | tert-butyl 6-{5-amino-6-[(pyridin-4-yl)amino]pyridin-2-yl}-1,6-diazaspiro[3.3]heptane-1-carboxylate | 1H NMR (400 MHz, DMSO-d6) δ 8.26 - 8.17 (m, 2H), 7.55 (dd, J = 4.9, 1.5 Hz, 2H), 7.01 (d, J = 8.1 Hz, 1H), 5.90 (d, J = 8.2 Hz, 1H), 4.31 (d, J = 8.7 Hz, 2H), 4.24 - 4.10 (m, 2H), 3.96 (d, J = 8.6 Hz, 2H), 3.78 - 3.67 (m, 2H), 1.35 - 1.14 (m, 9H). LCMS (Analytical Method A) Rt = 1.50 min, MS (ESIpos): m/z 383 [M+H]+, Purity = 91%. |
| Intermediate 33 | Intermediate 33-2 (96% Yield) | tert-butyl 1-{5-amino-6-[(pyridin-4-yl)amino]pyridin-2-yl}-1,6-diazaspiro[3.3]heptane-6-carboxylate | 1H NMR (500 MHz, DMSO-d6) δ 8.21 - 8.16 (m, 2H), 8.13 (s, 1H), 7.63 - 7.55 (m, 2H), 7.01 (d, J = 8.1 Hz, 1H), 5.85 (d, J = 8.2 Hz, 1H), 4.45 (s, 4H), 3.91 (d, J = 9.0 Hz, 2H), 3.64 (t, J = 6.9 Hz, 2H), 2.49 - 2.45 (m, 2H), 1.33 (s, 9H). LCMS (Analytical Method I) Rt = 0.65 min, MS (ESIpos): m/z 383 [M+H]+, Purity = 91%. |
| Intermediate 34 | Intermediate 34-2 (62% Yield) | tert-butyl (3R)-4-{5-amino-6-[(pyridazin-4-yl)amino]pyridin-2-yl}-3-methylpiperazine-1-carboxylate | 1H NMR (400 MHz, DMSO) δ 9.22 (d, J = 2.3 Hz, 1H), 8.77 (d, J = 5.6 Hz, 1H), 8.59 -8.50 (m, 1H), 7.79 - 7.69 (m, 1H), 7.06 (d, J = 8.4 Hz, 1H), 6.33 (d, J = 8.5 Hz, 1H), 4.60 - 4.43 (m, 2H), 4.41 - 4.29 (m, 2H), 4.29 -4.17 (m, 1H), 4.04 - 3.86 (m, 1H), 3.86 -3.75 (m, 2H), 3.64 - 3.40 (m, 3H), 1.43 (s, 9H), 0.96 (d, J = 6.5 Hz, 3H). LCMS (Analytical Method I) Rt = 0.62 min, MS (ESIpos): m/z 386.4 [M+H]+, Purity = 91%. |
| Intermediate 82 | Intermediate 82-1 (87% Yield) | tert-butyl 4-{5-amino-6-[(3-fluoropyridin-4-yl)amino]pyridin-2-yl}piperazine-1-carboxylate | 1H NMR (400 MHz, DMSO-d6) δ 8.35 (d, J = 3.4 Hz, 1H), 8.16 (d, J = 5.5 Hz, 1H), 7.95 - 7.81 (m, 2H), 7.09 (d, J = 8.4 Hz, 1H), 6.40 (d, J = 8.4 Hz, 1H), 4.60 (s, 2H), 3.45 (m, 4H), 3.27 - 3.16 (m, 4H), 1.42 (s, 9H). LCMS (Analytical Method I) Rt = 0.63 min, MS (ESIpos): m/z 389.3 [M+H]+, Purity = 96%. |
| Intermediate 84 | Intermediate 32 (93% Yield) | tert-butyl 3-{5-amino-6-[(pyridin-4-yl)amino]pyridin-2-yl}-3,8-diazabicyclo[3.2.1]octane-8-carboxylate | 1H NMR (400 MHz, DMSO-d6) δ 8.31 (s, 1H), 8.28 - 8.19 (m, 2H), 7.52 - 7.42 (m, 2H), 7.01 (d, J = 8.4 Hz, 1H), 6.22 (d, J = 8.4 Hz, 1H), 4.46 (s, 1H), 4.28 - 4.15 (m, 2H), 3.73 - 3.61 (m, 2H), 2.86 - 2.73 (m, 2H), 1.93 - 1.78 (m, 2H), 1.78 - 1.64 (m, 2H), 1.42 (s, 9H). LCMS (Analytical Method I) Rt = 0.73 min, MS (ESIpos): m/z 397.4 [M+H]+, Purity = 95%. |
| Intermediate 85-1 | Intermediate 80 (90% Yield) | tert-butyl (3R)-4-(5-amino-6-{[2-(difluoromethyl)pyridin-4-yl]amino}pyridin-2-yl)-3-methylpiperazine-1-carboxylate | 1H NMR (500 MHz, CDCl3) δ 8.30 (d, J = 5.7 Hz, 1H), 8.03 (s, 1H), 7.48 (s, 1H), 7.26 (s, 1H), 7.06 (d, J = 8.1 Hz, 1H), 6.53 (t, J = 55.7 Hz, 1H), 6.08 (s, 1H), 4.42 - 3.76 (m, 3H), 3.25 - 2.81 (m, 3H), 2.35 - 2.12 (m, 2H), 1.42 (s, 10H), 1.06 (d, J = 6.6 Hz, 3H). LCMS (Analytical Method I) Rt = 0.7 min, MS (ESIpos): m/z 435.4 [M+H]+, Purity = 99%. |
| Intermediate 86 | Intermediate 29 (77% Yield) | tert-butyl 6-{5-amino-6-[(pyridin-4-yl)amino]pyridin-2-yl}-3,6-diazabicyclo[3.1.1]heptane-3-carboxylate | 1H NMR (500 MHz, Methanol-d4) δ 8.20 (d, J = 6.3 Hz, 2H), 7.51 (d, J = 6.4 Hz, 2H), 7.14 (d, J = 7.5 Hz, 1H), 6.15 (dd, J = 8.0, 2.8 Hz, 1H), 4.40 - 4.25 (m, 2H), 4.16 - 3.97 (m, 2H), 3.63 - 3.56 (m, 1H), 3.37 - 3.25 (m, 2H), 2.72 (d, J = 6.0 Hz, 1H), 1.35 (s, 9H), 1.23 - 1.14 (m, 2H). LCMS (Analytical Method I) Rt = 0.48 min, MS (ESIpos): m/z 383.3 [M+H]+, Purity = 68%. |

### Synthesis of Tert-butyl (3S)-3-methyl-4-{5-nitro-6-[(pyrimidin-4-yl)aminolpyridin-2-yl)piperazine-1-carboxylate / Intermediate 87

A mixture of N-(6-chloro-3-nitro-2-pyridyl)pyrimidin-4-amine (Intermediate 26-1) (250 mg, 0.994 mmol), tert-butyl (3S)-3-methylpiperazine-1-carboxylate (200 mg, 0.999 mmol) and diisopropylethylamine (0.50 mL, 2.86 mmol) in acetonitrile (5 mL) was heated to 80° C. for 1.5 hours. The reaction was cooled and quenched into water. The aqueous layer was extracted into ethyl acetate (5 mL) three times, the combined organics washed with brine, dried over MgSO₄ and concentrated in vacuo. The product was purified by flash chromatography (25 g, silica), eluting with 20-100% ethyl acetate/heptane to yield the the title compound (151 mg, 0.353 mmol, 35% yield) as an orange. 1H NMR (500 MHz, Chloroform-d) δ 11.13 (s, 1H), 8.91 (d, J = 1.0 Hz, 1H), 8.60 (d, J = 5.8 Hz, 1H), 8.38 (d, J = 9.5 Hz, 1H), 8.15 (dd, J = 5.8, 1.2 Hz, 1H), 6.29 (d, J = 9.6 Hz, 1H), 4.65 - 4.49 (m, 1H), 4.27 - 4.06 (m, 2H), 4.06 - 3.94 (m, 1H), 3.49 - 3.39 (m, 1H), 3.31 - 3.23 (m, 1H), 3.23 - 3.01 (m, 1H), 1.51 (s, 9H), 1.33 (d, J = 6.7 Hz, 3H).LCMS (Analytical Method I) Rt = 0.93 min, MS (ESlpos): m/z 416.3 [M+H]+, Purity = 97%.

### Synthesis of Tert-butyl (3R)-3-methyl-4-{5-nitro-6-[(pyrimidin-4-yl)amino]pyridin-2-yl)piperazine-1-carboxylate / Intermediate 88

A solution of tert-butyl (3R)-3-methylpiperazine-1-carboxylate (0.32 g, 1.59 mmol), N-ethyl-N-isopropyl-propan-2-amine (0.83 mL, 4.77 mmol) and N-(6-chloro-3-nitro-2-pyridyl)pyrimidin-4-amine (Intermediate 26-1) (0.40 g, 1.59 mmol) in acetonitrile (6.3 mL) was heated to 80° C. for two hours. The reaction mixture was concentrated in vacuo. The product was purified by flash chromatography (25 g, silica), eluting with 0 - 10% MeOH in DCM) to yield the title compound (496 mg, 1.09 mmol, 68% yield) as a brown solid. 1H NMR (500 MHz, DMSO) δ 11.04 (s, 1H), 8.88 (d, J = 0.9 Hz, 1H), 8.75 (d, J = 5.8 Hz, 1H), 8.34 (d, J = 9.6 Hz, 1H), 8.21 (dd, J = 5.8, 1.2 Hz, 1H), 6.67 (d, J = 9.7 Hz, 1H), 4.76 -4.57 (m, 1H), 4.26 -4.14 (m, 1H), 4.06 - 3.80 (m, 3H), 1.45 (s, 9H), 1.23 (d, J = 6.7 Hz, 3H). One signal obscured. LCMS (Analytical Method I) Rt = 0.94 min, MS (ESlpos): m/z 416.3 [M+H]+, Purity = 91%.

### Synthesis of Tert-butyl (1R,4R)-5-{5-nitro-6-[(pyrimidin-4-yl)amino]pyridin-2-yl}-2,5-diazabicyclo[2.2.1]heptane-2-carboxylate / Intermediate 89

Tert-butyl (1R,4R)-2,5-diazabicyclo[2.2.1]heptane-2-carboxylate (200 mg, 1.01 mmol) and N-(6-chloro-3-nitro-2-pyridyl)pyrimidin-4-amine (Intermediate 26-1) (333 mg, 1.06 mmol) were dissolved in IPA (1.3 mL) and DIPEA (0.53 mL, 3.03 mmol) and then stirred at 100° C. for 1.5 hrs. The mixture was diluted with sat. aq. NaHCOs (3 ml) and the resulting precipitate collected by vacuum filtration to yield the title compound as a brown solid (868 mg, 100% yield).LCMS (Analytical Method I) Rt = 0.85 min, MS (ESlpos): m/z 414.3 [M+H]+, Purity = 78%.

### Synthesis of Tert-butyl (1R,4R)-5-{5-nitro-6-[(pyrimidin-4-yl)amino]pyridin-2-yl}-2,5-diazabicyclof2.2.21octane-2-carboxylate / Intermediate 90

Tert-butyl (1R,4R)-2,5-diazabicyclo[2.2.2]octane-2-carboxylate (200 mg, 0.942 mmol) and N-(6-chloro-3-nitro-2-pyridyl)pyrimidin-4-amine (Intermediate 26-1) (326 mg, 1.04 mmol) were dissolved in IPA (1.3 mL) and DIPEA (0.49 mL, 2.83 mmol) and then stirred at 100° C. for 1.5 hrs. The mixture was diluted with sat. aq. NaHCOs (3 ml) and the resulting precipitate collected by vacuum filtration to yield the title compound as a brown solid (739 mg, 100% yield).LCMS (Analytical Method I) Rt = 0.91 min, MS (ESlpos): m/z 428.3 [M+H]+, Purity = 63%.

### Synthesis of Tert-butyl (1R,4R)-5-[4-nitro-3-(pyrimidin-4-ylamino)phenyl]-2,5-diazabicyclo[2.2.1]heptane-2-carboxylate / intermediate 91

A mixture of N-(5-fluoro-2-nitro-phenyl)pyrimidin-4-amine (Intermediate 3-1) (175 mg, 0.747 mmol), tert-butyl (1R,4R)-2,5-diazabicyclo[2.2.1]heptane-2-carboxylate (148 mg, 0.747 mmol) and DIPEA (0.39 mL, 2.24 mmol) in THF (2.99 mL) was stirred and heated at 70° C. in a sealed tube overnight. Saturated aqueous NaHCOs (10 ml) was add to the reaction mixture and the aqueous phase was extracted with EtOAc (3 × 25 ml). The combined organic layers were washed with water (25 ml) and brine (25 ml), dried over MgSO₄ and the solvent was removed in vacuo. The crude was purified by flash chromatography eluting with 0-10% MeOH in DCM to afford the title compound (120 mg, 37% yield) 1H NMR (500 MHz, DMSO) δ 10.38 - 10.17 (m, 1H), 8.72 - 8.67 (m, 1H), 8.42 (d, J = 5.9 Hz, 1H), 8.05 (d, J = 9.5 Hz, 1H), 7.65 - 7.39 (m, 1H), 7.12 (dd, J = 5.9, 1.2 Hz, 1H), 6.67 - 6.45 (m, 1H), 4.83 - 4.69 (m, 1H), 4.59 - 4.44 (m, 1H), 3.68 - 3.59 (m, 1H), 3.47 - 3.35 (m, 1H), 3.27 - 3.17 (m, 2H), 2.07 - 1.93 (m, 2H), 1.44 - 1.33 (m, 9H).

### Synthesis of Tert-butyl (3R)-3-methyl-4-{4-nitro-3-[(pyrimidin-4-yl)aminolphenyl)piperazine-1-carboxylate / Intermediate 92

A mixture of N-(5-fluoro-2-nitro-phenyl)pyrimidin-4-amine (Intermediate 3-1) (163 mg, 0.694 mmol), tert-butyl (3R)-3-methylpiperazine-1-carboxylate (139 mg, 0.694 mmol) and DIPEA (0.29 mL, 1.67 mmol) in DMSO (2.78 mL) was stirred and heated at 100° C. in a sealed tube overnight. Saturated aqueous NaHCOs (10 ml) was added and the aqueous phase was extracted with EtOAc (3 × 25 ml). The combined organic layers were washed with water (25 ml) and brine (25 ml), dried over MgSO₄ and the solvent was removed in vacuo. The residue was purified by flash chromatography eluting with DCM/MeOH 0-10% to afford the title compound (113 mg, 28% yield). LCMS (Analytical Method I) Rt = 0.85 min, MS (ESlpos): m/z 415.4 [M+H]+, Purity = 42%.

### Synthesis of Tert-butyl (1R,4R)-5-{4-nitro-3-[(pyrimidin-4-yl)amino]phenyl}-2,5-diazabicyclo[2.2.2]octane-2-carboxylate / Intermediate 93

N-fluoro-2-nitro-phenyl)pyrimidin-4-amine (Intermediate 3-1) (204 mg, 0.871 mmol) and tert-butyl (1R,4R)-2,5-diazabicyclo[2.2.2]octane-2-carboxylate (185 mg, 0.871 mmol) were dissolved in DMSO (3.48 mL) in a sealed vial before the addition of DIPEA (0.37 mL, 2.09 mmol). The mixture was heated to 100° C. for 3 h. The mixture was cooled and NaHCOs (25 ml) added and the aqueous extracted with EtOAc (3 × 25 ml). The combined organic layers were washed with brine (25 ml), and then concentrated in vacuo. The crude was purified by flash chromatography (25 g, silica) eluting with 0-100% EtOAc/Heptane to afford the title compound (245 mg, 66% yield). 1H NMR (500 MHz, DMSO) δ 10.31 (d, J = 7.0 Hz, 1H), 8.71 (s, 1H), 8.41 (d, J = 5.9 Hz, 1H), 8.05 (d, J = 9.6 Hz, 1H), 7.76 - 7.54 (m, 1H), 7.29 - 7.06 (m, 1H), 6.63 (s, 1H), 4.41 (d, J = 12.4 Hz, 1H), 4.27 (m, 1H), 3.76 - 3.56 (m, 1H), 3.57 - 3.41 (m, 3H), 1.97 - 1.72 (m, 4H), 1.41 (s, 9H).LCMS (Analytical Method I) Rt = 0.83 min, MS (ESlpos): m/z 427.4 [M+H]+, Purity = 87%.

### Synthesis of Tert-butyl 4-{6-[(6-methylpyrimidin-4-yl)amino]-5-nitropyridin-2-yl)piperazine-1-carboxylate / Intermediate 94

A solution of tert-butyl 4-(6-amino-5-nitro-2-pyridyl)piperazine-1-carboxylate (Intermediate 4-1) (400 mg, 1.24 mmol), Cs₂CO₃ (806 mg, 2.47 mmol), Xantphos (36 mg, 0.0619 mmol), 4-chloro-6-methylpyrimidine (167 mg, 1.30 mmol) and Pd₂(dba)₃ (28 mg, 0.0309 mmol) in 1,4-dioxane (7 mL) was heated at 120° C. for 17 hrs. The reaction was re-treated with Pd₂(dba)₃ (28 mg, 0.0309 mmol) and Xantphos (36 mg, 0.0619 mmol) and stirred at 120° C. for a further 21 hrs. The reaction mixture was cooled to RT, poured into water and extracted with EtOAc (3×). The organic phases were combined, dried over Na₂SO₄, passed through a phase separator and concentrated in vacuo. The compound was purified by flash chromatography (25 g, silica) eluting with 0-100% EtOAc/heptane to yield the title compound as a yellow solid (241 mg, 37% yield). 1H NMR (400 MHz, DMSO-d6) δ 10.82 (s, 1H), 8.73 (s, 1H), 8.31 (d, J = 9.5 Hz, 1H), 8.09 (s, 1H), 6.64 (d, J = 9.6 Hz, 2H), 3.89 - 3.79 (m, 4H), 3.60 - 3.51 (m, 4H), 2.49 (s, 3H), 1.46 (s, 9H).LCMS (Analytical Method I) Rt = 0.88 min, MS (ESlpos): m/z 416 [M+H]+, Purity = 79%.

### Synthesis of Tert-butyl 4-{6-[(2-methylpyrimidin-4-yl)amino]-5-nitropyridin-2-yl}piperazine-1-carboxylate / Intermediate 95

A solution of tert-butyl 4-(6-amino-5-nitro-2-pyridyl)piperazine-1-carboxylate (Intermediate 4-1) (400 mg, 1.24 mmol), Cs₂CO₃ (806 mg, 2.47 mmol), Xantphos (36 mg, 0.0619 mmol), 4-chloro-2-methyl-pyrimidine (167 mg, 1.30 mmol) and Pd₂(dba)₃ (28 mg, 0.0309 mmol) in 1,4-dioxane (7 mL) was heated at 120° C. for 17 hrs. The reaction was re-treated with Pd₂(dba)₃ (28 mg, 0.0309 mmol) and Xantphos (36 mg, 0.0619 mmol) and stirred at 120° C. for a further 21 hrs. The reaction mixture was cooled to RT, poured into water and extracted with EtOAc (3×). The organic phases were combined, dried over Na₂SO₄, passed through a phase separator and concentrated in vacuo. The compound was purified by flash chromatography (50 g, silica), eluting with 0-10% MeOH /DCM. The product was purified again via flash chromatography (25 g, silica) eluting with 0-100% EtOAc/heptane to yield the title compound as a yellow solid (253 mg, 49% yield). 1H NMR (400 MHz, DMSO-d6) δ 10.85 (s, 1H), 8.61 (d, J = 5.7 Hz, 1H), 8.32 (d, J = 9.6 Hz, 1H), 8.00 (d, J = 5.8 Hz, 1H), 6.64 (d, J = 9.6 Hz, 1H), 3.89 - 3.75 (m, 4H), 3.59 - 3.48 (m, 4H), 2.55 (s, 3H), 1.46 (s, 9H).LCMS (Analytical Method I) Rt = 0.85 min, MS (ESlpos): m/z 416 [M+H]+, Purity = 100%.

### Synthesis of 1-(6-amino-5-nitropyridin-2-yl)azetidin-3-ol / Intermediate 96-1

A suspension of N-ethyl-N-isopropyl-propan-2-amine (1.8 mL, 10.2 mmol), 6-chloro-3-nitro-pyridin-2-amine (600 mg, 3.39 mmol) in acetonitrile (4 mL) was heated to 100° C. for 6 h. The reaction was cooled and the precipitate collected by filtration, washing with MeCN (~2 × 5 mL) and dried in vacuo to yield the title compound (665 mg, 3.16 mmol, 93% Yield) as a yellow solid. 1H NMR (500 MHz, DMSO-d6) δ 8.04 (d, J = 9.3 Hz, 1H), 7.88 - 7.66 (m, 1H), 5.89 - 5.79 (m, 1H), 4.64 - 4.50 (m, 1H), 4.37 -4.22 (m, 2H), 3.91 - 3.77 (m, 2H).LCMS (Analytical Method I) Rt = 0.44 min, MS (ESlpos): m/z 211.1 [M+H]+, Purity = 100%.

### Synthesis of 6-{3-[(tert-butyldimethylsilyl)oxy]azetidin-1-yl}-3-nitropyridin-2-amine / Intermediate 96-2

To a solution of 1-(6-amino-5-nitro-2-pyridyl)azetidin-3-ol (Intermediate 96-1) (843 mg, 4.01 mmol) and imidazole (682 mg, 10.0 mmol) in DCM (10 mL) was added tert-butyl-chloro-dimethylsilane (906 mg, 6.01 mmol) and the mixture stirred at RT for 16 hrs. The mixture was filtered and the collected solid was washed with DCM and water, then dried in vacuo to yield the title compound as an orange solid (1.48 g, 97% yield). 1H NMR (400 MHz, Chloroform-d) δ 8.06 (d, J = 9.2 Hz, 1H), 5.59 (d, J = 9.2 Hz, 1H), 4.69 - 4.64 (m, 1H), 4.35 - 4.16 (m, 2H), 3.86 (dd, J = 10.9, 4.4 Hz, 2H), 0.82 (s, 9H), -0.00 (s, 6H).LCMS (Analytical Method I) Rt = 1.14 min, MS (ESlpos): m/z 325.2 [M+H]+, Purity = 100%.

### Synthesis of 6-{3-[(tert-butyldimethylsilyl)oxy]azetidin-1-yl}-3-nitro-N-(pyridin-4-yl)pyridin-2-amine / intermediate 96

To a nitrogen sparged solution of cesium carbonate (2.78 g, 8.22 mmol), (5-diphenylphosphanyl-9,9-dimethyl-xanthen-4-yl)-diphenyl-phosphane (119 mg, 0.205 mmol), 4-iodopyridine (884 mg, 4.31 mmol) and 6-[3-[tert-butyl(dimethyl)silyl]oxyazetidin-1-yl]-3-nitro-pyridin-2-amine;hydrochloride (intermediate 96-2) (1.48 g, 4.11 mmol) in 1,4-dioxane (7.2 mL) was added (1{E},4{E})-1,5-diphenylpenta-1,4-dien-3-one;palladium (94 mg, 0.103 mmol) and the solution sparged with nitrogen. The mixture was heated to 100° C. The mixture was cooled and the supernatant liquid decanted, washing the solids with methanol. The solution was concentrated in vacuo and the product purified by flash chromatography (50 g, silica), eluting with 0-20% MeOH/DCM to yield the title compound (835 mg, 1.77 mmol, 43% yield) as a yellow solid. 1H NMR (500 MHz, Chloroform-d) δ 10.88 (s, 1H), 8.40 - 8.34 (m, 2H), 8.17 (d, J = 9.3 Hz, 1H), 7.61 - 7.55 (m, 2H), 5.73 (d, J = 9.3 Hz, 1H), 4.73 - 4.66 (m, 1H), 4.43 - 4.17 (m, 2H), 4.06 - 3.87 (m, 2H), 0.81 (s, 9H), -0.00 (s, 6H).LCMS (Analytical Method I) Rt = 0.88 min, MS (ESlpos): m/z 402.3 [M+H]+, Purity = 86%.

### Synthesis of N2,N2-dibenzyl-5-nitropyridine-2,6-diamine / Intermediate 97-1

A suspension of 6-chloro-3-nitropyridin-2-amine (5.00 g, 28.8 mmol), dibenzylamine (14 mL, 72.0 mmol), and DIPEA (15 mL, 86.4 mmol) in MeCN (100 mL) was stirred at 80° C. for 8 h. The mixture was concentrated in vacuo then Et₂O was added. The mixture was filtered and the filtrate was concentrated in vacuo. The residue was purified via flash chromatography (340 g, silica), eluting with DCM to yield the title compound as a yellow solid (9.02 g, 91% yield). 1H NMR (400 MHz, Chloroform-d) δ 8.18 (d, J = 9.4 Hz, 1H), 7.40 - 7.27 (m, 6H), 7.24 - 7.15 (m, 4H), 6.03 (d, J = 9.4 Hz, 1H), 4.99 - 4.60 (m, 4H).LCMS (Analytical Method I) Rt = 1.06 min, MS (ESlpos): m/z 335.2 [M+H]+, Purity = 97%.

### Synthesis of N2,N2-dibenzyl-5-nitro-N6-(pyridin-4-yl)pyridine-2,6-diamine / Intermediate 97-2

A mixture of N6,N6-dibenzyl-3-nitro-pyridine-2,6-diamine (intermediate 97-1) (250 mg, 0.748 mmol), 4-iodopyridine (169 mg, 0.822 mmol), Pd₂dba₃ (17 mg, 0.0187 mmol), cesium carbonate (0.49 g, 1.50 mmol) and Xantphos (22 mg, 0.0374 mmol) in 1,4-dioxane (7 mL) was degassed by sparging with nitrogen. The reaction was heated to 100° C. overnight. The mixture was cooled and filtered through a pad of celite, washing with EtOAc (60 mL) and concentrated in vacuo. The residue was purified by flash chromatography (25 g, silica), eluting with 0-5% MeOH/DCM to yield the title compound (298 mg, 0.724 mmol, 97% Yield) as a yellow solid 1H NMR (500 MHz, DMSO-d6) δ 10.58 (s, 1H), 8.28 (d, J = 9.5 Hz, 1H), 8.21 - 8.16 (m, 2H), 7.48 - 7.43 (m, 2H), 7.40 - 7.32 (m, 4H), 7.31 - 7.23 (m, 6H), 6.50 (d, J = 9.5 Hz, 1H), 5.14 - 4.74 (m, 4H). LCMS (Analytical Method I) Rt = 0.82 min, MS (ESlpos): m/z 412.3 [M+H]+, Purity = 98%.

### Synthesis of N,N-dibenzyl-2-(4-fluorophenyl)-3-(pyridin-4-yl)-3H-imidazo[4,5-b]pyridin-5-amine / intermediate 97-3

Na₂S₂O₄ (5.66 g, 32.2 mmol) was added to a suspension of N6,N6-dibenzyl-3-nitro-N2-(4-pyridyl)pyridine-2,6-diamine (Intermediate 97-2) (98%, 4.50 g, 10.7 mmol) in EtOH (7.8 mL) and DMSO (39.2 mL). The reaction was gently warmed then 4-fluorobenzaldehyde (1.4 mL, 12.8 mmol) was added and the reaction stirred at 100° C. for 22 hrs. The mixture was diluted with 1 M NaOH, extracted with EtOAc (3×), passed through a phase separator and concentrated in vacuo. The residue was purified by flash chromatography (200 g, silica), eluting with 0-100% DCM/heptane then 0-5% MeOH/DCM. The fractions were combined and concentrated in vacuo. The product was triturated with Et₂O to yield the title compound as a brown solid (3.3 g, 57% yield). 1H NMR (500 MHz, Chloroform-d) δ 8.57 - 8.48 (m, 2H), 7.89 (d, J = 8.9 Hz, 1H), 7.52 - 7.43 (m, 2H), 7.38 -7.32 (m, 4H), 7.28 (m, 4H), 7.25 - 7.21 (m, 4H), 7.13 - 7.02 (m, 2H), 6.67 (d, J = 9.0 Hz, 1H), 4.85 (s, 4H). LCMS (Analytical Method J) Rt = 0.99 min, MS (ESlpos): m/z 486.3 [M+H]+, Purity = 96%.

### Synthesis of 2-(4-fluorophenyl)-3-(pyridin-4-yl)-3H-imidazo[4,5-b]pyridin-5-amine / Intermediate 97-4

To a solution of N,N-dibenzyl-2-(4-fluorophenyl)-3-(4-pyridyl)imidazo[4,5-b]pyridin-5-amine (Intermediate 97-3) (90%, 3.30 g, 6.12 mmol) in TFA (21 mL, 0.283 mol), was added triflic acid (2.0 mL, 22.6 mmol). The mixture was stirred at 80° C. for 8 hrs, then poured into ice-cold water and extracted with DCM (2×). The organic layer was discarded and the aq. layer was basified with 1 M NaOH and extracted with DCM (3×) to yield the title compound yellow solid (1.4 g, 4.59 mmol, 75% yield). 1H NMR (400 MHz, Methanol-d4) δ 8.71 - 8.63 (m, 2H), 7.80 (d, J = 8.7 Hz, 1H), 7.54 - 7.43 (m, 4H), 7.19 -7.10 (m, 2H), 6.63 (d, J = 8.7 Hz, 1H).LCMS (Analytical Method I) Rt = 0.55 min, MS (ESlpos): m/z 306.2 [M+H]+, Purity = 100%.

### Synthesis of 4-[2-(4-fluorophenyl)-5-iodo-3H-imidazo[4,5-b]pyridin-3-yl]pyridine / Intermediate 97

To a solution of 2-(4-fluorophenyl)-3-(4-pyridyl)imidazo[4,5-b]pyridin-5-amine (Intermediate 97-4) (150 mg, 0.491 mmol) in diiodomethane (5.0 mL, 62.1 mmol) at 60° C. was added tert-butyl nitrite (90%, 0.15 mL, 1.14 mmol) and the mixture was then stirred at that RT for 1 hr. The mixture was concentrated in vacuo. The residue was taken up in DCM, washed with NaHCO_{3(aq)}, passed through a phase separator and concentrated in vacuo. The crude product was purified via flash chromatography (10 g, silica), eluting with 0-5% MeOH/DCM to yield the title compound as a yellow solid (96 mg, 37% yield). 1H NMR (500 MHz, CDCl3) δ 8.87 - 8.78 (m, 2H), 7.93 - 7.78 (m, 3H), 7.65 - 7.51 (m, 3H), 7.22 - 7.13 (m, 2H).LCMS (Analytical Method H) Rt = 0.60 min, MS (ESlpos): m/z 417.1 [M+H]+, Purity = 62%.

### Synthesis of Tert-butyl (3R)-4-{3-[2-(difluoromethyl)pyridin-4-yl]-2-(4-fluorophenyl)-3H-imidazo[4,5-blpyridin-5-yl)-3-methylpiperazine-1-carboxylate / Intermediate 85

tert-Butyl (3R)-4-[5-amino-6-(4-pyridylamino)-2-pyridyl]-3-methyl-piperazine-1-carboxylate (Intermediate 85-1) (675 mg, 1.76 mmol) and 4-fluorobenzaldehyde (207 uL, 1.93 mmol) were dissolved in ethanol (13 mL) and stirred for 15 minutes. Cerium ammonium nitrate (96 mg, 0.176 mmol) and hydrogen peroxide (35%, 307 uL, 3.51 mmol) were added and the reaction was stirred overnight. The was quenched into water and the aqueous layer was extracted into ethyl acetate (5 mL) three times, the combined organics washed with brine, dried over MgSO₄ and concentrated in vacuo. The residue was purified by preparative HPLC (Method A1) to the title compound (705 mg, 1.22 mmol, 69% Yield) as a white solid. 1H NMR (400 MHz, DMSO) δ 8.78 (d, J = 5.3 Hz, 1H), 8.01 (d, J = 8.9 Hz, 1H), 7.85 (d, J = 1.7 Hz, 1H), 7.55 (m, 3H), 7.42 - 7.24 (m, 2H), 7.03 (t, J = 54.7 Hz, 1H), 6.91 (d, J = 9.0 Hz, 1H), 4.54 - 4.39 (m, 1H), 4.34 (d, J = 4.2 Hz, 2H), 4.03 - 3.87 (m, 1H), 3.84 - 3.69 (m, 2H), 3.09 - 3.01 (m, 1H), 1.06 - 1.05 (m, 3H), 1.05 - 1.02 (m, 9H).LCMS (Analytical Method I) Rt = 1.10 min, MS (ESlpos): m/z 539.4 [M+H]+, Purity = 93%.

### Synthesis of 1-tert-butyl 2-methyl (2R)-4-[2-(2,4-difluorophenyl)-3-(pyridin-4-yl)-3H-imidazo[4,5-b]pyridin-5-yl]piperazine-1,2-dicarboxylate / Intermediate 69-3

A suspension of O1-tert-butyl O2-methyl-(2R)-4-[5-nitro-6-(4-pyridylamino)-2-pyridyl]piperazine-1,2-dicarboxylate (Intermediate 69-2) (353 mg, 0.770 mmol), Sodium dithionite (456 mg, 2.62 mmol) and 2,4-difluorobenzaldehyde (101 uL, 0.924 mmol) in DMSO (1.9 mL) and ethanol (1.9 mL) was heated at 100° C. under air for 40 hours. The reaction was cooled and loaded directly onto an SCX-2 ion exchange cartridge (10 g) primed with methanol. The cartridge was washing with methanol, then 2 M NH₃ in MeOH. The basic fraction was concentrated in vacuo. The residue was dissolved in DCM (3.2 mL) then DIPEA (0.26 mL, 1.47 mmol) and boc anhydride (213 mg, 0.977 mmol) were added. The mixture was stirred at RT for 2 days, then partitioned with water. The aqueous was extracted with DCM (2×) and the organics were combined and concentrated in vacuo to yield the title compound (237 mg, 59% yield). LCMS (Analytical Method I) Rt = 0.95 min, MS (ESlpos): m/z 551.4 [M+H]+, Purity = 67%.

### Synthesis of (2R)-1-[(tert-butoxy)carbonyl]-4-[2-(2,4-difluorophenyl)-3-(pyridin-4-yl)-3H-imidazo[4,5-b]pyridin-5-yl]piperazine-2-carboxylic Acid / Intermediate 69-4

1-tert-butyl 2-methyl (2R)-4-[2-(2,4-difluorophenyl)-3-(pyridin-4-yl)-3H-imidazo[4,5-b]pyridin-5-yl]piperazine-1,2-dicarboxylate (Intermediate 69-3) (237 mg, 0.430 mmol) was dissolved in a mixture of THF (2.1 mL) and water (2.1 mL), then lithium hydroxide (52 mg, 2.15 mmol) was added. The mixture was stirred at RT for 2 hrs then acidified with 2 M HCl and extracted with DCM (2×). The combined organic layers were dried and concentrated in vacuo to yield the title compound (118 mg, 34% yield).LCMS (Analytical Method I) Rt = 0.82 min, MS (ESlpos): m/z 537.3 [M+H]+, Purity = 66%.

### Synthesis of Tert-butyl (2R)-2-carbamoyl-4-[2-(2,4-difluorophenyl)-3-(pyridin-4-yl)-3H-imidazo[4,5-b]pyridin-5-yl]piperazine-1-carboxylate / Intermediate 69

(2R)[(tert-butoxy)carbonyl]-4-[2-(2,4-difluorophenyl)-3-(pyridin-4-yl)-3H-imidazo[4,5-b]pyridin-5-yl]piperazine-2-carboxylic acid (Intermediate 69-4) was dissolved in DMF (3 mL), then DIPEA (85 mg, 0.660 mmol), ammonium chloride (71 mg, 1.32 mmol) and HATU (125 mg, 0.330 mmol) were added. The mixture was stirred at RT for 1 hr, then diluted with water and extracted with DCM (2×). The organics were combined, dried and concentrated in vacuo.LCMS (Analytical Method I) Rt = 0.74 min, MS (ESlpos): m/z 536.3 [M+H]+, Purity = 46%.

### Synthesis of Tert-butyl 4-[6-bromo-2-(4-fluorophenyl)-3-(pyridin-4-yl)-3H-imidazo[4,5-b]pyridin-5-yllpiperazine-1-carboxylate / Intermediate 98-1

To a stirred solution of 1-[6-bromo-2-(4-fluorophenyl)-3-(pyridin-4-yl)-3H-imidazo[4,5-b]pyridin-5-yl]piperazine (Compound 97 of Table 1) (421 mg, 0.874 mmol) in DCM (10 mL), DIPEA (0.46 mL, 2.62 mmol) was added followed by boc anhydride (381 mg, 1.75 mmol). The mixture was stirred at RT overnight, then quenched with sat. NaHCOs (10 mL). The aqueous was extracted with DCM (10 mL), the combined organics were filtered through a phase separator and concentrated in vacuo. The product was purified by flash chromatography (25 g, silica), eluting with 0-5% MeOH/DCM to yield the title compound as a yellow solid (335 mg, 69% yield) as a pale yellow solid 1H NMR (500 MHz, Methanol-d4) δ 8.72 - 8.66 (m, 2H), 8.33 (s, 1H), 7.62 - 7.55 (m, 2H), 7.54 - 7.49 (m, 2H), 7.23 - 7.16 (m, 2H), 3.66 - 3.53 (m, 4H), 3.28 - 3.23 (m, 4H), 1.48 (s, 9H).LCMS (Analytical Method I) Rt = 1.09 min, MS (ESlpos): m/z 553.2, 555.1 [M+H]+, Purity = 100%.

### Synthesis of Tert-butyl 4-[6-fluoro-2-(4-fluorophenyl)-3-(pyridin-4-yl)-3H-imidazo[4,5-b]pyridin-5-yllpiperazine-1-carboxylate / Intermediate 98

To a stirred solution of tert-butyl 4-[6-bromo-2-(4-fluorophenyl)-3-(4-pyridyl)imidazo[4,5-b]pyridin-5-yl]piperazine-1-carboxylate (Intermediate 98-1) (100 mg, 0.181 mmol) in THF (0.2 mL) at 0° C. was added 1.3 M isopropylmagnesium chloride;LiCl salt (181 uL, 0.235 mmol) and the mixture was stirred at 0° C. for 1 hr. The solvent was removed by flowing nitrogen onto the reaction and DCM (0.2 mL) was added. The mixture was cooled to -40° C. and a solution of N-fluoro-N-(phenylsulfonyl)benzenesulfonamide (115 mg, 0.365 mmol) in DCM (0.6 mL) and perfluorodecaline (0.31 mL, 1.29 mmol) was added. The reaction was stirred at RT for 18 hrs. Water was added and the mixture extracted with DCM (3×). The combined organics were filtered through a phase separator and concentrated in vacuo. The residue was purified by preparative HPLC (Method A2) to yield the title compound as an off-white solid (42 mg, 47% yield). 1H NMR (500 MHz, Methanol-d4) δ 8.71 - 8.65 (m, 2H), 7.83 (d, J = 12.5 Hz, 1H), 7.59 - 7.54 (m, 2H), 7.52 - 7.49 (m, 2H), 7.22 - 7.15 (m, 2H), 3.62 - 3.51 (m, 4H), 3.44 - 3.39 (m, 4H), 1.48 (s, 9H).LCMS (Analytical Method I) Rt = 1.00 min, MS (ESlpos): m/z 493.3 [M+H]+, Purity = 100%.

### Example 1.5 - Synthesis of Further Compounds

### Synthesis of 1-[2-(2,4-difluorophenyl)-3-(pyridin-4-yl)-3H-imidazo[4,5-b]pyridin-5-yl]piperazine / Compound 34 of Table 1

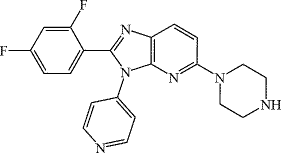 [SMILES: Fc1ccc(-c2nc3ccc(N4CCNCC4)nc3n2-c2ccncc2)c(F)c1]

A suspension of tert-butyl 4-[5-nitro-6-(4-pyridylamino)-2-pyridyl]piperazine-1-carboxylate (Intermediate 4) (1 g, 2.50 mmol), 2,4-difluorobenzaldehyde (328 uL, 3.00 mmol) and Na₂S₂O₄ (1.5 g, 8.49 mmol) in DMSO (6.2 mL) was heated at 100° C. under air overnight in a pressure vial. DCM (~5 mL) was added, resulting in precipitation of a solid. Water (10 mL) was added and the organic layer separated. The aqueous was extracted with DCM (2 × 10 mL). The combined organics were passed through a phase separating frit and the solvent removed in vacuo affording a crude solid. The solid was dissolved in MeOH and purified using preparative HPLC (Method A1) to yield the title compound (200 mg, 20% yield) 1H NMR (500 MHz, DMSO) δ 8.71 - 8.60 (m, 2H), 8.00 (d, J = 9.0 Hz, 1H), 7.85 - 7.72 (m, 1H), 7.47 - 7.37 (m, 2H), 7.36 - 7.22 (m, 2H), 6.94 (d, J = 9.0 Hz, 1H), 3.49 - 3.39 (m, 4H), 2.86 - 2.72 (m, 4H), 2.34 (s, 1H). LCMS (Analytical Method A) Rt = 1.32 min, MS (ESlpos): m/z 393.3 [M+H]+, Purity = 100%.

Each of the compounds listed in Table 1.5.4 were prepared according to the method of Compound 34 of Table 1 using the intermediate listed in the “Synthesis” column with appropriate aldehyde derivatives for such compounds. The final compounds were purified by preparative HPLC Methods, A1, A2 or B1. If required, further purification using KP-NH column (gradient 0-50% MeOH/ DCM) or SCX cartridge (3N NH3 in MeOH) was carried out.

TABLE 1.5.4

| | | | |
|---|---|---|---|
| Example | Synthesis | Structure/ Name | Data |
| 35-SS | Intermediate 71 (19% yield) | 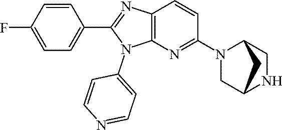 [SMILES: Fc1ccc(-c2nc3ccc(N4C[C@@H]5C[C@H]4CN5)nc3n2-c2ccncc2)cc1] | 1H NMR (400 MHz, DMSO-d6) δ 8.76 - 8.63 (m, 2H), 7.90 (d, J = 8.8 Hz, 1H), 7.53 - 7.46 (m, 2H), 7.46 -7.37 (m, 2H), 7.33 - 7.18 (m, 2H), 6.56 (d, J = 8.8 Hz, 1H), 4.63 (s, 1H), 3.63 (s, 1H), 3.44 (m, 1H), 3.21 (m, 1H), 2.88 (d, J = 7.7 Hz, 1H), 2.77 (d, J = 9.4 Hz, 1H), 1.78 - 1.61 (m, 2H). LCMS (Analytical Method A) Rt = 1.38 min, MS (ESIpos): m/z 387.3 [M+H]+, Purity = 98%. |
| | | (1S,4S)-2-[2-(4-fluorophenyl)-3-(pyridin-4-yl)-3H-imidazo [4,5-b]pyridin-5-yl]-2,5-diazabicyclo[2.2.1]heptane | |
| 37 | Intermediate 4 (34% yield) | 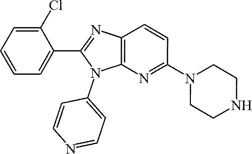 [SMILES: Clc1ccccc1-c1nc2ccc(N3CCNCC3)nc2n1-c1ccncc1] | 1H NMR (400 MHz, DMSO-d6) δ 8.66 - 8.54 (m, 2H), 7.99 (d, J = 9.0 Hz, 1H), 7.76 - 7.67 (m, 1H), 7.57 -7.46 (m, 3H), 7.36 - 7.29 (m, 2H), 6.94 (d, J = 9.0 Hz, 1H), 3.47 - 3.42 (m, 4H), 2.84 - 2.75 (m, 4H). LCMS (Analytical Method B) Rt = 2.43 min, MS (ESIpos): m/z 391.3, 393.2 [M+H]+, Purity = 98%. |
| | | 1-[2-(2-chlorophenyl)-3-(pyridin-4-yl)-3H-imidazo[4,5-b]pyridin-5-yl]piperazine | |
| 38-RS | Intermediate 32 (25% yield) | 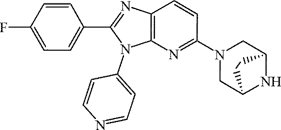 [SMILES: Fc1ccc(-c2nc3ccc(N4C[C@H]5CC[C@@H](C4)N5)nc3n2-c2ccncc2)cc1] | 1H NMR (400 MHz, Methanol-d4) δ 8.59 - 8.48 (m, 2H), 7.80 (d, J = 9.0 Hz, 1H), 7.47 - 7.41 (m, 2H), 7.41 -7.37 (m, 2H), 7.11 - 7.02 (m, 2H), 6.72 (d, J = 9.0 Hz, 1H), 3.89 - 3.81 (m, 2H), 3.51 (s, 2H), 2.96 - 2.89 (m, 2H), 1.78 - 1.63 (m, 4H). LCMS (Analytical Method B) Rt = 2.70 min, MS (ESIpos): m/z 401.3 [M+H]+, Purity = 96%. |
| | | (1R,5S)-3-[2-(4-fluorophenyl)-3-(pyridin-4-yl)-3H-imidazo [4,5-b]pyridin-5-yl]-3,8-diazabicyclo[3.2.1]octane | |
| 39 | Intermediate 4 (21% yield) | 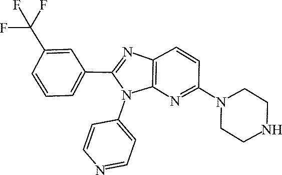 [SMILES: FC(F)(F)c1cccc(-c2nc3ccc(N4CCNCC4)nc3n2-c2ccncc2)c1] | 1H NMR (500 MHz, DMSO-d6) δ 8.77 - 8.68 (m, 2H), 8.00 (d, J = 9.0 Hz, 1H), 7.84 - 7.76 (m, 2H), 7.74 -7.67 (m, 1H), 7.65 - 7.58 (m, 1H), 7.54 - 7.44 (m, 2H), 6.93 (d, J = 9.0 Hz, 1H), 3.46 - 3.39 (m, 4H), 2.82 -2.73 (m, 4H). LCMS (Analytical Method B) Rt = 2.91 min, MS (ESIpos): m/z 425.3 [M+H]+, Purity = 97%. |
| | | 1-[3-(pyridin-4-yl)-2-[3-(trifluoromethyl)phenyl]-3H-imidazo[4,5-b]pyridin-5-yl]piperazine | |
| 40 | Intermediate 4 (62% yield) | 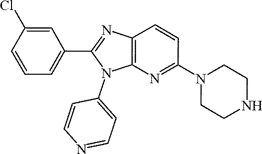 [SMILES: Clc1cccc(-c2nc3ccc(N4CCNCC4)nc3n2-c2ccncc2)c1] | 1H NMR (400 MHz, DMSO) δ 8.79 -8.65 (m, 2H), 7.98 (d, J = 8.9 Hz, 1H), 7.58 - 7.52 (m, 1H), 7.52 - 7.45 (m, 3H), 7.41 (t, J = 7.9 Hz, 1H), 7.36 - 7.31 (m, 1H), 6.92 (d, J = 9.0 Hz, 1H), 3.44 - 3.39 (m, 4H), 2.82 - 2.73 (m, 4H). LCMS (Analytical Method B) Rt = 1.58 min, MS (ESIpos): m/z 391.3, 393.3 [M+H]+, Purity = 99%. |
| | | 1-[2-(3-chlorophenyl)-3-(pyridin-4-yl)-3H-imidazo[4,5-b]pyridin-5-yl]piperazine | |
| 41-RS | Intermediate 35 (7% yield) | 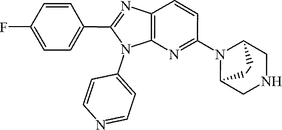 [SMILES: Fc1ccc(-c2nc3ccc(N4[C@@H]5CC[C@H]4CNC5)nc3n2-c2ccncc2)cc1] | 1H NMR (400 MHz, Methanol-d4) δ 8.73 - 8.58 (m, 2H), 7.93 (d, J = 8.8 Hz, 1H), 7.64 - 7.52 (m, 2H), 7.52 -7.42 (m, 2H), 7.24 - 7.10 (m, 2H), 6.88 (d, J = 8.9 Hz, 1H), 4.55 (m, 4H), 3.13 (d, J = 12.6 Hz, 2H), 2.80 - 2.71 (m, 2H), 2.12 - 1.98 (m, 4H). LCMS (Analytical Method B) Rt = 2.65 min, MS (ESlpos): m/z 401.3 [M+H]+, Purity = 96%. |
| | | (1R,5S)-8-[2-(4-fluorophenyl)-3-(pyridin-4-yl)-3H-imidazo[4,5-b]pyridin-5-yl]-3,8-diazabicyclo[3.2.1]octane | |
| 42 | Intermediate 4 (21% yield) | 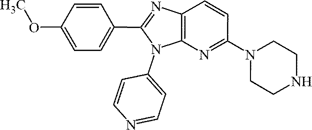 [SMILES: COc1ccc(-c2nc3ccc(N4CCNCC4)nc3n2-c2ccncc2)cc1] | 1H NMR (500 MHz, DMSO-d6) δ 8.75 - 8.68 (m, 2H), 7.93 (d, J = 8.9 Hz, 1H), 7.45 - 7.42 (m, 2H), 7.42 -7.38 (m, 2H), 7.00 - 6.94 (m, 2H), 6.87 (d, J = 8.9 Hz, 1H), 3.78 (s, 3H), 3.41 - 3.36 (m, 4H), 2.80 - 2.74 (m, 4H). LCMS (Analytical Method B) Rt = 2.37 min, MS (ESIpos): m/z 387.3 [M+H]+, Purity = 97%. |
| | | 1-[2-(4-methoxyphenyl)-3-(pyridin-4-yl)-3H-imidazo[4,5-b]pyridin-5-yl]piperazine | |
| 44 | Intermediate 4 (17% yield) | 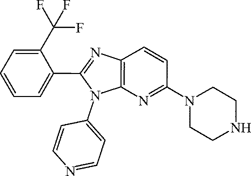 [SMILES: FC(F)(F)c1ccccc1-c1nc2ccc(N3CCNCC3)nc2n1-c1ccncc1] | 1H NMR (500 MHz, DMSO-d6) δ 8.65 - 8.51 (m, 2H), 8.00 (d, J = 9.0 Hz, 1H), 7.92 - 7.87 (m, 1H), 7.76 -7.69 (m, 2H), 7.64 - 7.58 (m, 1H), 7.32 - 7.27 (m, 2H), 6.94 (d, J = 9.0 Hz, 1H), 3.46 - 3.40 (m, 4H), 2.82 -2.75 (m, 4H). LCMS (Analytical Method B) Rt = 2.57 min, MS (ESIpos): m/z 425.3 [M+H]+, Purity = 97%. |
| | | 1-[3-(pyridin-4-yl)-2-[2-(trifluoromethyl)phenyl]-3H-imidazo[4,5-b]pyridin-5-yl]piperazine | |
| 45 | Intermediate 4 (35% yield) | 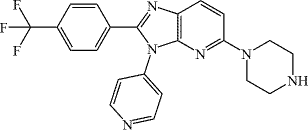 [SMILES: FC(F)(F)c1ccc(-c2nc3ccc(N4CCNCC4)nc3n2-c2ccncc2)cc1] | 1H NMR (400 MHz, DMSO-d6) δ 8.72 (d, J = 6.1 Hz, 2H), 7.99 (d, J = 9.0 Hz, 1H), 7.77 (d, J = 8.3 Hz, 2H), 7.67 (d, J = 8.1 Hz, 2H), 7.47 (d, J = 6.1 Hz, 2H), 6.92 (d, J = 9.0 Hz, 1H), 3.44 - 3.40 (m, 4H), 2.79 - 2.71 (m, 4H). LCMS (Analytical Method B) Rt = 2.94 min, MS (ESIpos): m/z 425.3 [M+H]+, Purity = 98%. |
| | | 1-[3-(pyridin-4-yl)-2-[4-(trifluoromethyl)phenyl]-3H-imidazo[4,5-b]pyridin-5-yl]piperazine | |
| 46 | Intermediate 4 (35% yield) | 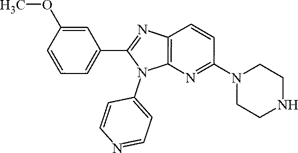 [SMILES: COc1cccc(-c2nc3ccc(N4CCNCC4)nc3n2-c2ccncc2)c1] | 1H NMR (400 MHz, DMSO-d6) δ 8.75 - 8.68 (m, 2H), 7.97 (d, J = 8.9 Hz, 1H), 7.44 (dd, J = 4.6, 1.6 Hz, 2H), 7.30 (t, J = 8.0 Hz, 1H), 7.06 -7.03 (m, 1H), 7.02 - 6.95 (m, 2H), 6.90 (d, J = 9.0 Hz, 1H), 3.69 (s, 3H), 3.44 - 3.39 (m, 4H), 2.82 - 2.73 (m, 4H). LCMS (Analytical Method B) Rt = 2.40 min, MS (ESIpos): m/z 387.3 [M+H]+, Purity = 99%. |
| | | 1-[2-(3-methoxyphenyl)-3-(pyridin-4-yl)-3H-imidazo[4,5-b]pyridin-5-yl]piperazine | |
| 47 | Intermediate 4 (16% yield) | 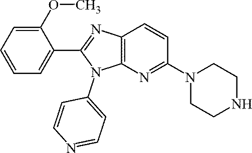 [SMILES: COc1ccccc1-c1nc2ccc(N3CCNCC3)nc2n1-c1ccncc1] | 1H NMR (500 MHz, DMSO-d6) δ 8.59 (d, J = 6.2 Hz, 2H), 7.93 (d, J = 8.9 Hz, 1H), 7.65 (dd, J = 7.5, 1.7 Hz, 1H), 7.48 (ddd, J = 8.4, 7.5, 1.8 Hz, 1H), 7.31 - 7.26 (m, 2H), 7.12 (td, J = 7.5, 0.9 Hz, 1H), 6.94 (d, J = 8.0 Hz, 1H), 6.88 (d, J = 9.0 Hz, 1H), 3.21 (s, 3H), 2.83 - 2.75 (m, 4H). One signal obscured. LCMS (Analytical Method B) Rt = 2.27 min, MS (ESIpos): m/z 387.4 [M+H]+, Purity = 99%. |
| | | 1-[2-(2-methoxyphenyl)-3-(pyridin-4-yl)-3H-imidazo[4,5-b]pyridin-5-yl]piperazine | |
| 49 | Intermediate 36 (11 % yield) | 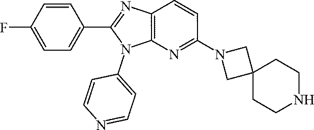 [SMILES: Fc1ccc(-c2nc3ccc(N4CC5(CCNCC5)C4)nc3n2-c2ccncc2)cc1] | 1H NMR (400 MHz, Chloroform-d) δ 8.71 - 8.68 (m, 2H), 7.88 (d, J = 8.6 Hz, 1H), 7.54 - 7.44 (m, 2H), 7.38 -7.32 (m, 2H), 7.05 (t, J = 8.7 Hz, 2H), 6.33 (d, J = 8.7 Hz, 1H), 3.76 (s, 4H), 2.91 - 2.79 (m, 4H), 1.84 - 1.76 (m, 4H). LCMS (Analytical Method A) Rt = 1.50 min, MS (ESIpos): m/z 415.3 [M+H]+, Purity = 100%. |
| | | 2-[2-(4-fluorophenyl)-3-(pyridin-4-yl)-3H-imidazo[4,5-b]pyridin-5-yl]-2,7-diazaspiro[3.5]nonane | |
| 56 | Intermediate 12 (13% yield) | 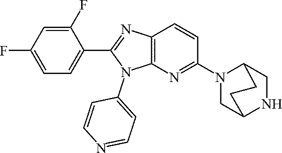 [SMILES: Fc1ccc(-c2nc3ccc(N4CC5CCC4CN5)nc3n2-c2ccncc2)c(F)c1] | 1H NMR (500 MHz, Chloroform-d) δ 8.63 - 8.52 (m, 2H), 7.85 (d, J = 8.9 Hz, 1H), 7.66 - 7.58 (m, 1H), 7.28 -7.21 (m, 2H), 7.01 - 6.88 (m, 1H), 6.74 - 6.62 (m, 1H), 6.41 (d, J = 8.9 Hz, 1H), 4.54 (s, 1H), 3.65 (dt, J = 10.4, 2.6 Hz, 1H), 3.56 - 3.45 (m, 1H), 3.25 (dt, J = 11.0, 2.6 Hz, 1H), 3.20 (s, 1H), 3.16 - 3.11 (m, 1H), 2.03 - 1.90 (m, 2H), 1.89 - 1.82 (m, 1H), 1.77 - 1.69 (m, 1H). LCMS (Analytical Method A) Rt = 1.52 min, MS (ESlpos): m/z 419.3 [M+H]+, Purity = 98%. |
| | | rac-(1R,4R)-2-[2-(2,4-difluorophenyl)-3-(pyridin-4-yl)-3H-imidazo[4,5-b]pyridin-5-yl]-2,5-diazabicyclo[2.2.2]octane | |
| 57 | Intermediate 12 (29% yield) | 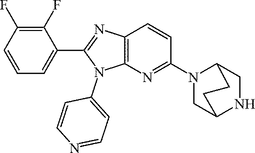 [SMILES: Fc1cccc(-c2nc3ccc(N4CC5CCC4CN5)nc3n2-c2ccncc2)c1F] | 1H NMR (400 MHz, Chloroform-d) δ 8.66 - 8.52 (m, 2H), 7.87 (d, J = 8.9 Hz, 1H), 7.44 - 7.31 (m, 1H), 7.29 -7.24 (m, 2H), 7.17 - 7.10 (m, 1H), 6.43 (d, J = 8.9 Hz, 1H), 4.58 - 4.46 (m, 1H), 3.65 (dt, J = 10.4, 2.6 Hz, 1H), 3.52 (dd, J = 10.4, 1.7 Hz, 1H), 3.29 - 3.23 (m, 1H), 3.21 - 3.11 (m, 2H), 2.05 -1.82 (m, 3H), 1.79 - 1.71 (m, 1H). LCMS (Analytical Method A) Rt = 1.52 min, MS (ESIpos): m/z 419.3 [M+H]+, Purity = 98%. |
| | | rac-(1R,4R)-2-[2-(2,3-difluorophenyl)-3-(pyridin-4-yl)-3H-imidazo[4,5-b]pyridin-5-yl]-2,5-diazabicyclo[2.2.2]octane | |
| 59 | Intermediate 12 (19% yield) | 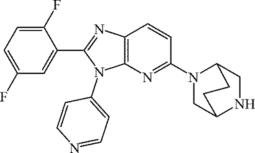 [SMILES: Fc1ccc(F)c(-c2nc3ccc(N4CC5CCC4CN5)nc3n2-c2ccncc2)c1] | 1H NMR (500 MHz, Chloroform-d) δ 8.63 - 8.53 (m, 2H), 7.86 (d, J = 8.9 Hz, 1H), 7.41 (ddd, J = 8.4, 5.3, 3.2 Hz, 1H), 7.30 - 7.25 (m, 2H), 7.05 (ddt, J = 9.1, 7.2, 3.5 Hz, 1H), 6.88 (td, J = 9.1,4.3 Hz, 1H), 6.43 (d, J = 8.9 Hz, 1H), 4.53 (s, 1H), 3.64 (dt, J = 10.4, 2.6 Hz, 1H), 3.51 (dd, J = 10.4, 1.8 Hz, 1H), 3.25 (dt, J = 11.0, 2.7 Hz, 1H), 3.20 - 3.06 (m, 2H), 2.01 - 1.91 (m, 2H), 1.89 - 1.80 (m, 1H), 1.80 - 1.63 (m, 1H). LCMS (Analytical Method A) Rt = 1.43 min, MS (ESIpos): m/z 419.3 [M+H]+, Purity = 99%. |
| | | rac-(1R,4R)-2-[2-(2,5-difluorophenyl)-3-(pyridin-4-yl)-3H-imidazo[4,5-b]pyridin-5-yl]-2,5-diazabicyclo[2.2.2]octane | |
| 60-R | Intermediate 37 (25% yield) | 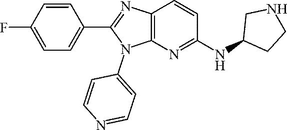 [SMILES: Fc1ccc(-c2nc3ccc(N[C@@H]4CCNC4)nc3n2-c2ccncc2)cc1] | 1H NMR (400 MHz, Chloroform-d) δ 8.70 - 8.57 (m, 2H), 7.79 (d, J = 8.7 Hz, 1H), 7.46 - 7.36 (m, 2H), 7.32 -7.23 (m, 2H), 7.06 - 6.92 (m, 2H), 6.38 (d, J = 8.7 Hz, 1H), 4.68 -4.56 (m, 1H), 4.25 - 4.08 (m, 1H), 3.12 (dd, J = 11.3, 6.1 Hz, 1H), 3.03 (ddd, J = 10.9, 7.9, 6.2 Hz, 1H), 2.88 (ddd, J = 10.9, 8.3, 6.1 Hz, 1H), 2.80 (dd, J = 11.3, 3.8 Hz, 1H), 2.21 - 2.04 (m, 1H), 1.66 - 1.58 (m, 1H). LCMS (Analytical Method A) Rt = 1.38 min, MS (ESIpos): m/z 375.3 [M+H]+, Purity = 98%. |
| | | (3R)-N-[2-(4-fluorophenyl)-3-(pyridin-4-yl)-3H-imidazo[4,5-b]pyridin-5-yl]pyrrolidin-3-amine | |
| 61 | Intermediate 12 (21% yield) | 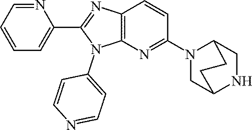 [SMILES: c1ccc(-c2nc3ccc(N4CC5CCC4CN5)nc3n2-c2ccncc2)nc1] | 1H NMR (500 MHz, Chloroform-d) δ 8.60 - 8.56 (m, 2H), 8.29 (ddd, J = 4.8, 1.7, 0.9 Hz, 1H), 8.04 - 7.99 (m, 1H), 7.85 (d, J = 8.9 Hz, 1H), 7.71 (td, J = 7.8, 1.8 Hz, 1H), 7.28 - 7.21 (m, 2H), 7.17 - 7.13 (m, 1H),6.41 (d, J = 8.9 Hz, 1H), 4.53 - 4.45 (m, 1H), 3.62 (dt, J = 10.4, 2.7 Hz, 1H), 3.49 (dd, J = 10.4, 1.9 Hz, 1H), 3.23 (dt, J = 11.0, 2.7 Hz, 1H), 3.17 - 3.13 (m, 1H), 3.11 (dd, J = 11.1, 1.9 Hz, 1H), 2.00 - 1.87 (m, 2H), 1.87 - 1.78 (m, 1H), 1.76 - 1.67 (m, 1H). LCMS (Analytical Method A) Rt = 1.08 min, MS (ESIpos): m/z 384.3 [M+H]+, Purity = 97%. |
| | | rac-(1R,4R)-2-[2-(pyridin-2-yl)-3-(pyridin-4-yl)-3H-imidazo[4,5-b]pyridin-5-yl]-2,5-diazabicyclo[2.2.2]octane | |
| 62 | Intermediate 12 (13% yield) | 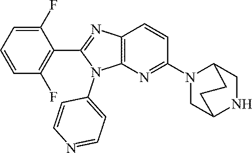 [SMILES: Fc1cccc(F)c1-c1nc2ccc(N3CC4CCC3CN4)nc2n1-c1ccncc1] | 1H NMR (500 MHz, Chloroform-d) δ 8.60 - 8.51 (m, 2H), 7.89 (d, J = 8.9 Hz, 1H), 7.42 - 7.31 (m, 1H), 7.30 -7.25 (m, 2H), 6.93 - 6.83 (m, 2H), 6.43 (d, J = 8.9 Hz, 1H), 4.53 (s, 1H), 3.65 - 3.60 (m, 1H), 3.54 - 3.48 (m, 1H), 3.30-3.21 (m, 1H), 3.17 - 3.11 (m, 2H), 2.01 - 1.90 (m, 2H), 1.90 -1.82 (m, 1H), 1.77 - 1.70 (m, 1H). LCMS (Analytical Method A) Rt = 1.44 min, MS (ESIpos): m/z 419.3 [M+H]+, Purity = 98%. |
| | | rac-(1R,4R)-2-[2-(2,6-difluorophenyl)-3-(pyridin-4-yl)-3H-imidazo[4,5-b]pyridin-5-yl]-2,5-diazabicyclo[2.2.2]octane | |
| 63 | Intermediate 12 (35% yield) | 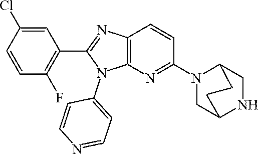 [SMILES: Fc1ccc(Cl)cc1-c1nc2ccc(N3CC4CCC3CN4)nc2n1-c1ccncc1] | 1H NMR (400 MHz, Chloroform-d) δ 8.62 - 8.53 (m, 2H), 7.85 (d, J = 8.9 Hz, 1H), 7.72 (dd, J = 6.0, 2.7 Hz, 1H), 7.35 - 7.28 (m, 1H), 7.28 - 7.23 (m, 2H), 6.84 (t, J = 9.1 Hz, 1H), 6.42 (d, J = 8.9 Hz, 1H), 4.56 - 4.45 (m, 1H), 3.63 (dt, J = 10.4, 2.5 Hz, 1H), 3.50 (dd, J = 10.4, 1.6 Hz, 1H), 3.28 - 3.19 (m, 1H), 3.17 - 3.09 (m, 2H), 2.02 - 1.80 (m, 3H), 1.77 - 1.66 (m, 1H). LCMS (Analytical Method A) Rt = 1.68 min, MS (ESIpos): m/z 435.3 [M+H]+, Purity = 97%. |
| | | rac-(1R,4R)-2-[2-(5-chloro-2-fluorophenyl)-3-(pyridin-4-yl)-3H-imidazo[4,5-b]pyridin-5-yl]-2,5-diazabicyclo[2.2.2]octane | |
| 64 | Intermediate 12 (8% yield) | 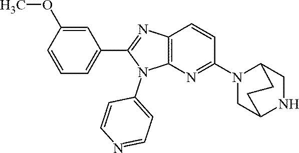 [SMILES: COc1cccc(-c2nc3ccc(N4CC5CCC4CN5)nc3n2-c2ccncc2)c1] | 1H NMR (500 MHz, Chloroform-d) δ 8.62 - 8.58 (m, 2H), 7.85 (d, J = 8.8 Hz, 1H), 7.32 - 7.27 (m, 2H), 7.18 -7.13 (m, 1H), 7.09 - 7.03 (m, 1H), 6.93 - 6.88 (m, 1H), 6.88 - 6.82 (m, 1H), 6.40 (d, J = 8.9 Hz, 1H), 4.50 (s, 1H), 3.65 - 3.58 (m, 1H), 3.50 (dd, J = 10.3, 1.8 Hz, 1H), 3.24 (dt, J = 11.0, 2.6 Hz, 1H), 3.15 (s, 1H), 3.12 (dd, J = 11.0, 1.9 Hz, 1H), 1.94 (dd, J = 8.0, 2.5 Hz, 2H), 1.89 - 1.82 (m, 1H), 1.77 - 1.68 (m, 1H). LCMS (Analytical Method A) Rt = 1.41 min, MS (ESIpos): m/z 413.3 [M+H]+, Purity = 100%. |
| | | rac-(1R,4R)-2-[2-(3-methoxyphenyl)-3-(pyridin-4-yl)-3H-imidazo[4,5-b]pyridin-5-yl]-2,5-diazabicyclo[2.2.2]octane | |
| 65 | Intermediate 12 (48% yield) | 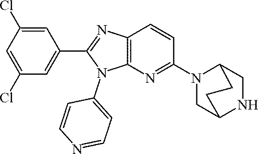 [SMILES: Clc1cc(Cl)cc(-c2nc3ccc(N4CC5CCC4CN5)nc3n2-c2ccncc2)c1] | 1H NMR (400 MHz, Chloroform-d) δ 8.72 - 8.60 (m, 2H), 7.84 (d, J = 8.9 Hz, 1H), 7.34 - 7.31 (m, 2H), 7.31 -7.26 (m, 3H), 6.43 (d, J = 8.9 Hz, 1H), 4.53 - 4.44 (m, 1H), 3.62 (dt, J = 10.3, 2.5 Hz, 1H), 3.50 (dd, J = 10.4, 1.7 Hz, 1H), 3.29 - 3.19 (m, 1H), 3.18 - 3.07 (m, 2H), 2.00 - 1.81 (m, 3H), 1.78 - 1.69 (m, 1H). LCMS (Analytical Method A) Rt = 1.99 min, MS (ESIpos): m/z 452.3 [M+H]+, Purity = 96%. |
| | | rac-(1R,4R)-2-[2-(3,5-dichlorophenyl)-3-(pyridin-4-yl)-3H-imidazo[4,5-b]pyridin-5-yl]-2,5-diazabicyclo[2.2.2]octane | |
| 35-RR | Intermediate 38 (14% yield) | 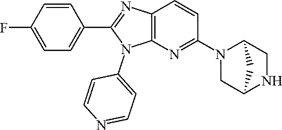 [SMILES: Fc1ccc(-c2nc3ccc(N4C[C@H]5C[C@@H]4CN5)nc3n2-c2ccncc2)cc1] | 1H NMR (400 MHz, Chloroform-d) δ 8.67 - 8.56 (m, 2H), 7.80 (d, J = 8.8 Hz, 1H), 7.46 - 7.37 (m, 2H), 7.34 -7.23 (m, 2H), 7.03 - 6.91 (m, 2H), 6.34 (d, J = 8.8 Hz, 1H), 4.72 - 4.59 (m, 1H), 3.87 - 3.75 (m, 1H), 3.53 (dd, J = 9.6, 2.0 Hz, 1H), 3.24 (d, J = 9.6 Hz, 1H), 3.09 - 2.93 (m, 2H), 1.85 (d, J = 9.7 Hz, 1H), 1.77 (d, J = 9.7 Hz, 1H). LCMS (Analytical Method A) Rt = 1.39 min, MS (ESIpos): m/z 387.2 [M+H]+, Purity = 98%. |
| | | (1R,4R)-2-[2-(4-fluorophenyl)-3-(pyridin-4-yl)-3H-imidazo[4,5-b]pyridin-5-yl]-2,5-diazabicyclo[2.2.1]heptane | |
| 66 | Intermediate 12 (42% yield) | 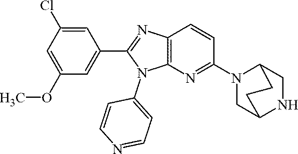 [SMILES: COc1cc(Cl)cc(-c2nc3ccc(N4CC5CCC4CN5)nc3n2-c2ccncc2)c1] | 1H NMR (500 MHz, Chloroform-d) δ 8.67 - 8.60 (m, 2H), 7.84 (d, J = 8.9 Hz, 1H), 7.32 - 7.25 (m, 2H), 7.05 (t, J = 1.6 Hz, 1H), 6.88 - 6.81 (m, 1H), 6.80 (dd, J = 2.3, 1.4 Hz, 1H), 6.41 (d, J = 8.9 Hz, 1H), 4.53 - 4.44 (m, 1H), 3.65 (s, 3H), 3.63 - 3.59 (m, 1H), 3.50 (dd, J = 10.3, 1.9 Hz, 1H), 3.24 (dt, J = 11.0, 2.7 Hz, 1H), 3.17 - 3.14 (m, 1H), 3.12 (dd, J = 11.0, 1.9 Hz, 1H), 2.00 - 1.89 (m, 2H), 1.89 - 1.81 (m, 1H), 1.78 - 1.65 (m, 1H). LCMS (Analytical Method A) Rt = 1.80 min, MS (ESIpos): m/z 447.2 [M+H]+, Purity = 100%. |
| | | rac-(1R,4R)-2-[2-(3-chloro-5-methoxyphenyl)-3-(pyridin-4-yl)-3H-imidazo[4,5-b]pyridin-5-yl]-2,5-diazabicyclo[2.2.2]octane | |
| 36-SS | Intermediate 39 (36% yield) | 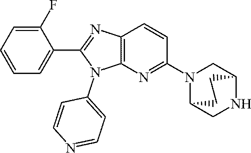 [SMILES: Fc1ccccc1-c1nc2ccc(N3C[C@@H]4CC[C@H]3CN4)nc2n1-c1ccncc1] | 1H NMR (400 MHz, DMSO-d6) δ 8.63 (dd, J = 4.6, 1.6 Hz, 2H), 7.95 (d, J = 8.9 Hz, 1H), 7.72 (td, J = 7.5, 1.7 Hz, 1H), 7.58 - 7.46 (m, 1H), 7.43 - 7.28 (m, 3H), 7.24 - 7.12 (m, 1H), 6.62 (d, J = 9.0 Hz, 1H), 4.46 (s, 1H), 3.65 - 3.44 (m, 2H), 3.12 - 2.95 (m, 3H), 1.94 - 1.76 (m, 3H), 1.72 -1.59 (m, 1H). LCMS (Analytical Method A) Rt = 1.42 min, MS (ESIpos): m/z 401 [M+H]+, Purity = 100%. |
| | | (1S,4S)-2-[2-(2-fluorophenyl)-3-(pyridin-4-yl)-3H-imidazo [4,5-b]pyridin-5-yl]-2,5-diazabicyclo[2.2.2]octane | |
| 36-RR | Intermediate 40 (47% yield) | 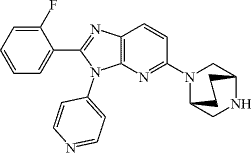 [SMILES: Fc1ccccc1-c1nc2ccc(N3C[C@H]4CC[C@@H]3CN4)nc2n1-c1ccncc1] | 1H NMR (400 MHz, DMSO-d6) δ 8.63 (dd, J = 4.6, 1.6 Hz, 2H), 7.95 (d, J = 8.9 Hz, 1H), 7.72 (td, J = 7.5, 1.7 Hz, 1H), 7.60 - 7.48 (m, 1H), 7.41 - 7.31 (m, 3H), 7.23 - 7.16 (m, 1H), 6.62 (d, J = 8.9 Hz, 1H), 4.46 (s, 1H), 3.63 - 3.42 (m, 2H), 3.13 - 2.94 (m, 3H), 1.93 - 1.76 (m, 3H), 1.72 -1.59 (m, 1H). LCMS (Analytical Method A) Rt = 1.41 min, MS (ESIpos): m/z 401 [M+H]+, Purity = 100%. |
| | | (1R,4R)-2-[2-(2-fluorophenyl)-3-(pyridin-4-yl)-3H-imidazo[4,5-b]pyridin-5-yl]-2,5-diazabicyclo[2.2.2]octane | |
| 60-S | Intermediate 41 (22% yield) | 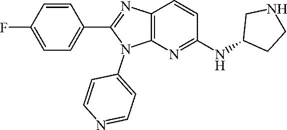 [SMILES: Fc1ccc(-c2nc3ccc(N[C@H]4CCNC4)nc3n2-c2ccncc2)cc1] | 1H NMR (500 MHz, Chloroform-d) δ 8.63 (d, J = 5.9 Hz, 2H), 7.79 (d, J = 8.6 Hz, 1H), 7.45 - 7.38 (m, 2H), 7.27 (d, J = 5.9 Hz, 2H), 6.98 (t, J = 8.5 Hz, 2H), 6.38 (d, J = 8.7 Hz, 1H), 4.66 - 4.58 (m, 1H), 4.20 - 4.11 (m, 1H), 3.13 (dd, J = 11.3, 6.1 Hz, 1H), 3.06 - 2.99 (m, 1H), 2.94 - 2.84 (m, 1H), 2.80 (dd, J = 11.2, 3.6 Hz, 1H), 2.19 - 2.08 (m, 1H), 1.64 - 1.57 (m, 1H). LCMS (Analytical Method A) Rt = 1.37 min, MS (ESIpos): m/z 375.2 [M+H]+, Purity = 100%. |
| | | (3S)-N-[2-(4-fluorophenyl)-3-(pyridin-4-yl)-3H-imidazo[4,5-b]pyridin-5-yl]pyrrolidin-3-amine | |
| 67 | Intermediate 12 (49% yield) | 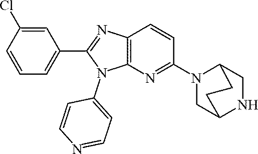 [SMILES: Clc1cccc(-c2nc3ccc(N4CC5CCC4CN5)nc3n2-c2ccncc2)c1] | 1H NMR (500 MHz, Chloroform-d) δ 8.68 - 8.60 (m, 2H), 7.84 (d, J = 8.9 Hz, 1H), 7.59 (s, 1H), 7.32 - 7.24 (m, 3H), 7.17 (d, J = 5.0 Hz, 2H), 6.41 (d, J = 8.9 Hz, 1H), 4.53 - 4.46 (m, 1H), 3.62 (dt, J = 10.2, 2.4 Hz, 1H), 3.50 (dd, J = 10.3, 1.5 Hz, 1H), 3.24 (dt, J = 11.0, 2.6 Hz, 1H), 3.15 (s, 1H), 3.12 (dd, J = 11.0, 1.6 Hz, 1H), 2.00 - 1.89 (m, 2H), 1.89 - 1.81 (m, 1H), 1.77 - 1.69 (m, 1H). LCMS (Analytical Method A) Rt = 1.67 min, MS (ESIpos): m/z 417.2 [M+H]+, Purity = 95%. |
| | | rac-(1R,4R)-2-[2-(3-chlorophenyl)-3-(pyridin-4-yl)-3H-imidazo[4,5-b]pyridin-5-yl]-2,5-diazabicyclo[2.2.2]octane | |
| 68 | Intermediate 12 (41% yield) | 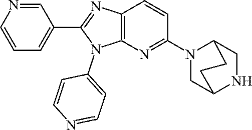 [SMILES: c1cncc(-c2nc3ccc(N4CC5CCC4CN5)nc3n2-c2ccncc2)c1] | 1H NMR (500 MHz, Chloroform-d) δ 8.71 - 8.67 (m, 1H), 8.67 - 8.59 (m, 2H), 8.53 (dd, J = 4.8, 1.6 Hz, 1H), 7.85 (d, J = 8.9 Hz, 1H), 7.73 (dt, J = 8.0, 1.9 Hz, 1H), 7.29 - 7.25 (m, 2H), 7.21 (ddd, J = 8.0, 4.8, 0.8 Hz, 1H), 6.41 (d, J = 8.9 Hz, 1H), 4.49 (s, 1H), 3.61 (dt, J = 10.3, 2.7 Hz, 1H), 3.49 (dd, J = 10.3, 1.9 Hz, 1H), 3.23 (dt, J = 11.0, 2.7 Hz, 1H), 3.16 - 3.07 (m, 2H), 1.98- 1.88 (m, 2H), 1.88-1.79 (m, 1H), 1.78 - 1.65 (m, 1H). LCMS (Analytical Method B) Rt = 2.01 min, MS (ESIpos): m/z 384.3 [M+H]+, Purity = 100%. |
| | | rac-(1R,4R)-2-[2-(pyridin-3-yl)-3-(pyridin-4-yl)-3H-imidazo[4,5-b]pyridin-5-yl]-2,5-diazabicyclo[2.2.2]octane | |
| 69 | Intermediate 12 (20% yield) | 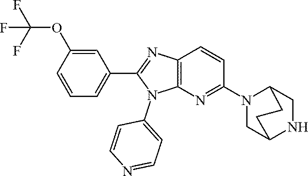 [SMILES: FC(F)(F)Oc1cccc(-c2nc3ccc(N4CC5CCC4CN5)nc3n2-c2ccncc2)c1] | 1H NMR (500 MHz, Chloroform-d) δ 8.69 - 8.55 (m, 2H), 7.85 (d, J = 8.9 Hz, 1H), 7.37 (d, J = 8.0 Hz, 1H), 7.33 - 7.29 (m, 2H), 7.29 - 7.26 (m, 2H), 7.16 (d, J = 8.2 Hz, 1H), 6.42 (d, J = 8.9 Hz, 1H), 4.50 (s, 1H), 3.62 (dt, J = 10.3, 2.6 Hz, 1H), 3.50 (dd, J = 10.3, 1.8 Hz, 1H), 3.24 (dt, J = 11.0, 2.7 Hz, 1H), 3.17 - 3.07 (m, 2H), 2.00 -1.88 (m, 2H), 1.88 - 1.80 (m, 1H), 1.77 - 1.68 (m, 1H). LCMS (Analytical Method A) Rt = 1.89 min, MS (ESIpos): m/z 467.2 [M+H]+, Purity = 95%. |
| | | rac-(1R,4R)-2-[3-(pyridin-4-yl)-2-[3-(trifluoromethoxy)phenyl]-3H-imidazo[4,5-b]pyridin-5-yl]-2,5-diazabicyclo[2.2.2]octane | |
| 71-RR | Intermediate 38 (42% yield) | 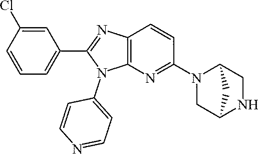 [SMILES: Clc1cccc(-c2nc3ccc(N4C[C@H]5C[C@@H]4CN5)nc3n2-c2ccncc2)c1] | 1H NMR (500 MHz, Chloroform-d) δ 8.66 - 8.61 (m, 2H), 7.82 (d, J = 8.8 Hz, 1H), 7.62 - 7.56 (m, 1H), 7.33 -7.26 (m, 3H), 7.18 - 7.15 (m, 2H), 6.36 (d, J = 8.8 Hz, 1H), 4.68 - 4.63 (m, 1H), 3.79 - 3.74 (m, 1H), 3.57 -3.52 (m, 1H), 3.22 - 3.18 (m, 1H), 3.05 - 2.96 (m, 2H), 1.87 - 1.72 (m, 2H). LCMS (Analytical Method A) Rt = 1.59 min, MS (ESIpos): m/z 403.2 [M+H]+, Purity = 98%. |
| | | (1R,4R)-2-[2-(3-chlorophenyl)-3-(pyridin-4-yl)-3H-imidazo[4,5-b]pyridin-5-yl]-2,5-diazabicyclo[2.2.1]heptane | |
| 72-RR | Intermediate 38 (11 % yield) | 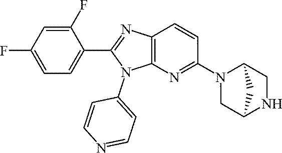 [SMILES: Fc1ccc(-c2nc3ccc(N4C[C@H]5C[C@@H]4CN5)nc3n2-c2ccncc2)c(F)c1] | 1H NMR (400 MHz, Chloroform-d) δ 8.64 - 8.50 (m, 2H), 7.82 (d, J = 8.8 Hz, 1H), 7.69 - 7.60 (m, 1H), 7.29 -7.23 (m, 2H), 6.99 - 6.91 (m, 1H), 6.73 - 6.63 (m, 1H), 6.37 (d, J = 8.8 Hz, 1H), 4.76 - 4.57 (m, 1H), 3.81 -3.74 (m, 1H), 3.58 - 3.52 (m, 1H), 3.24 - 3.17 (m, 1H), 3.06 - 2.97 (m, 2H), 1.91 - 1.68 (m, 2H). LCMS (Analytical Method B) Rt = 2.60 min, MS (ESIpos): m/z 405.3 [M+H]+, Purity = 97%. |
| | | (1R,4R)-2-[2-(2,4-difluorophenyl)-3-(pyridin-4-yl)-3H-imidazo[4,5-b]pyridin-5-yl]-2,5-diazabicyclo[2.2.1]heptane | |
| 73-RR | Intermediate 38 (37% yield) | 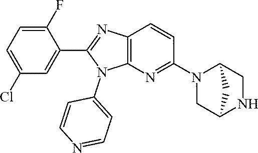 [SMILES: Fc1ccc(Cl)cc1-c1nc2ccc(N3C[C@H]4C[C@@H]3CN4)nc2n1-c1ccncc1] | 1H NMR (400 MHz, DMSO) δ 8.70 -8.61 (m, 2H), 7.95 (d, J = 8.8 Hz, 1H), 7.84 -7.76 (m, 1H), 7.66 - 7.57 (m, 1H), 7.47 - 7.39 (m, 2H), 7.31 -7.22 (m, 1H), 6.61 (d, J = 8.8 Hz, 1H), 4.72 - 4.61 (m, 1H), 3.69 - 3.60 (m, 1H), 3.47 (dd, J = 9.5, 1.9 Hz, 1H), 3.27 - 3.23 (m, 1H), 2.90 (d, J = 8.1 Hz, 1H), 2.79 (d, J = 9.6 Hz, 1H), 1.77 (d, J = 9.0 Hz, 1H), 1.66 (d, J = 8.9 Hz, 1H). LCMS (Analytical Method A) Rt = 1.56 min, MS (ESIpos): m/z 421.3, 423.3 [M+H]+, Purity = 99%. |
| | | (1R,4R)-2-[2-(5-chloro-2-fluorophenyl)-3-(pyridin-4-yl)-3H-imidazo[4,5-b]pyridin-5-yl]-2,5-diazabicyclo[2.2.1]heptane | |
| 75 | Intermediate 4 (10% yield) | 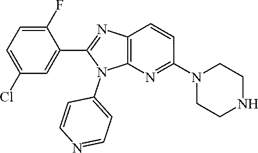 [SMILES: Fc1ccc(Cl)cc1-c1nc2ccc(N3CCNCC3)nc2n1-c1ccncc1] | 1H NMR (400 MHz, Chloroform-d) δ 8.64 - 8.54 (m, 2H), 7.88 (d, J = 9.0 Hz, 1H), 7.73 (dd, J = 6.0, 2.7 Hz, 1H), 7.33 (ddd, J = 8.8, 4.3, 2.7 Hz, 1H), 7.30 - 7.24 (m, 2H), 6.91 - 6.80 (m, 1H), 6.70 (d, J = 9.0 Hz, 1H), 3.53 - 3.42 (m, 4H), 2.97 - 2.87 (m, 4H). LCMS (Analytical Method B) Rt = 2.77 min, MS (ESIpos): m/z 409.3 [M+H]+, Purity = 100%. |
| | | 1-[2-(5-chloro-2-fluorophenyl)-3-(pyridin-4-yl)-3H-imidazo[4,5-b]pyridin-5-yl]piperazine | |
| 76 | Intermediate 12 (6% yield) | 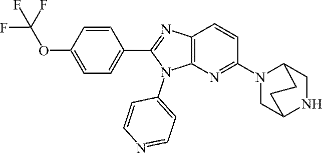 [SMILES: FC(F)(F)Oc1ccc(-c2nc3ccc(N4CC5CCC4CN5)nc3n2-c2ccncc2)cc1] | 1H NMR (400 MHz, Chloroform-d) δ 8.69 - 8.56 (m, 2H), 7.84 (d, J = 8.9 Hz, 1H), 7.51 - 7.43 (m, 2H), 7.32 -7.26 (m, 2H), 7.15 - 7.08 (m, 2H), 6.41 (d, J = 8.9 Hz, 1H), 4.57 - 4.43 (m, 1H), 3.66 - 3.59 (m, 1H), 3.52 -3.45 (m, 1H), 3.28 - 3.20 (m, 1H), 3.18 - 3.07 (m, 2H), 2.01 - 1.80 (m, 3H), 1.80 - 1.64 (m, 1H). LCMS (Analytical Method A) Rt = 1.92 min, MS (ESIpos): m/z 467.3 [M+H]+, Purity = 95%. |
| | | rac-(1R,4R)-2-[3-(pyridin-4-yl)-2-[4-(trifluoromethoxy)phenyl]-3H-imidazo[4,5-b]pyridin-5-yl]-2,5-diazabicyclo[2.2.2]octane | |
| 77-RS | Intermediate 32 (49% yield) | 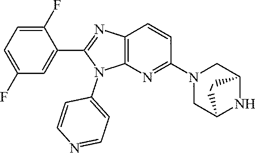 [SMILES: Fc1ccc(F)c(-c2nc3ccc(N4C[C@H]5CC[C@@H](C4)N5)nc3n2-c2ccncc2)c1] | 1H NMR (400 MHz, Chloroform-d) δ 8.64 - 8.48 (m, 2H), 7.86 (d, J = 9.0 Hz, 1H), 7.47 - 7.34 (m, 1H), 7.34 -7.23 (m, 2H), 7.06 (ddt, J = 9.1, 7.2, 3.5 Hz, 1H), 6.88 (td, J = 9.1, 4.3 Hz, 1H), 6.58 (d, J = 9.0 Hz, 1H), 3.85 (dd, J = 12.0, 2.2 Hz, 2H), 3.66 -3.52 (m, 2H), 2.98 (dd, J = 11.9, 2.1 Hz, 2H), 1.81 - 1.64 (m, 4H). LCMS (Analytical Method A) Rt = 1.50 min, MS (ESIpos): m/z 419.3 [M+H]+, Purity = 97% |
| | | (1R,5S)-3-[2-(2,5-difluorophenyl)-3-(pyridin-4-yl)-3H-imidazo[4,5-b]pyridin-5-yl]-3,8-diazabicyclo[3.2.1]octane | |
| 78-RS | Intermediate 32 (43% yield) | 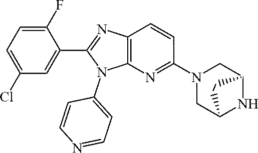 | 1H NMR (400 MHz, Chloroform-d) δ 8.63 - 8.51 (m, 2H), 7.85 (d, J = 9.0 Hz, 1H), 7.73 (dd, J = 6.0, 2.7 Hz, 1H), 7.32 (ddd, J = 8.8, 4.3, 2.7 Hz, 1H), 7.29 - 7.26 (m, 2H), 6.89 - 6.81 (m, 1H), 6.59 (d, J = 9.0 Hz, 1H), 3.85 (dd, J = 12.0, 2.2 Hz, 2H), 3.65 - 3.50 (m, 2H), 2.99 (dd, J = 12.0, 2.1 Hz, 2H), 1.79 - 1.65 (m, 4H). LCMS (Analytical Method A) Rt = 1.69 min, MS (ESIpos): m/z 435.3 [M+H]+, Purity = 95%. |
| | | (1R,5S)-3-[2-(5-chloro-2-fluorophenyl)-3-(pyridin-4-yl)-3H-imidazo[4,5-b]pyridin-5-yl]-3,8-diazabicyclo[3.2.1]octane | |
| 87-RR | Intermediate 38 (9% yield) | 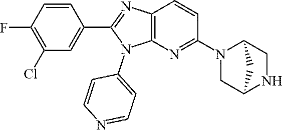 [SMILES: Fc1ccc(-c2nc3ccc(N4C[C@H]5C[C@@H]4CN5)nc3n2-c2ccncc2)cc1Cl] | 1H NMR (500 MHz, Chloroform-d) δ 8.74 - 8.61 (m, 2H), 7.80 (d, J = 8.8 Hz, 1H), 7.65 (dd, J = 7.0, 2.2 Hz, 1H), 7.31 - 7.26 (m, 2H), 7.18 - 7.15 (m, 1H), 7.05 - 6.99 (m, 1H), 6.36 (d, J = 8.8 Hz, 1H), 4.69 -4.60 (m, 1H), 3.77 (s, 1H), 3.54 (dd, J = 9.5, 2.1 Hz, 1H), 3.19 (d, J = 9.4 Hz, 1H), 3.06 - 2.95 (m, 2H), 1.88 - 1.80 (m, 1H), 1.80 - 1.71 (m, 1H). LCMS (Analytical Method A) Rt = 1.74 min, MS (ESIpos): m/z 421.2 [M+H]+, Purity = 97%. |
| | | (1R,4R)-2-[2-(3-chloro-4-fluorophenyl)-3-(pyridin-4-yl)-3H-imidazo[4,5-b]pyridin-5-yl]-2,5-diazabicyclo[2.2.1]heptane | |
| 91 | Intermediate 42 (19% yield) | 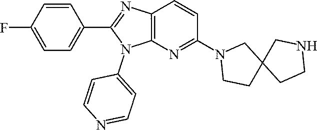 [SMILES: Fc1ccc(-c2nc3ccc(N4CCC5(CCNC5)C4)nc3n2-c2ccncc2)cc1] | 1H NMR (500 MHz, Chloroform-d) δ 8.65 - 8.59 (m, 2H), 7.81 (d, J = 8.8 Hz, 1H), 7.47 - 7.38 (m, 2H), 7.35 -7.27 (m, 2H), 7.03 - 6.91 (m, 2H), 6.35 (d, J = 8.8 Hz, 1H), 3.55 - 3.42 (m, 2H), 3.38 (d, J = 10.1 Hz, 1H), 3.33 (d, J = 10.1 Hz, 1H), 3.04 - 2.94 (m, 2H), 2.85 (d, J = 10.8 Hz, 1H), 2.79 (d, J = 10.8 Hz, 1H), 1.97-1.88 (m, 2H), 1.83 - 1.67 (m, 2H). LCMS (Analytical Method A) Rt = 1.81 min, MS (ESIpos): m/z 415.2 [M+H]+, Purity = 96%. |
| | | 2-[2-(4-fluorophenyl)-3-(pyridin-4-yl)-3H-imidazo[4,5-b]pyridin-5-yl]-2,7-diazaspiro[4.4]nonane | |
| 92 | Intermediate 42 (28% yield) | 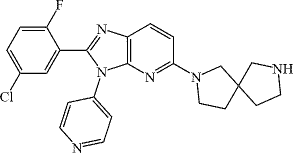 [SMILES: Fc1ccc(Cl)cc1-c1nc2ccc(N3CCC4(CCNC4)C3)nc2n1-c1ccncc1] | 1H NMR (500 MHz, Chloroform-d) δ 8.62 - 8.55 (m, 2H), 7.83 (d, J = 8.8 Hz, 1H), 7.74 - 7.70 (m, 1H), 7.34 -7.29 (m, 3H), 6.90 - 6.80 (m, 1H), 6.38 (d, J = 8.9 Hz, 1H), 3.59 - 3.44 (m, 2H), 3.39 (d, J = 10.2 Hz, 1H), 3.35 (d, J = 10.2 Hz, 1H), 3.00 (d, J = 6.6 Hz, 2H), 2.86 (d, J = 10.8 Hz, 1H), 2.79 (d, J = 10.8 Hz, 1H), 1.94 (d, J = 6.8 Hz, 2H), 1.84 - 1.68 (m, 2H). LCMS (Analytical Method A) Rt = 2.05 min, MS (ESIpos): m/z 449.2/ 451.2 [M+H]+, Purity = 97%. |
| | | 2-[2-(5-chloro-2-fluorophenyl)-3-(pyridin-4-yl)-3H-imidazo[4,5-b]pyridin-5-yl]-2,7-diazaspiro[4.4]nonane | |
| 93-RR | Intermediate 38 (8% yield) | 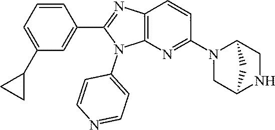 [SMILES: c1cc(-c2nc3ccc(N4C[C@H]5C[C@@H]4CN5)nc3n2-c2ccncc2)cc(C2CC2)c1] | 1H NMR (500 MHz, Chloroform-d) δ 8.63 - 8.55 (m, 2H), 7.82 (d, J = 8.8 Hz, 1H), 7.32 - 7.26 (m, 2H), 7.18 (s, 1H), 7.15 - 7.09 (m, 2H), 7.07 -7.01 (m, 1H), 6.34 (d, J = 8.8 Hz, 1H), 4.69 - 4.59 (m, 1H), 3.79 - 3.70 (m, 1H), 3.55 (dd, J = 9.5, 2.1 Hz, 1H), 3.20 (d, J = 9.7 Hz, 1H), 3.05 -2.94 (m, 2H), 1.87 - 1.72 (m, 3H), 0.89 - 0.80 (m, 2H), 0.55 - 0.46 (m, 2H). LCMS (Analytical Method A) Rt = 1.68 min, MS (ESIpos): m/z 409.2 [M+H]+, Purity = 100%. |
| | | (1R,4R)-2-[2-(3-cyclopropylphenyl)-3-(pyridin-4-yl)-3H-imidazo[4,5-b]pyridin-5-yl]-2,5-diazabicyclo[2.2.1]heptane | |
| 94-RR | Intermediate 38 (16% yield) | 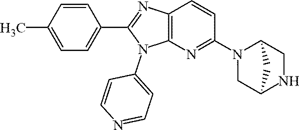 [SMILES: Cc1ccc(-c2nc3ccc(N4C[C@H]5C[C@@H]4CN5)nc3n2-c2ccncc2)cc1] | 1H NMR (500 MHz, Chloroform-d) δ 8.63 - 8.55 (m, 2H), 7.81 (d, J = 8.7 Hz, 1H), 7.32 (d, J = 8.2 Hz, 2H), 7.31 - 7.28 (m, 2H), 7.08 (d, J = 7.9 Hz, 2H), 6.33 (d, J = 8.8 Hz, 1H), 4.71 - 4.60 (m, 1H), 3.81 - 3.72 (m, 1H), 3.55 (dd, J = 9.5, 2.0 Hz, 1H), 3.20 (d, J = 9.4 Hz, 1H), 3.06 - 2.93 (m, 2H), 2.30 (s, 3H), 1.88 -1.79 (m, 1H), 1.79 - 1.72 (m, 1H). LCMS (Analytical Method A) Rt = 1.47 min, MS (ESIpos): m/z 383.2 [M+H]+, Purity = 100%. |
| | | (1R,4R)-2-[2-(4-methylphenyl)-3-(pyridin-4-yl)-3H-imidazo[4,5-b]pyridin-5-yl]-2,5-diazabicyclo[2.2.1]heptane | |
| 56-RR | Intermediate 40 (3% yield) | 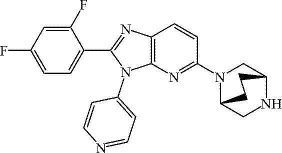 [SMILES: Fc1ccc(-c2nc3ccc(N4C[C@H]5CC[C@@H]4CN5)nc3n2-c2ccncc2)c(F)c1] | 1H NMR (500 MHz, Chloroform-d) δ 8.73 - 8.55 (m, 2H), 7.92 (d, J = 8.9 Hz, 1H), 7.72 (td, J = 8.3, 6.4 Hz, 1H), 7.35 - 7.29 (m, 2H), 7.07 - 6.98 (m, 1H), 6.76 (ddd, J = 11.0, 8.8, 2.4 Hz, 1H), 6.49 (d, J = 8.9 Hz, 1H), 4.59 (s, 1H), 3.70 (dt, J = 10.3, 2.6 Hz, 1H), 3.61 - 3.55 (m, 1H), 3.32 (dt, J = 10.9, 2.6 Hz, 1H), 3.25 - 3.16 (m, 2H), 2.00 (ddd, J = 17.5, 6.4, 3.2 Hz, 2H), 1.97 - 1.87 (m, 1H), 1.86 -1.75 (m, 1H). LCMS (Analytical Method A) Rt = 1.52 min, MS (ESIpos): m/z 419 [M+H]+, Purity = 100%. |
| | | (1R,4R)-2-[2-(2,4-difluorophenyl)-3-(pyridin-4-yl)-3H-imidazo[4,5-b]pyridin-5-yl]-2,5-diazabicyclo[2.2.2]octane | |
| 96-RR | Intermediate 38 (47% yield) | 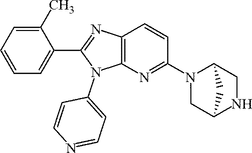 [SMILES: Cc1ccccc1-c1nc2ccc(N3C[C@H]4C[C@@H]3CN4)nc2n1-c1ccncc1] | 1H NMR (500 MHz, Chloroform-d) δ 8.53 - 8.45 (m, 2H), 7.83 (d, J = 8.7 Hz, 1H), 7.30 - 7.25 (m, 2H), 7.25 -7.21 (m, 2H), 7.17 - 7.12 (m, 2H), 6.37 (d, J = 8.8 Hz, 1H), 4.69 (s, 1H), 3.78 (s, 1H), 3.58 (dd, J = 9.5, 1.8 Hz, 1H), 3.22 (d, J = 9.5 Hz, 1H), 3.04 (s, 2H), 2.06 (s, 3H), 1.86 (d, J = 9.3 Hz, 1H), 1.78 (d, J = 9.5 Hz, 1H). LCMS (Analytical Method B) Rt = 2.62 min, MS (ESIpos): m/z 383.4 [M+H]+, Purity = 100%. |
| | | (1R,4R)-2-[2-(2-methylphenyl)-3-(pyridin-4-yl)-3H-imidazo[4,5-b]pyridin-5-yl]-2,5-diazabicyclo[2.2.1]heptane | |
| 100 | Intermediate 4 (47% yield) | 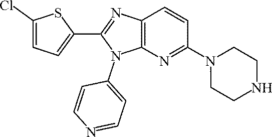 [SMILES: Clc1ccc(-c2nc3ccc(N4CCNCC4)nc3n2-c2ccncc2)s1] | 1H NMR (500 MHz, Chloroform-d) δ 8.81 - 8.68 (m, 2H), 7.81 (d, J = 8.9 Hz, 1H), 7.44 - 7.33 (m, 2H), 6.69 (d, J = 4.0 Hz, 1H), 6.63 (d, J = 9.0 Hz, 1H), 6.54 (d, J = 4.0 Hz, 1H), 3.46 - 3.38 (m, 4H), 2.92 - 2.85 (m, 4H). LCMS (Analytical Method A) Rt = 1.63 min, MS (ESIpos): m/z 397.1, 399.1 [M+H]+, Purity = 100%. |
| | | 1-[2-(5-chlorothiophen-2-yl)-3-(pyridin-4-yl)-3H-imidazo[4,5-b]pyridin-5-yl]piperazine | |
| 56-SS | Intermediate 39 (9% yield) | 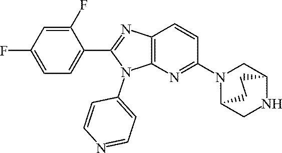 [SMILES: Fc1ccc(-c2nc3ccc(N4C[C@@H]5CC[C@H]4CN5)nc3n2-c2ccncc2)c(F)c1] | 1H NMR (500 MHz, Chloroform-d) δ 8.69 - 8.61 (m, 2H), 7.92 (d, J = 8.9 Hz, 1H), 7.72 (td, J = 8.3, 6.3 Hz, 1H), 7.32 (dd, J = 4.6, 1.6 Hz, 2H), 7.06 - 6.99 (m, 1H), 6.76 (ddd, J = 11.0, 8.7, 2.4 Hz, 1H), 6.49 (d, J = 8.9 Hz, 1H), 4.60 (s, 1H), 3.71 (dt, J = 10.4, 2.6 Hz, 1H), 3.58 (dd, J = 10.4, 1.9 Hz, 1H), 3.32 (dt, J = 11.0, 2.7 Hz, 1H), 3.27 - 3.17 (m, 2H), 2.09 - 1.97 (m, 1H), 1.97 - 1.87 (m, 2H), 1.85 - 1.75 (m, 1H). LCMS (Analytical Method A) Rt = 1.52 min, MS (ESIpos): m/z 419 [M+H]+, Purity = 100%. |
| | | (1S,4S)-2-[2-(2,4-difluorophenyl)-3-(pyridin-4-yl)-3H-imidazo[4,5-b]pyridin-5-yl]-2,5-diazabicyclo[2.2.2]octane | |
| 102 | Intermediate 4 (41% yield) | 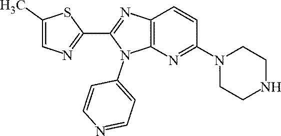 [SMILES: Cc1cnc(-c2nc3ccc(N4CCNCC4)nc3n2-c2ccncc2)s1] | 1H NMR (500 MHz, Chloroform-d) δ 8.74 - 8.62 (m, 2H), 7.85 (d, J = 9.0 Hz, 1H), 7.45 - 7.30 (m, 2H), 7.30 -7.26 (m, 1H), 6.67 (d, J = 9.0 Hz, 1H), 3.51 - 3.41 (m, 4H), 2.96 - 2.85 (m, 4H), 2.44 (s, 3H). LCMS (Analytical Method A) Rt = 1.25 min, MS (ESIpos): m/z 378.1 [M+H]+, Purity = 98%. |
| | | 1-[2-(5-methyl-1,3-thiazol-2-yl)-3-(pyridin-4-yl)-3H-imidazo[4,5-b]pyridin-5-yl]piperazine | |
| 103 | Intermediate 4 (56% yield) | 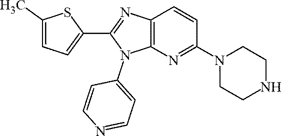 [SMILES: Cc1ccc(-c2nc3ccc(N4CCNCC4)nc3n2-c2ccncc2)s1] | 1H NMR (500 MHz, Chloroform-d) δ 8.77 - 8.68 (m, 2H), 7.81 (d, J = 8.9 Hz, 1H), 7.44 - 7.36 (m, 2H), 6.66 -6.58 (m, 2H), 6.56 - 6.47 (m, 1H), 3.40 (m, 4H), 2.94 - 2.81 (m, 4H), 2.47 - 2.34 (m, 3H). LCMS (Analytical Method A) Rt = 1.47 min, MS (ESIpos): m/z 377.1 [M+H]+, Purity = 99%. |
| | | 1-[2-(5-methylthiophen-2-yl)-3-(pyridin-4-yl)-3H-imidazo[4,5-b]pyridin-5-yl]piperazine | |
| 104-RR | Intermediate 40 (52% yield) | 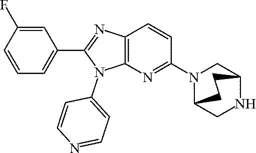 [SMILES: Fc1cccc(-c2nc3ccc(N4C[C@H]5CC[C@@H]4CN5)nc3n2-c2ccncc2)c1] | 1H NMR (400 MHz, DMSO-d6) δ 8.71 (dd, J = 4.5, 1.6 Hz, 2H), 7.94 (d, J = 8.9 Hz, 1H), 7.48 - 7.37 (m, 3H), 7.32 - 7.16 (m, 3H), 6.61 (d, J = 8.9 Hz, 1H), 4.43 (s, 1H), 3.60 -3.41 (m, 2H), 3.10 - 2.94 (m, 3H), 1.91 - 1.75 (m, 3H), 1.71 - 1.60 (m, 1H). LCMS (Analytical Method A) Rt = 1.52 min, MS (ESIpos): m/z 401 [M+H]+, Purity = 99%. |
| | | (1R,4R)-2-[2-(3-fluorophenyl)-3-(pyridin-4-yl)-3H-imidazo[4,5-b]pyridin-5-yl]-2,5-diazabicyclo[2.2.2]octane | |
| 104-SS | Intermediate 39 (41% yield) | 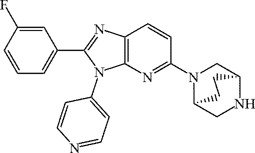 [SMILES: Fc1cccc(-c2nc3ccc(N4C[C@@H]5CC[C@H]4CN5)nc3n2-c2ccncc2)c1] | 1H NMR (400 MHz, DMSO-d6) δ 8.71 (dd, J = 4.6, 1.6 Hz, 2H), 7.94 (d, J = 8.9 Hz, 1H), 7.52 - 7.34 (m, 3H), 7.34 - 7.15 (m, 3H), 6.61 (d, J = 8.9 Hz, 1H), 4.43 (s, 1H), 3.60 -3.40 (m, 2H), 3.10 - 2.95 (m, 3H), 1.90 - 1.72 (m, 3H), 1.71 - 1.61 (m, 1H). LCMS (Analytical Method A) Rt = 1.52 min, MS (ESIpos): m/z 401 [M+H]+, Purity = 98%. |
| | | (1S,4S)-2-[2-(3-fluorophenyl)-3-(pyridin-4-yl)-3H-imidazo[4,5-b]pyridin-5-yl]-2,5-diazabicyclo[2.2.2]octane | |
| 21-RR | Intermediate 40 (53% yield) | 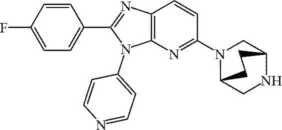 [SMILES: Fc1ccc(-c2nc3ccc(N4C[C@H]5CC[C@@H]4CN5)nc3n2-c2ccncc2)cc1] | 1H NMR (400 MHz, DMSO-d6) δ 8.69 (dd, J = 4.6, 1.6 Hz, 2H), 7.92 (d, J = 8.9 Hz, 1H), 7.52 - 7.45 (m, 2H), 7.42 (dd, J = 4.6, 1.6 Hz, 2H), 7.29 - 7.20 (m, 2H), 6.59 (d, J = 8.9 Hz, 1H), 4.43 (s, 1H), 3.60 - 3.41 (m, 2H), 3.11 - 2.94 (m, 3H), 1.93- 1.73 (m, 3H), 1.72 - 1.58 (m, 1H). LCMS (Analytical Method A) Rt = 1.49 min, MS (ESIpos): m/z 401 [M+H]+, Purity = 100%. |
| | | (1R,4R)-2-[2-(4-fluorophenyl)-3-(pyridin-4-yl)-3H-imidazo[4,5-b]pyridin-5-yl]-2,5-diazabicyclo[2.2.2]octane | |
| 21-SS | Intermediate 39 (26% yield) | 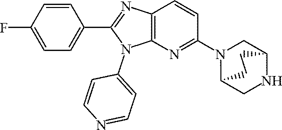 [SMILES: Fc1ccc(-c2nc3ccc(N4C[C@@H]5CC[C@H]4CN5)nc3n2-c2ccncc2)cc1] | 1H NMR (500 MHz, DMSO-d6) δ 8.74 - 8.65 (m, 2H), 8.32 (s, 1H), 7.95 (d, J = 8.9 Hz, 1H), 7.53 - 7.46 (m, 2H), 7.43 (dd, J = 4.6, 1.6 Hz, 2H), 7.30 - 7.20 (m, 2H), 6.62 (d, J = 8.9 Hz, 1H), 4.50 (s, 1H), 3.69 -3.04 (m, 5H), 1.99 - 1.77 (m, 3H), 1.75 - 1.64 (m, 1H). LCMS (Analytical Method A) Rt = 1.49 min, MS (ESIpos): m/z 401 [M+H]+, Purity = 98%. |
| | | (1S,4S)-2-[2-(4-fluorophenyl)-3-(pyridin-4-yl)-3H-imidazo[4,5-b]pyridin-5-yl]-2,5-diazabicyclo[2.2.2]octane | |
| 107 | Intermediate 4 (23% yield) | 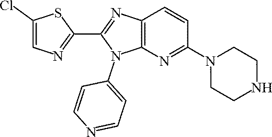 [SMILES: Clc1cnc(-c2nc3ccc(N4CCNCC4)nc3n2-c2ccncc2)s1] | 1H NMR (500 MHz, Chloroform-d) δ 8.76 - 8.63 (m, 2H), 7.84 (d, J = 9.0 Hz, 1H), 7.47 - 7.32 (m, 3H), 6.69 (d, J = 9.1 Hz, 1H), 3.51 - 3.35 (m, 4H), 2.92 - 2.80 (m, 4H). LCMS (Analytical Method B) Rt = 2.95 min, MS (ESIpos): m/z 398.3, 400.3 [M+H]+, Purity = 95%. |
| | | 1-[2-(5-chloro-1,3-thiazol-2-yl)-3-(pyridin-4-yl)-3H-imidazo[4,5-b]pyridin-5-yl]piperazine | |
| 108-S | Intermediate 43 (45% yield) | 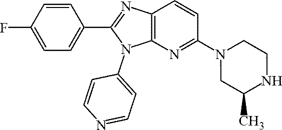 [SMILES: C[C@H]1CN(c2ccc3nc(-c4ccc(F)cc4)n(-c4ccncc4)c3n2)CCN1] | 1H NMR (500 MHz, DMSO-d6) δ 8.74 - 8.66 (m, 2H), 7.94 (d, J = 8.9 Hz, 1H), 7.53 - 7.47 (m, 2H), 7.44 -7.41 (m, 2H), 7.29 - 7.21 (m, 2H), 6.90 (d, J = 9.0 Hz, 1H), 4.14 - 4.00 (m, 2H), 2.97 - 2.87 (m, 1H), 2.73 -2.65 (m, 3H), 2.38 - 2.32 (m, 1H), 2.24 (s, 1H), 1.01 (d, J = 6.3 Hz, 3H). LCMS (Analytical Method A) Rt = 1.44 min, MS (ESIpos): m/z 389 [M+H]+, Purity = 97%. |
| | | (3S)-1-[2-(4-fluorophenyl)-3-(pyridin-4-yl)-3H-imidazo[4,5-b]pyridin-5-yl]-3-methylpiperazine | |
| 108-R | Intermediate 44 (47% yield) | 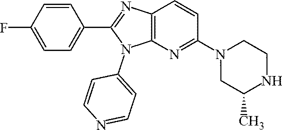 [SMILES: C[C@@H]1CN(c2ccc3nc(-c4ccc(F)cc4)n(-c4ccncc4)c3n2)CCN1] | 1H NMR (500 MHz, DMSO-d6) δ 8.73 - 8.67 (m, 2H), 7.94 (d, J = 8.9 Hz, 1H), 7.54 - 7.47 (m, 2H), 7.46 -7.40 (m, 2H), 7.26 (t, J = 8.9 Hz, 2H), 6.90 (d, J = 9.0 Hz, 1H), 4.12 - 4.00 (m, 2H), 2.96 - 2.87 (m, 1H), 2.72 -2.65 (m, 3H), 2.39 - 2.30 (m, 1H), 2.24 (s, 1H), 1.01 (d, J = 6.3 Hz, 3H). LCMS (Analytical Method A) Rt = 1.44 min, MS (ESIpos): m/z 389 [M+H]+, Purity = 97%. |
| | | (3R)-1-[2-(4-fluorophenyl)-3-(pyridin-4-yl)-3H-imidazo[4,5-b]pyridin-5-yl]-3-methylpiperazine | |
| 111 | Intermediate 27 (20% yield) | 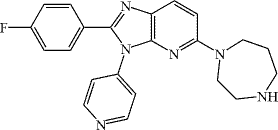 [SMILES: Fc1ccc(-c2nc3ccc(N4CCCNCC4)nc3n2-c2ccncc2)cc1] | 1H NMR (500 MHz, Chloroform-d) δ 8.61 (d, J = 6.1 Hz, 2H), 7.81 (d, J = 8.9 Hz, 1H), 7.54 - 7.37 (m, 2H), 7.30 - 7.22 (m, 2H), 7.02 - 6.93 (m, 2H), 6.52 (d, J = 8.9 Hz, 1H), 3.76 -3.60 (m, 4H), 3.04 - 2.87 (m, 2H), 2.83 - 2.69 (m, 2H), 2.29 (s, 1H), 1.95 - 1.76 (m, 2H). LCMS (Analytical Method A) Rt = 1.44 min, MS (ESIpos): m/z 389.2 [M+H]+, Purity = 97%. |
| | | 1-[2-(4-fluorophenyl)-3-(pyridin-4-yl)-3H-imidazo[4,5-b]pyridin-5-yl]-1,4-diazepane | |
| 113 | Intermediate 30 (5% yield) | 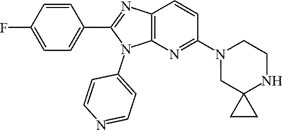 [SMILES: Fc1ccc(-c2nc3ccc(N4CCNC5(CC5)C4)nc3n2-c2ccncc2)cc1] | 1H NMR (500 MHz, Chloroform-d) δ 8.73 - 8.66 (m, 2H), 7.91 (d, J = 8.9 Hz, 1H), 7.53 - 7.47 (m, 2H), 7.36 -7.31 (m, 2H), 7.10 - 7.02 (m, 2H), 6.69 (d, J = 8.9 Hz, 1H), 3.59 - 3.51 (m, 2H), 3.37 (s, 2H), 3.14 - 3.03 (m, 2H), 0.68 - 0.63 (m, 2H), 0.61 - 0.56 (m, 2H). LCMS (Analytical Method A) Rt = 1.51 min, MS (ESIpos): m/z 401 [M+H]+, Purity = 97%. |
| | | 7-[2-(4-fluorophenyl)-3-(pyridin-4-yl)-3H-imidazo[4,5-b]pyridin-5-yl]-4,7-diazaspiro[2.5]octane | |
| 120 | Intermediate 4 (37% yield) | 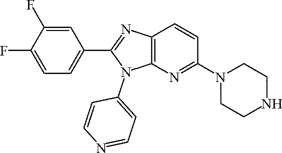 [SMILES: Fc1ccc(-c2nc3ccc(N4CCNCC4)nc3n2-c2ccncc2)cc1F] | 1H NMR (500 MHz, DMSO-d6) δ 8.78 - 8.66 (m, 2H), 7.97 (d, J = 9.0 Hz, 1H), 7.56 (ddd, J = 11.4, 7.8, 2.1 Hz, 1H), 7.52 - 7.42 (m, 3H), 7.28 -7.17 (m, 1H), 6.91 (d, J = 9.0 Hz, 1H), 3.44 - 3.39 (m, 4H), 2.81 - 2.75 (m, 4H). LCMS (Analytical Method A) Rt = 1.50 min, MS (ESIpos): m/z 393.3 [M+H]+, Purity = 97%. |
| | | 1-[2-(3,4-difluorophenyl)-3-(pyridin-4-yl)-3H-imidazo[4,5-b]pyridin-5-yl]piperazine | |
| 121-RR | Intermediate 72 (18% yield) | 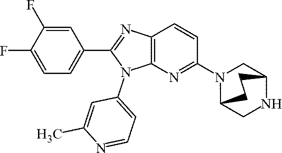 [SMILES: Cc1cc(-n2c(-c3ccc(F)c(F)c3)nc3ccc(N4C[C@H]5CC[C@@H]4CN5)nc32)ccn1] | 1H NMR (400 MHz, Methanol-d4) δ 8.53 (d, J = 5.5 Hz, 1H), 7.89 (d, J = 8.9 Hz, 1H), 7.47 (ddd, J = 11.1, 7.6, 2.1 Hz, 1H), 7.39 (d, J = 1.9 Hz, 1H), 7.36 - 7.27 (m, 2H), 7.27 - 7.22 (m, 1H), 6.66 (d, J = 9.0 Hz, 1H), 4.58 (br s, 1H), 3.72 (dt, J = 10.7, 2.6 Hz, 1H), 3.57 (dd, J = 10.8, 2.0 Hz, 1H), 3.23 - 3.15 (m, 2H), 3.10 (dd, J = 11.2, 1.9 Hz, 1H), 2.56 (s, 3H), 2.09 - 1.72 (m, 4H). LCMS (Analytical Method A) Rt = 1.50 min, MS (ESIpos): m/z 433.4 [M+H]+, Purity = 100%. |
| | | (1R,4R)-2-[2-(3,4-difluorophenyl)-3-(2-methylpyridin-4-yl)-3H-imidazo[4,5-b]pyridin-5-yl]-2,5-diazabicyclo[2.2.2]octane | |
| 122-RR | Intermediate 72 (14% yield) | 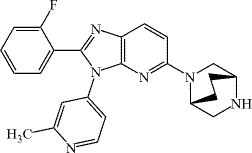 [SMILES: Cc1cc(-n2c(-c3ccccc3F)nc3ccc(N4C[C@H]5CC[C@@H]4CN5)nc32)ccn1] | 1H NMR (400 MHz, Methanol-d4) δ 8.44 (d, J = 5.5 Hz, 1H), 7.91 (d, J = 8.9 Hz, 1H), 7.71 (td, J = 7.4, 1.7 Hz, 1H), 7.60 - 7.51 (m, 1H), 7.37 (td, J = 7.6, 1.0 Hz, 1H), 7.32 (d, J = 1.9 Hz, 1H), 7.27 (dd, J = 5.4, 1.9 Hz, 1H), 7.16 - 7.08 (m, 1H), 6.68 (d, J = 9.0 Hz, 1H), 4.62 (s, 1H), 3.75 (dt, J = 10.6, 2.5 Hz, 1H), 3.60 (dd, J = 10.8, 2.0 Hz, 1H), 3.26 - 3.18 (m, 2H), 3.13 (dd, J = 11.2, 1.9 Hz, 1H), 2.49 (s, 3H), 2.12 - 1.75 (m, 4H). LCMS (Analytical Method A) Rt = 1.20 min, MS (ESIpos): m/z 415.3 [M+H]+, Purity = 100%. |
| | | (1R,4R)-2-[2-(2-fluorophenyl)-3-(2-methylpyridin-4-yl)-3H-imidazo[4,5-b]pyridin-5-yl]-2,5-diazabicyclo[2.2.2]octane | |
| 123-RR | Intermediate 72 (31% yield) | 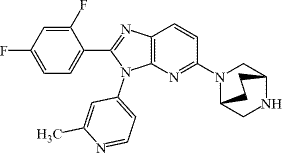 [SMILES: Cc1cc(-n2c(-c3ccc(F)cc3F)nc3ccc(N4C[C@H]5CC[C@@H]4CN5)nc32)ccn1] | 1H NMR (400 MHz, Methanol-d4) δ 8.46 (d, J = 5.5 Hz, 1H), 7.91 (d, J = 8.9 Hz, 1H), 7.75 (td, J = 8.4, 6.3 Hz, 1H), 7.34 (d, J = 1.9 Hz, 1H), 7.27 (dd, J = 5.5, 1.8 Hz, 1H), 7.21 - 7.13 (m, 1H), 7.02 (ddd, J = 10.4, 9.0, 2.4 Hz, 1H), 6.68 (d, J = 9.0 Hz, 1H), 4.62 (s, 1H), 3.75 (dt, J = 10.8, 2.6 Hz, 1H), 3.60 (dd, J = 10.8, 1.9 Hz, 1H), 3.27 - 3.20 (m, 2H), 3.14 (dd, J = 11.2, 1.9 Hz, 1H), 2.51 (s, 3H), 2.11 - 1.78 (m, 4H). LCMS (Analytical Method A) Rt = 1.28 min, MS (ESIpos): m/z 433.3 [M+H]+, Purity = 95%. |
| | | (1R,4R)-2-[2-(2,4-difluorophenyl)-3-(2-methylpyridin-4-yl)-3H-imidazo[4,5-b]pyridin-5-yl]-2,5-diazabicyclo[2.2.2]octane | |
| 124-RR | Intermediate 72 (21% yield) | 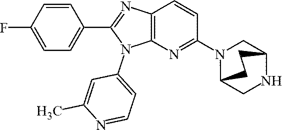 [SMILES: Cc1cc(-n2c(-c3ccc(F)cc3)nc3ccc(N4C[C@H]5CC[C@@H]4CN5)nc32)ccn1] | 1H NMR (400 MHz, Methanol-d4) δ 8.51 (d, J = 5.4 Hz, 1H), 7.91 (d, J = 8.9 Hz, 1H), 7.59 - 7.47 (m, 2H), 7.35 (d, J = 1.9 Hz, 1H), 7.28 (dd, J = 5.5, 1.7 Hz, 1H), 7.21 - 7.13 (m, 2H), 6.67 (d, J = 9.0 Hz, 1H), 4.64 (s, 1H), 3.76 (dt, J = 10.9, 2.6 Hz, 1H), 3.61 (dd, J = 11.0, 1.9 Hz, 1H), 3.34 (m, 1H), 3.29 - 3.24 (m, 1H), 3.19 (dd, J = 11.3, 1.9 Hz, 1H), 2.54 (s, 3H), 2.14 - 1.79 (m, 4H). LCMS (Analytical Method A) Rt = 1.26 min, MS (ESIpos): m/z 415.3 [M+H]+, Purity = 99%. |
| | | (1R,4R)-2-[2-(4-fluorophenyl)-3-(2-methylpyridin-4-yl)-3H-imidazo[4,5-b]pyridin-5-yl]-2,5-diazabicyclo[2.2.2]octane | |
| 125 | Intermediate 2-2 (22% yield) | 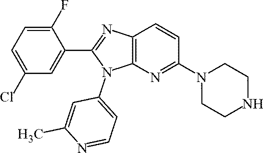 [SMILES: Cc1cc(-n2c(-c3cc(Cl)ccc3F)nc3ccc(N4CCNCC4)nc32)ccn1] | 1H NMR (400 MHz, Methanol-d4) δ 8.48 (d, J = 5.5 Hz, 1H), 7.95 (d, J = 9.0 Hz, 1H), 7.79 (dd, J = 6.0, 2.7 Hz, 1H), 7.56 (ddd, J = 8.9, 4.3, 2.7 Hz, 1H), 7.38 (d, J = 1.9 Hz, 1H), 7.29 (dd, J = 5.5, 1.8 Hz, 1H), 7.13 (t, J = 9.2 Hz, 1H), 6.96 (d, J = 9.1 Hz, 1H), 3.65 - 3.52 (m, 4H), 2.98 -2.86 (m, 4H), 2.53 (s, 3H). LCMS (Analytical Method B) Rt = 2.86 min, MS (ESIpos): m/z 423.3, 425.2 [M+H]+, Purity = 97%. |
| | | 1-[2-(5-chloro-2-fluorophenyl)-3-(2-methylpyridin-4-yl)-3H-imidazo[4,5-b]pyridin-5-yl]piperazine | |
| 126 | Intermediate 73 (17% yield) | 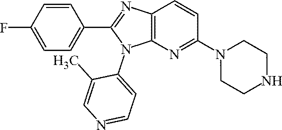 [SMILES: Cc1cnccc1-n1c(-c2ccc(F)cc2)nc2ccc(N3CCNCC3)nc21] | 1H NMR (400 MHz, Methanol-d4) δ 8.65 (s, 1H), 8.49 (d, J = 5.3 Hz, 1H), 7.93 (d, J = 9.0 Hz, 1H), 7.54 - 7.44 (m, 2H), 7.28 (d, J = 5.3 Hz, 1H), 7.17 - 7.08 (m, 2H), 6.91 (d, J = 9.0 Hz, 1H), 3.51 - 3.44 (m, 4H), 2.90 -2.85 (m, 4H), 2.13 (s, 3H). LCMS (Analytical Method A) Rt = 1.48 min, MS (ESIpos): m/z 389.2 [M+H]+, Purity = 97%. |
| | | 1-[2-(4-fluorophenyl)-3-(3-methylpyridin-4-yl)-3H-imidazo[4,5-b]pyridin-5-yl]piperazine | |
| 127-RR | Intermediate 38 (41% yield) | 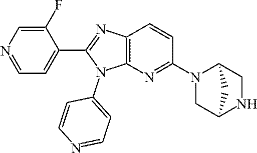 [SMILES: Fc1cnccc1-c1nc2ccc(N3C[C@H]4C[C@@H]3CN4)nc2n1-c1ccncc1] | 1H NMR (400 MHz, Methanol-d4) δ 8.67 - 8.62 (m, 2H), 8.56 (dd, J = 5.0, 0.9 Hz, 1H), 8.49 (d, J = 2.0 Hz, 1H), 7.94 (d, J = 8.9 Hz, 1H), 7.77 (dd, J = 6.0, 5.1 Hz, 1H), 7.56 - 7.50 (m, 2H), 6.68 (d, J = 8.9 Hz, 1H), 4.82 - 4.81 (m, 1H), 3.87 - 3.81 (m, 1H), 3.60 (dd, J = 9.9, 2.2 Hz, 1H), 3.39 (d, J = 9.7 Hz, 1H), 3.06 - 2.95 (m, 2H), 1.96 (d, J = 10.0 Hz, 1H), 1.82 (d, J = 9.7 Hz, 1H). LCMS (Analytical Method A) Rt = 1.11 min, MS (ESIpos): m/z 388.2 [M+H]+, Purity = 100%. |
| | | (1R,4R)-2-[2-(3-fluoropyridin-4-yl)-3-(pyridin-4-yl)-3H-imidazo[4,5-b]pyridin-5-yl]-2,5-diazabicyclo[2.2.1]heptane | |
| 128-RR | Intermediate 38 (24% yield) | 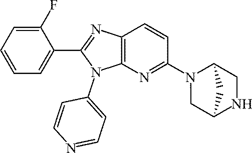 [SMILES: Fc1ccccc1-c1nc2ccc(N3C[C@H]4C[C@@H]3CN4)nc2n1-c1ccncc1] | 1H NMR (500 MHz, Chloroform-d) δ 8.63 - 8.45 (m, 2H), 7.83 (d, J = 8.8 Hz, 1H), 7.75 - 7.57 (m, 1H), 7.44 -7.32 (m, 1H), 7.30 - 7.25 (m, 2H), 7.23 - 7.13 (m, 1H), 6.98 - 6.88 (m, 1H), 6.36 (d, J = 8.8 Hz, 1H), 4.76 -4.63 (m, 1H), 3.83 - 3.68 (m, 1H), 3.56 (dd, J = 9.5, 2.0 Hz, 1H), 3.21 (d, J = 9.4 Hz, 1H), 3.10 - 2.91 (m, 2H), 1.84 (d, J = 9.6 Hz, 1H), 1.76 (d, J = 9.6 Hz, 1H). LCMS (Analytical Method A) Rt = 1.31 min, MS (ESIpos): m/z 387.2 [M+H]+, Purity = 99.%. |
| | | (1R,4R)-2-[2-(2-fluorophenyl)-3-(pyridin-4-yl)-3H-imidazo[4,5-b]pyridin-5-yl]-2,5-diazabicyclo[2.2.1]heptane | |
| 129-RR | Intermediate 38 (23% yield) | 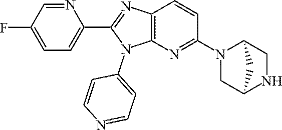 [SMILES: Fc1ccc(-c2nc3ccc(N4C[C@H]5C[C@@H]4CN5)nc3n2-c2ccncc2)nc1] | 1H NMR (500 MHz, Chloroform-d) δ 8.67 - 8.53 (m, 2H), 8.13 (d, J = 2.8 Hz, 1H), 8.08 (dd, J = 8.8, 4.4 Hz, 1H), 7.81 (d, J = 8.8 Hz, 1H), 7.43 (td, J = 8.4, 2.9 Hz, 1H), 7.27 - 7.23 (m, 2H), 6.36 (d, J = 8.8 Hz, 1H), 4.69 - 4.61 (m, 1H), 3.87 - 3.77 (m, 1H), 3.52 (dd, J = 9.6, 1.9 Hz, 1H), 3.24 (d, J = 9.6 Hz, 1H), 3.08 - 2.96 (m, 2H), 1.83 (d, J = 9.6 Hz, 1H), 1.77 (d, J = 9.6 Hz, 1H). LCMS (Analytical Method A) Rt = 1.14 min, MS (ESIpos): m/z 388.2 [M+H]+, Purity = 97%. |
| | | (1R,4R)-2-[2-(5-fluoropyridin-2-yl)-3-(pyridin-4-yl)-3H-imidazo[4,5-b]pyridin-5-yl]-2,5-diazabicyclo[2.2.1]heptane | |
| 130-RR | Intermediate 38 (14% yield) | 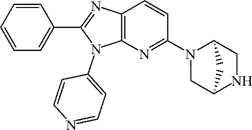 [SMILES: c1ccc(-c2nc3ccc(N4C[C@H]5C[C@@H]4CN5)nc3n2-c2ccncc2)cc1] | 1H NMR (500 MHz, Chloroform-d) δ 8.60 (d, J = 6.1 Hz, 2H), 7.82 (d, J = 8.7 Hz, 1H), 7.50 - 7.36 (m, 2H), 7.34 - 7.25 (m, 5H), 6.34 (d, J = 8.8 Hz, 1H), 4.69 - 4.62 (m, 1H), 3.80 -3.73 (m, 1H), 3.58 - 3.51 (m, 1H), 3.20 (d, J = 9.4 Hz, 1H), 3.05 - 2.96 (m, 2H), 1.84 (d, J = 9.4 Hz, 1H), 1.76 (d, J = 9.5 Hz, 1H). LCMS (Analytical Method A) Rt = 1.30 min, MS (ESIpos): m/z 369.2 [M+H]+, Purity = 95%. |
| | | (1R,4R)-2-[2-phenyl-3-(pyridin-4-yl)-3H-imidazo[4,5-b]pyridin-5-yl]-2,5-diazabicyclo[2.2.1]heptane | |
| 131-RS | Intermediate 45 (10% yield) | 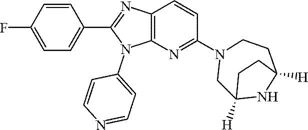 [SMILES: Fc1ccc(-c2nc3ccc(N4CC[C@@H]5CC[C@H](C4)N5)nc3n2-c2ccncc2)cc1] | 1H NMR (500 MHz, Chloroform-d) δ 8.66 - 8.54 (m, 2H), 7.79 (d, J = 8.9 Hz, 1H), 7.48 - 7.34 (m, 2H), 7.33 -7.23 (m, 2H), 7.06 - 6.88 (m, 2H), 6.55 (d, J = 9.0 Hz, 1H), 4.40 (d, J = 13.6 Hz, 1H), 3.85 - 3.74 (m, 1H), 3.74 - 3.65 (m, 1H), 3.63 - 3.58 (m, 1H), 3.58 - 3.52 (m, 1H), 3.09 (dd, J = 13.8, 2.7 Hz, 1H), 1.97 - 1.85 (m, 1H), 1.85 - 1.78 (m, 2H), 1.68 - 1.52 (m, 2H), 1.27 - 1.15 (m, 1H). LCMS (Analytical Method A) Rt = 1.49 min, MS (ESIpos): m/z 415.2 [M+H]+, Purity = 95%. |
| | | (1R,6S)-3-[2-(4-fluorophenyl)-3-(pyridin-4-yl)-3H-imidazo[4,5-b]pyridin-5-yl]-3,9-diazabicyclo[4.2.1]nonane | |
| 132-RR | Intermediate 74 (19% yield) | 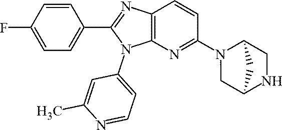 [SMILES: Cc1cc(-n2c(-c3ccc(F)cc3)nc3ccc(N4C[C@H]5C[C@@H]4CN5)nc32)ccn1] | 1H NMR (400 MHz, Chloroform-d) δ 8.49 (d, J = 5.4 Hz, 1H), 7.80 (d, J = 8.7 Hz, 1H), 7.48 - 7.36 (m, 2H), 7.18 - 7.10 (m, 1H), 7.05 (dd, J = 5.4, 1.8 Hz, 1H), 7.01 - 6.91 (m, 2H), 6.33 (d, J = 8.8 Hz, 1H), 4.69 - 4.60 (m, 1H), 3.85 - 3.80 (m, 1H), 3.54 (dd, J = 9.7, 2.0 Hz, 1H), 3.25 (d, J = 9.6 Hz, 1H), 3.07 - 2.98 (m, 2H), 2.50 (s, 3H), 1.85 (d, J = 9.5 Hz, 1H), 1.78 (d, J = 9.5 Hz, 1H). LCMS (Analytical Method A) Rt = 1.33 min, MS (ESIpos): m/z 401.2 [M+H]+, Purity = 100%. |
| | | (1R,4R)-2-[2-(4-fluorophenyl)-3-(2-methylpyridin-4-yl)-3H-imidazo[4,5-b]pyridin-5-yl]-2,5-diazabicyclo[2.2.1]heptane | |
| 133-S formic acid salt | Intermediate 75 (35% yield) | 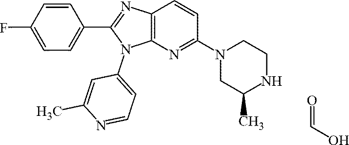 [SMILES: Cc1cc(-n2c(-c3ccc(F)cc3)nc3ccc(N4CCN[C@@H](C)C4)nc32)ccn1 ; O=CO] | 1H NMR (500 MHz, Chloroform-d) δ 8.59 (d, J = 5.4 Hz, 1H), 8.41 (s, 1H), 7.96 (d, J = 8.8 Hz, 1H), 7.55 - 7.48 (m, 2H), 7.21 (d, J = 1.7 Hz, 1H), 7.12 (dd, J = 5.4, 1.8 Hz, 1H), 7.10 -7.02 (m, 2H), 6.75 (d, J = 8.9 Hz, 1H), 4.26 - 4.16 (m, 2H), 3.30 (d, J = 12.3 Hz, 1H), 3.27 - 3.14 (m, 2H), 3.03 (td, J = 12.1,3.3 Hz, 1H), 2.94 (dd, J = 13.4, 10.6 Hz, 1H), 2.59 (s, 3H), 1.34 (d, J = 6.5 Hz, 3H). LCMS (Analytical Method A) Rt = 1.37 min, MS (ESIpos): m/z 403 [M+H]+, Purity = 100%. |
| | | (3S)-1-[2-(4-fluorophenyl)-3-(2-methylpyridin-4-yl)-3H-imidazo[4,5-b]pyridin-5-yl]-3-methylpiperazine formic acid salt | |
| 134 | Intermediate 76 (8% yield) | 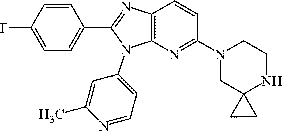 [SMILES: Cc1cc(-n2c(-c3ccc(F)cc3)nc3ccc(N4CCNC5(CC5)C4)nc32)ccn1] | 1H NMR (500 MHz, Chloroform-d) δ 8.57 (d, J = 5.4 Hz, 1H), 7.91 (d, J = 8.9 Hz, 1H), 7.55 - 7.48 (m, 2H), 7.23 (d, J = 1.5 Hz, 1H), 7.10 (dd, J = 5.4, 1.7 Hz, 1H), 7.08 - 7.02 (m, 2H), 6.69 (d, J = 8.9 Hz, 1H), 3.64 -3.55 (m, 2H), 3.43 (s, 2H), 3.15 -3.08 (m, 2H), 2.58 (s, 3H), 0.79 -0.71 (m, 2H), 0.66 - 0.60 (m, 2H). LCMS (Analytical Method A) Rt = 1.46 min, MS (ESIpos): m/z 415 [M+H]+, Purity = 100%. |
| | | 7-[2-(4-fluorophenyl)-3-(2-methylpyridin-4-yl)-3H-imidazo[4,5-b]pyridin-5-yl]-4,7-diazaspiro[2.5]octane | |
| 133-R formic acid salt | Intermediate 77 (40% yield) | 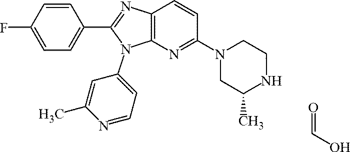 [SMILES: Cc1cc(-n2c(-c3ccc(F)cc3)nc3ccc(N4CCN[C@H](C)C4)nc32)ccn1 ; O=CO] | 1H NMR (500 MHz, Chloroform-d) δ 8.59 (d, J = 5.4 Hz, 1H), 8.42 (s, 1H), 7.95 (d, J = 8.8 Hz, 1H), 7.55 - 7.48 (m, 2H), 7.21 (d, J = 1.8 Hz, 1H), 7.12 (dd, J = 5.4, 1.7 Hz, 1H), 7.10 -7.03 (m, 2H), 6.75 (d, J = 8.9 Hz, 1H), 4.26 - 4.14 (m, 2H), 3.27 (d, J = 12.2 Hz, 1H), 3.23 - 3.10 (m, 2H), 3.02 (td, J = 12.0, 3.3 Hz, 1H), 2.89 (dd, J = 13.2, 10.6 Hz, 1H), 2.59 (s, 3H), 1.32 (d, J = 6.5 Hz, 3H). LCMS (Analytical Method A) Rt = 1.39 min, MS (ESIpos): m/z 403 [M+H]+, Purity = 100%. |
| | | (3R)-1-[2-(4-fluorophenyl)-3-(2-methylpyridin-4-yl)-3H-imidazo[4,5-b]pyridin-5-yl]-3-methylpiperazine formic acid salt | |
| 135-RS formic acid salt | Intermediate 46 (24% yield) | 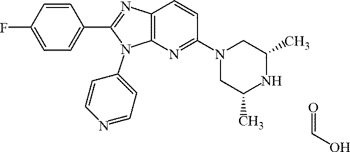 [SMILES: C[C@@H]1CN(c2ccc3nc(-c4ccc(F)cc4)n(-c4ccncc4)c3n2)C[C@H](C)N1 ; O=CO] | 1H NMR (500 MHz, Chloroform-d) δ 8.73 - 8.69 (m, 2H), 8.41 (s, 1H), 7.93 (d, J = 8.9 Hz, 1H), 7.54 - 7.47 (m, 2H), 7.37 - 7.32 (m, 2H), 7.10 -7.02 (m, 2H), 6.74 (d, J = 8.9 Hz, 1H), 4.18 (dd, J = 12.8, 2.3 Hz, 2H), 3.08 - 2.99 (m, 2H), 2.65 - 2.56 (m, 2H), 1.22 (d, J = 6.4 Hz, 6H). LCMS (Analytical Method A) Rt = 1.54 min, MS (ESIpos): m/z 403 [M+H]+, Purity = 100%. |
| | | (3R,5S)-1-[2-(4-fluorophenyl)-3-(pyridin-4-yl)-3H-imidazo[4,5-b]pyridin-5-yl]-3,5-dimethylpiperazine formic acid salt | |
| 136 formic acid salt | Intermediate 47 (47% yield) | 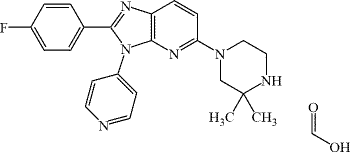 [SMILES: CC1(C)CN(c2ccc3nc(-c4ccc(F)cc4)n(-c4ccncc4)c3n2)CCN1 ; O=CO] | 1H NMR (400 MHz, Chloroform-d) δ 8.75 (d, J = 6.0 Hz, 2H), 8.51 (s, 1H), 7.98 (d, J = 8.8 Hz, 1H), 7.58 - 7.47 (m, 2H), 7.35 (dd, J = 4.7, 1.5 Hz, 2H), 7.09 (t, J = 8.6 Hz, 2H), 6.75 (d, J = 8.9 Hz, 1H), 3.85 - 3.76 (m, 2H), 3.58 (s, 2H), 3.27 - 3.15 (m, 2H), 1.43 (s, 6H). LCMS (Analytical Method A) Rt = 1.52 min, MS (ESIpos): m/z 403 [M+H]+, Purity = 100%. |
| | | 1-[2-(4-fluorophenyl)-3-(pyridin-4-yl)-3H-imidazo[4,5-b]pyridin-5-yl]-3,3-dimethylpiperazine; formic acid | |
| 137 | Intermediate 27 (29% yield) | 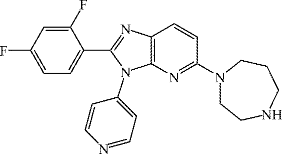 [SMILES: Fc1ccc(-c2nc3ccc(N4CCCNCC4)nc3n2-c2ccncc2)c(F)c1] | 1H NMR (400 MHz, Chloroform-d) δ 8.69 - 8.60 (m, 2H), 7.90 (d, J = 8.9 Hz, 1H), 7.72 (td, J = 8.3, 6.4 Hz, 1H), 7.35 - 7.28 (m, 2H), 7.02 (td, J = 8.0, 2.0 Hz, 1H), 6.80 - 6.73 (m, 1H), 6.62 (d, J = 9.0 Hz, 1H), 3.84 -3.72 (m, 4H), 3.13 - 2.98 (m, 2H), 2.94 - 2.80 (m, 2H), 2.01 - 1.90 (m, 2H). LCMS (Analytical Method B) Rt = 2.64 min, MS (ESIpos): m/z 407.3 [M+H]+, Purity = 100%. |
| | | 1-[2-(2,4-difluorophenyl)-3-(pyridin-4-yl)-3H-imidazo[4,5-b]pyridin-5-yl]-1,4-diazepane | |
| 138 | Intermediate 76 (21% yield) | 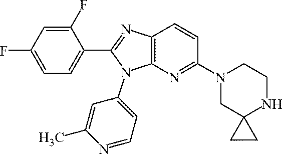 [SMILES: Cc1cc(-n2c(-c3ccc(F)cc3F)nc3ccc(N4CCNC5(CC5)C4)nc32)ccn1] | 1H NMR (400 MHz, DMSO-d6) δ 8.48 (d, J = 5.4 Hz, 1H), 7.94 (d, J = 9.0 Hz, 1H), 7.80 - 7.72 (m, 1H), 7.34 - 7.24 (m, 3H), 7.08 (dd, J = 5.4, 1.7 Hz, 1H), 6.89 (d, J = 9.0 Hz, 1H), 3.52 - 3.47 (m, 2H), 3.38 (s, 2H), 2.86 - 2.79 (m, 2H), 2.45 (s, 3H), 2.38 - 2.33 (m, 1H), 0.46 (m, 4H). LCMS (Analytical Method B) Rt = 2.79 min, MS (ESIpos): m/z 433.4 [M+H]+, Purity = 100%. |
| | | 7-[2-(2,4-difluorophenyl)-3-(2-methylpyridin-4-yl)-3H-imidazo[4,5-b]pyridin-5-yl]-4,7-diazaspiro[2.5]octane | |
| 135-SS | Intermediate 48 (57% yield) | 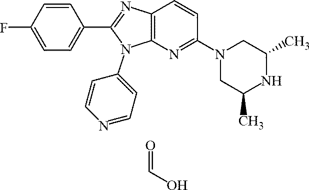 | 1H NMR (400 MHz, Chloroform-d) δ 8.75 - 8.69 (m, 2H), 8.47 (s, 1H), 7.95 (d, J = 8.9 Hz, 1H), 7.54 - 7.47 (m, 2H), 7.36 - 7.29 (m, 2H), 7.11 -7.03 (m, 2H), 6.73 (d, J = 8.9 Hz, 1H), 3.89 - 3.81 (m, 2H), 3.61 - 3.48 (m, 4H), 1.37 (d, J = 6.4 Hz, 6H). LCMS (Analytical Method A) Rt = 1.56 min, MS (ESIpos): m/z 403 [M+H]+, Purity = 100%. |
| | | (3S,5S)-1-[2-(4-fluorophenyl)-3-(pyridin-4-yl)-3H-imidazo[4,5-b]pyridin-5-yl]-3,5-dimethylpiperazine; formic acid | |
| 139 | Intermediate 5 (32% yield) | 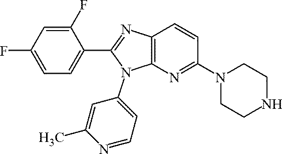 [SMILES: Cc1cc(-n2c(-c3ccc(F)cc3F)nc3ccc(N4CCNCC4)nc32)ccn1] | 1H NMR (500 MHz, Methanol-d4) δ 8.48 (d, J = 5.5 Hz, 1H), 7.94 (d, J = 9.0 Hz, 1H), 7.76 (td, J = 8.4, 6.3 Hz, 1H), 7.35 (d, J = 2.0 Hz, 1H), 7.27 (dd, J = 5.5, 1.7 Hz, 1H), 7.20 - 7.15 (m, 1H), 7.03 (ddd, J = 10.4, 9.0, 2.5 Hz, 1H), 6.95 (d, J = 9.0 Hz, 1H), 3.61 - 3.54 (m, 4H), 2.96 - 2.88 (m, 4H), 2.52 (s, 3H). LCMS (Analytical Method B) Rt = 2.54 min, MS (ESIpos): m/z 407.4 [M+H]+, Purity = 99%. |
| | | 1-[2-(2,4-difluorophenyl)-3-(2-methylpyridin-4-yl)-3H-imidazo[4,5-b]pyridin-5-yl]piperazine | |
| 140 | Intermediate 5 (34% yield) | 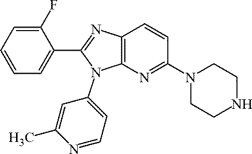 [SMILES: Cc1cc(-n2c(-c3ccccc3F)nc3ccc(N4CCNCC4)nc32)ccn1] | 1H NMR (500 MHz, Methanol-d4) δ 8.45 (d, J = 5.5 Hz, 1H), 7.94 (d, J = 9.0 Hz, 1H), 7.72 (td, J = 7.4, 1.8 Hz, 1H), 7.61 - 7.53 (m, 1H), 7.37 (td, J = 7.6, 1.0 Hz, 1H), 7.33 (d, J = 2.0 Hz, 1H), 7.27 (dd, J = 5.5, 2.0 Hz, 1H), 7.16 - 7.09 (m, 1H), 6.95 (d, J = 9.0 Hz, 1H), 3.61 - 3.54 (m, 4H), 2.96 - 2.89 (m, 4H), 2.50 (s, 3H). LCMS (Analytical Method A) Rt = 1.20 min, MS (ESIpos): m/z 389.2 [M+H]+, Purity = 99%. |
| | | 1-[2-(2-fluorophenyl)-3-(2-methylpyridin-4-yl)-3H-imidazo[4,5-b]pyridin-5-yl]piperazine | |
| 141 | Intermediate 4 (47% yield) | 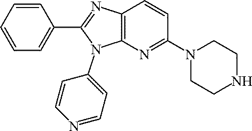 [SMILES: c1ccc(-c2nc3ccc(N4CCNCC4)nc3n2-c2ccncc2)cc1] | 1H NMR (400 MHz, Methanol-d4) δ 8.66 - 8.61 (m, 2H), 7.92 (d, J = 9.0 Hz, 1H), 7.55 - 7.38 (m, 7H), 6.93 (d, J = 9.0 Hz, 1H), 3.59 - 3.51 (m, 4H), 2.95 - 2.88 (m, 4H). LCMS (Analytical Method A) Rt = 1.24 min, MS (ESIpos): m/z 357.2 [M+H]+, Purity = 100%. |
| | | 1-[2-phenyl-3-(pyridin-4-yl)-3H-imidazo[4,5-b]pyridin-5-yl]piperazine | |
| 142-RR | Intermediate 78 (48% yield) | 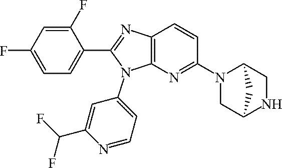 [SMILES: Fc1ccc(-c2nc3ccc(N4C[C@H]5C[C@@H]4CN5)nc3n2-c2ccnc(C(F)F)c2)c(F)c1] | 1H NMR (500 MHz, Methanol-d4) δ 8.67 (d, J = 5.4 Hz, 1H), 7.93 - 7.87 (m, 2H), 7.81 (td, J = 8.4, 6.3 Hz, 1H), 7.50 (dd, J = 5.4, 2.0 Hz, 1H), 7.21 (td, J = 8.2, 1.9 Hz, 1H), 7.03 (ddd, J = 10.5, 9.0, 2.5 Hz, 1H), 6.90 - 6.61 (m, 2H), 4.80 - 4.74 (m, 1H), 3.86 - 3.80 (m, 1H), 3.61 (dd, J = 9.7, 2.2 Hz, 1H), 3.37 (d, J = 9.8 Hz, 1H), 3.01 (s, 2H), 1.96 (d, J = 9.7 Hz, 1H), 1.82 (d, J = 9.8 Hz, 1H). LCMS (Analytical Method A) Rt = 1.87 min, MS (ESIpos): m/z 455.2 [M+H]+, Purity = 98%. |
| | | (1R,4R)-2-{3-[2-(difluoromethyl)pyridin-4-yl]-2-(2,4-difluorophenyl)-3H-imidazo[4,5-b]pyridin-5-yl}-2,5-diazabicyclo[2.2.1]heptane | |
| 143-RR | Intermediate 78 (55% yield) | 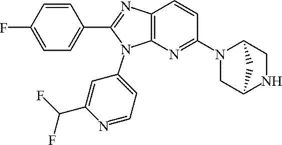 [SMILES: Fc1ccc(-c2nc3ccc(N4C[C@H]5C[C@@H]4CN5)nc3n2-c2ccnc(C(F)F)c2)cc1] | 1H NMR (500 MHz, Methanol-d4) δ 8.68 (d, J = 5.3 Hz, 1H), 7.92 (d, J = 1.9 Hz, 1H), 7.88 (d, J = 8.8 Hz, 1H), 7.58 - 7.51 (m, 2H), 7.50 - 7.45 (m, 1H), 7.23 - 7.14 (m, 2H), 6.77 (t, J = 55.1 Hz, 1H), 6.61 (d, J = 8.8 Hz, 1H), 4.78 - 4.73 (m, 1H), 3.84 - 3.79 (m, 1H), 3.59 (dd, J = 9.7, 2.2 Hz, 1H), 3.35 (d, J = 9.8 Hz, 1H), 3.00 (s, 2H), 1.95 (d, J = 8.7 Hz, 1H), 1.81 (d, J = 9.8 Hz, 1H). LCMS (Analytical Method A) Rt = 1.86 min, MS (ESIpos): m/z 437.2 [M+H]+, Purity = 100%. |
| | | (1R,4R)-2-{3-[2-(difluoromethyl)pyridin-4-yl]-2-(4-fluorophenyl)-3H-imidazo[4,5-b]pyridin-5-yl}-2,5-diazabicyclo[2.2.1]heptane | |
| 145-S | Intermediate 49 (9% yield) | 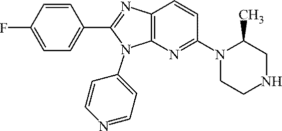 [SMILES: C[C@H]1CNCCN1c1ccc2nc(-c3ccc(F)cc3)n(-c3ccncc3)c2n1] | 1H NMR (400 MHz, Chloroform-d) δ 8.70 (dd, J = 4.7, 1.6 Hz, 2H), 7.91 (d, J = 8.9 Hz, 1H), 7.56 - 7.46 (m, 2H), 7.38 - 7.31 (m, 2H), 7.06 (t, J = 8.6 Hz, 2H), 6.69 (d, J = 9.0 Hz, 1H), 4.44 - 4.34 (m, 1H), 4.01 - 3.91 (m, 1H), 3.15 - 3.00 (m, 3H), 2.93 (d, J = 12.2 Hz, 1H), 2.84 (td, J= 11.8, 3.5 Hz, 1H), 1.22 (d, J = 6.7 Hz, 3H). LCMS (Analytical Method A) Rt = 1.48 min, MS (ESIpos): m/z 389 [M+H]+, Purity = 100%. |
| | | (2S)-1-[2-(4-fluorophenyl)-3-(pyridin-4-yl)-3H-imidazo[4,5-b]pyridin-5-yl]-2-methylpiperazine | |
| 145-R | Intermediate 79 (20% yield) | 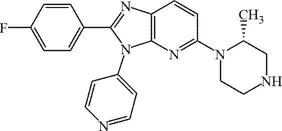 [SMILES: C[C@@H]1CNCCN1c1ccc2nc(-c3ccc(F)cc3)n(-c3ccncc3)c2n1] | 1H NMR (500 MHz, Chloroform-d) δ 8.72 - 8.66 (m, 2H), 7.92 (d, J = 8.9 Hz, 1H), 7.53 - 7.48 (m, 2H), 7.37 -7.32 (m, 2H), 7.09 - 7.02 (m, 2H), 6.69 (d, J = 9.0 Hz, 1H), 4.46 - 4.32 (m, 1H), 4.04 - 3.91 (m, 1H), 3.17 -3.10 (m, 1H), 3.06 (td, J = 12.2, 3.6 Hz, 2H), 2.94 (d, J = 12.2 Hz, 1H), 2.85 (td, J = 12.0, 3.6 Hz, 1H), 1.23 (d, J = 6.7 Hz, 3H). LCMS (Analytical Method A) Rt = 1.47 min, MS (ESIpos): m/z 389 [M+H]+, Purity = 100%. |
| | | (2R)-1-[2-(4-fluorophenyl)-3-(pyridin-4-yl)-3H-imidazo[4,5-b]pyridin-5-yl]-2-methylpiperazine | |
| 150 | Intermediate 94 (7% yield) | 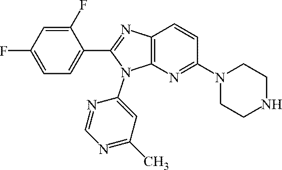 [SMILES: Cc1cc(-n2c(-c3ccc(F)cc3F)nc3ccc(N4CCNCC4)nc32)ncn1] | 1H NMR (400 MHz, Methanol-d4) δ 8.74 (s, 1H), 8.23 (s, 1H), 7.98 (d, J = 9.0 Hz, 1H), 7.81 (td, J = 8.4, 6.4 Hz, 1H), 7.18 (td, J = 8.4, 2.1 Hz, 1H), 7.05 - 6.96 (m, 2H), 3.72 - 3.61 (m, 4H), 3.06 -2.93 (m, 4H), 2.71 (s, 3H). LCMS (Analytical Method A) Rt = 1.66 min, MS (ESIpos): m/z 408 [M+H]+, Purity = 95%. |
| | | 4-[2-(2,4-difluorophenyl)-5-(piperazin-1-yl)-3H-imidazo[4,5-b]pyridin-3-yl]-6-methylpyrimidine | |
| 151 | Intermediate 50 (17% yield) | 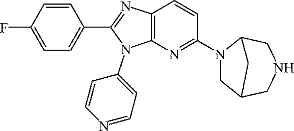 [SMILES: Fc1ccc(-c2nc3ccc(N4CC5CNCC4C5)nc3n2-c2ccncc2)cc1] | 1H NMR (400 MHz, DMSO-d6) δ 8.71 - 8.65 (m, 2H), 7.89 (d, J = 8.8 Hz, 1H), 7.52 - 7.46 (m, 2H), 7.44 -7.41 (m, 2H), 7.28 - 7.21 (m, 2H), 6.55 (d, J = 8.9 Hz, 1H), 4.15 - 4.08 (m, 1H), 3.49 - 3.40 (m, 2H), 2.97 -2.89 (m, 1H), 2.77 - 2.66 (m, 2H), 2.55 - 2.52 (m, 1H), 2.34 - 2.29 (m, 1H), 1.97 - 1.90 (m, 1H), 1.79 - 1.73 (m, 1H). LCMS (Analytical Method A) Rt = 1.48 min, MS (ESIpos): m/z 401.2 [M+H]+, Purity = 99%. |
| | | 6-[2-(4-fluorophenyl)-3-(pyridin-4-yl)-3H-imidazo[4,5-b]pyridin-5-yl]-3,6-diazabicyclo[3.2.1]octane | |
| 152-RR | Intermediate 78 (49% yield) | 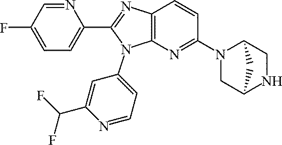 [SMILES: Fc1ccc(-c2nc3ccc(N4C[C@H]5C[C@@H]4CN5)nc3n2-c2ccnc(C(F)F)c2)nc1] | 1H NMR (500 MHz, Methanol-d4) δ 8.68 (d, J = 5.3 Hz, 1H), 8.25 (d, J = 2.9 Hz, 1H), 8.21 (dd, J = 8.8, 4.4 Hz, 1H), 7.91 (d, J = 8.9 Hz, 1H), 7.85 (d, J = 1.9 Hz, 1H), 7.78 (td, J = 8.6, 2.9 Hz, 1H), 7.52 - 7.48 (m, 1H), 6.79 (t, J = 55.1 Hz, 1H), 6.64 (d, J = 8.9 Hz, 1H), 4.77 - 4.72 (m, 1H), 3.84 - 3.79 (m, 1H), 3.58 (dd, J = 9.8, 2.2 Hz, 1H), 3.35 (d, J = 9.5 Hz, 1H), 2.99 (s, 2H), 1.94 (d, J = 9.6 Hz, 1H), 1.80 (d, J = 9.7 Hz, 1H). LCMS (Analytical Method A) Rt = 1.70 min, MS (ESIpos): m/z 438.2 [M+H]+, Purity = 96%. |
| | | (1R,4R)-2-{3-[2-(difluoromethyl)pyridin-4-yl]-2-(5-fluoropyridin-2-yl)-3H-imidazo[4,5-b]pyridin-5-yl}-2,5-diazabicyclo[2.2.1]heptane | |
| 153 | Intermediate 95 (34% yield) | 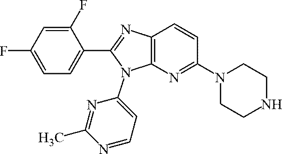 [SMILES: Cc1nccc(-n2c(-c3ccc(F)cc3F)nc3ccc(N4CCNCC4)nc32)n1] | 1H NMR (500 MHz, Chloroform-d) δ 8.81 (d, J = 5.5 Hz, 1H), 8.09 (d, J = 5.5 Hz, 1H), 7.94 (d, J = 8.9 Hz, 1H), 7.86 - 7.76 (m, 1H), 7.08 - 7.00 (m, 1H), 6.79 - 6.69 (m, 2H), 3.64 - 3.52 (m, 4H), 3.08 -2.97 (m, 4H), 2.39 (s, 3H). LCMS (Analytical Method A) Rt = 1.68 min, MS (ESIpos): m/z 408 [M+H]+, Purity = 100%. |
| | | 4-[2-(2,4-difluorophenyl)-5-(piperazin-1-yl)-3H-imidazo[4,5-b]pyridin-3-yl]-2-methylpyrimidine | |
| 156 | Intermediate 51 (4% yield) | 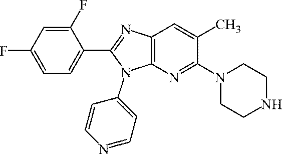 [SMILES: Cc1cc2nc(-c3ccc(F)cc3F)n(-c3ccncc3)c2nc1N1CCNCC1] | 1H NMR (500 MHz, Methanol-d4) δ 8.69 - 8.55 (m, 2H), 7.93 (s, 1H), 7.85 - 7.74 (m, 1H), 7.56 - 7.46 (m, 2H), 7.25- 7.14 (m, 1H), 7.11 - 6.94 (m, 1H), 3.20 - 3.08 (m, 4H), 3.08 -2.94 (m, 4H), 2.46 (s, 3H). LCMS (Analytical Method B) Rt = 2.81 min, MS (ESIpos): m/z 407.3 [M+H]+, Purity = 100%. |
| | | 1-[2-(2,4-difluorophenyl)-6-methyl-3-(pyridin-4-yl)-3H-imidazo[4,5-b]pyridin-5-yl]piperazine | |
| 157-R formic acid salt | Intermediate 52 (45% yield) | 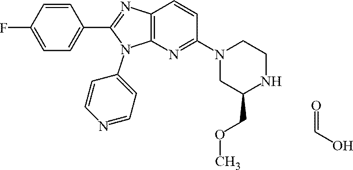 [SMILES: COC[C@H]1CN(c2ccc3nc(-c4ccc(F)cc4)n(-c4ccncc4)c3n2)CCN1 ; O=CO] | 1H NMR (500 MHz, Methanol-d4) δ 8.69 (dd, J = 4.7, 1.5 Hz, 2H), 8.49 (s, 1H), 8.00 (d, J = 8.9 Hz, 1H), 7.60 - 7.53 (m, 2H), 7.54 - 7.48 (m, 2H), 7.23 - 7.16 (m, 2H), 7.04 (d, J = 9.0 Hz, 1H), 4.36 (d, J = 12.1 Hz, 2H), 3.62 (dd, J = 10.3, 4.4 Hz, 1H), 3.55 (dd, J = 10.2, 6.4 Hz, 1H), 3.48 -3.34 (m, 5H), 3.22 - 3.01 (m, 3H). LCMS (Analytical Method A) Rt = 1.47 min, MS (ESIpos): m/z 419 [M+H]+, Purity = 100%. |
| | | (3R)-1-[2-(4-fluorophenyl)-3-(pyridin-4-yl)-3H-imidazo[4,5-b]pyridin-5-yl]-3-(methoxymethyl)piperazine formic acid salt | |
| 157-S formic acid salt | Intermediate 53 (44% yield) | 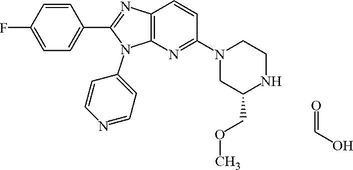 [SMILES: COC[C@@H]1CN(c2ccc3nc(-c4ccc(F)cc4)n(-c4ccncc4)c3n2)CCN1 ; O=CO] | 1H NMR (500 MHz, MeOD) δ 8.67 (dd, J = 4.7, 1.5 Hz, 2H), 8.45 (s, 1H), 8.00 (d, J = 8.9 Hz, 1H), 7.55 (m, 2H), 7.52 - 7.48 (m, 2H), 7.22 -7.15 (m, 2H), 7.03 (d, J = 9.0 Hz, 1H), 4.37 (d, J = 13.5 Hz, 2H), 3.63 (dd, J = 10.3, 4.3 Hz, 1H), 3.56 (dd, J = 10.4, 6.4 Hz, 1H), 3.44 (s, 3H), 3.38 - 3.33 (m, 2H), 3.24 - 3.04 (m, 3H). LCMS (Analytical Method A) Rt = 1.48 min, MS (ESIpos): m/z 419 [M+H]+, Purity = 98%. |
| | | (3S)-1-[2-(4-fluorophenyl)-3-(pyridin-4-yl)-3H-imidazo[4,5-b]pyridin-5-yl]-3-(methoxymethyl)piperazine formic acid salt | |
| 169-R | Intermediate 80 (48% yield) | 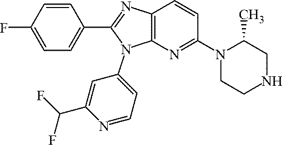 [SMILES: C[C@@H]1CNCCN1c1ccc2nc(-c3ccc(F)cc3)n(-c3ccnc(C(F)F)c3)c2n1] | 1H NMR (400 MHz, MeOD) δ 8.69 (d, J = 5.4 Hz, 1H), 7.96 - 7.86 (m, 2H), 7.56 (dd, J = 8.7, 5.3 Hz, 2H), 7.47 (d, J = 4.4 Hz, 1H), 7.19 (t, J = 8.7 Hz, 2H), 6.89 (d, J= 9.0 Hz, 1H), 6.77 (t, J = 55.0 Hz, 1H), 4.53 - 4.43 (m, 1H), 4.01 (d, J = 12.3 Hz, 1H), 3.15 - 2.95 (m, 3H), 2.89 (d, J= 12.7 Hz, 1H), 2.76 (td, J = 12.6, 3.7 Hz, 1H), 1.23 (d, J = 6.8 Hz, 3H). LCMS (Analytical Method A) Rt = 1.96 min, MS (ESIpos): m/z 439 [M+H]+, Purity = 98%. |
| | | (2R)-1-{3-[2-(difluoromethyl)pyridin-4-yl]-2-(4-fluorophenyl)-3H-imidazo[4,5-b]pyridin-5-yl}-2-methylpiperazine | |
| 169-S | Intermediate 81 (35% yield) | 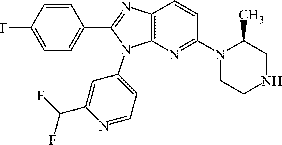 [SMILES: C[C@H]1CNCCN1c1ccc2nc(-c3ccc(F)cc3)n(-c3ccnc(C(F)F)c3)c2n1] | 1H NMR (400 MHz, MeOD) δ 8.69 (d, J = 5.4 Hz, 1H), 7.95 - 7.86 (m, 2H), 7.60 - 7.52 (m, 2H), 7.50 - 7.44 (m, 1H), 7.24 - 7.13 (m, 2H), 6.93 -6.60 (m, 2H), 4.55 - 4.43 (m, 1H), 4.06 - 3.95 (m, 1H), 3.16 - 2.95 (m, 3H), 2.90 (d, J = 12.7 Hz, 1H), 2.77 (td, J = 12.6, 3.7 Hz, 1H), 1.23 (d, J = 6.8 Hz, 3H). LCMS (Analytical Method A) Rt = 2.23 min, MS (ESIpos): m/z 439.2 [M+H]+, Purity = 99%. |
| | | (2S)-1-{3-[2-(difluoromethyl)pyridin-4-yl]-2-(4-fluorophenyl)-3H-imidazo[4,5-b]pyridin-5-yl}-2-methylpiperazine | |
| 171-RR | Intermediate 54 (13% yield) | 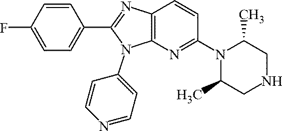 [SMILES: C[C@@H]1CNC[C@@H](C)N1c1ccc2nc(-c3ccc(F)cc3)n(-c3ccncc3)c2n1] | 1H NMR (500 MHz, MeOD) δ 8.69 -8.63 (m, 2H), 7.97 (d, J = 8.8 Hz, 1H), 7.62 - 7.53 (m, 2H), 7.53 - 7.48 (m, 2H), 7.23 - 7.15 (m, 2H), 6.99 (d, J = 8.8 Hz, 1H), 4.01 - 3.92 (m, 2H), 3.23 (dd, J = 12.8, 3.8 Hz, 2H), 2.80 (dd, J = 12.8, 5.0 Hz, 2H), 1.14 (d, J = 6.4 Hz, 6H). LCMS (Analytical Method A) Rt = 1.51 min, MS (ESIpos): m/z 403 [M+H]+, Purity = 100%. |
| | | (2R,6R)-1-[2-(4-fluorophenyl)-3-(pyridin-4-yl)-3H-imidazo[4,5-b]pyridin-5-yl]-2,6-dimethylpiperazine | |
| 172-S | Intermediate 55 (54% yield) | 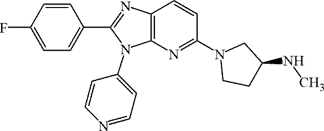 [SMILES: CN[C@H]1CCN(c2ccc3nc(-c4ccc(F)cc4)n(-c4ccncc4)c3n2)C1] | 1H NMR (500 MHz, MeOD) δ 8.69 -8.62 (m, 2H), 7.89 (d, J = 8.8 Hz, 1H), 7.59 - 7.49 (m, 4H), 7.23 - 7.13 (m, 2H), 6.61 (d, J = 8.9 Hz, 1H), 3.74 (dd, J = 10.4, 6.0 Hz, 1H), 3.70 - 3.63 (m, 1H), 3.55 - 3.48 (m, 1H), 3.42 - 3.34 (m, 2H), 2.44 (s, 3H), 2.33 - 2.23 (m, 1H), 1.99 - 1.89 (m, 1H). LCMS (Analytical Method A) Rt = 1.39 min, MS (ESIpos): m/z 389 [M+H]+, Purity = 100%. |
| | | (3S)-1-[2-(4-fluorophenyl)-3-(pyridin-4-yl)-3H-imidazo[4,5-b]pyridin-5-yl]-N-methylpyrrolidin-3-amine | |
| 173 | Intermediate 56 (22% yield) | 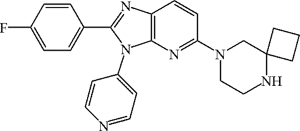 [SMILES: Fc1ccc(-c2nc3ccc(N4CCNC5(CCC5)C4)nc3n2-c2ccncc2)cc1] | 1H NMR (500 MHz, DMSO-d6) δ 8.74 - 8.69 (m, 2H), 7.94 (d, J = 8.9 Hz, 1H), 7.51 (dd, J = 8.9, 5.4 Hz, 2H), 7.46 - 7.42 (m, 2H), 7.26 (t, J = 8.9 Hz, 2H), 6.94 (d, J = 9.0 Hz, 1H), 3.44 (s, 2H), 3.43 - 3.38 (m, 2H), 2.72 - 2.67 (m, 2H), 1.91 - 1.85 (m, 2H), 1.79 - 1.70 (m, 4H). LCMS (Analytical Method A) Rt = 1.59 min, MS (ESIpos): m/z 415.3 [M+H]+, Purity = 98%. |
| | | 8-[2-(4-fluorophenyl)-3-(pyridin-4-yl)-3H-imidazo[4,5-b]pyridin-5-yl]-5,8-diazaspiro[3.5]nonane | |
| 174-S | Intermediate 55 (24% yield) | 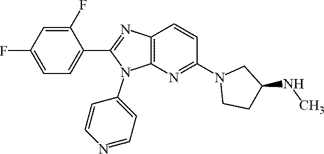 [SMILES: CN[C@H]1CCN(c2ccc3nc(-c4ccc(F)cc4F)n(-c4ccncc4)c3n2)C1] | 1H NMR (400 MHz, MeOD) δ 8.61 (dd, J = 4.7, 1.5 Hz, 2H), 7.89 (d, J = 8.9 Hz, 1H), 7.81 - 7.71 (m, 1H), 7.51 (dd, J = 4.7, 1.6 Hz, 2H), 7.17 (td, J = 8.3, 1.8 Hz, 1H), 7.06 - 6.97 (m, 1H), 6.62 (d, J = 8.9 Hz, 1H), 3.78 - 3.70 (m, 1H), 3.70 - 3.62 (m, 1H), 3.51 (dt, J = 10.1, 7.3 Hz, 1H), 3.42 - 3.33 (m, 2H), 2.43 (s, 3H), 2.32 - 2.22 (m, 1H), 1.98 - 1.87 (m, 1H). LCMS (Analytical Method A) Rt = 1.43 min, MS (ESIpos): m/z 407 [M+H]+, Purity = 100%. |
| | | (3S)-1-[2-(2,4-difluorophenyl)-3-(pyridin-4-yl)-3H-imidazo[4,5-b]pyridin-5-yl]-N-methylpyrrolidin-3-amine | |
| 172-R | Intermediate 57 (51% yield) | 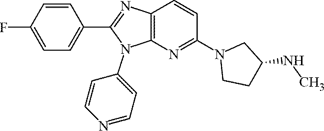 [SMILES: CN[C@@H]1CCN(c2ccc3nc(-c4ccc(F)cc4)n(-c4ccncc4)c3n2)C1] | 1H NMR (400 MHz, MeOD) δ 8.64 (dd, J = 4.7, 1.6 Hz, 2H), 7.87 (d, J = 8.8 Hz, 1H), 7.57 - 7.47 (m, 4H), 7.17 (t, J = 8.8 Hz, 2H), 6.59 (d, J = 8.9 Hz, 1H), 3.73 (dd, J = 10.2, 5.8 Hz, 1H), 3.68 - 3.60 (m, 1H), 3.54 -3.45 (m, 1H), 3.41 - 3.33 (m, 2H), 2.42 (s, 3H), 2.33 - 2.21 (m, 1H), 1.99 - 1.86 (m, 1H). LCMS (Analytical Method A) Rt = 1.39 min, MS (ESIpos): m/z 389 [M+H]+, Purity = 100%. |
| | | (3R)-1-[2-(4-fluorophenyl)-3-(pyridin-4-yl)-3H-imidazo[4,5-b]pyridin-5-yl]-N-methylpyrrolidin-3-amine | |
| 174-R | Intermediate 57 (21% yield) | 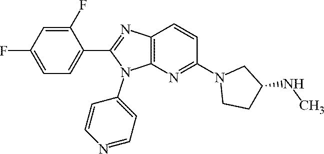 [SMILES: CN[C@@H]1CCN(c2ccc3nc(-c4ccc(F)cc4F)n(-c4ccncc4)c3n2)C1] | 1H NMR (400 MHz, MeOD) δ 8.61 (dd, J = 4.7, 1.6 Hz, 2H), 7.89 (d, J = 8.9 Hz, 1H), 7.81 - 7.71 (m, 1H), 7.51 (dd, J = 4.7, 1.6 Hz, 2H), 7.18 (td, J = 8.1, 1.6 Hz, 1H), 7.06 -6.96 (m, 1H), 6.62 (d, J = 8.9 Hz, 1H), 3.79 - 3.71 (m, 1H), 3.71 - 3.62 (m, 1H), 3.56 - 3.46 (m, 1H), 3.42 - 3.33 (m, 2H), 2.43 (s, 3H), 2.33 - 2.20 (m, 1H), 2.01 - 1.86 (m, 1H). LCMS (Analytical Method A) Rt = 1.44 min, MS (ESIpos): m/z 407 [M+H]+, Purity = 100%. |
| | | (3R)-1-[2-(2,4-difluorophenyl)-3-(pyridin-4-yl)-3H-imidazo[4,5-b]pyridin-5-yl]-N-methylpyrrolidin-3-amine | |
| 175-R | Intermediate 80 (2% yield) | 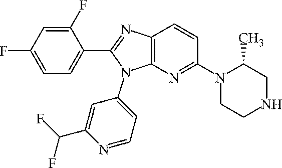 [SMILES: C[C@@H]1CNCCN1c1ccc2nc(-c3ccc(F)cc3F)n(-c3ccnc(C(F)F)c3)c2n1] | 1H NMR (400 MHz, DMSO) δ 8.75 (d, J = 5.4 Hz, 1H), 8.00 (d, J = 9.0 Hz, 1H), 7.87 - 7.75 (m, 2H), 7.54 -7.48 (m, 1H), 7.38 - 7.27 (m, 2H), 7.00 (t, J = 54.7 Hz, 1H), 6.90 (d, J = 9.1 Hz, 1H), 4.43 - 4.30 (m, 1H), 3.98 - 3.82 (m, 1H), 3.01 - 2.88 (m, 2H), 2.87 - 2.74 (m, 2H), 2.66 - 2.57 (m, 1H), 1.15 (d, J = 6.6 Hz, 3H). LCMS (Analytical Method A) Rt = 1.96 min, MS (ESIpos): m/z 457.3 [M+H]+, Purity = 99%. |
| | | (2R)-1-{3-[2-(difluoromethyl)pyridin-4-yl]-2-(2,4-difluorophenyl)-3H-imidazo[4,5-b]pyridin-5-yl}-2-methylpiperazine | |
| 178 | Intermediate 58 (15% yield) | 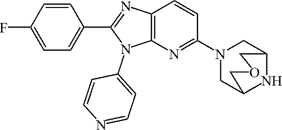 [SMILES: Fc1ccc(-c2nc3ccc(N4CC5COCC(C4)N5)nc3n2-c2ccncc2)cc1] | 1H NMR (500 MHz, DMSO) δ 8.75 -8.67 (m, 2H), 7.94 (d, J = 8.9 Hz, 1H), 7.56 - 7.48 (m, 2H), 7.48 - 7.42 (m, 2H), 7.30 - 7.18 (m, 2H), 6.82 (d, J = 9.0 Hz, 1H), 4.15 (d, J = 12.4 Hz, 2H), 3.82- 3.68 (m, 4H), 3.23 - 3.11 (m, 2H), 2.96 - 2.85 (m, 2H). LCMS (Analytical Method A) Rt = 1.36 min, MS (ESIpos): m/z 417.3 [M+H]+, Purity = 100%. |
| | | 7-[2-(4-fluorophenyl)-3-(pyridin-4-yl)-3H-imidazo[4,5-b]pyridin-5-yl]-3-oxa-7,9-diazabicyclo[3.3.1]nonane | |
| 182-RS | Intermediate 59 (48% yield) | 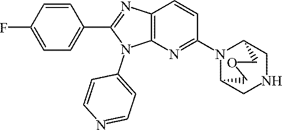 [SMILES: Fc1ccc(-c2nc3ccc(N4[C@@H]5CNC[C@H]4COC5)nc3n2-c2ccncc2)cc1] | 1H NMR (400 MHz, DMSO) δ 8.77 -8.65 (m, 2H), 7.99 (d, J = 8.9 Hz, 1H), 7.53 - 7.47 (m, 2H), 7.44 - 7.39 (m, 2H), 7.31 - 7.20 (m, 2H), 6.90 (d, J = 9.0 Hz, 1H), 4.09 (s, 2H), 4.06 (d, J = 11.3 Hz, 2H), 3.82 (d, J = 11.3 Hz, 2H), 3.09 (d, J = 13.6 Hz, 2H), 2.98 (d, J = 13.9 Hz, 2H). LCMS (Analytical Method A) Rt = 1.48 min, MS (ESIpos): m/z 417.3 [M+H]+, Purity = 100%. |
| | | (1R,5S)-9-[2-(4-fluorophenyl)-3-(pyridin-4-yl)-3H-imidazo[4,5-b]pyridin-5-yl]-3-oxa-7,9-diazabicyclo[3.3.1]nonane | |
| 187-R | Intermediate 79 (25% yield) | 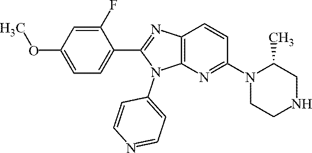 [SMILES: COc1ccc(-c2nc3ccc(N4CCNC[C@H]4C)nc3n2-c2ccncc2)c(F)c1] | 1H NMR (400 MHz, DMSO) δ 8.67 -8.64 (m, 2H), 7.95 (d, J = 9.0 Hz, 1H), 7.63 (t, J = 8.6 Hz, 1H), 7.40 -7.36 (m, 2H), 6.97 - 6.92 (m, 1H), 6.87 - 6.82 (m, 2H), 4.39 - 4.29 (m, 1H), 3.91 (d, J = 11.3 Hz, 1H), 3.82 (s, 3H), 3.00 - 2.76 (m, 4H), 2.60 (td, J = 11.8, 3.4 Hz, 1H), 1.13 (d, J = 6.6 Hz, 3H). LCMS (Analytical Method A) Rt = 1.49 min, MS (ESIpos): m/z 419.4 [M+H]+, Purity = 99%. |
| | | (2R)-1-[2-(2-fluoro-4-methoxyphenyl)-3-(pyridin-4-yl)-3H-imidazo[4,5-b]pyridin-5-yl]-2-methylpiperazine | |
| 188-R | Intermediate 79 (34% yield) | 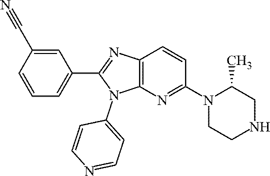 [SMILES: C[C@@H]1CNCCN1c1ccc2nc(-c3cccc(C#N)c3)n(-c3ccncc3)c2n1] | 1H NMR (500 MHz, DMSO) δ 8.74 -8.71 (m, 2H), 7.98 (d, J = 9.0 Hz, 1H), 7.93 - 7.88 (m, 2H), 7.71 (dt, J = 8.0, 1.3 Hz, 1H), 7.61 (t, J = 8.1 Hz, 1H), 7.48 - 7.45 (m, 2H), 6.88 (d, J = 9.1 Hz, 1H), 4.38 - 4.28 (m, 1H), 3.91 (d, J = 11.2 Hz, 1H), 2.96 (d, J = 12.7 Hz, 1H), 2.89 (td, J = 12.4, 3.4 Hz, 1H), 2.84 - 2.76 (m, 2H), 2.59 (td, J = 11.9, 3.4 Hz, 1H), 2.31 (s, 1H), 1.12 (d, J = 6.6 Hz, 3H). LCMS (Analytical Method A) Rt = 1.40 min, MS (ESIpos): m/z 396.4 [M+H]+, Purity = 100%. |
| | | 3-{5-[(2R)-2-methylpiperazin-1-yl]-3-(pyridin-4-yl)-3H-imidazo[4,5-b]pyridin-2-yl}benzonitrile | |
| 191-RR | Intermediate 60 (15% yield) | 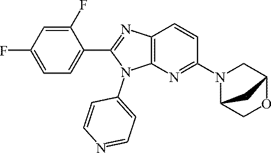 [SMILES: Fc1ccc(-c2nc3ccc(N4C[C@H]5C[C@@H]4CO5)nc3n2-c2ccncc2)c(F)c1] | 1H NMR (500 MHz, DMSO) δ 8.66 (dd, J = 4.7, 1.5 Hz, 2H), 7.99 (d, J = 8.8 Hz, 1H), 7.83 - 7.74 (m, 1H), 7.42 (dd, J = 4.6, 1.6 Hz, 2H), 7.35 -7.25 (m, 2H), 6.67 (d, J = 8.8 Hz, 1H), 4.89 - 4.84 (m, 1H), 4.69 - 4.64 (m, 1H), 3.80 (d, J = 7.3 Hz, 1H), 3.68 (d, J = 7.3 Hz, 1H), 3.53 - 3.47 (m, 1H), 3.32 - 3.30 (m, 1H), 1.95 -1.84 (m, 2H). LCMS (Analytical Method B) Rt = 2.83 min, MS (ESIpos): m/z 406.3 [M+H]+, Purity = 100%. |
| | | (1R,4R)-5-[2-(2,4-difluorophenyl)-3-(pyridin-4-yl)-3H-imidazo[4,5-b]pyridin-5-yl]-2-oxa-5-azabicyclo[2.2.1]heptane | |
| 192-R | Intermediate 79 (22% yield) | 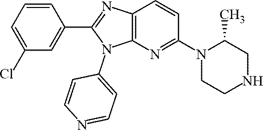 [SMILES: C[C@@H]1CNCCN1c1ccc2nc(-c3cccc(Cl)c3)n(-c3ccncc3)c2n1] | 1H NMR (500 MHz, DMSO) δ 8.75 -8.71 (m, 2H), 7.97 (d, J = 9.0 Hz, 1H), 7.56 (t, J = 1.8 Hz, 1H), 7.50 (ddd, J = 8.0, 2.1, 1.0 Hz, 1H), 7.48 - 7.46 (m, 2H), 7.41 (t, J = 7.9 Hz, 1H), 7.33 (dt, J = 7.8, 1.2 Hz, 1H), 6.87 (d, J = 9.1 Hz, 1H), 4.36 - 4.28 (m, 1H), 3.90 (d, J = 11.3 Hz, 1H), 2.96 (d, J = 12.6 Hz, 1H), 2.88 (td, J = 12.4, 3.4 Hz, 1H), 2.84 - 2.76 (m, 2H), 2.59 (td, J = 12.0, 3.5 Hz, 1H), 1.12 (d, J = 6.6 Hz, 3H). LCMS (Analytical Method A) Rt = 1.71 min, MS (ESIpos): m/z 405.3, 407.2 [M+H]+, Purity = 97%. |
| | | (2R)-1-[2-(3-chlorophenyl)-3-(pyridin-4-yl)-3H-imidazo[4,5-b]pyridin-5-yl]-2-methylpiperazine | |
| 193-R | Intermediate 79 (14% yield) | 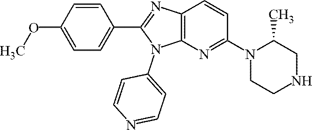 [SMILES: COc1ccc(-c2nc3ccc(N4CCNC[C@H]4C)nc3n2-c2ccncc2)cc1] | 1H NMR (500 MHz, DMSO) δ 8.72 -8.67 (m, 2H), 7.91 (d, J = 8.9 Hz, 1H), 7.45 - 7.40 (m, 2H), 7.41 - 7.36 (m, 2H), 6.98 - 6.93 (m, 2H), 6.80 (d, J = 9.0 Hz, 1H), 4.29 (s, 1H), 3.86 (d, J = 12.5 Hz, 1H), 3.78 (s, 3H), 2.94 (d, J = 11.5 Hz, 1H), 2.86 (td, J = 12.4, 3.4 Hz, 1H), 2.81 - 2.74 (m, 2H), 2.59 (d, J = 11.5 Hz, 1H), 1.10 (d, J = 6.6 Hz, 3H). LCMS (Analytical Method A) Rt = 1.37 min, MS (ESIpos): m/z 401.4 [M+H]+, Purity = 96%. |
| | | (2R)-1-[2-(4-methoxyphenyl)-3-(pyridin-4-yl)-3H-imidazo[4,5-b]pyridin-5-yl]-2-methylpiperazine | |
| 194 | Intermediate 61 (29% yield) | 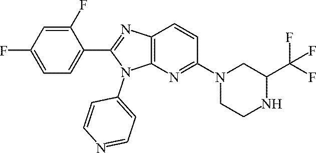 [SMILES: Fc1ccc(-c2nc3ccc(N4CCNC(C(F)(F)F)C4)nc3n2-c2ccncc2)c(F)c1] | 1H NMR (400 MHz, DMSO) δ 8.81 -8.46 (m, 2H), 8.03 (d, J = 8.9 Hz, 1H), 7.86 - 7.69 (m, 1H), 7.50 - 7.37 (m, 2H), 7.37 - 7.21 (m, 2H), 7.01 (d, J = 9.0 Hz, 1H), 4.30 (dd, J = 12.6, 2.9 Hz, 1H), 3.95 (d, J = 12.6 Hz, 1H), 3.45 (d, J = 7.9 Hz, 1H), 3.16 -2.88 (m, 4H), 2.79 - 2.68 (m, 1H). LCMS (Analytical Method A) Rt = 2.05 min, MS (ESIpos): m/z 461.2 [M+H]+, Purity = 100%. |
| | | 1-[2-(2,4-difluorophenyl)-3-(pyridin-4-yl)-3H-imidazo[4,5-b]pyridin-5-yl]-3-(trifluoromethyl)piperazine | |
| 195 | Intermediate 62 (3% yield) | 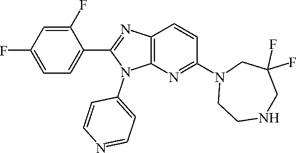 [SMILES: Fc1ccc(-c2nc3ccc(N4CCNCC(F)(F)C4)nc3n2-c2ccncc2)c(F)c1] | 1H NMR (400 MHz, MeOD) δ 8.62 (d, J = 5.5 Hz, 2H), 7.96 (d, J = 9.0 Hz, 1H), 7.79 (td, J = 8.4, 6.4 Hz, 1H), 7.50 (d, J = 6.3 Hz, 2H), 7.20 (td, J = 8.3, 2.1 Hz, 1H), 7.08 - 7.00 (m, 1H), 6.97 (d, J = 9.0 Hz, 1H), 4.26 (t, J = 12.5 Hz, 2H), 3.92 - 3.83 (m, 2H), 3.11 - 2.98 (m, 4H). LCMS (Analytical Method A) Rt = 1.52 min, MS (ESIpos): m/z 443.3 [M+H]+, Purity = 89%. |
| | | 1-[2-(2,4-difluorophenyl)-3-(pyridin-4-yl)-3H-imidazo[4,5-b]pyridin-5-yl]-6,6-difluoro-1,4-diazepane | |
| 196-R | Intermediate 79 (9% yield) | 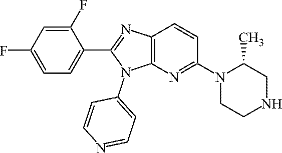 [SMILES: C[C@@H]1CNCCN1c1ccc2nc(-c3ccc(F)cc3F)n(-c3ccncc3)c2n1] | 1H NMR (500 MHz, MeOD) δ 8.65 -8.59 (m, 2H), 7.93 (d, J = 9.0 Hz, 1H), 7.78 (td, J = 8.4, 6.3 Hz, 1H), 7.51 - 7.46 (m, 2H), 7.18 (td, J = 8.2, 2.2 Hz, 1H), 7.06 - 6.99 (m, 1H), 6.91 (d, J = 9.1 Hz, 1H), 4.52 - 4.45 (m, 1H), 4.08 - 4.01 (m, 1H), 3.12 -3.03 (m, 2H), 3.03 - 2.97 (m, 1H), 2.94 - 2.88 (m, 1H), 2.81 - 2.72 (m, 1H), 1.23 (d, J = 6.8 Hz, 3H). LCMS (Analytical Method A) Rt = 1.49 min, MS (ESIpos): m/z 407.4 [M+H]+, Purity = 97%. |
| | | (2R)-1-[2-(2,4-difluorophenyl)-3-(pyridin-4-yl)-3H-imidazo[4,5-b]pyridin-5-yl]-2-methylpiperazine | |
| 199 | Intermediate 63 (10% yield) | 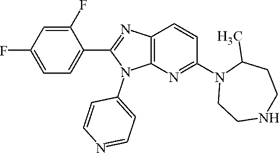 [SMILES: CC1CCNCCN1c1ccc2nc(-c3ccc(F)cc3F)n(-c3ccncc3)c2n1] | 1H NMR (400 MHz, DMSO) δ 8.68 -8.61 (m, 2H), 7.92 (d, J = 9.0 Hz, 1H), 7.81 - 7.73 (m, 1H), 7.42 - 7.36 (m, 2H), 7.34 - 7.24 (m, 2H), 6.73 (d, J = 9.1 Hz, 1H), 4.24 (s, 1H), 3.92 (d, J = 13.4 Hz, 1H), 3.25 - 3.16 (m, 1H), 2.96 - 2.85 (m, 2H), 2.66 - 2.57 (m, 1H), 2.26 (dd, J = 13.5, 9.8 Hz, 1H), 2.12 (dt, J = 13.4, 6.1 Hz, 1H), 1.68 - 1.56 (m, 1H), 1.12 (d, J = 6.2 Hz, 3H). LCMS (Analytical Method A) Rt = 1.57 min, MS (ESIpos): m/z 421.3 [M+H]+, Purity = 96%. |
| | | 1-[2-(2,4-difluorophenyl)-3-(pyridin-4-yl)-3H-imidazo[4,5-b]pyridin-5-yl]-7-methyl-1,4-diazepane | |
| 202-R | Intermediate 79 (23% yield) | 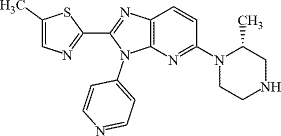 [SMILES: Cc1cnc(-c2nc3ccc(N4CCNC[C@H]4C)nc3n2-c2ccncc2)s1] | 1H NMR (400 MHz, DMSO) δ 8.82 -8.68 (m, 2H), 7.97 (d, J = 9.0 Hz, 1H), 7.63 - 7.52 (m, 2H), 7.47 (d, J = 1.2 Hz, 1H), 6.90 (d, J = 9.1 Hz, 1H), 4.35 - 4.25 (m, 1H), 3.95 - 3.85 (m, 1H), 2.99 - 2.92 (m, 1H), 2.91 -2.82 (m, 1H), 2.81 - 2.75 (m, 2H), 2.62 - 2.56 (m, 1H), 2.50 - 2.47 (m, 3H), 1.12 (d, J = 6.6 Hz, 3H). LCMS (Analytical Method A) Rt = 1.32 min, MS (ESIpos): m/z 392.2 [M+H]+, Purity = 99%. |
| | | (2R)-2-methyl-1-[2-(5-methyl-1,3-thiazol-2-yl)-3-(pyridin-4-yl)-3H-imidazo[4,5-b]pyridin-5-yl]piperazine | |
| 203-S | Intermediate 64 (3% yield) | 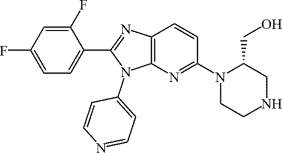 [SMILES: OC[C@@H]1CNCCN1c1ccc2nc(-c3ccc(F)cc3F)n(-c3ccncc3)c2n1] | 1H NMR (400 MHz, MeOD) δ 8.62 (d, J = 5.6 Hz, 2H), 7.94 (d, J = 9.0 Hz, 1H), 7.78 (td, J = 8.4, 6.4 Hz, 1H), 7.48 (d, J = 6.2 Hz, 2H), 7.18 (td, J = 8.4, 2.2 Hz, 1H), 7.06 - 6.98 (m, 1H), 6.94 (d, J = 9.1 Hz, 1H), 4.47 - 4.37 (m, 1H), 4.21 - 4.09 (m, 1H), 3.98 - 3.88 (m, 1H), 3.78 - 3.64 (m, 1H), 3.39 - 3.33 (m, 1H), 3.25 -3.06 (m, 2H), 3.02 - 2.90 (m, 1H), 2.91 - 2.77 (m, 1H). LCMS (Analytical Method B) Rt = 2.26 min, MS (ESIpos): m/z 423.3 [M+H]+, Purity = 97%. |
| | | [(2S)-1-[2-(2,4-difluorophenyl)-3-(pyridin-4-yl)-3H-imidazo[4,5-b]pyridin-5-yl]piperazin-2-yl]methanol | |

### Synthesis of 1-[2-(2,5-difluorophenyl)-3-(pyridin-4-yl)-3H-imidazo[4,5-b]pyridin-5-yl]piperazine / Compound 85 of Table 1

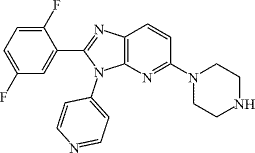 [SMILES: Fc1ccc(F)c(-c2nc3ccc(N4CCNCC4)nc3n2-c2ccncc2)c1]

Tert-butyl 4-[5-nitro-6-(4-pyridylamino)-2-pyridyl]piperazine-1-carboxylate (Intermediate 4) (100 mg, 0.250 mmol) and 2,5-difluorobenzaldehyde (41 uL, 0.375 mmol) were dissolved in a solution of DMSO (1.6 mL) and EtOH (0.2 mL) then Na₂S₂O₄ (132 mg, 0.749 mmol) was added. The mixture was sealed and stirred at 100° C. for 16 h. Air was bubbled through the mixture for 10 min then it was stirred at 100° C. for 4 h. The mixture was cooled to room temperature and 4 M aq HCl in dioxane (0.5 mL) was added. The reaction was left standing for 2 days. The reaction was basified then extracted with EtOAc. The organics were combined, filtered, and the filtrate was concentrated in vacuo. The crude product was purified by preparative HPLC (Method A1), then lyophilised to afford the title compound (9 mg, 9%) as a yellow solid. 1H NMR (500 MHz, DMSO-d6) δ 8.68 - 8.65 (m, 2H), 8.00 (d,J = 9.0 Hz, 1H), 7.61 - 7.56 (m, 1H), 7.45 -7.39 (m, 3H), 7.30 - 7.24 (m, 1H), 6.95 (d,J = 9.0 Hz, 1H), 3.46 - 3.42 (m, 4H), 2.81 - 2.75 (m, 4H). LCMS (Analytical Method B) Rt = 2.51 min, MS (ESIpos): m/z 393.3 [M+H]+, Purity = 99%.

Each of the compounds listed in Table 1.5.5 were prepared according to the method of Compound 85 of Table 1 using the intermediate listed in the “Synthesis” column and with appropriate aldehyde derivatives for such compounds. Ethanol is a co-solvent and was not used in all examples. Final compounds were purified by preparative HPLC Methods, A1, A2 or B1. If required, further purification using KP-NH column (gradient 0-50% MeOH/ DCM) or SCX cartridge (3N NH3 in MeOH) was carried out.

TABLE 1.5.5

| | | | |
|---|---|---|---|
| Example | Synthesis | Structure/ Name | Data |
| 166 | Intermediate 26 (2% yield) | 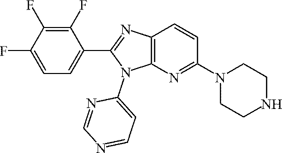 [SMILES: Fc1ccc(-c2nc3ccc(N4CCNCC4)nc3n2-c2ccncn2)c(F)c1F] | 1H NMR (400 MHz, DMSO) δ 9.11 (d, J = 5.5 Hz, 1H), 8.95 (s, 1H), 8.38 - 8.30 (m, 1H), 8.03 (d, J = 9.0 Hz, 1H), 7.67 - 7.56 (m, 1H), 7.52 - 7.42 (m, 1H), 7.00 (d, J = 9.0 Hz, 1H), 3.55 - 3.50 (m, 4H), 2.85 - 2.79 (m, 4H). LCMS (Analytical Method B) Rt = 2.81 min, MS (ESIpos): m/z 412.3 [M+H]+, Purity = 100%. |
| | | 4-[5-(piperazin-1-yl)-2-(2,3,4-trifluorophenyl)-3H-imidazo[4,5-b]pyridin-3-yl]pyrimidine | |
| 167 | Intermediate 26 (8% yield) | 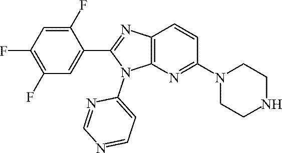 [SMILES: Fc1cc(F)c(-c2nc3ccc(N4CCNCC4)nc3n2-c2ccncn2)cc1F] | 1H NMR (400 MHz, DMSO) δ 9.10 (d, J = 5.7 Hz, 1H), 8.94 (d, J = 1.0 Hz, 1H), 8.32 (dd, J = 5.5, 1.3 Hz, 1H), 8.03 (d, J = 9.0 Hz, 1H), 7.98 - 7.86 (m, 1H), 7.68 - 7.54 (m, 1H), 6.99 (d, J = 9.1 Hz, 1H), 3.54 - 3.48 (m, 4H), 2.87 - 2.76 (m, 4H). LCMS (Analytical Method B) Rt = 2.81 min, MS (ESIpos): m/z 412.3 [M+H]+, Purity = 98%. |
| | | 4-[5-(piperazin-1-yl)-2-(2,4,5-trifluorophenyl)-3H-imidazo[4,5-b]pyridin-3-yl]pyrimidine | |
| 180-R | Intermediate 80 (15% yield) | 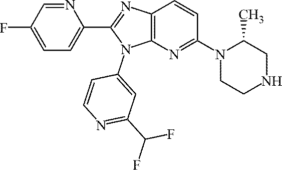 [SMILES: C[C@@H]1CNCCN1c1ccc2nc(-c3ccc(F)cn3)n(-c3ccnc(C(F)F)c3)c2n1] | 1H NMR (400 MHz, DMSO) δ 8.75 (d, J = 5.3 Hz, 1H), 8.35 (d, J = 2.9 Hz, 1H), 8.28 (dd, J = 8.8, 4.6 Hz, 1H), 8.00 (d, J = 9.0 Hz, 1H), 7.94 (td, J = 8.8, 2.9 Hz, 1H), 7.82 (d, J = 1.8 Hz, 1H), 7.56 (dd, J = 5.3, 1.9 Hz, 1H), 7.17 - 6.88 (m, 2H), 4.36 - 4.28 (m, 1H), 3.90 (d, J = 12.5 Hz, 1H), 2.98 - 2.85 (m, 2H), 2.83 - 2.73 (m, 2H), 2.64 - 2.56 (m, 1H), 1.13 (d, J = 6.6 Hz, 3H). LCMS (Analytical Method A) Rt = 1.78 min, MS (ESIpos): m/z 440.3 [M+H]+, Purity = 99%. |
| | | (2R)-1-{3-[2-(difluoromethyl)pyridin-4-yl]-2-(5-fluoropyridin-2-yl)-3H-imidazo[4,5-b]pyridin-5-yl}-2-methylpiperazine | |
| 181-R | Intermediate 80 (20% yield) | 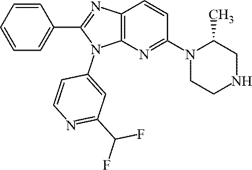 [SMILES: C[C@@H]1CNCCN1c1ccc2nc(-c3ccccc3)n(-c3ccnc(C(F)F)c3)c2n1] | 1H NMR (400 MHz, DMSO) δ 8.76 (d, J = 5.3 Hz, 1H), 7.97 (d, J = 8.9 Hz, 1H), 7.85 (d, J = 1.8 Hz, 1H), 7.53 - 7.38 (m, 6H), 7.01 (t, J = 55 Hz, 1H), 6.88 - 6.84 (m, 1H), 4.34 (s, 1H), 3.92 - 3.85 (m, 1H), 2.99 -2.86 (m, 2H), 2.86 - 2.74 (m, 2H), 2.65 - 2.57 (m, 1H), 1.14 (d, J = 6.6 Hz, 3H). LCMS (Analytical Method A) Rt = 1.86 min, MS (ESIpos): m/z 421.3 [M+H]+, Purity = 100%. |
| | | (2R)-1-{3-[2-(difluoromethyl)pyridin-4-yl]-2-phenyl-3H-imidazo[4,5-b]pyridin-5-yl}-2-methylpiperazine | |

### Synthesis of 7-[2-(3-fluorophenyl)-3-(pyridin-4-yl)-3H-imidazo[4,5-b]pyridin-5-yl]-4,7-diazaspiro[2.5]octane / Compound 146 of Table 1

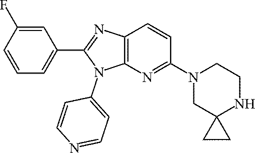 [SMILES: Fc1cccc(-c2nc3ccc(N4CCNC5(CC5)C4)nc3n2-c2ccncc2)c1]

3-Fluorobenzaldehyde (45 uL, 0.416 mmol) was added to a solution of tert-butyl 7-[5-amino-6-(4-pyridylamino)-2-pyridyl]-4,7-diazaspiro[2.5]octane-4-carboxylate (Intermediate 83) (150 mg, 0.378 mmol) in EtOH (7.5 mL). The reaction was stirred for 15 minutes then cerium ammonium nitrate (21 mg, 0.0378 mmol) was added followed by hydrogen peroxide (35%, 66 uL, 0.757 mmol). The reaction was stirred at ambient for 2 days then cooled and quenched into water. The aqueous layer was extracted into EtOAc three times, the combined organics washed with brine, dried over MgSO₄ and concentrated in vacuo. The residue was purified by preparative HPLC (Method A1). The residue was dissolved in DCM (7.5 mL) and TFA (0.075 mL) added. The solution was stirred for 4h, then quenched with aqueous ammonium hydroxide until no longer acidic. The solvent was removed in vacuo and the crude purified using preparative HPLC (Method A1) to afford the title compound (13 mg, 8.3% yield). 1H NMR (400 MHz, DMSO-d6) δ 8.77 - 8.67 (m, 2H), 7.95 (d, J = 9.0 Hz, 1H), 7.49 - 7.39 (m, 3H), 7.34 - 7.25 (m, 2H), 7.25 - 7.20 (m, 1H), 6.89 (d, J = 9.0 Hz, 1H), 3.53 - 3.45 (m, 2H), 3.35 (s, 2H), 2.88 - 2.77 (m, 2H), 2.38 - 2.28 (m, 1H), 0.52 - 0.38 (m, 4H). LCMS (Analytical Method A) Rt = 1.48 min, MS (ESlpos): m/z 401.2 [M+H]+, Purity = 100%.

Each of as the compounds listed in Table 1.5.6 were prepared according to the method of Compound 146 of Table 1 using the intermediate listed in the “Synthesis” column and with appropriate aldehyde derivatives for such compounds. The final compounds were purified by preparative HPLC Methods A1, or A2.

TABLE 1.5.6

| | | | |
|---|---|---|---|
| Example | Synthesis | Structure/ Name | Data |
| 31 | Intermediate 28 (22% yield) | 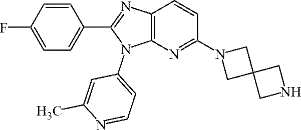 [SMILES: Cc1cc(-n2c(-c3ccc(F)cc3)nc3ccc(N4CC5(CNC5)C4)nc32)ccn1] | 1H NMR (400 MHz, Methanol-d4) δ 8.39 (d, J = 5.5 Hz, 1H), 7.77 (d, J = 8.7 Hz, 1H), 7.46 - 7.36 (m, 2H), 7.27 (d, J = 1.6 Hz, 1H), 7.12 (dd, J = 5.4, 1.7 Hz, 1H), 7.07 - 6.99 (m, 2H), 6.36 (d, J = 8.7 Hz, 1H), 4.02 (s, 4H), 3.74 (s, 4H), 2.45 (s, 3H). LCMS (Analytical Method A) Rt = 1.22 min, MS (ESlpos): m/z 401.2 [M+H]+, Purity = 97%. |
| | | 2-[2-(4-fluorophenyl)-3-(2-methylpyridin-4-yl)-3H-imidazo[4,5-b]pyridin-5-yl]-2,6-diazaspiro[3.3]heptane | |
| 53-RS | Intermediate 86 (13% yield) | 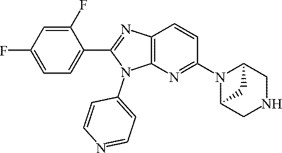 [SMILES: Fc1ccc(-c2nc3ccc(N4[C@@H]5CNC[C@H]4C5)nc3n2-c2ccncc2)c(F)c1] | 1H NMR (400 MHz, Chloroform-d) δ 8.61 - 8.53 (m, 2H), 7.87 (d, J = 8.6 Hz, 1H), 7.72 - 7.59 (m, 1H), 7.25 -7.21 (m, 2H), 7.02 - 6.92 (m, 1H), 6.76 - 6.60 (m, 1H), 6.41 (d, J = 8.6 Hz, 1H), 4.26 (d, J = 5.5 Hz, 2H), 3.48 (d, J= 12.4 Hz, 2H), 2.92 (d, J = 12.6 Hz, 2H), 2.78 - 2.69 (m, 1H), 1.67 (d, J = 8.3 Hz, 1H). LCMS (Analytical Method B) Rt = 2.40 min, MS (ESlpos): m/z 405.3 [M+H]+, Purity = 96%. |
| | | (1R,5S)-6-[2-(2,4-difluorophenyl)-3-(pyridin-4-yl)-3H-imidazo[4,5-b]pyridin-5-yl]-3,6-diazabicyclo[3.1.1]heptane | |
| 58-RS | Intermediate 86 (18% yield) | 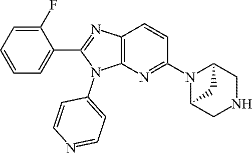 [SMILES: Fc1ccccc1-c1nc2ccc(N3[C@@H]4CNC[C@H]3C4)nc2n1-c1ccncc1] | 1H NMR (400 MHz, Chloroform-d) δ 8.59 - 8.49 (m, 2H), 7.87 (d, J = 8.6 Hz, 1H), 7.65 (td, J = 7.5, 1.7 Hz, 1H), 7.42 - 7.32 (m, 1H), 7.25 - 7.20 (m, 3H), 6.92 (t, J = 9.2 Hz, 1H), 6.40 (d, J = 8.6 Hz, 1H), 4.27 - 4.22 (m, 2H), 3.48 (d, J = 12.9 Hz, 2H), 2.91 (d, J = 12.6 Hz, 2H), 2.72 (q, J = 6.7 Hz, 1H), 1.66 (d, J = 8.3 Hz, 1H). LCMS (Analytical Method A) Rt = 1.37 min, MS (ESlpos): m/z 387.3 [M+H]+, Purity = 94%. |
| | | (1R,5S)-6-[2-(2-fluorophenyl)-3-(pyridin-4-yl)-3H-imidazo [4,5-b]pyridin-5-yl]-3,6-diazabicyclo[3.1.1]heptane | |
| 147 | Intermediate 83 (8% yield) | 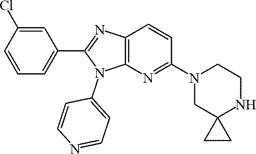 [SMILES: Clc1cccc(-c2nc3ccc(N4CCNC5(CC5)C4)nc3n2-c2ccncc2)c1] | 1H NMR (400 MHz, DMSO-d6) δ 8.76 - 8.67 (m, 2H), 7.95 (d, J = 9.0 Hz, 1H), 7.56 (t, J = 1.8 Hz, 1H), 7.50 (ddd, J = 8.0, 2.1, 1.1 Hz, 1H), 7.47 -7.44 (m, 2H), 7.41 (t, J = 7.9 Hz, 1H), 7.33 (dt, J = 7.7, 1.2 Hz, 1H), 6.89 (d, J = 9.0 Hz, 1H), 3.55 - 3.43 (m, 2H), 3.38 - 3.33 (m, 2H), 2.87 - 2.77 (m, 2H), 2.36 - 2.28 (m, 1H), 0.51 - 0.39 (m, 4H). LCMS (Analytical Method A) Rt = 1.69 min, MS (ESlpos): m/z 417.2 [M+H]+, Purity = 99%. |
| | | 7-[2-(3-chlorophenyl)-3-(pyridin-4-yl)-3H-imidazo[4,5-b]pyridin-5-yl]-4,7-diazaspiro[2.5]octane | |
| 148 | Intermediate 83 (1% yield) | 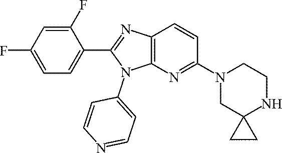 [SMILES: Fc1ccc(-c2nc3ccc(N4CCNC5(CC5)C4)nc3n2-c2ccncc2)c(F)c1] | 1H NMR (400 MHz, Chloroform-d) δ 9.87 (s, 1H), 8.64 (dd, J = 4.8, 1.5 Hz, 2H), 7.94 (d, J = 8.9 Hz, 1H), 7.76 -7.53 (m, 1H), 7.26 (dd, J = 4.8, 1.5 Hz, 2H), 6.99 (td, J = 8.2, 2.1 Hz, 1H), 6.76 - 6.61 (m, 2H), 3.96 - 3.81 (m, 2H), 3.66 (s, 2H), 3.38 - 3.19 (m, 2H), 1.27 - 1.22 (m, 2H), 0.88 - 0.80 (m, 2H). LCMS (Analytical Method B) Rt = 2.69 min, MS (ESlpos): m/z 419.4 [M+H]+, Purity = 98%. |
| | | 7-[2-(2,4-difluorophenyl)-3-(pyridin-4-yl)-3H-imidazo[4,5-b]pyridin-5-yl]-4,7-diazaspiro[2.5]octane | |
| 149 | Intermediate 83 (23% yield) | 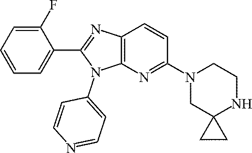 [SMILES: Fc1ccccc1-c1nc2ccc(N3CCNC4(CC4)C3)nc2n1-c1ccncc1] | 1H NMR (500 MHz, DMSO-d6) δ 8.69 - 8.54 (m, 2H), 7.97 (d, J = 9.0 Hz, 1H), 7.81 - 7.69 (m, 1H), 7.61 - 7.49 (m, 1H), 7.43 - 7.31 (m, 3H), 7.27 -7.12 (m, 1H), 6.91 (d, J = 9.0 Hz, 1H), 3.58 - 3.45 (m, 2H), 3.37 (s, 2H), 2.92 - 2.76 (m, 2H), 0.52 - 0.42 (m, 4H). LCMS (Analytical Method B) Rt = 2.57 min, MS (ESlpos): m/z 401.4 [M+H]+, Purity = 99%. |
| | | 7-[2-(2-fluorophenyl)-3-(pyridin-4-yl)-3H-imidazo[4,5-b]pyridin-5-yl]-4,7-diazaspiro[2.5]octane | |
| 158 | Intermediate 31 (13% yield) | 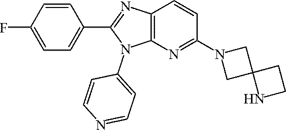 [SMILES: Fc1ccc(-c2nc3ccc(N4CC5(CCN5)C4)nc3n2-c2ccncc2)cc1] | 1H NMR (400 MHz, Methanol-d4) δ 8.70 - 8.60 (m, 2H), 7.89 (d, J = 8.7 Hz, 1H), 7.53 (ddd, J = 8.9, 5.2, 2.5 Hz, 2H), 7.50 - 7.46 (m, 2H), 7.22 -7.10 (m, 2H), 6.49 (d, J = 8.7 Hz, 1H), 4.19 (d, J = 9.7 Hz, 2H), 4.07 (d, J = 9.9 Hz, 2H), 3.46 (t, J = 7.4 Hz, 2H), 2.59 (t, J = 7.4 Hz, 2H). LCMS (Analytical Method A) Rt = 1.36 min, MS (ESlpos): m/z 387 [M+H]+, Purity = 100%. |
| | | 6-[2-(4-fluorophenyl)-3-(pyridin-4-yl)-3H-imidazo[4,5-b]pyridin-5-yl]-1,6-diazaspiro[3.3]heptane | |
| 160 | Intermediate 82 (6% yield) | 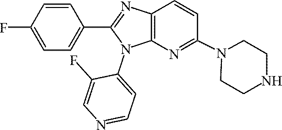 [SMILES: Fc1ccc(-c2nc3ccc(N4CCNCC4)nc3n2-c2ccncc2F)cc1] | 1H NMR (500 MHz, Methanol-d4) δ 8.66 (d, J = 1.9 Hz, 1H), 8.59 (d, J = 5.1 Hz, 1H), 7.93 (d, J = 9.0 Hz, 1H), 7.72 - 7.64 (m, 1H), 7.58 - 7.50 (m, 2H), 7.20 - 7.11 (m, 2H), 6.93 (d, J = 9.0 Hz, 1H), 3.56 - 3.49 (m, 4H), 2.95 - 2.84 (m, 4H). LCMS (Analytical Method A) Rt = 1.65 min, MS |
| | | 1-[2-(4-fluorophenyl)-3-(3-fluoropyridin-4-yl)-3H-imidazo[4,5-b]pyridin-5-yl]piperazine | (ESlpos): m/z 393.2 [M+H]+, Purity = 98%. |
| 162 | Intermediate 84 (10% yield) | 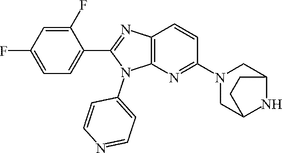 [SMILES: Fc1ccc(-c2nc3ccc(N4CC5CCC(C4)N5)nc3n2-c2ccncc2)c(F)c1] | 1H NMR (500 MHz, DMSO-d6) δ 8.73 - 8.57 (m, 2H), 7.96 (d, J = 9.0 Hz, 1H), 7.86 - 7.69 (m, 1H), 7.44 - 7.35 (m, 2H), 7.35 - 7.22 (m, 2H), 6.80 (d, J = 9.1 Hz, 1H), 3.83 (dd, J = 11.7, 2.0 Hz, 2H), 3.53 - 3.41 (m, 2H), 2.90 (dd, J = 11.6, 2.0 Hz, 2H), 1.72 - 1.53 (m, 4H). LCMS (Analytical Method A) Rt = 1.48 min, MS (ESlpos): m/z 419.2 [M+H]+, Purity = 99%. |
| | | 3-[2-(2,4-difluorophenyl)-3-(pyridin-4-yl)-3H-imidazo[4,5-b]pyridin-5-yl]-3,8-diazabicyclo[3.2.1]octane | |
| 163 | Intermediate 33 (9% yield) | 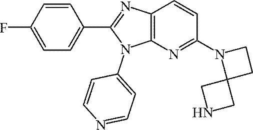 [SMILES: Fc1ccc(-c2nc3ccc(N4CCC45CNC5)nc3n2-c2ccncc2)cc1] | 1H NMR (500 MHz, Methanol-d4) δ 8.73 - 8.64 (m, 2H), 7.93 (d, J = 8.7 Hz, 1H), 7.57 - 7.54 (m, 2H), 7.54 -7.51 (m, 2H), 7.18 (t, J = 8.8 Hz, 2H), 6.52 (d, J = 8.7 Hz, 1H), 4.50 (d, J = 10.4 Hz, 2H), 3.90 (t, J = 7.1 Hz, 2H), 3.57 (d, J = 10.3 Hz, 2H), 2.59 (t, J = 7.1 Hz, 2H). LCMS (Analytical Method A) Rt = 1.53 min, MS (ESlpos): m/z 387 [M+H]+, Purity = 99%. |
| | | 1-[2-(4-fluorophenyl)-3-(pyridin-4-yl)-3H-imidazo[4,5-b]pyridin-5-yl]-1,6-diazaspiro[3.3]heptane | |
| 164 | Intermediate 33 (3% yield) | 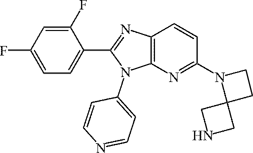 [SMILES: Fc1ccc(-c2nc3ccc(N4CCC45CNC5)nc3n2-c2ccncc2)c(F)c1] | 1H NMR (500 MHz, Methanol-d4) δ 8.73 - 8.56 (m, 2H), 7.96 (d, J = 8.7 Hz, 1H), 7.84 - 7.72 (m, 1H), 7.56 -7.46 (m, 2H), 7.24 - 7.13 (m, 1H), 7.09 - 6.98 (m, 1H), 6.55 (d, J = 8.8 Hz, 1H), 4.53 (d, J = 10.4 Hz, 2H), 3.92 (t, J = 7.1 Hz, 2H), 3.59 (d, J = 10.1 Hz, 2H), 2.65 - 2.54 (m, 2H). LCMS (Analytical Method A) Rt = 1.57 min, MS (ESlpos): m/z 405 [M+H]+, Purity = 97%. |
| | | 1-[2-(2,4-difluorophenyl)-3-(pyridin-4-yl)-3H-imidazo[4,5-b]pyridin-5-yl]-1,6-diazaspiro[3.3]heptane | |
| 165 | Intermediate 31 (5% yield) | 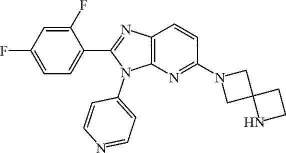 [SMILES: Fc1ccc(-c2nc3ccc(N4CC5(CCN5)C4)nc3n2-c2ccncc2)c(F)c1] | 1H NMR (400 MHz, Methanol-d4) δ 8.62 (dd, J = 4.8, 1.5 Hz, 2H), 7.91 (d, J = 8.7 Hz, 1H), 7.81 - 7.70 (m, 1H), 7.48 (dd, J = 4.8, 1.5 Hz, 2H), 7.22 -7.09 (m, 1H), 7.05 - 6.93 (m, 1H), 6.52 (d, J = 8.7 Hz, 1H), 4.21 (d, J = 9.4 Hz, 2H), 4.09 (d, J = 9.7 Hz, 2H), 3.46 (t, J = 7.4 Hz, 2H), 2.60 (t, J = 7.3 Hz, 2H). LCMS (Analytical Method A) Rt = 1.41 min, MS (ESlpos): m/z 405 [M+H]+, Purity = 99%. |
| | | 6-[2-(2,4-difluorophenyl)-3-(pyridin-4-yl)-3H-imidazo[4,5-b]pyridin-5-yl]-1,6-diazaspiro[3.3]heptane | |
| 168 | Intermediate 4 (1% yield) | 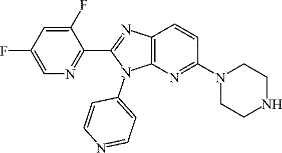 [SMILES: Fc1cnc(-c2nc3ccc(N4CCNCC4)nc3n2-c2ccncc2)c(F)c1] | 1H NMR (400 MHz, MeOD) δ 8.59 -8.36 (m, 2H), 8.31 - 8.20 (m, 1H), 7.96 (d, J = 9.0 Hz, 1H), 7.75 - 7.65 (m, 1H), 7.42 - 7.34 (m, 2H), 6.99 (d, J = 9.0 Hz, 1H), 3.81 - 3.64 (m, 4H), 3.19 - 3.14 (m, 4H). LCMS (Analytical Method B) Rt = 2.13 min, MS (ESlpos): m/z 394.4 [M+H]+, Purity = 100%. |
| | | 1-[2-(3,5-difluoropyridin-2-yl)-3-(pyridin-4-yl)-3H-imidazo[4,5-b]pyridin-5-yl]piperazine | |

### Synthesis of 2-(2,4-difluorophenyl)-6-(piperazin-1-yl)-1-(pyrimidin-4-yl)-1H-1,3-benzodiazole / Compound 179 of Table 1

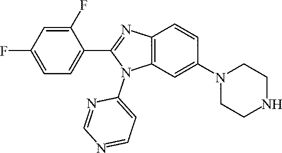 [SMILES: Fc1ccc(-c2nc3ccc(N4CCNCC4)cc3n2-c2ccncn2)c(F)c1]

A suspension of 2,4-difluorobenzaldehyde (0.024 mL, 0.180 mmol), Na₂S₂O₄ (89 mg, 0.509 mmol) and tert-butyl 4-[4-nitro-3-(pyrimidin-4-ylamino)phenyl]piperazine-1-carboxylate (Intermediate 3-2) (60 mg, 0.150 mmol) in DMSO (0.6 mL) and EtOH (0.2 mL) was heated at 100° C. under air overnight. The mixture was filtered and the filtrate was purified using preparative HPLC (Method A1) to afford the title compound (10 mg, 18% yield). 1H NMR (500 MHz, DMSO) δ 9.18 (d, J = 1.0 Hz, 1H), 8.93 (d, J = 5.5 Hz, 1H), 7.84 (td, J = 8.8, 6.6 Hz, 1H), 7.66 (d, J = 8.9 Hz, 1H), 7.53 (dd, J = 5.5, 1.2 Hz, 1H), 7.34 - 7.24 (m, 3H), 7.13 (dd, J = 8.9, 2.3 Hz, 1H), 3.13 - 3.04 (m, 4H), 2.90 - 2.80 (m, 4H). LCMS (Analytical Method A) Rt = 1.51 min, MS (ESlpos): m/z 393.2 [M+H]+, Purity = 100%.

Each of as the compounds listed in Table 1.5.7 were prepared according to the method of Compound 179 of Table 1 using the intermediate listed in the “Synthesis” column and with appropriate aldehyde derivatives for such compounds. The ethanol is a co-solvent and was not used in all examples. The final compounds were purified by preparative HPLC Methods A1, or A2

TABLE 1.5.7

| | | | |
|---|---|---|---|
| Example | Synthesis | Structure/ Name | Data |
| 185-RR | Intermediate 91 (23% yield) | 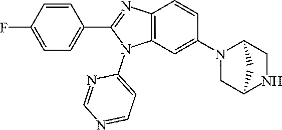 [SMILES: Fc1ccc(-c2nc3ccc(N4C[C@H]5C[C@@H]4CN5)cc3n2-c2ccncn2)cc1] | 1H NMR (400 MHz, DMSO) δ 9.26 (d, J = 0.9 Hz, 1H), 8.93 (d, J = 5.5 Hz, 1H), 7.58 (d, J = 8.8 Hz, 1H), 7.52 - 7.47 (m, 2H), 7.43 (dd, J = 5.5, 1.2 Hz, 1H), 7.29 - 7.23 (m, 2H), 6.79 (d, J = 2.0 Hz, 1H), 6.72 (dd, J = 8.8, 2.2 Hz, 1H), 4.40 (s, 1H), 3.61 (s, 1H), 3.53 (d, J = 6.7 Hz, 1H), 2.93 (d, J = 8.4 Hz, 1H), 2.91 -2.79 (m, 2H), 1.80 (d, J = 9.2 Hz, 1H), 1.66 (d, J = 8.9 Hz, 1H). LCMS (Analytical Method A) Rt = 1.5 min, MS (ESIpos): m/z 387.3 [M+H]+, Purity = 99%. |
| | | 6-[(1R,4R)-2,5-diazabicyclo[2.2.1]heptan-2-yl]-2-(4-fluorophenyl)-1-(pyrimidin-4-yl)-1H-1,3-benzodiazole | |
| 186-R | Intermediate 92 (23% yield) | 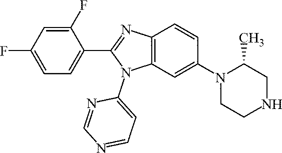 [SMILES: C[C@@H]1CNCCN1c1ccc2nc(-c3ccc(F)cc3F)n(-c3ccncn3)c2c1] | 1H NMR (400 MHz, DMSO) δ 9.20 (d, J = 0.9 Hz, 1H), 8.93 (d, J = 5.5 Hz, 1H), 7.89 - 7.80 (m, 1H), 7.68 (d, J = 8.9 Hz, 1H), 7.50 (dd, J = 5.5, 1.2 Hz, 1H), 7.36 - 7.23 (m, 3H), 7.12 (dd, J = 9.0, 2.2 Hz, 1H), 3.92 - 3.82 (m, 1H), 3.14 - 3.04 (m, 1H), 3.00 - 2.89 (m, 3H), 2.75 (dd, J = 9.9 Hz, 2H), 0.96 (d, J = 6.5 Hz, 3H). LCMS (Analytical Method A) Rt = 1.63 min, MS (ESIpos): m/z 407.4 [M+H]+, Purity = 97%. |
| | | 2-(2,4-difluorophenyl)-6-[(2R)-2-methylpiperazin-1-yl]-1-(pyrimidin-4-yl)-1H-1,3-benzodiazole | |
| 189-RR | Intermediate 93 (18% yield) | 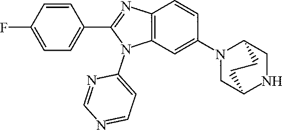 [SMILES: Fc1ccc(-c2nc3ccc(N4C[C@H]5CC[C@@H]4CN5)cc3n2-c2ccncn2)cc1] | 1H NMR (400 MHz, DMSO) δ 9.25 (s, 1H), 8.93 (d, J = 5.4 Hz, 1H), 7.58 (d, J = 8.8 Hz, 1H), 7.54 - 7.36 (m, 3H), 7.25 (t, J = 8.8 Hz, 2H), 6.99 - 6.73 (m, 2H), 3.92 (s, 1H), 3.48 (d, J = 9.7 Hz, 1H), 3.32 (s, 1H), 3.11 (d, J = 10.2 Hz, 1H), 3.01 (d, J = 10.6 Hz, 2H), 1.84 (m, 3H), 1.65 (t, J = 9.7 Hz, 1H). LCMS (Analytical Method B) Rt = 2.63 min, MS (ESIpos): m/z 401.4 [M+H]+, Purity = 97%. |
| | | 6-[(1R,4R)-2,5-diazabicyclo[2.2.2]octan-2-yl]-2-(4-fluorophenyl)-1-(pyrimidin-4-yl)-1H-1,3-benzodiazole | |
| 190-RR | Intermediate 93 (10% yield) | 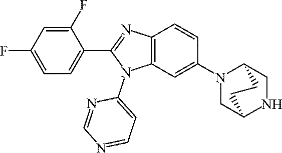 [SMILES: Fc1ccc(-c2nc3ccc(N4C[C@H]5CC[C@@H]4CN5)cc3n2-c2ccncn2)c(F)c1] | 1H NMR (500 MHz, DMSO) δ 9.16 (d, J = 0.9 Hz, 1H), 8.92 (d, J = 5.5 Hz, 1H), 7.82 (td, J = 8.8, 6.6 Hz, 1H), 7.61 (d, J = 8.9 Hz, 1H), 7.52 (dd, J = 5.5, 1.2 Hz, 1H), 7.41 - 7.14 (m, 2H), 6.95 (d, J = 2.2 Hz, 1H), 6.85 (dd, J = 9.0, 2.3 Hz, 1H), 3.96 (s, 1H), 3.51 (d, J = 9.6 Hz, 1H), 3.39 - 3.35 (m, 1H), 3.13 (d, J = 10.7 Hz, 1H), 3.08 - 2.98 (m, 2H), 2.01 - 1.76 (m, 3H), 1.74 - 1.56 (m, 1H). LCMS (Analytical Method A) Rt = 1.68 min, MS (ESIpos): m/z 419.4 [M+H]+, Purity = 95%. |
| | | 6-[(1R,4R)-2,5-diazabicyclo[2.2.2]octan-2-yl]-2-(2,4-difluorophenyl)-1-(pyrimidin-4-yl)-1H-1,3-benzodiazole | |

### Synthesis of 4-[2-(2,4-difluorophenyl)-5-(piperazin-1-yl)-3H-imidazo[4,5-b]pyridin-3-yl]pyrimidine / Compound 109 of Table 1

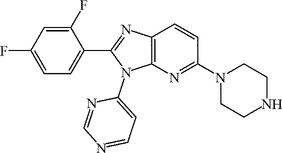 [SMILES: Fc1ccc(-c2nc3ccc(N4CCNCC4)nc3n2-c2ccncn2)c(F)c1]

A mixture of tert-butyl 4-[5-nitro-6-(pyrimidin-4-ylamino)-2-pyridyl]piperazine-1-carboxylate (Intermediate 26) (67%, 66 mg, 0.110 mmol) and Na₂S₂O₄ (58 mg, 0.329 mmol) in DMSO (0.5 mL) and EtOH (0.1 mL) was gently warmed for 3 minutes. 2,4-difluorobenzaldehyde (22 uL, 0.176 mmol) was added and the reaction heated to 100° C. for 18 hours. Additional Na₂S₂O₄ (58 mg, 0.329 mmol) and 2,4-difluorobenzaldehyde (22 uL, 0.176 mmol) were added and heating continued for 18 hours. The reaction was diluted with MeCN/water (1:1, 0.5 mL) and intractable material removed by filtration. The residue was purified by preparative HPLC (Method A1). The residue was further purified by chromatography (5 g, KP-amine), eluting with 0-7% MeOH/DCM. The relevant fractions were combined and concentrated in vacuo to yield the title compound (6.0 mg, 14% yield). 1H NMR (400 MHz, DMSO-d6) δ 9.08 (d, J = 5.6 Hz, 1H), 8.92 (d, J = 0.9 Hz, 1H), 8.28 (dd, J = 5.5, 1.2 Hz, 1H), 8.01 (d, J = 9.0 Hz, 1H), 7.85 - 7.79 (m, 1H), 7.31 - 7.22 (m, 2H), 6.97 (d, J = 9.0 Hz, 1H), 3.51 - 3.47 (m, 4H), 2.85 - 2.77 (m, 4H). LCMS (Analytical Method A) Rt = 1.62 min, MS (ESlpos): m/z 394.3 [M+H]+, Purity = 100%.

Each of the compounds listed in Table 1.5.8 were prepared according to the method of Compound 109 of Table 1 using the intermediate listed in the “Synthesis” column and with appropriate aldehyde derivatives for such compounds. The ethanol is a co-solvent and was not used in all examples. The final compounds were purified by preparative HPLC Methods A1, or A2. If required further purification with KP-NH column was carried out.

TABLE 1.5.8

| | | | |
|---|---|---|---|
| Example | Synthesis | Structure/ Name | Data |
| 112 | Intermediate 26 (20% yield) | 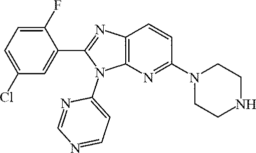 [SMILES: Fc1ccc(Cl)cc1-c1nc2ccc(N3CCNCC3)nc2n1-c1ccncn1] | 1H NMR (500 MHz, DMSO-d6) δ 9.10 (d, J = 5.7 Hz, 1H), 8.93 (d, J = 1.0 Hz, 1H), 8.31 (dd, J = 5.5, 1.3 Hz, 1H), 8.03 (d, J = 9.0 Hz, 1H), 7.83 (dd, J = 6.1, 2.7 Hz, 1H), 7.67 - 7.54 (m, 1H), 7.28 (dd, J = 10.0, 8.9 Hz, 1H), 6.99 (d, J = 9.1 Hz, 1H), 3.56 - 3.47 (m, 4H), 2.87 -2.77 (m, 4H). LCMS (Analytical Method A) Rt = 1.79 min, MS (ESIpos): m/z 410.1, 412.1 [M+H]+, Purity = 98%. |
| | | 4-[2-(5-chloro-2-fluorophenyl)-5-(piperazin-1-yl)-3H-imidazo[4,5-b]pyridin-3-yl]pyrimidine | |
| 114-RR | Intermediate 89 (27% yield) | 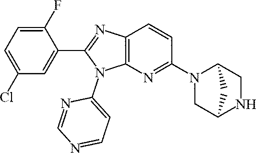 [SMILES: Fc1ccc(Cl)cc1-c1nc2ccc(N3C[C@H]4C[C@@H]3CN4)nc2n1-c1ccncn1] | 1H NMR (400 MHz, DMSO-d6) δ 9.08 (d, J = 5.6 Hz, 1H), 8.91 (d, J = 0.9 Hz, 1H), 8.37 (dd, J = 5.6, 1.3 Hz, 1H), 7.98 (d, J = 8.9 Hz, 1H), 7.87 - 7.77 (m, 1H), 7.64 - 7.54 (m, 1H), 7.27 (dd, J = 10.0, 8.9 Hz, 1H), 6.65 (d, J = 8.5 Hz, 1H), 4.76 (s, 1H), 3.69 (s, 1H), 3.53 (dd, J = 9.5, 2.0 Hz, 1H), 3.28 - 3.25 (m, 1H), 2.94 (d, J = 8.0 Hz, 1H), 2.83 (d, J = 9.5 Hz, 1H), 1.81 (d, J = 8.9 Hz, 1H), 1.70 (d, J = 9.1 Hz, 1H). LCMS (Analytical Method A) Rt = 1.84 min, MS (ESIpos): m/z 422.1, 424.1 [M+H]+, Purity = 97%. |
| | | (1R,4R)-2-[2-(5-chloro-2-fluorophenyl)-3-(pyrimidin-4-yl)-3H-imidazo[4,5-b]pyridin-5-yl]-2,5-diazabicyclo[2.2.1]heptane | |
| 115-RR | Intermediate 90 (28% yield) | 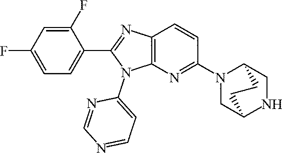 [SMILES: Fc1ccc(-c2nc3ccc(N4C[C@H]5CC[C@@H]4CN5)nc3n2-c2ccncn2)c(F)c1] | 1H NMR (400 MHz, DMSO-d6) δ 9.08 (d, J = 5.6 Hz, 1H), 8.91 (d, J = 1.0 Hz, 1H), 8.31 (dd, J = 5.5, 1.1 Hz, 1H), 7.98 (d, J = 8.9 Hz, 1H), 7.90 - 7.76 (m, 1H), 7.35 - 7.16 (m, 2H), 6.68 (d, J = 9.0 Hz, 1H), 4.53 (s, 1H), 3.63 (d, J = 10.5 Hz, 1H), 3.53 (d, J = 9.0 Hz, 1H), 3.16 - 2.99 (m, 3H), 2.01 - 1.61 (m, 4H). LCMS (Analytical Method A) Rt = 1.73 min, MS (ESIpos): m/z 420.2 [M+H]+, Purity = 99%. |
| | | (1R,4R)-2-[2-(2,4-difluorophenyl)-3-(pyrimidin-4-yl)-3H-imidazo[4,5-b]pyridin-5-yl]-2,5-diazabicyclo[2.2.2]octane | |
| 116-RR | Intermediate 90 (23% yield) | 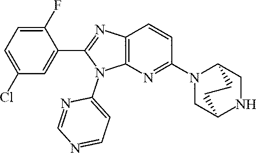 [SMILES: Fc1ccc(Cl)cc1-c1nc2ccc(N3C[C@H]4CC[C@@H]3CN4)nc2n1-c1ccncn1] | 1H NMR (400 MHz, DMSO-d6) δ 9.09 (d, J = 5.6 Hz, 1H), 8.91 (d, J = 1.0 Hz, 1H), 8.34 (dd, J = 5.6, 1.1 Hz, 1H), 7.99 (d, J = 9.0 Hz, 1H), 7.84 - 7.77 (m, 1H), 7.63 - 7.55 (m, 1H), 7.26 (dd, J = 10.0, 8.9 Hz, 1H), 6.70 (d, J = 8.7 Hz, 1H), 4.54 (s, 1H), 3.63 (d, J = 10.5 Hz, 1H), 3.53 (d, J = 10.2 Hz, 1H), 3.17 -2.99 (m, 3H), 1.95 - 1.60 (m, 4H). LCMS (Analytical Method A) Rt = 1.92 min, MS (ESIpos): m/z 436.1, 438.1 [M+H]+, Purity = 99%. |
| | | (1R,4R)-2-[2-(5-chloro-2-fluorophenyl)-3-(pyrimidin-4-yl)-3H-imidazo[4,5-b]pyridin-5-yl]-2,5-diazabicyclo[2.2.2]octane | |
| 117-RR | Intermediate 90 (17% yield) | 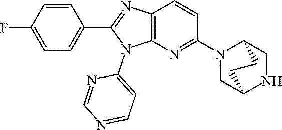 [SMILES: Fc1ccc(-c2nc3ccc(N4C[C@H]5CC[C@@H]4CN5)nc3n2-c2ccncn2)cc1] | 1H NMR (500 MHz, DMSO-d6) δ 9.12 (d, J = 5.4 Hz, 1H), 9.02 (d, J = 1.0 Hz, 1H), 8.16 (dd, J = 5.4, 1.2 Hz, 1H), 7.94 (d, J = 8.9 Hz, 1H), 7.52 - 7.44 (m, 2H), 7.29 - 7.16 (m, 2H), 6.63 (d, J = 8.9 Hz, 1H), 4.48 (s, 1H), 3.58 (d, J = 10.4 Hz, 1H), 3.49 (dd, J = 10.3, 1.7 Hz, 1H), 3.13 - 2.96 (m, 3H), 1.94 -1.78 (m, 3H), 1.72 - 1.60 (m, 1H). LCMS (Analytical Method A) Rt = 1.62 min, MS (ESIpos): m/z 402.2 [M+H]+, Purity = 99%. |
| | | (1R,4R)-2-[2-(4-fluorophenyl)-3-(pyrimidin-4-yl)-3H-imidazo[4,5-b]pyridin-5-yl]-2,5-diazabicyclo[2.2.2]octane | |
| 118-RR | Intermediate 89 (22% yield) | 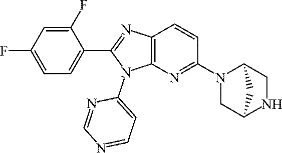 [SMILES: Fc1ccc(-c2nc3ccc(N4C[C@H]5C[C@@H]4CN5)nc3n2-c2ccncn2)c(F)c1] | 1H NMR (400 MHz, DMSO-d6) δ 9.07 (d, J = 5.6 Hz, 1H), 8.91 (d, J = 1.0 Hz, 1H), 8.35 (dd, J = 5.5, 1.2 Hz, 1H), 7.96 (d, J = 8.8 Hz, 1H), 7.87 - 7.75 (m, 1H), 7.33 - 7.15 (m, 2H), 6.63 (d, J = 8.9 Hz, 1H), 4.75 (s, 1H), 3.68 (s, 1H), 3.53 (dd, J = 9.4, 2.0 Hz, 1H), 3.29 - 3.24 (m, 1H), 2.94 (d, J = 8.0 Hz, 1H), 2.83 (d, J = 9.5 Hz, 1H), 1.81 (d, J = 9.3 Hz, 1H), 1.70 (d, J = 9.3 Hz, 1H). LCMS (Analytical Method A) Rt = 1.64 min, MS (ESIpos): m/z 406.1 [M+H]+, Purity = 99%. |
| | | (1R,4R)-2-[2-(2,4-difluorophenyl)-3-(pyrimidin-4-yl)-3H-imidazo[4,5-b]pyridin-5-yl]-2,5-diazabicyclo[2.2.1]heptane | |
| 119-RR | Intermediate 89 (31% yield) | 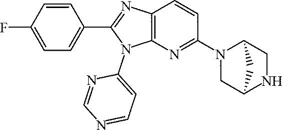 [SMILES: Fc1ccc(-c2nc3ccc(N4C[C@H]5C[C@@H]4CN5)nc3n2-c2ccncn2)cc1] | 1H NMR (500 MHz, DMSO-d6) δ 9.12 (d, J = 5.5 Hz, 1H), 9.02 (d, J = 0.9 Hz, 1H), 8.18 (dd, J = 5.4, 1.3 Hz, 1H), 7.93 (d, J = 8.8 Hz, 1H), 7.54 - 7.43 (m, 2H), 7.27 - 7.18 (m, 2H), 6.59 (d, J = 8.8 Hz, 1H), 4.69 (s, 1H), 3.65 (s, 1H), 3.48 (dd, J = 9.3, 2.0 Hz, 1H), 3.25 (d, J = 9.5 Hz, 1H), 2.91 (dd, J = 9.5, 1.7 Hz, 1H), 2.79 (d, J = 9.5 Hz, 1H), 1.77 (d, J = 9.3 Hz, 1H), 1.67 (d, J = 9.1 Hz, 1H). LCMS (Analytical Method A) Rt = 1.54 min, MS (ESIpos): m/z 388.1 [M+H]+, Purity = 99%. |
| | | (1R,4R)-2-[2-(4-fluorophenyl)-3-(pyrimidin-4-yl)-3H-imidazo[4,5-b]pyridin-5-yl]-2,5-diazabicyclo[2.2.1]heptane | |
| 183-R | Intermediate 88 (9% yield) | 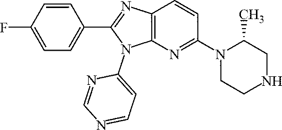 [SMILES: C[C@@H]1CNCCN1c1ccc2nc(-c3ccc(F)cc3)n(-c3ccncn3)c2n1] | 1H NMR (500 MHz, DMSO) δ 9.13 (d, J = 5.4 Hz, 1H), 9.03 (d, J = 1.0 Hz, 1H), 8.15 (dd, J = 5.4, 1.3 Hz, 1H), 7.97 (d, J = 9.0 Hz, 1H), 7.54 - 7.48 (m, 2H), 7.27 - 7.20 (m, 2H), 6.87 (d, J = 9.1 Hz, 1H), 4.39 - 4.32 (m, 1H), 3.93 (d, J = 10.7 Hz, 1H), 3.01 - 2.87 (m, 2H), 2.87 - 2.76 (m, 2H), 2.66 - 2.57 (m, 1H), 1.14 (d, J = 6.6 Hz, 3H). LCMS (Analytical Method A) Rt = 1.66 min, MS (ESIpos): m/z 390.3 [M+H]+, Purity = 97%. |
| | | 4-[2-(4-fluorophenyl)-5-[(2R)-2-methylpiperazin-1-yl]-3H-imidazo[4,5-b]pyridin-3-yl]pyrimidine | |
| 184-R | Intermediate 88 (22% yield) | 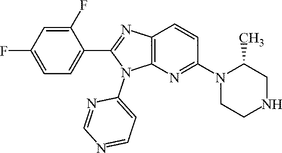 [SMILES: C[C@@H]1CNCCN1c1ccc2nc(-c3ccc(F)cc3F)n(-c3ccncn3)c2n1] | 1H NMR (500 MHz, DMSO-d6) δ 9.08 (d, J = 5.5 Hz, 1H), 8.92 (d, J = 1.0 Hz, 1H), 8.27 (dd, J = 5.5, 1.2 Hz, 1H), 7.99 (d, J = 9.0 Hz, 1H), 7.85 - 7.78 (m, 1H), 7.31 - 7.22 (m, 2H), 6.91 (d, J = 9.1 Hz, 1H), 4.43 - 4.37 (m, 1H), 4.01 - 3.93 (m, 1H), 3.02 - 2.92 (m, 2H), 2.88 - 2.78 (m, 2H), 2.67 - 2.58 (m, 1H), 1.16 (d, J = 6.6 Hz, 3H). LCMS (Analytical Method B) Rt =2.78 min, MS (ESIpos): m/z 408.3 [M+H]+, Purity = 100%. |
| | | 4-[2-(2,4-difluorophenyl)-5-[(2R)-2-methylpiperazin-1-yl]-3H-imidazo[4,5-b]pyridin-3-yl]pyrimidine | |
| 184-S | Intermediate 87 (13% yield) | 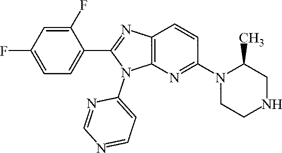 [SMILES: C[C@H]1CNCCN1c1ccc2nc(-c3ccc(F)cc3F)n(-c3ccncn3)c2n1] | 1H NMR (500 MHz, DMSO-d6) δ 9.08 (d, J = 5.5 Hz, 1H), 8.92 (d, J = 1.0 Hz, 1H), 8.27 (dd, J = 5.5, 1.2 Hz, 1H), 8.00 (d, J = 9.0 Hz, 1H), 7.82 (td, J = 8.5, 6.6 Hz, 1H), 7.32 - 7.21 (m, 2H), 6.91 (d, J = 9.1 Hz, 1H), 4.44 - 4.37 (m, 1H), 4.01 -3.94 (m, 1H), 3.03 - 2.92 (m, 2H), 2.88 - 2.79 (m, 2H), 2.67 - 2.59 (m, 1H), 1.16 (d, J = 6.6 Hz, 3H). LCMS (Analytical Method A) Rt = 1.64 min, MS (ESIpos): m/z 408.3 [M+H]+, Purity = 100%. |
| | | 4-[2-(2,4-difluorophenyl)-5-[(2S)-2-methylpiperazin-1-yl]-3H-imidazo[4,5-b]pyridin-3-yl]pyrimidine | |
| 197-R | Intermediate 88 (38% yield) | 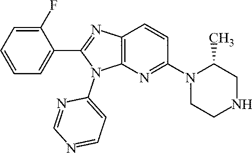 [SMILES: C[C@@H]1CNCCN1c1ccc2nc(-c3ccccc3F)n(-c3ccncn3)c2n1] | 1H NMR (500 MHz, DMSO-d6) δ 9.08 (d, J = 5.5 Hz, 1H), 8.89 (d, J = 1.0 Hz, 1H), 8.25 (dd, J = 5.5, 1.2 Hz, 1H), 8.00 (d, J = 9.0 Hz, 1H), 7.77 (td, J = 7.6, 1.7 Hz, 1H), 7.57 - 7.48 (m, 1H), 7.35 (td, J = 7.6, 1.0 Hz, 1H), 7.18 (dd, J = 10.8, 8.3 Hz, 1H), 6.91 (d, J = 9.1 Hz, 1H), 4.43 - 4.36 (m, 1H), 4.01 - 3.94 (m, 1H), 2.99 (d, J = 12.5 Hz, 1H), 2.94 (dd, J = 12.4, 3.4 Hz, 1H), 2.85 (dd, J = 12.2, 3.5 Hz, 1H), 2.81 (d, J = 12.1 Hz, 1H), 2.66 -2.61 (m, 1H), 1.16 (d, J = 6.6 Hz, 3H). LCMS (Analytical Method A) Rt = 1.60 min, MS (ESlpos): m/z 390.3 [M+H]+, Purity = 98%. |
| | | 4-[2-(2-fluorophenyl)-5-[(2R)-2-methylpiperazin-1-yl]-3H-imidazo[4,5-b]pyridin-3-yl]pyrimidine | |
| 198-R | Intermediate 88 (35% yield) | 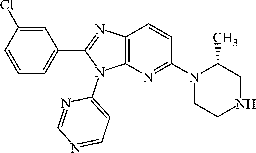 [SMILES: C[C@@H]1CNCCN1c1ccc2nc(-c3cccc(Cl)c3)n(-c3ccncn3)c2n1] | 1H NMR (500 MHz, DMSO-d6) δ 9.14 (d, J = 5.5 Hz, 1H), 9.03 (d, J = 1.0 Hz, 1H), 8.17 (dd, J = 5.4, 1.3 Hz, 1H), 7.98 (d, J = 9.0 Hz, 1H), 7.54 (t, J = 1.8 Hz, 1H), 7.48 (ddd, |
| | | 4-[2-(3-chlorophenyl)-5-[(2R)-2-methylpiperazin-1-yl]-3H-imidazo[4,5-b]pyridin-3-yl]pyrimidine | J = 8.0, 2.1, 1.1 Hz, 1H), 7.40 (t, J = 7.9 Hz, 1H), 7.34 (dt, J = 7.8, 1.3 Hz, 1H), 6.89 (d, J = 9.1 Hz, 1H), 4.40 - 4.33 (m, 1H), 3.98 - 3.91 (m, 1H), 2.97 (d, J = 12.8 Hz, 1H), 2.92 (td, J= 12.3, 3.4 Hz, 1H), 2.83 (dd, J = 12.2, 3.5 Hz, 1H), 2.79 (d, J = 11.9 Hz, 1H), 2.65 - 2.57 (m, 1H), 1.14 (d, J = 6.6 Hz, 3H). LCMS (Analytical Method A) Rt = 1.79 min, MS (ESIpos): m/z 406.3,408.3 [M+H]+, Purity = 99%. |
| 201-R | Intermediate 88 (22% yield) | 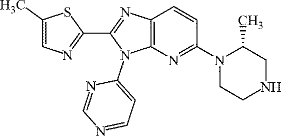 [SMILES: Cc1cnc(-c2nc3ccc(N4CCNC[C@H]4C)nc3n2-c2ccncn2)s1] | 1H NMR (400 MHz, DMSO) δ 9.19 (d, J = 0.9 Hz, 1H), 9.10 (d, J = 5.3 Hz, 1H), 8.03 - 7.91 (m, 2H), 7.43 (d, J = 1.1 Hz, 1H), 6.89 (d, J = 9.2 Hz, 1H), 4.35 - 4.24 (m, 1H), 3.95 - 3.85 (m, 1H), 2.98 - 2.90 (m, 1H), 2.91 - 2.81 (m, 1H), 2.80 -2.73 (m, 2H), 2.61 - 2.53 (m, 1H), 2.48 (s, 3H), 1.10 (d, J = 6.6 Hz, 3H). LCMS (Analytical Method A) Rt = 1.42 min, MS (ESIpos): m/z 393.2 [M+H]+, Purity = 97%. |
| | | 4-[2-(5-methyl-1,3-thiazol-2-yl)-5-[(2R)-2-methylpiperazin-1-yl]-3H-imidazo[4,5-b]pyridin-3-yl]pyrimidine | |

### Synthesis of 1-[2-(2,4-difluorophenyl)-7-methyl-3-(pyridin-4-yl)-3H-imidazo[4,5-b]pyridin-5-yllpiperazine / Compound 51 of Table 1

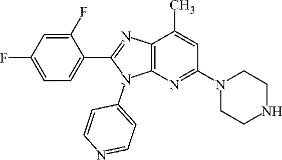 [SMILES: Cc1cc(N2CCNCC2)nc2c1nc(-c1ccc(F)cc1F)n2-c1ccncc1]

Na₂S₂O₄ (363 mg, 2.06 mmol) was added to a suspension of tert-butyl 4-[4-methyl-5-nitro-6-(4-pyridylamino)-2-pyridyl]piperazine-1-carboxylate (Intermediate 65) (95%, 300 mg, 0.688 mmol) in EtOH (0.5 mL) and DMSO (3 mL). The reaction was gently warmed then 2,4-difluorobenzaldehyde (90 µL, 0.823 mmol) was added and the reaction heated to 100° C. for 18 hours. The reaction was cooled and diluted with EtOAc (2 mL). The mixture was washed with NaOH (2 mL, 1 M), and the aqueous layer extracted with EtOAc (3 × 3 mL). The combined organics were passed through a phase separating frit and concentrated in vacuo. The residue was purified by preparative HPLC (Method A1) to yield the title compound (107 mg, 38% yield). 1H NMR (400 MHz, Methanol-d4) δ 8.64 - 8.57 (m, 2H), 7.80 (td, J = 8.4, 6.3 Hz, 1H), 7.50 - 7.44 (m, 2H), 7.24 - 7.14 (m, 1H), 7.01 (ddd, J = 10.4, 9.0, 2.4 Hz, 1H), 6.80 - 6.74 (m, 1H), 3.59 - 3.52 (m, 4H), 2.95 - 2.87 (m, 4H), 2.65 - 2.59 (m, 3H). LCMS (Analytical Method A) Rt ═1.41 min, MS (ESlpos): m/z 407.2 [M+H]+, Purity = 100%.

Each of the compounds listed in Table 1.5.9 were prepared according to the method of Compound 51 of Table 1 using the intermediate listed in the “Synthesis” column and the appropriate aldehyde derivative for such compounds. The ethanol is a co-solvent and was not used in all examples. The final compounds were purified by preparative HPLC Methods A1, or A2.

TABLE 1.5.9

| | | | |
|---|---|---|---|
| Example | Synthesis | Structure/ Name | Data |
| 48 | Intermediate 65 (29% yield) | 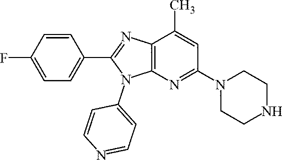 [SMILES: Cc1cc(N2CCNCC2)nc2c1nc(-c1ccc(F)cc1)n2-c1ccncc1] | 1H NMR (400 MHz, DMSO-d6) δ 8.74 - 8.64 (m, 2H), 7.54 - 7.45 (m, 2H), 7.43 - 7.38 (m, 2H), 7.28 - 7.21 (m, 2H), 6.74 (s, 1H), 3.41 - 3.36 (m, 4H), 2.79 - 2.72 (m, 4H), 2.54 (s, 3H). LCMS (Analytical Method B) Rt = 2.66 min, MS (ESIpos): m/z 389.3 [M+H]+, Purity = 96%. |
| | | 1-[2-(4-fluorophenyl)-7-methyl-3-(pyrid in-4-yl)-3H-imidazo [4,5-b]pyridin-5-yl]piperazine | |
| 50 | Intermediate 65 (47% yield) | 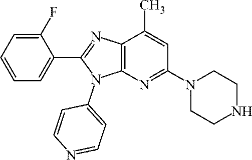 [SMILES: Cc1cc(N2CCNCC2)nc2c1nc(-c1ccccc1F)n2-c1ccncc1] | 1H NMR (400 MHz, Methanol-d4) δ 8.65 - 8.53 (m, 2H), 7.76 (td, J = 7.4, 1.7 Hz, 1H), 7.61 - 7.51 (m, 1H), 7.48 - 7.43 (m, 2H), 7.38 (td, J = 7.6, 1.0 Hz, 1H), 7.16 - 7.05 (m, 1H), 6.78 (s, 1H), 3.61 - 3.48 (m, 4H), 2.98 - 2.83 (m, 4H), 2.63 (s, 3H). LCMS (Analytical Method B) Rt = 2.53 min, MS (ESIpos): m/z 389.3 [M+H]+, Purity = 100%. |
| | | 1-[2-(2-fluorophenyl)-7-methyl-3-(pyrid in-4-yl)-3H-imidazo [4,5-b]pyridin-5-yl]piperazine | |
| 52 | Intermediate 65 (27% yield) | 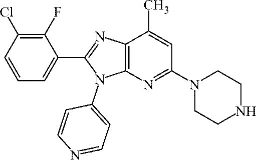 [SMILES: Cc1cc(N2CCNCC2)nc2c1nc(-c1cccc(Cl)c1F)n2-c1ccncc1] | 1H NMR (500 MHz, Methanol-d4) δ 8.65 - 8.59 (m, 2H), 7.71 - 7.62 (m, 2H), 7.50 - 7.45 (m, 2H), 7.36 (td, J = 8.1, 0.9 Hz, 1H), 6.81 - 6.77 (m, 1H), 3.61 - 3.52 (m, 4H), 2.95 - 2.88 (m, 4H), 2.66 - 2.59 (m, 3H). LCMS (Analytical Method A) Rt = 1.68 min, MS (ESIpos): m/z 423.3, 425.2 [M+H]+, Purity = 98%. |
| | | 1-[2-(3-chloro-2-fluorophenyl)-7-methyl-3-(pyridin-4-yl)-3H-imidazo[4,5-b]pyridin-5-yl]piperazine | |
| 54 | Intermediate 65 (37% yield) | 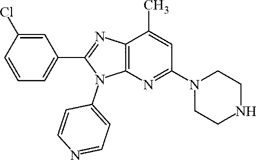 [SMILES: Cc1cc(N2CCNCC2)nc2c1nc(-c1cccc(Cl)c1)n2-c1ccncc1] | 1H NMR (400 MHz, Methanol-d4) δ 8.72 - 8.59 (m, 2H), 7.65 - 7.58 (m, 1H), 7.53 - 7.43 (m, 3H), 7.43 - 7.30 (m, 2H), 6.81 - 6.70 (m, 1H), 3.60 -3.48 (m, 4H), 2.96 - 2.83 (m, 4H), 2.67 - 2.54 (m, 3H). LCMS (Analytical Method B) Rt = 3.00 min, MS (ESIpos): m/z 405.3, 407.2 [M+H]+, Purity = 97%. |
| | | 1-[2-(3-chlorophenyl)-7-methyl-3-(pyridin-4-yl)-3H-imidazo [4,5-b]pyridin-5-yl]piperazine | |
| 55 | Intermediate 65 (31% yield) | 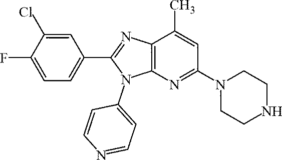 [SMILES: Cc1cc(N2CCNCC2)nc2c1nc(-c1ccc(F)c(Cl)c1)n2-c1ccncc1] | 1H NMR (500 MHz, Methanol-d4) δ 8.72 - 8.62 (m, 2H), 7.73 (dd, J = 7.0, 2.2 Hz, 1H), 7.53 - 7.48 (m, 2H), 7.39 (ddd, J = 8.6, 4.5, 2.2 Hz, 1H), 7.29 (t, J = 8.8 Hz, 1H), 6.81 - 6.71 (m, 1H), 3.58 - 3.50 (m, 4H), 2.94 -2.87 (m, 4H), 2.65 - 2.60 (m, 3H). LCMS (Analytical Method A) Rt = 1.79 min, MS (ESIpos): m/z 423.2, 425.3 [M+H]+, Purity = 100%. |
| | | 1-[2-(3-chloro-4-fluorophenyl)-7-methyl-3-(pyridin-4-yl)-3H-imidazo[4,5-b]pyridin-5-yl]piperazine | |
| 79-RS | Intermediate 66 (17% yield) | 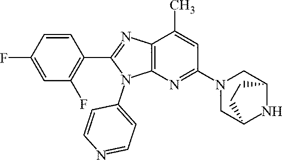 [SMILES: Cc1cc(N2C[C@H]3CC[C@@H](C2)N3)nc2c1nc(-c1ccc(F)cc1F)n2-c1ccncc1] | 1H NMR (500 MHz, Chloroform-d) δ 8.67 - 8.61 (m, 2H), 7.78 - 7.70 (m, 1H), 7.33 - 7.29 (m, 2H), 7.06 - 6.99 (m, 1H), 6.79 - 6.70 (m, 1H), 6.46 (s, 1H), 3.92 (dd, J = 11.9, 2.0 Hz, 2H), 3.66 (s, 2H), 3.03 (dd, J = 12.0, 1.7 Hz, 2H), 2.65 (s, 3H), 1.80 (s, 4H). LCMS (Analytical Method A) Rt = 1.63 min, MS (ESIpos): m/z 433.3 [M+H]+, Purity = 99%. |
| | | (1R,5S)-3-[2-(2,4-difluorophenyl)-7-methyl-3-(pyridin-4-yl)-3H-imidazo [4,5-b]pyridin-5-yl]-3,8-diazabicyclo[3.2.1]octane | |
| 80-RS | Intermediate 66 (56% yield) | 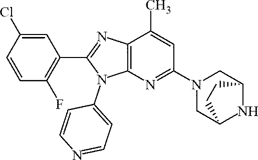 [SMILES: Cc1cc(N2C[C@H]3CC[C@@H](C2)N3)nc2c1nc(-c1cc(Cl)ccc1F)n2-c1ccncc1] | 1H NMR (400 MHz, Chloroform-d) δ 8.67 - 8.61 (m, 2H), 7.81 (dd, J = 6.0, 2.7 Hz, 1H), 7.38 (ddd, J = 8.8, 4.3, 2.7 Hz, 1H), 7.35 - 7.30 (m, 2H), 6.90 (t, J = 9.0 Hz, 1H), 6.49 - 6.42 (m, 1H), 3.92 (dd, J = 12.0, 2.2 Hz, 2H), 3.65 (s, 2H), 3.03 (dd, J = 12.0, 2.1 Hz, 2H), 2.68 - 2.58 (m, 3H), 1.84 - 1.74 (m, 4H). LCMS (Analytical Method A) Rt = 1.88 min, MS (ESIpos): m/z 449.3, 451.2 [M+H]+, Purity = 94%. |
| | | (1R,5S)-3-[2-(5-chloro-2-fluorophenyl)-7-methyl-3-(pyridin-4-yl)-3H-imidazo [4,5-b]pyridin-5-yl]-3,8-diazabicyclo[3.2.1]octane | |
| 81-RS | Intermediate 66 (53% yield) | 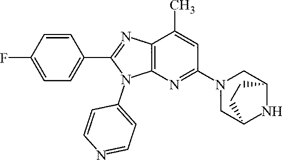 [SMILES: Cc1cc(N2C[C@H]3CC[C@@H](C2)N3)nc2c1nc(-c1ccc(F)cc1)n2-c1ccncc1] | 1H NMR (500 MHz, Chloroform-d) δ 8.71 - 8.64 (m, 2H), 7.53 - 7.45 (m, 2H), 7.36 - 7.31 (m, 2H), 7.08 - 7.01 (m, 2H), 6.46 - 6.41 (m, 1H), 3.89 (dd, J = 11.9, 2.2 Hz, 2H), 3.65 (s, 2H), 3.01 (dd, J = 11.9, 2.1 Hz, 2H), 2.65 (s, 3H), 1.79 (s, 4H). LCMS (Analytical Method A) Rt = 1.57 min, MS (ESIpos): m/z 415.3 [M+H]+, Purity = 99%. |
| | | (1R,5S)-3-[2-(4-fluorophenyl)-7-methyl-3-(pyridin-4-yl)-3H-imidazo[4,5-b]pyridin-5-yl]-3,8-diazabicyclo[3.2.1]octane | |
| 82-SS | Intermediate 67 (60% yield) | 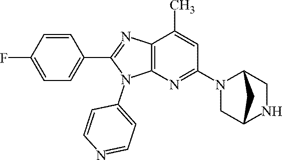 [SMILES: Cc1cc(N2C[C@@H]3C[C@H]2CN3)nc2c1nc(-c1ccc(F)cc1)n2-c1ccncc1] | 1H NMR (500 MHz, Chloroform-d) δ 8.66 (d, J = 6.1 Hz, 2H), 7.49 (dd, J = 8.7, 5.3 Hz, 2H), 7.34 (d, J = 6.1 Hz, 2H), 7.04 (t, J = 8.6 Hz, 2H), 6.23 (s, 1H), 4.71 (s, 1H), 3.82 (s, 1H), 3.60 (dd, J = 9.5, 1.8 Hz, 1H), 3.26 (d, J = 9.4 Hz, 1H), 3.12 - 3.03 (m, 2H), 2.63 (s, 3H), 1.89 (d, J = 9.5 Hz, 1H), 1.82 (d, J = 9.5 Hz, 1H). LCMS (Analytical Method A) Rt = 1.47 min, MS (ESIpos): m/z 401.3 [M+H]+, Purity = 100%. |
| | | (1S,4S)-2-[2-(4-fluorophenyl)-7-methyl-3-(pyridin-4-yl)-3H-imidazo[4,5-b]pyridin-5-yl]-2,5-diazabicyclo[2.2.1]heptane | |
| 83-SS | Intermediate 67 (42% yield) | 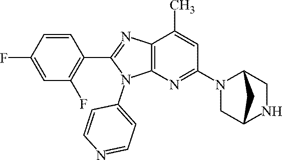 [SMILES: Cc1cc(N2C[C@@H]3C[C@H]2CN3)nc2c1nc(-c1ccc(F)cc1F)n2-c1ccncc1] | 1H NMR (400 MHz, Chloroform-d) δ 8.72 - 8.52 (m, 2H), 7.74 (td, J = 8.3, 6.4 Hz, 1H), 7.39 - 7.28 (m, 2H), 7.08 - 6.96 (m, 1H), 6.74 (ddd, J = 10.0, 8.8, 2.4 Hz, 1H), 6.25 (s, 1H), 4.74 (s, 1H), 3.84 (s, 1H), 3.62 (dd, J = 9.5, 2.1 Hz, 1H), 3.27 (d, J = 9.5 Hz, 1H), 3.09 (d, J = 1.6 Hz, 2H), 2.68 - 2.54 (m, 3H), 1.90 (d, J = 9.7 Hz, 1H), 1.83 (d, J = 9.5 Hz, 1H). LCMS (Analytical Method A) Rt = 1.51 min, MS (ESIpos): m/z 419.3 [M+H]+, Purity = 99%. |
| | | (1S,4S)-2-[2-(2,4-difluorophenyl)-7-methyl-3-(pyridin-4-yl)-3H-imidazo [4,5-b]pyridin-5-yl]-2,5-diazabicyclo[2.2.1]heptane | |
| 84-SS | Intermediate 67 (66% yield) | 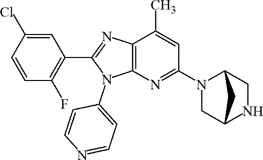 [SMILES: Cc1cc(N2C[C@@H]3C[C@H]2CN3)nc2c1nc(-c1cc(Cl)ccc1F)n2-c1ccncc1] | 1H NMR (400 MHz, Chloroform-d) δ 8.64 (d, J = 5.5 Hz, 2H), 7.81 (dd, J = 5.9, 2.6 Hz, 1H), 7.42 - 7.30 (m, 3H), 6.90 (t, J = 9.1 Hz, 1H), 6.26 (s, 1H), 4.74 (s, 1H), 3.84 (s, 1H), 3.62 (d, J = 8.2 Hz, 1H), 3.27 (d, J = 9.6 Hz, 1H), 3.15 - 3.02 (m, 2H), 2.64 (s, 3H), 1.90 (d, J = 9.5 Hz, 1H), 1.83 (d, J = 9.7 Hz, 1H). LCMS (Analytical Method A) Rt = 1.70 min, MS (ESIpos): m/z 435.3, 437.2 [M+H]+, Purity = 99%. |
| | | (1S,4S)-2-[2-(5-chloro-2-fluorophenyl)-7-methyl-3-(pyridin-4-yl)-3H-imidazo[4,5-b]pyridin-5-yl]-2,5-diazabicyclo[2.2.1]heptane | |
| 82-RR | Intermediate 68 (71% yield) | 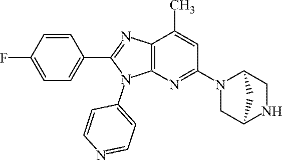 [SMILES: Cc1cc(N2C[C@H]3C[C@@H]2CN3)nc2c1nc(-c1ccc(F)cc1)n2-c1ccncc1] | 1H NMR (500 MHz, Chloroform-d) δ 8.71 - 8.61 (m, 2H), 7.53 - 7.45 (m, 2H), 7.37 - 7.30 (m, 2H), 7.09 - 6.99 (m, 2H), 6.23 (s, 1H), 4.71 (s, 1H), 3.82 (s, 1H), 3.61 (dd, J = 9.5, 2.1 Hz, 1H), 3.25 (d, J = 9.3 Hz, 1H), 3.11 - 3.02 (m, 2H), 2.64 (s, 3H), 1.89 (d, J = 9.7 Hz, 1H), 1.81 (d, J = 9.5 Hz, 1H). LCMS (Analytical Method A) Rt = 1.52 min, MS (ESIpos): m/z 401.3 [M+H]+, Purity = 97%. |
| | | (1R,4R)-2-[2-(4-fluorophenyl)-7-methyl-3-(pyridin-4-yl)-3H-imidazo[4,5-b]pyridin-5-yl]-2,5-diazabicyclo[2.2.1]heptane | |
| 83-RR | Intermediate 68 (48% yield) | 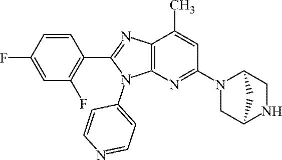 [SMILES: Cc1cc(N2C[C@H]3C[C@@H]2CN3)nc2c1nc(-c1ccc(F)cc1F)n2-c1ccncc1] | 1H NMR (500 MHz, Chloroform-d) δ 8.67 - 8.59 (m, 2H), 7.74 (td, J = 8.3, 6.4 Hz, 1H), 7.35 - 7.29 (m, 2H), 7.06 - 6.99 (m, 1H), 6.74 (ddd, J = 10.0, 8.8, 2.4 Hz, 1H), 6.25 (s, 1H), 4.73 (s, 1H), 3.83 (s, 1H), 3.62 (dd, J = 9.5, 2.1 Hz, 1H), 3.26 (d, J = 9.4 Hz, 1H), 3.12 - 3.03 (m, 2H), 2.66 -2.60 (m, 3H), 1.90 (d, J = 9.6 Hz, 1H), 1.82 (d, J = 9.5 Hz, 1H). LCMS (Analytical Method A) Rt = 1.73 min, MS (ESIpos): m/z 419.2 [M+H]+, Purity = 97%. |
| | | (1R,4R)-2-[2-(2,4-difluorophenyl)-7-methyl-3-(pyridin-4-yl)-3H-imidazo[4,5-b]pyridin-5-yl]-2,5-diazabicyclo[2.2.1]heptane | |

### Synthesis of (3R)-N-[2-(4-fluorophenyl)-3-(pyridin-4-yl)-3H-imidazo[4,5-b]pyridin-5-yl]-1-methylpyrrolidin-3-amine / Compound 70-R of Table 1

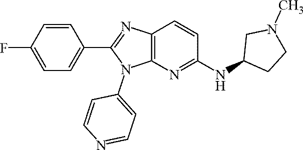 [SMILES: CN1CC[C@@H](Nc2ccc3nc(-c4ccc(F)cc4)n(-c4ccncc4)c3n2)C1]

(3R)-N-[2-(4-fluorophenyl)-3-(pyridin-4-yl)-3H-imidazo[4,5-b]pyridin-5-yl]pyrrolidin-3-amine (Compound 60-R of Table 1) (30 mg, 0.0785 mmol) was dissolved in DCM (1 mL), MeOH (0.2 mL) and acetic acid (0.05 mL). Formaldehyde (37% in water) (37%, 64 mg, 0.785 mmol) was added and the reaction stirred for 3 hours. Sodium triacetoxyborohydride (166 mg, 0.785 mmol) was added and the reaction stirred for 20 hours. Additional formaldehyde (37% in water) (37%, 64 mg, 0.785 mmol) and sodium triacetoxyborohydride (166 mg, 0.785 mmol) was added and the solution stirred at ambient for 1 h. The reaction was quenched into water. The aqueous layer was extracted into DCM 3 times, the combined organics washed with brine, passed through a phase separating filter and concentrated in vacuo to yield the crude solid which was purified by preparative HPLC (Method A1) to yield the title compound (5.4 mg, 25% yield). 1H NMR (500 MHz, Chloroform-d) δ 8.66 - 8.55 (m, 2H), 7.76 (d, J = 8.7 Hz, 1H), 7.43 - 7.35 (m, 2H), 7.29 - 7.23 (m, 2H), 7.00 - 6.92 (m, 2H), 6.35 (d, J = 8.7 Hz, 1H), 4.75 (d, J = 7.2 Hz, 1H), 4.33 - 4.22 (m, 1H), 2.77 - 2.68 (m, 1H), 2.59 (dd, J = 9.7, 6.4 Hz, 1H), 2.52 (dd, J = 9.7, 3.4 Hz, 1H), 2.29 (m, 5H), 1.67 - 1.56 (m, 1H). LCMS (Analytical Method A) Rt = 1.38 min, MS (ESIpos): m/z 389.3 [M+H]+, Purity = 100%.

Each of the compounds listed in Table 1.5.10 were prepared according to the method of Compound 70-R of Table 1 using the intermediates listed in the “Synthesis” column and the appropriate alkylating agent for such compounds. The final compounds were purified by preparative HPLC Methods A1, or A2.

TABLE 1.5.10

| | | | |
|---|---|---|---|
| Example | Synthesis | Structure/ Name | Data |
| 70-S | 60-S (16% yield) | 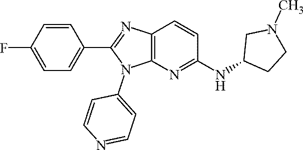 [SMILES: CN1CC[C@H](Nc2ccc3nc(-c4ccc(F)cc4)n(-c4ccncc4)c3n2)C1] | 1H NMR (500 MHz, Chloroform-d) δ 8.66 - 8.59 (m, 2H), 7.77 (d, J = 8.7 Hz, 1H), 7.50 - 7.36 (m, 2H), 7.33 -7.22 (m, 2H), 7.03 - 6.89 (m, 2H), 6.36 (d, J = 8.7 Hz, 1H), 4.77 (d, J = 7.3 Hz, 1H), 4.35 - 4.22 (m, 1H), 2.78 - 2.70 (m, 1H), 2.61 (dd, J = 9.7, 6.4 Hz, 1H), 2.53 (dd, J = 9.7, 3.4 Hz, 1H), 2.34 - 2.25 (m, 5H), 1.67 - 1.61 (m, 1H). LCMS (Analytical Method A) Rt = 1.39 min, MS (ESIpos): m/z 389.3 [M+H]+, Purity = 100%. |
| | | (3S)-N-[2-(4-fluorophenyl)-3-(pyridin-4-yl)-3H-imidazo[4,5-b]pyridin-5-yl]-1-methylpyrrolidin-3-amine | |
| 86-RR | 35-RR (53% yield) | 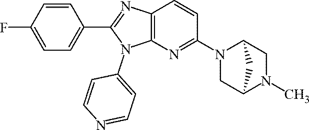 [SMILES: CN1C[C@H]2C[C@@H]1CN2c1ccc2nc(-c3ccc(F)cc3)n(-c3ccncc3)c2n1] | 1H NMR (500 MHz, Chloroform-d) δ 8.68 - 8.53 (m, 2H), 7.80 (d, J = 8.8 Hz, 1H), 7.45 - 7.43 (m, 2H), 7.31 -7.24 (m, 2H), 7.02 - 6.89 (m, 2H), 6.36 (d, J = 8.8 Hz, 1H), 4.65 - 4.46 (m, 1H), 3.53 (d, J = 9.7 Hz, 1H), 3.45 (s, 1H), 3.31 (dd, J = 9.7, 2.2 Hz, 1H), 2.92 - 2.85 (m, 1H), 2.63 - 2.53 (m, 1H), 2.34 (s, 3H), 1.99 - 1.87 (m, 1H), 1.82 - 1.71 (m, 1H). LCMS (Analytical Method B) Rt = 2.73 min, MS (ESIpos): m/z 401.3 [M+H]+, Purity = 99%. |
| | | (1R,4R)-2-[2-(4-fluorophenyl)-3-(pyridin-4-yl)-3H-imidazo[4,5-b]pyridin-5-yl]-5-methyl-2,5-diazabicyclo[2.2.1]heptane | |
| 89-RR | 82-RR (94% yield) | 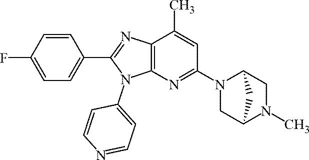 [SMILES: Cc1cc(N2C[C@H]3C[C@@H]2CN3C)nc2c1nc(-c1ccc(F)cc1)n2-c1ccncc1] | 1H NMR (500 MHz, Chloroform-d) δ 8.70 - 8.61 (m, 2H), 7.53 - 7.43 (m, 2H), 7.37 - 7.30 (m, 2H), 7.10 - 6.99 (m, 2H), 6.24 (s, 1H), 4.62 (s, 1H), 3.59 (d, J = 9.7 Hz, 1H), 3.51 (s, 1H), 3.36 (dd, J = 9.7, 2.2 Hz, 1H), 2.96 (dd, J = 9.6, 2.1 Hz, 1H), 2.67 - 2.59 (m, 4H), 2.40 (s, 3H), 1.97 (d, J = 9.7 Hz, 1H), 1.83 (d, J = 9.6 Hz, 1H). LCMS (Analytical Method A) Rt = 1.70 min, MS (ESIpos): m/z 415.3 [M+H]+, Purity = 95%. |
| | | (1R,4R)-2-[2-(4-fluorophenyl)-7-methyl-3-(pyridin-4-yl)-3H-imidazo[4,5-b]pyridin-5-yl]-5-methyl-2,5-diazabicyclo[2.2.1]heptane | |
| 90-RR | 83-RR (84% yield) | 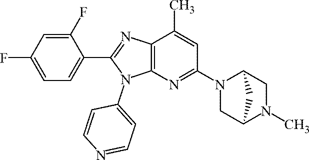 [SMILES: Cc1cc(N2C[C@H]3C[C@@H]2CN3C)nc2c1nc(-c1ccc(F)cc1F)n2-c1ccncc1] | 1H NMR (400 MHz, Chloroform-d) δ 8.67 - 8.57 (m, 2H), 7.73 (td, J = 8.3, 6.4 Hz, 1H), 7.35 - 7.28 (m, 2H), 7.07 - 6.97 (m, 1H), 6.74 (ddd, J = 10.0, 8.8, 2.4 Hz, 1H), 6.26 (s, 1H), 4.64 (s, 1H), 3.60 (d, J = 9.5 Hz, 1H), 3.51 (s, 1H), 3.37 (dd, J = 9.7, 2.2 Hz, 1H), 2.97 (dd, J = 9.5, 2.1 Hz, 1H), 2.67 -2.57 (m, 4H), 2.40 (s, 3H), 1.98 (d, J = 9.5 Hz, 1H), 1.84 (d, J = 9.5 Hz, 1H). LCMS (Analytical Method A) Rt = 1.75 min, MS (ESIpos): m/z 433.3 [M+H]+, Purity = 96%. |
| | | (1R,4R)-2-[2-(2,4-difluorophenyl)-7-methyl-3-(pyridin-4-yl)-3H-imidazo[4,5-b]pyridin-5-yl]-5-methyl-2,5-diazabicyclo[2.2.1]heptane | |
| 95 | 91 (69% yield) | 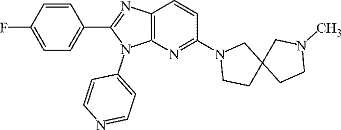 [SMILES: CN1CCC2(CCN(c3ccc4nc(-c5ccc(F)cc5)n(-c5ccncc5)c4n3)C2)C1] | 1H NMR (500 MHz, Chloroform-d) δ 8.66 - 8.57 (m, 2H), 7.80 (d, J = 8.8 Hz, 1H), 7.46 - 7.38 (m, 2H), 7.33 -7.27 (m, 2H), 7.02 - 6.91 (m, 2H), 6.34 (d, J = 8.8 Hz, 1H), 3.52 - 3.38 (m, 3H), 3.33 (d, J = 10.1 Hz, 1H), 2.63 (td, J = 8.5, 8.1, 5.8 Hz, 1H), 2.56 - 2.47 (m, 2H), 2.38 (d, J = 9.3 Hz, 1H), 2.29 (s, 3H), 2.02 - 1.88 (m, 2H), 1.88 - 1.73 (m, 2H). LCMS (Analytical Method A) Rt = 1.53 min, MS (ESIpos): m/z 429.2 [M+H]+, Purity = 98%. |
| | | 2-[2-(4-fluorophenyl)-3-(pyridin-4-yl)-3H-imidazo[4,5-b]pyridin-5-yl]-7-methyl-2,7-diazaspiro[4.4]nonane | |
| 101 | 92 (66% yield) | 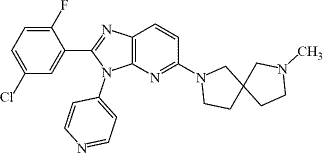 [SMILES: CN1CCC2(CCN(c3ccc4nc(-c5cc(Cl)ccc5F)n(-c5ccncc5)c4n3)C2)C1] | 1H NMR (500 MHz, Chloroform-d) δ 8.63 - 8.54 (m, 2H), 7.83 (d, J = 8.8 Hz, 1H), 7.76 - 7.69 (m, 1H), 7.34 -7.27 (m, 3H), 6.89 - 6.81 (m, 1H), 6.37 (d, J = 8.9 Hz, 1H), 3.53 - 3.41 (m, 3H), 3.35 (d, J = 10.1 Hz, 1H), 2.68 - 2.59 (m, 1H), 2.55 (d, J = 9.4 Hz, 1H), 2.53 - 2.48 (m, 1H), 2.39 (d, J = 9.3 Hz, 1H), 2.30 (s, 3H), 2.01 -1.89 (m, 2H), 1.89 - 1.73 (m, 2H). LCMS (Analytical Method A) Rt = 1.77 min, MS (ESIpos): m/z 463.2, 465.2 [M+H]+, Purity = 100%. |
| | | 2-[2-(5-chloro-2-fluorophenyl)-3-(pyridin-4-yl)-3H-imidazo[4,5-b]pyridin-5-yl]-7-methyl-2,7-diazaspiro[4.4]nonane | |
| 155 | 149 (73% yield) | 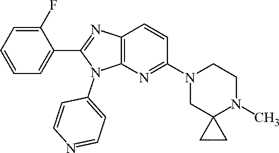 [SMILES: CN1CCN(c2ccc3nc(-c4ccccc4F)n(-c4ccncc4)c3n2)CC12CC2] | 1H NMR (400 MHz, DMSO-d6) δ 8.68 - 8.54 (m, 2H), 7.98 (d, J = 9.0 Hz, 1H), 7.74 (td, J = 7.5, 1.8 Hz, 1H), 7.63 - 7.50 (m, 1H), 7.44 - 7.33 (m, 3H), 7.30 - 7.14 (m, 1H), 6.93 (d, J = 9.0 Hz, 1H), 3.66 - 3.49 (m, 2H), 3.42 - 3.35 (m, 2H), 2.93 - 2.82 (m, 2H), 2.35 - 2.25 (m, 3H), 0.67 - 0.57 (m, 2H), 0.52 - 0.35 (m, 2H). LCMS (Analytical Method A) Rt = 1.42 min, MS (ESIpos): m/z 415.3 [M+H]+, Purity = 100%. |
| | | 7-[2-(2-fluorophenyl)-3-(pyridin-4-yl)-3H-imidazo[4,5-b]pyridin-5-yl]-4-methyl-4,7-diazaspiro[2.5]octane | |

### Synthesis of 4-[2-(2,4-difluorophenyl)-5-[(2R)-2-methylpiperazin-1-yl]-3H-imidazo[4,5-b]pyridin-3-yl]pyridazine / Compound 176-R of Table 1

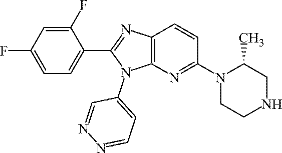 [SMILES: C[C@@H]1CNCCN1c1ccc2nc(-c3ccc(F)cc3F)n(-c3ccnnc3)c2n1]

2,4-Difluorobenzaldehyde (87 uL, 0.713 mmol) was added to a solution of tert-butyl (3R)-4-[5-amino-6-(pyridazin-4-ylamino)-2-pyridyl]-3-methyl-piperazine-1-carboxylate (intermediate 34) (77% purity, 325 mg, 0.649 mmol) in Ethanol (4.8 mL). The reaction was stirred for 15 minutes then hydrogen peroxide (35%, 114 uL, 1.30 mmol) was added followed by cerium ammonium nitrate (35 mg, 0.0649 mmol). The reaction was stirred overnight then quenched into water. The aqueous layer was extracted into ethyl acetate three times (~5 mL), the combined organics washed with brine, dried over MgSO₄ and concentrated in vacuo. The intermediate was purified by preparative HPLC (method A1) to yield tert-butyl (3R)-4-[2-(2,4-difluorophenyl)-3-(pyridazin-4-yl)-3H-imidazo[4,5-b]pyridin-5-yl]-3-methylpiperazine-1-carboxylate. The residue was dissolved in DCM (3 mL). TFA (0.1 mL) was added and the reaction stirred overnight. The mixture was concentrated in vacuo and the product purified by preparative HPLC (method A1) to yield to yield the title compound (22 mg, 0.0528 mmol, 8% Yield) as a pale yellow solid. 1H NMR (400 MHz, DMSO) δ 9.40 (dd, J = 2.7, 1.1 Hz, 1H), 9.35 (dd, J = 5.7, 1.0 Hz, 1H), 8.01 (d, J = 9.0 Hz, 1H), 7.89 - 7.76 (m, 1H), 7.70 (dd, J = 5.7, 2.7 Hz, 1H), 7.40 - 7.26 (m, 2H), 6.92 (d, J = 9.1 Hz, 1H), 4.44 - 4.27 (m, 1H), 4.00 -3.87 (m, 1H), 3.04 -2.87 (m, 2H), 2.87 -2.76 (m, 2H), 2.62 (dd, J = 11.3, 4.1 Hz, 1H), 1.14 (d, J = 6.6 Hz, 3H). LCMS (Analytical Method A) Rt = 1.58 min, MS (ESIpos): m/z 408.3 [M+H]+, Purity = 100%.

### Synthesis of 2-(5-chloro-2-fluoro-phenyl)-5-[(2R)-2-methylpiperazin-1-yl]-3-pyridazin-4-yl-imidazo[4,5-b]pyridine / Compound 177-R of Table 1

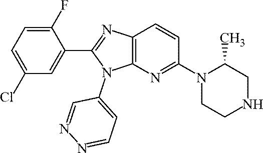 [SMILES: C[C@@H]1CNCCN1c1ccc2nc(-c3cc(Cl)ccc3F)n(-c3ccnnc3)c2n1]

5-Chloro-2-fluorobenzaldehyde (75 uL, 0.549 mmol) was added to a solution of tert-butyl (3R)-4-[5-amino-6-(pyridazin-4-ylamino)-2-pyridyl]-3-methyl-piperazine-1-carboxylate (intermediate 34) (77% purity, 325 mg, 0.649 mmol) in ethanol (4.8 mL). The reaction was stirred for 15 minutes then cerium ammonium nitrate (35 mg, 0.0649 mmol) was added followed by hydrogen peroxide (35%, 114 uL, 1.30 mmol). The reaction was stirred overnight then quenched into water. The aqueous layer was extracted into ethyl acetate three times (~5 mL), the combined organics washed with brine, dried over MgSO₄ and concentrated in vacuo. The intermediate was purified by preparative HPLC (method A1) to yield tert-butyl (3R)-4-[2-(5-chloro-2-fluorophenyl)-3-(pyridazin-4-yl)-3H-imidazo[4,5-b]pyridin-5-yl]-3-methylpiperazine-1-carboxylate. The residue was dissolved in DCM (3 mL). TFA (0.1 mL) was added and the reaction stirred overnight. The mixture was concentrated in vacuo and the product purified by preparative HPLC (method A1) to yield the title compound (28 mg, 0.0649 mmol, 7% Yield) as a pale yellow solid. 1H NMR (400 MHz, DMSO) δ 9.44 (dd, J = 2.7, 1.0 Hz, 1H), 9.36 (dd, J = 5.7, 1.0 Hz, 1H), 8.02 (d, J = 9.0 Hz, 1H), 7.83 (dd, J = 6.1, 2.7 Hz, 1H), 7.75 (dd, J = 5.7, 2.7 Hz, 1H), 7.66 (ddd, J = 8.9, 4.4, 2.8 Hz, 1H), 7.38 - 7.26 (m, 1H), 6.93 (d, J = 9.2 Hz, 1H), 4.43 - 4.31 (m, 1H), 4.01 - 3.89 (m, 1H), 3.03 - 2.89 (m, 2H), 2.86 - 2.77 (m, 2H), 2.65 - 2.57 (m, 1H), 1.15 (d, J = 6.6 Hz, 3H). LCMS (Analytical Method A) Rt = 1.75 min, MS (ESIpos): m/z 424.2, 426.2 [M+H]+, Purity = 99%.

### Synthesis of rac-5-{[2-(4-fluorophenyl)-3-(pyridin-4-yl)-3H-imidazo[4,5-b]pyridin-5-yl]amino}piperidin-3-ol / Compound 43 of Table 1

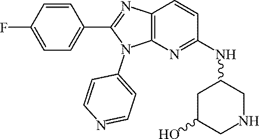 [SMILES: OC1CNCC(Nc2ccc3nc(-c4ccc(F)cc4)n(-c4ccncc4)c3n2)C1]

Tert-butyl (1R,5S)-6-[5-nitro-6-(4-pyridylamino)-2-pyridyl]-3,6-diazabicyclo[3.1.1]heptane-3-carboxylate (Intermediate 29) (70% purity, 88 mg, 0.15 mmol) and Na₂S₂O₄ (78 mg, 0.45 mmol) were heated for five minutes to 100° C. in DMSO (1 mL) and EtOH (0.2 mL). 4-Fluorobenzaldehyde (24 µL, 0.22 mmol) was added and the mixture was stirred at 100° C. for 30 h. The reaction mixture was cooled and quenched into water (2 mL), the aqueous layer then extracted into EtOAc (3 × 5 mL). The combined organic extracts were washed with saturated potassium carbonate solution (2 × 5 mL), brine (5 mL), passed through a phase separating filter paper and concentrated in vacuo. The residue was purified by preparative HPLC (Method A1) to yield the title compound (7 mg, 11% yield). 1H NMR (500 MHz, DMSO-d6) δ 8.73 - 8.65 (m, 2H), 7.80 (d, J = 8.7 Hz, 1H), 7.51 - 7.45 (m, 2H), 7.44 - 7.40 (m, 2H), 7.28 - 7.20 (m, 2H), 6.64 (d, J = 7.5 Hz, 1H), 6.59 (d, J = 8.8 Hz, 1H), 4.53 - 4.47 (m, 1H), 3.97 - 3.87 (m, 1H), 3.72 - 3.61 (m, 1H), 2.87 - 2.78 (m, 1H), 2.76 - 2.68 (m, 1H), 2.46 - 2.39 (m, 1H), 1.79 (s, 1H), 1.64 (s, 1H). LCMS (Analytical Method B) Rt = 2.14 min, MS (ESIpos): m/z 405.3 [M+H]+, Purity = 99%.

### Synthesis of 1-[2-(4-fluorophenyl)-6-methyl-3-(pyridin-4-yl)-3H-imidazo[4,5-b]pyridin-5-yl]piperazine / Compound 74 of Table 1

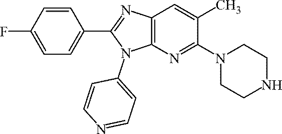 [SMILES: Cc1cc2nc(-c3ccc(F)cc3)n(-c3ccncc3)c2nc1N1CCNCC1]

Tert-butyl 4-[3-methyl-5-nitro-6-(4-pyridylamino)-2-pyridyl]piperazine-1-carboxylate (Intermediate 51) (75 mg, 0.18 mmol) and Na₂S₂O₄ (96 mg, 0.543 mmol) were heated for five minutes at 100° C. in DMSO (1 mL) and EtOH (0.2 mL). 4-Fluorobenzaldehyde (34 mg, 0.27 mmol) was added and the mixture was stirred at 100° C. for 21 h. The reaction was cooled and quenched into water (2 mL). The aqueous layer was extracted into EtOAc (3 × 5 mL) and once in DCM (5 mL), the combined organic extracts were washed with saturated potassium carbonate solution (2 × 5 mL), brine (5 mL), and passed through a phase separating filter and concentrated in vacuo. The residue was purified by preparative HPLC (Method A1) to yield the title compound (26 mg, 35% yield). 1H NMR (400 MHz, Chloroform-d) δ 8.71 - 8.56 (m, 2H), 7.78 (s, 1H), 7.51 - 7.40 (m, 2H), 7.40 - 7.24 (m, 2H), 7.11 - 6.92 (m, 2H), 3.08 - 3.00 (m, 4H), 3.00 - 2.93 (m, 4H), 2.36 (s, 3H). LCMS (Analytical Method B) Rt = 2.86 min, MS (ESIpos): m/z 389.3 [M+H]+, Purity = 97%.

### Synthesis of 1-[6-chloro-2-(4-fluorophenyl)-3-(pyridin-4-yl)-3H-imidazo[4,5-b]pyridin-5-yl]piperazine / Compound 88 of Table 1

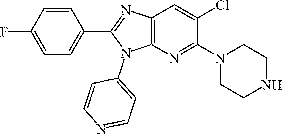 [SMILES: Fc1ccc(-c2nc3cc(Cl)c(N4CCNCC4)nc3n2-c2ccncc2)cc1]

To a stirred solution of 2-(4-fluorophenyl)-5-piperazin-1-yl-3-(4-pyridyl)imidazo[4,5-b]pyridine (Compound 17 of Table 1) (30 mg, 77 µmol) in MeCN (2 mL), NCS (12 mg, 92 µmol) was added and the mixture was allowed to stir at 60° C. for 3 h. The reaction was quenched with 1 M NaOH (10 mL) and the product was extracted with DCM (2 × 10 mL). The combined organic layers were dried by filtering through a Telos phase separator and then concentrated in vacuo. The residue was purified by trituration with Et₂O followed by preparative HPLC (Method B1). The product was dissolved in DCM (20 mL) and washed with 1 M NaOH (5 mL). The organic layer was concentrated in vacuo and lyophilised to yield the title compound (9 mg, 28% yield). 1H NMR (400 MHz, Chloroform-d) δ 8.76 - 8.68 (m, 2H), 8.06 (s, 1H), 7.56 - 7.48 (m, 2H), 7.37 - 7.31 (m, 2H), 7.13 - 7.03 (m, 2H), 3.33 - 3.23 (m, 4H), 3.09 - 2.98 (m, 4H). LCMS (Analytical Method A) Rt = 1.71 min, MS (ESIpos): m/z 409.2, 411.2 [M+H]+, Purity = 97%.

### Synthesis of 1-[6-bromo-2-(4-fluorophenyl)-3-(pyridin-4-yl)-3H-imidazo[4,5-b]pyridin-5-yl]piperazine / Compound 97 of Table 1

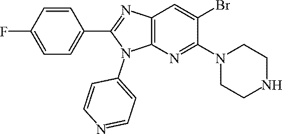 [SMILES: Fc1ccc(-c2nc3cc(Br)c(N4CCNCC4)nc3n2-c2ccncc2)cc1]

To a stirred solution of 2-(4-fluorophenyl)-5-piperazin-1-yl-3-(4-pyridyl)imidazo[4,5-b]pyridine (Compound 17 of Table 1) (300 mg, 0.77 mmol) in MeCN (7 mL), NBS (164 mg, 0.92 mmol) was added and the mixture was allowed to stir at 60° C. for 2 h. The reaction was quenched with 1 M NaOH (10 mL) and the product was extracted with DCM (2 × 10 mL). The combined organic layers were dried by filtering through a Telos phase separator and then concentrated in vacuo. The residue was purified by flash chromatography (25 g, silica), eluting with 0 - 30% MeOH in DCM to yield the title compound (159 mg, 44% yield). 1H NMR (500 MHz, Chloroform-d) δ 8.76 - 8.69 (m, 2H), 8.25 (s, 1H), 7.56 - 7.49 (m, 2H), 7.37 - 7.30 (m, 2H), 7.09 (t, J = 8.6 Hz, 2H), 3.32 - 3.21 (m, 4H), 3.10 - 3.00 (m, 4H). LCMS (Analytical Method A) Rt = 1.72 min, MS (ESIpos): m/z 453.1, 455.1 [M+H]+, Purity = 100%.

### Synthesis of 1-[2-(4-fluorophenyl)-3-(pyridin-4-yl)-3H-imidazo[4,5-b]pyridin-5-yl]azetidin-3-ol / Compound 98 of Table 1

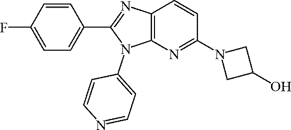 [SMILES: OC1CN(c2ccc3nc(-c4ccc(F)cc4)n(-c4ccncc4)c3n2)C1]

6-[Tert-butyl(dimethyl)silyl]oxyazetidin-1-yl]-3-nitro-N-(4-pyridyl)pyridin-2-amine (Intermediate 96) (200 mg, 0.5 mmol) and Na₂S₂O₄ (263 mg, 1.5 mmol) were heated in DMSO (3 mL) and EtOH (0.3 mL) to 100° C. for five minutes. 4-Fluorobenzaldehyde (80 µL, 0.747 mmol) was added. The solution was heated to 100° C. for 21 h. The reaction was cooled and quenched into water (2 mL). The solvent was removed in vacuo and DCM (5 mL) was added the organic extracts were washed with saturated potassium carbonate solution (2 × 5 mL) brine (5 mL), and passed through a phase separating filter and concentrated in vacuo. The residue was purified by preparative HPLC (Method A1) to yield the title compound (23 mg, 12% yield). 1H NMR (500 MHz, Chloroform-d) δ 8.67 - 8.55 (m, 2H), 7.83 (d, J = 8.6 Hz, 1H), 7.46 - 7.38 (m, 2H), 7.30 - 7.24 (m, 2H), 7.03 - 6.94 (m, 2H), 6.30 (d, J = 8.6 Hz, 1H), 4.78 - 4.67 (m, 1H), 4.24 (dd, J = 9.7, 6.4 Hz, 2H), 3.81 (dd, J = 9.8, 4.5 Hz, 2H), 2.13 (d, J = 6.4 Hz, 1H). LCMS (Analytical Method B) Rt = 2.36 min, MS (ESIpos): m/z 362.3 [M+H]+, Purity = 98%.

### Synthesis of (1R,4R)-2-[2-(4-fluorophenyl)-3-(pyridin-4-yl)-3H-imidazo[4,5-b]pyridin-5-yl]-5-(oxetan-3-yl)-2,5-diazabicyclo[2.2.1]heptane / Compound 99-RR of Table 1

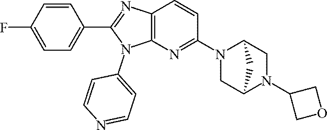 [SMILES: Fc1ccc(-c2nc3ccc(N4C[C@H]5C[C@@H]4CN5C4COC4)nc3n2-c2ccncc2)cc1]

To a solution of 5-[(1R,4R)-2,5-diazabicyclo[2.2.1]heptan-2-yl]-2-(4-fluorophenyl)-3-(4-pyridyl)imidazo[4,5-b]pyridine (Compound 35-RR of Table 1) (30 mg, 77.6 µmol) and oxetan-3-one (6.0 µL, 93.2 µmol) in DCE (1 mL) was added acetic acid (1.8 µL, 31.1 µmol). The reaction was then stirred 2 h at ambient before the addition of sodium triacetoxyborohydride (33 mg, 0.155 mmol). The mixture was then stirred a further 16 h. The reaction was diluted with NaHCO₃ (aq) solution and DCM and the phases separated. The isolated organics were concentrated in vacuo and purified by preparative HPLC (Method A1). To afford the title compound (15 mg, 43% yield). 1H NMR (500 MHz, Chloroform-d) δ 8.66 - 8.75 (m, 2H), 7.91 (d, J = 8.7 Hz, 1H), 7.47 - 7.56 (m, 2H), 7.34 - 7.40 (m, 2H), 7.03 - 7.14 (m, 2H), 6.43 (d, J = 8.8 Hz, 1H), 4.70 - 4.76 (m, 2H), 4.67 (t, J = 6.4 Hz, 1H), 4.55 (t, J = 6.1 Hz, 1H), 4.47 (t, J = 5.9 Hz, 1H), 3.98 (p, J = 6.1 Hz, 1H), 3.58 (s, 1H), 3.43 (dd, J = 9.6, 2.0 Hz, 1H), 3.35 (d, J = 9.6 Hz, 1H), 2.98 (dd, J = 9.5, 2.0 Hz, 1H), 2.90 (d, J = 9.6 Hz, 1H), 1.98 (d, J = 9.6 Hz, 1H), 1.90 (d, J = 9.6 Hz, 1H). LCMS (Analytical Method A) Rt = 1.39 min, MS (ESIpos): m/z 443.3 [M+H]+, Purity = 99%.

### Synthesis of (1R,4R)-2-[2-(4-fluorophenyl)-3-(pyridin-4-yl)-3H-imidazo[4,5-b]pyridin-5-yl]-5-(2-methoxyethyl)-2,5-diazabicyclo[2.2.1]heptane / Compound 105-RR of Table 1

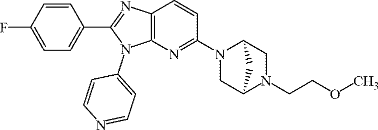 [SMILES: COCCN1C[C@H]2C[C@@H]1CN2c1ccc2nc(-c3ccc(F)cc3)n(-c3ccncc3)c2n1]

To a solution of 5-[(1R,4R)-2,5-diazabicyclo[2.2.1]heptan-2-yl]-2-(4-fluorophenyl)-3-(4-pyridyl)imidazo[4,5-b]pyridine (Compound 35-RR of Table 1) (30 mg, 77.6 µmol) in MeCN (0.5 mL) and DCM (1 mL) stirring at room temperature was added 1-bromo-2-methoxyethane (7.3 µL, 77.6 µmol) followed by triethylamine (22 µL, 0.155 mmol). The reaction was then stirred at RT for a total of 36 h diluted with chloroform (1 mL), warmed to 50° C. and stirred a further 16 h. The reaction was concentrated in vacuo and the residue purified by preparative HPLC (Method A1) to afford the title compound (7.7 mg, 22% yield). 1H NMR (500 MHz, Chloroform-d) δ 8.68 (d, J = 6.1 Hz, 2H), 7.86 (d, J = 8.7 Hz, 1H), 7.44 - 7.53 (m, 2H), 7.31 - 7.39 (m, 2H), 6.98 - 7.10 (m, 2H), 6.41 (d, J = 8.8 Hz, 1H), 4.63 (s, 1H), 3.70 (s, 1H), 3.61 (d, J = 9.7 Hz, 1H), 3.41 - 3.49 (m, 2H), 3.38 (dd, J = 9.8, 2.0 Hz, 1H), 3.34 (s, 3H), 3.10 (d, J = 9.0 Hz, 1H), 2.68 - 2.82 (m, 2H), 2.64 (d, J = 9.4 Hz, 1H), 2.02 (d, J = 9.5 Hz, 1H), 1.85 (d, J = 9.5 Hz, 1H). LCMS (Analytical Method A) Rt = 1.5 min, MS (ESIpos): m/z 445.3 [M+H]+, Purity = 99%.

### Synthesis of 4-[2-(4-fluorophenyl)-5-[3-(pyrrolidin-1-yl)azetidin-1-yl]-3H-imidazo[4,5-b]pyridin-3-yl]pyridine / Compound 106 of Table 1

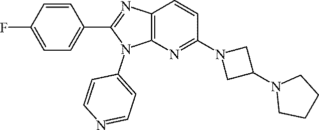 [SMILES: Fc1ccc(-c2nc3ccc(N4CC(N5CCCC5)C4)nc3n2-c2ccncc2)cc1]

A solution of 1-[2-(4-fluorophenyl)-3-(4-pyridyl)imidazo[4,5-b]pyridin-5-yl]azetidin-3-ol (Compound 98 of Table 1) (23 mg, 63.6 µmol) in DCM (1 mL) was cooled to 0° C. and Dess-Martin periodinane (54 mg, 0.127 mmol) added portionwise. The solution was allowed to warm to ambient temperature and stirred for 2 h. Additional Dess-Martin periodinane (54 mg, 0.127 mmol) was added and the solution stirred for 2 h. The reaction was quenched into water (1 mL). The organic was separated and the aqueous extracted with DCM (2 × 1 mL). Acetic acid (0.05 mL) followed by sodium triacetoxyborohydride (27 mg, 0.127 mmol) were added to the combined organics along with pyrrolidine (5.8 µL, 70.0 µmol) and the reaction stirred for 2 h. The mixture was dried in vacuo and the crude solid was purified by preparative HPLC (Method A1) to afford the title compound (3.1 mg, 12% yield). 1H NMR (500 MHz, Chloroform-d) δ 8.66 - 8.54 (m, 2H), 7.80 (d, J = 8.6 Hz, 1H), 7.46 - 7.37 (m, 2H), 7.33 - 7.24 (m, 2H), 7.03 - 6.91 (m, 2H), 6.27 (d, J = 8.7 Hz, 1H), 4.10 - 3.95 (m, 2H), 3.86 (dd, J = 8.3, 5.1 Hz, 2H), 3.43 - 3.30 (m, 1H), 2.54 - 2.41 (m, 4H), 1.80 - 1.72 (m, 4H). LCMS (Analytical Method B) Rt = 3.10 min, MS (ESIpos): m/z 415.3 [M+H]+, Purity = 100%.

### Synthesis of 1-[6-fluoro-2-(4-fluorophenyl)-3-(pyridin-4-yl)-3H-imidazo[4,5-b]pyridin-5-yl]piperazine/ Compound 110 of Table 1

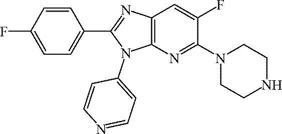 [SMILES: Fc1ccc(-c2nc3cc(F)c(N4CCNCC4)nc3n2-c2ccncc2)cc1]

To a stirred solution of tert-butyl 4-[6-fluoro-2-(4-fluorophenyl)-3-(4-pyridyl)imidazo[4,5-b]pyridin-5-yl]piperazine-1-carboxylate (Intermediate 98) (100%, 40 mg, 81.2 µmol) in DCM (2 mL), TFA (0.12 mL, 1.62 mmol) was added, and the mixture was allowed to stir for 3 h. The mixture was then quenched with NaOH (2 mL, 1 M). The organic layer was separated, and the aqueous layer was extracted with DCM (2 mL). The combined organic layers were filtered through hydrophobic frit and concentrated in vacuo. The crude solid was purified by flash chromatography eluting with 0-50% MeOH/DCM to afford the title compound (10 mg, 30% yield). 1H NMR (400 MHz, Methanol-d4) δ 8.71 - 8.63 (m, 2H), 7.80 (d, J = 12.6 Hz, 1H), 7.60 -7.53 (m, 2H), 7.53 - 7.48 (m, 2H), 7.19 (t, J = 8.8 Hz, 2H), 3.45 - 3.39 (m, 4H), 3.01 - 2.91 (m, 4H). LCMS (Analytical Method B) Rt = 2.80 min, MS (ESIpos): m/z 393.3 [M+H]+, Purity = 97%.

### Synthesis of (1S,6R)-3-[2-(4-fluorophenyl)-3-(pyridin-4-yl)-3H-imidazo[4,5-b]pyridin-5-yl]-3,8-diazabicyclo[4.2.0]octane / Compound 154-SR of Table 1

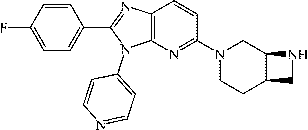 [SMILES: Fc1ccc(-c2nc3ccc(N4CC[C@@H]5CN[C@@H]5C4)nc3n2-c2ccncc2)cc1]

tert-Butyl (1S,6R)-3-[5-nitro-6-(4-pyridylamino)-2-pyridyl]-3,8-diazabicyclo[4.2.0]octane-8-carboxylate (Intermediate 70) (235 mg, 0.551 mmol), 4-fluorobenzaldehyde (65 uL, 0.606 mmol) and Na₂S₂O₄ (300 mg, 1.72 mmol) were dissolved in DMSO (4 mL) and ethanol (0.4 mL). The reaction was heated to 100° C. for 20 hrs. The reaction was cooled and diluted with 1 M NaOH. The aqueous layer was extracted with DCM and the organics were combined and concentrated in vacuo. The intermediate product was purified via preparative HPLC (method A2) to yield tert-butyl(1S,6R)-3-[2-(4-fluorophenyl)-3-(4-pyridyl)imidazo[4,5-b]pyridin-5-yl]-3,8-diazabicyclo[4.2.0]octane-8-carboxylate. The residue was dissolved in DCM (1 mL) and TFA (0.3 mL) was added. The reaction was stirred for 6h. Additional TFA (0.3 mL) was added and stirring continued for 2h. The mixture was concentrated in vacuo and the product was purified by preparative HPLC (Method A1) to afford the title compound (8 mg, 3%). 1H NMR (400 MHz, DMSO-d6) δ 8.73 - 8.66 (m, 2H), 7.95 - 7.90 (m, 1H), 7.53 - 7.47 (m, 2H), 7.46 - 7.41 (m, 2H), 7.29 - 7.22 (m, 2H), 6.70 (d, J = 9.0 Hz, 1H), 4.13 - 4.05 (m, 1H), 3.86 - 3.78 (m, 1H), 3.77 - 3.70 (m, 1H), 3.69 - 3.53 (m, 3H), 3.10 - 3.04 (m, 1H), 2.78 - 2.68 (m, 1H), 1.99 -1.90 (m, 1H), 1.85 - 1.77 (m, 1H). LCMS (Analytical Method A) Rt = 1.47 min, MS (ESIpos): m/z 401.2 [M+H]+, Purity = 98%.

### Synthesis of 1-{4-[2-(2,4-difluorophenyl)-3-(pyridin-4-yl)-3H-imidazo[4,5-b]pyridin-5-yl]piperazin-1-yl}ethan-1-one / Compound 159 of Table 1

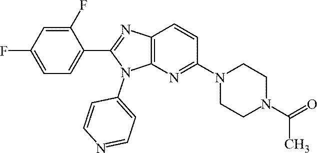 [SMILES: CC(=O)N1CCN(c2ccc3nc(-c4ccc(F)cc4F)n(-c4ccncc4)c3n2)CC1]

To a solution of 2-(2,4-difluorophenyl)-5-piperazin-1-yl-3-(4-pyridyl)imidazo[4,5-b]pyridine (Compound 34 of Table 1) (15 mg, 38.2 µmol) in DCM (1 mL) was added acetyl chloride (3.3 µL, 45.9 µmol) followed by DIPEA (8.0 µL, 45.9 µmol) and the solution stirred for 1 h. The crude was purified using flash chromatography eluting with 0-0.5% MeOH in DCM to afford the title compound (11 mg, 67% yield). 1H NMR (500 MHz, DMSO-d6) δ 8.74 - 8.62 (m, 2H), 8.05 (d, J = 8.9 Hz, 1H), 7.86 - 7.75 (m, 1H), 7.45 - 7.38 (m, 2H), 7.36 - 7.23 (m, 2H), 7.01 (d, J = 9.0 Hz, 1H), 3.63 - 3.53 (m, 6H), 3.54 - 3.46 (m, 2H), 2.05 (s, 3H). LCMS (Analytical Method B) Rt = 2.59 min, MS (ESIpos): m/z 435.3 [M+H]+, Purity = 100%.

### Synthesis of 1-[2-(2,4-difluorophenyl)-3-(pyridin-4-yl)-3H-imidazo[4,5-b]pyridin-5-yl]-4-methanesulfonylpiperazine / Compound 161 of Table 1

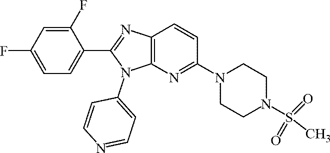 [SMILES: CS(=O)(=O)N1CCN(c2ccc3nc(-c4ccc(F)cc4F)n(-c4ccncc4)c3n2)CC1]

To a solution of 2-(2,4-difluorophenyl)-5-piperazin-1-yl-3-(4-pyridyl)imidazo[4,5-b]pyridine (Compound 34 of Table 1) (15 mg, 38.2 µmol) in DCM (1 mL) was added DIPEA (8.0 µL, 45.9 µmol) followed by methanesulfonyl chloride (3.6 µL, 45.9 µmol) and the solution stirred for 1 h. The crude was purified using flash chromatography eluting with 0-0.5% MeOH in DCM and additionally by preparative HPLC (Method A1) to afford the title compound (8.3 mg, 44% yield). 1H NMR (400 MHz, DMSO-d6) δ 8.73 - 8.60 (m, 2H), 8.06 (d, J = 8.9 Hz, 1H), 7.87 - 7.72 (m, 1H), 7.47 - 7.37 (m, 2H), 7.37 - 7.23 (m, 2H), 7.04 (d, J = 9.0 Hz, 1H), 3.74 - 3.58 (m, 4H), 3.28 - 3.15 (m, 4H), 2.90 (s, 3H). LCMS (Analytical Method B) Rt = 2.85 min, MS (ESIpos): m/z 471.3 [M+H]+, Purity = 96%.

### Synthesis of 4-{5-[(8aR)-octahydropyrrolo[1,2-a]pyrazin-2-yl]-2-(4-fluorophenyl)-3H-imidazo[4,5-b]pyridin-3-yl}pyridine / Compound 170-R of Table 1

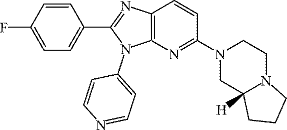 [SMILES: Fc1ccc(-c2nc3ccc(N4CCN5CCC[C@@H]5C4)nc3n2-c2ccncc2)cc1]

A mixture of 2-(4-fluorophenyl)-5-iodo-3-(4-pyridyl)imidazo[4,5-b]pyridine (Intermediate 97) (78% purity, 40 mg, 75.0 µmol), (8aR)-1,2,3,4,6,7,8,8a-octahydropyrrolo[1,2-a]pyrazine (12 mg, 98.9 µmol), Pd₂dba₃ (1.7 mg, 1.87 µmol), NaOtBu (14 mg, 0.150 mmol) and BINAP (2.3 mg, 3.75 µmol) in toluene (1.25 mL) was degassed by sparging with nitrogen. The reaction was heated to 100° C. for 24 h. The mixture was retreated with Pd₂dba₃ (1.7 mg, 1.87 µmol), BINAP (2.3 mg, 3.75 µmol), and (8aR)-1,2,3,4,6,7,8,8a-octahydropyrrolo[1,2-a]pyrazine (12 mg, 98.9 µmol) and stirred at 100° C. for 4 h. The mixture was filtered through celite washing with EtOAc (30 mL). The filtrate was extracted with HCl (2 × 30 mL, 2 M). The aqueous layer was basified with NaOH (30 mL), extracted with DCM (3 × 80 mL), and the combined organics filtered through a hydrophobic frit and evaporated in vacuo. The residue was purified using preparative HPLC (Method A1) followed by flash chromatography (12 g KP-NH) eluting with 0-3% MeOH/DCM to afford the title compound (6.0 mg, 18% yield). 1H NMR (500 MHz, MeOD) δ 8.68 - 8.63 (m, 2H), 7.91 (d, J = 9.0 Hz, 1H), 7.55 - 7.50 (m, 2H), 7.50 - 7.46 (m, 2H), 7.20 - 7.13 (m, 2H), 6.94 (d, J = 9.0 Hz, 1H), 4.46 - 4.37 (m, 1H), 4.34 - 4.26 (m, 1H), 3.18 - 3.07 (m, 2H), 3.01 (ddd, J = 12.8, 11.8, 3.3 Hz, 1H), 2.66 (dd, J = 12.4, 10.4 Hz, 1H), 2.30 (td, J = 11.5, 3.4 Hz, 1H), 2.21 (app q, J = 9.0 Hz, 1H), 2.17 - 2.09 (m, 1H), 1.98 - 1.90 (m, 1H), 1.90 - 1.77 (m, 2H), 1.55 - 1.45 (m, 1H). LCMS (Analytical Method A) Rt = 1.43 min, MS (ESIpos): m/z 415.3 [M+H]+, Purity = 94%.

### Synthesis of 4-{5-[(8aS)-octahydropyrrolo[1,2-a]pyrazin-2-yl]-2-(4-fluorophenyl)-3H-imidazo[4,5-b]pyridin-3-yl}pyridine / Compound 170-S of Table 1

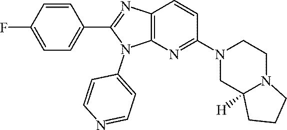 [SMILES: Fc1ccc(-c2nc3ccc(N4CCN5CCC[C@H]5C4)nc3n2-c2ccncc2)cc1]

A mixture of 2-(4-fluorophenyl)-5-iodo-3-(4-pyridyl)imidazo[4,5-b]pyridine (Intermediate 97) (30 mg, 0.0706 mmol), (8aS)-1,2,3,4,6,7,8,8a-octahydropyrrolo[1,2-a]pyrazine (12 mg, 93.2 µmol), Pd₂dba₃ (1.6 mg, 1.77 µmol), NaOtBu (14 mg, 0.141 mmol) and BINAP (2.2 mg, 3.53 µmol) in toluene (1.2 mL) was degassed by sparging with nitrogen. The reaction was heated to 100° C. for 2 h. The mixture was filtered through celite washing with EtOAc (30 mL). The filtrate was purified by flash chromatography (5 g, KP-NH) eluting with 0-30% (EtOAc/EtOH, 3:1) in heptane to yield a crude solid which was purified by preparative HPLC (Method A3). The solid was basified with NaOH (1 M), extracted with DCM (3 × 10 mL), filtered through a hydrophobic frit and evaporated in vacuo to afford the title compound (12 mg, 39% yield). 1H NMR (500 MHz, MeOD) δ 8.69 - 8.64 (m, 2H), 7.92 (d, J = 9.0 Hz, 1H), 7.57 - 7.51 (m, 2H), 7.51 - 7.47 (m, 2H), 7.17 (t, J = 8.8 Hz, 2H), 6.96 (d, J = 9.0 Hz, 1H), 4.47 - 4.39 (m, 1H), 4.35 - 4.27 (m, 1H), 3.19 - 3.08 (m, 2H), 3.01 (td, J = 12.8, 3.3 Hz, 1H), 2.67 (dd, J = 12.4, 10.5 Hz, 1H), 2.31 (td, J = 11.5, 3.4 Hz, 1H), 2.22 (q, J = 9.0 Hz, 1H), 2.20 - 2.10 (m, 1H), 2.00 - 1.91 (m, 1H), 1.91 - 1.78 (m, 2H), 1.58 - 1.45 (m, 1H). LCMS (Analytical Method A) Rt = 1.45 min, MS (ESIpos): m/z 415.3 [M+H]+, Purity = 95%.

### Synthesis of (2R)-1-{6-bromo-3-[2-(difluoromethyl)pyridin-4-yl]-2-(4-fluorophenyl)-3H-imidazo[4,5-b]pyridin-5-yl)-2-methylpiperazine / compound 144 of table 1

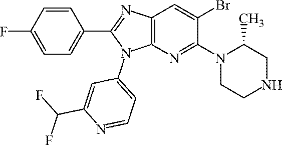 [SMILES: C[C@@H]1CNCCN1c1nc2c(cc1Br)nc(-c1ccc(F)cc1)n2-c1ccnc(C(F)F)c1]

To a stirred solution of NBS (260 mg, 1.46 mmol) in MeCN (10 mL) was added tert-butyl (3R)-4-[3-[2-(difluoromethyl)-4-pyridyl]-2-(4-fluorophenyl)imidazo[4,5-b]pyridin-5-yl]-3-methyl-piperazine-1-carboxylate (Intermediate 85) (705 mg,1.22 mmol) and the mixture was allowed to stir at 60° C. for 5 h. The mixture was partitioned between DCM (10 mL) and NaOH (15 mL, 1 M). The organic layer was separated and the aqueous layer was extracted with DCM (2 ×15 mL). The combined organics were filtered through a hydrophobic frit and evaporated in vacuo. The residue was purified by flash chromatography (25 g, silica) eluting with 0-10% MeOH/DCM to yield a crude solid which was purified by preparative HPLC (Method A1) to afford the title compound (35 mg, 5.6% yield). 1H NMR (500 MHz, DMSO) δ 8.81 (d, J = 5.3 Hz, 1H), 8.50 (s, 1H), 7.89 (d, J = 1.8 Hz, 1H), 7.61 - 7.56 (m, 2H), 7.56 - 7.54 (m, 1H), 7.37 - 7.27 (m, 2H), 7.04 (t, J = 54.7 Hz, 1H), 3.66 - 3.56 (m, 1H), 3.25 - 3.15 (m, 1H), 2.91 (dd, J = 12.1, 3.3 Hz, 1H), 2.86 - 2.74 (m, 3H), 2.57 - 2.53 (m, 1H), 0.99 (d, J = 6.3 Hz, 3H). LCMS (Analytical Method A) Rt = 2.19 min, MS (ESIpos): m/z 517.2,519.2 [M+H]+, Purity = 100%.

### Synthesis of (2R)-4-[2-(2,4-difluorophenyl)-3-(pyridin-4-yl)-3H-imidazo[4,5-b]pyridin-5-yl]piperazine-2-carboxamide. / Compound 200-R of Table 1

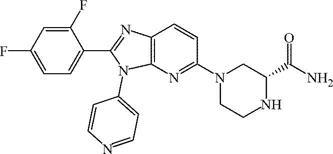 [SMILES: NC(=O)[C@H]1CN(c2ccc3nc(-c4ccc(F)cc4F)n(-c4ccncc4)c3n2)CCN1]

Tert-butyl (2R)-2-carbamoyl-4-[2-(2,4-difluorophenyl)-3-(4-pyridyl)imidazo[4,5-b]pyridin-5-yl]piperazine-1-carboxylate (Intermediate 69) (82 mg, 0.15 mmol) was dissolved in 4 M HCl in dioxane (8.0 mL, 0.15 mmol) and stirred at RT for 1 hours. Sat. aq. NaHCOs (25 ml) was slowly added and the product was extracted into DCM (2 × 25 ml). Combined organic layers were dried over magnesium sulfate and concentrated in vacuo. The remaining residue was purified by preparative HPLC (Method A1) to yield the title compound (24 mg, 36% yield). 1H NMR (400 MHz, DMSO) δ 8.75 - 8.56 (m, 2H), 8.00 (d, J = 9.0 Hz, 1H), 7.86 - 7.68 (m, 1H), 7.51 - 7.36 (m, 2H), 7.33 (d, J = 2.1 Hz, 1H), 7.30 (s, 1H), 7.29 - 7.24 (m, 1H), 7.13 (s, 1H), 6.96 (d, J = 9.0 Hz, 1H), 4.09 (dd, J = 12.4, 3.0 Hz, 1H), 3.88 (d, J = 12.4 Hz, 1H), 3.26 (d, J = 7.8 Hz, 1H), 3.09 - 2.88 (m, 3H), 2.81 - 2.63 (m, 1H), 2.54 (s, 1H). LCMS (Analytical Method B) Rt = 2.21 min, MS (ESIpos): m/z 436.3 [M+H]+, Purity = 100%.

### Example 2 - Activity of Compounds of General Formula (I)

The DUX4 repression of compounds of general formula (I) was assayed following a known protocol (the protocol of Example 2 of WO2019/115711). Several compounds were incubated with primary FSHD cells for 72 hours. Results are shown in Table 2.2, showing DUX4 Count % inhibition. Additional results are in Table 2.3, where compounds 98, 106, and 188-R fall outside of the bins.

TABLE 2.2

biological data for selected compounds of general formula (I)

| | | DUX4 Count % inhibition | |
|---|---|---|---|
| | Over 50%, below 80% | Over 80%, below 95% | Over 95% |
| Compound number in Table 1 | 5, 22, 25, 26, 28, 45, 47 | 1, 3, 4, 12, 13, 16, 17, 18, 19, 27, 29, 32, 42, 44 | 2, 6, 7, 8, 9, 10, 11, 15, 20, 21, 23, 24, 30, 33, 37, 38, 39, 40, 41, 43, 46 |

TABLE 2.3

biological data for selected compounds of general formula (I)
DUX4 Count % inhibition

| | | | |
|---|---|---|---|
| | Over 50%, below 80% | Over 80%, below 95% | Over 95% |
| Compound number in Table 1 | 22, 25, 47, 51, 68, 97, 102, 105-RR, 134, 149, 152-RR, 158, 166, 170-S, 178 | 1, 2, 3, 4, 5, 9, 12, 13, 17, 18, 26, 27, 28, 29, 32, 38, 39, 42, 44, 45, 56, 57, 59, 64, 35-RR, 66, 69, 70-R, 70-S, 71-RR, 76, 82-SS, 86-RR, 88, 83-RR, 94-RR, 99-RR, 103, 108-R, 110, 113, 120, 126, 127-RR, 130-RR, 133-R, 136, 138, 141, 145-S, 146, 147, 148, 153, 154-SR, 160, 163, 164, 165, 157-S, 168, 170-R, 172-R, 176-R, 189-RR, 190-RR, 191-RR, 198-R, 201-R, 202-R | 6, 7, 8, 10, 11, 15, 16, 19, 20, 21, 23, 24, 30, 33, 35, 37, 40, 41, 43, 46, 48, 49, 50, 52, 53-RS, 54, 55, 58-RS, 60-R, 61, 62, 63, 65, 36-SS, 36-RR, 60-S, 67, 72-RR, 73-RR, 74, 75, 77-RS, 78-RS, 79-RS, 80-RS, 81-RS, 83-SS, 84-SS, 85, 87-RR, 82-RR, 89-RR, 90-RR, 91, 92, 93-RR, 95, 56-RR, 96-RR, 100, 56-SS, 101, 104-RR, 104-SS, 21-RR, 21-SS, 107, 108-S, 109, 111, 112, 114-RR, 115-RR, 116-RR, 117-RR, 118-RR, 119-RR, 121-RR, 122-RR, 123-RR, 124-RR, 125, 128-RR, 129-RR, 131-RS, 132-RR, 133-S, 135-RS, 137, 135-SS, 139, 140, 142-RR, 143-RR, 145-R, 150, 151, 155, 156, 157-R, 159, 161, 162, 167, 169-R, 169-S, 171-RR, 172-S, 173, 174-S, 174-R, 175-R, 177-R, 179, 180-R, 181-R, 182-RS, 183-R, 184-R, 185-RR, 186-R, 187-R, 192-R, 193-R, 184-S, 194, 195, 196-R, 197-R, 199, 200-R, 203-S |

1. Compound of general formula (I-cyc) or (I): 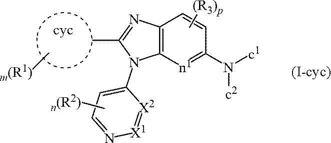 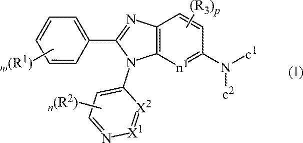
wherein
cyc is a phenyl ring, a 5-membered heteroaryl ring, or a 6-membered heteroaryl ring;
R¹ is H, halogen, nitrile, —C₁₋₄alkyl, —C₁₋₃alkyl-nitrile, —C₁₋₄haloalkyl, —C₁₋₃haloalkyl-nitrile, —O—C₁₋₄alkyl, —O—C₁₋₃alkyl-nitrile, —O—C₁₋₄haloalkyl, —O—C₁₋₃haloalkyl-nitrile, —S—C₁₋₄alkyl, —S—C₁₋ ₃alkyl-nitrile, —S—C₁₋₄haloalkyl, or —S—C₁₋₃haloalkyl-nitrile;
m is 0, 1,2, or 3;
n¹ is N, CH, or C(CH₃);
R² is H, halogen, nitrile, —C₁₋₄alkyl, —C₁₋₃alkyl-nitrile, —C₁₋₄haloalkyl, —C₁₋₃haloalkyl-nitrile, —O—C₁₋₄alkyl, —O—C₁₋₃alkyl-nitrile, —O—C₁₋₄haloalkyl, —O—C₁₋₃haloalkyl-nitrile, —S—C₁₋₄alkyl, —S—C₁₋ ₃alkyl-nitrile, —S—C₁₋₄haloalkyl, —S—C₁₋₃haloalkyl-nitrile, or R² together with Q forms a bridging moiety;
n is 0, 1, or 2;
R³ is halogen or C₁₋₄alkyl;
p is 0, 1, or 2;
X¹ is CH, C(R²), N, or C(Q);
X² is CH, C(R²), or N;
Q is H, halogen, C₁₋₆alkyl, —OH, —O—C₁₋₆alkyl, —O—C₁₋₆acyl, —NH₂, —NH—(C₁₋₆alkyl), —N(C₁₋ ₆alkyl)₂, —NH(C₁₋₈acyl), —N(C₁₋₈acyl)₂, —C₁₋₄alkyl—OH, —C₁₋₄alkyl—O—C₁₋₆alkyl, —C₁₋₄alkyl—O—C₁₋ ₆acyl, —C₁₋₄alkyl—NH₂, —C₁₋₄alkyl—NH—(C₁₋₆alkyl), —C₁₋₄alkyl—N(C₁₋₆alkyl)₂, —C₁₋₄alkyl—NH(C₁₋ ₈acyl),—C₁₋₄alkyl—N(C₁₋₈acyl)₂, —C₁₋₄alkyl—N—C(O)—NH—C₁₋₆alkyl, —C₁₋₄alkyl—N—C(O)—N(C₁₋ ₆alkyl)₂, —C₁₋₄alkyl—O—C(O)—NH—C₁₋₆alkyl, —C₁₋₄alkyl—O—C(O)—N(C₁₋₆alkyl)₂, —C₁₋₄alkyl—N—C(O)—O—C₁₋₆alkyl, or Q together with R² forms a bridging moiety selected from —NH—CH═CH—, —NH—(C₂₋₄alkyl)—, and —(C₁₋₃alkyl)—NH—(C₁₋₃alkyl)—;
c¹ is H and c² is C₄₋₈cycloalkyl, C₄₋₈heterocycloalkyl, C₄₋₈cycloalkyl-C₁₋₃alkyl, C₄₋ ₈heterocycloalkyl-C₁₋₃alkyl, C₁₋₃alkyl-C₄₋₈cycloalkyl, or C₁₋₃alkyl-C₄₋₈heterocycloalkyl, or c¹ and c² together form cyclic structure A;
A is a C₅₋₁₂cycloalkyl that can be cyclic, bicyclic, and tricyclic, and which is optionally unsaturated, and which is optionally substituted with halogen, C₁₋₄alkyl, C₂₋₄acyl, C₃₋₆cycloalkyl, C₃₋₆heterocycloalkyl, —O—C₁₋₄alkyl, —SO₂-C₁₋₄alkyl, hydroxyl, —C(═O)—NH₂, —C(═O)—NH(CH₃), —C(═O)—N(CH₃)₂, —NH₂, —NH(C₁₋₄alkyl), or —N(C₁₋₄alkyl)₂;
wherein each instance of acyl, alkyl, cycloalkyl, or heterocycloalkyl individually is optionally unsaturated, and optionally substituted with halogen, oxy, hydroxyl, methyl, ethyl, propyl, methoxy, ethoxy, or trifluoromethyl, or optionally interrupted by one or more heteroatoms;
or a salt thereof.

2. Compound according to claim 1, wherein
R¹ is H, fluorine, chlorine, —CH₃, —CF₃, —O—CH₃, or nitrile;
m is 0 or 1;
n¹ is N or CH;
R² is H, fluorine, chlorine, or forms a bridging moiety;
n is 0;
R³ is —CH₃;
p is 0 or 1;
X¹ is C(Q);
X² is CH;
Q is H, F, —CH₃, —CH₂F, —CHF₂, —CF₃, —OCH₃, —OCH₂F, —OCHF₂, —OCF₃, —NH—C(O)—CH₃, —NH—C(O)—cyclopropyl, —NH—C(O)—phenyl, —NH—C(O)—halophenyl, —NH—C(O)—piperidinyl, —NH—C(O)—pyridinyl, —NH—C(O)—morpholinyl, —NH—C(O)—oxanyl, —NH₂, —NH(CH₃), -NH(cyclopentyl), —CH₂—NH—C(O)—CH₃, —CH₂—N(CH₃)₂, —CH₂—NH₂, —CH₂—NH—(CH₃), —CH₂—NH—(cyclopentyl), or together with R² forms —NH—CH═CH—; and/or wherein
c¹ is H and c² is pyridyl, —CH₂—pyridyl, piperidinyl, N-methylpiperidinyl, —CH₂—piperidinyl, —CH₂—(N—methylpiperidinyl), cyclopentyl, hydroxycyclopentyl, —CH₂—cyclopentyl, —CH₂—hydroxycyclopentyl, pyrrolidinyl, N-methylpyrrolidinyl, —CH₂—pyrrolidinyl, —CH₂—(N—methylpyrrolidinyl), or c¹ and c² together form cyclic structure A.

3. Compound according to claim 1, wherein
R¹ is H, fluorine, or chlorine;
R² is H or forms a bridging moiety;
p is 0; and/or wherein
Q is H, —CH₃, —CHF₂, —OCH₃, —NH—C(O)—CH₃, —NH—C(O)—cyclopropyl, —NH—C(O)—phenyl, —NH—C(O)—halophenyl, —NH—C(O)—piperidinyl, —NH—C(O)—pyridinyl, —NH—C(O)—morpholinyl, —NH—C(O)—oxanyl, —NH₂, —CH₂—NH—(CH₃), or together with R² forms —NH—CH═CH—.

4. Compound according to claim 1, wherein A is optionally substituted and optionally unsaturated azetidinyl, pyrrolidinyl, imidazolidinyl, oxazolidinyl, piperidinyl, piperazinyl, morpholinyl, azacycloheptyl, diazacycloheptyl, or oxoazacycloheptyl;
wherein each optional substitution can be a substitution with halogen, C₁₋₆alkyl, C₃₋₆cycloalkyl, C₃₋₆heterocycloalkyl, —O—C₁₋₄alkyl, hydroxyl, —NH₂, —NH(C₁₋₄alkyl), or —N(C₁₋₄alkyl)₂; preferably each optional substitution is independently selected from methyl, dimethylamine, methoxyl, propyl, hydroxyl, a bridging C₁₋₃alkyl moiety, spiro azetidinyl, spiro N-methylazetidinyl, spiro oxetanyl, oxetanyl, spiro piperidinyl, difluoropiperidinyl, spiro N-methylpiperidinyl, spiro cyclopropyl, fused pyrrolidinyl, or fused N-methylpyrrolidinyl.

5. Compound according to claim 1, wherein it is of general formula (I-A): 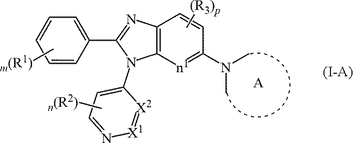
.

6. Compound according to claim 1, wherein it is of general formula (II) or (II-A): 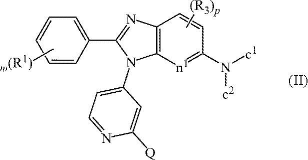 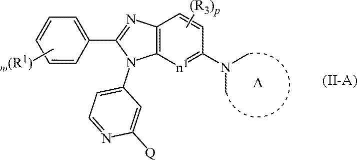
.

7. Compound according to claim 1, wherein it is of general formula (III) or (III-A): 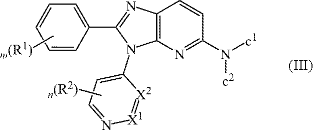 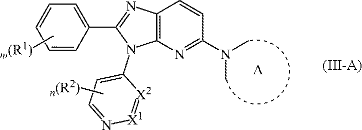
.

8. Compound according to claim 1, wherein A is bicyclic, spiro-cyclic, or bridged, preferably selected from A3-A9, A12, A13, A15-A19, A22, A25-A35, and A37-A42; more preferably it is bicyclic or bridged, even more preferably selected from A3-A6, A9, A25-A31, A33, and A41.

9. Compound according to claim 1, wherein m is 1 and wherein R¹ is ortho, meta, or para to the bicyclic core of the compound.

10. Compound of general formula (1) wherein the compound is selected from compounds 1-203 as listed in table 1.

11. Compound of general formula (1) wherein the compound is selected from compounds 5, 22, 25, 26, 28, 45, 47, 1, 3, 4, 12, 13, 16, 17, 18, 19, 27, 29, 32, 42, 44, 2, 6, 7, 8, 9, 10, 11, 15, 20, 21, 23, 24, 30, 33, 37, 38, 39, 40, 41, 43, and 46 as listed in table 1.

12-14. (canceled)

15. A method for reducing DUX4 expression in a subject in need thereof, the method comprising the step of administering an effective amount of a compound of general formula (I) as defined in claim 1.

16. The method of claim 15, wherein the method is for the treatment of a disease or condition associated with DUX4 expression, and wherein the compound of general formula (I) reduces DUX4 expression.

17. The method of claim 16, wherein the disease or condition associated with DUX4 expression is a muscular dystrophy or cancer.

18. The method of claim 15, wherein the disease or condition associated with DUX4 expression is a muscular dystrophy.

19. The method of claim 15, wherein the disease or condition associated with DUX4 expression is facioscapulohumeral muscular dystrophy (FSHD).
